(19) United States
(12) Patent Application Publication
(10) Pub. No.: US 2012/0196761 A1
(43) Pub. Date: Aug. 2, 2012

(54) DIAGNOSIS OF IN SITU AND INVASIVE BREAST CANCER

(75) Inventors: XIAO-JUN MA, San Diego, CA (US); Mark Erlander, Carlsbad, CA (US); Sgroi Dennis, Winchester, MA (US)

(73) Assignees: GENERAL HOSPITAL CORPORATION, Boston, MA (US); BIOTHERANOSTICS, INC., San Diego, CA (US)

(21) Appl. No.: 13/196,818

(22) Filed: Aug. 2, 2011

Related U.S. Application Data
(63) Continuation of application No. PCT/US2010/022929, filed on Feb. 2, 2010.
(60) Provisional application No. 61/149,012, filed on Feb. 2, 2009.

(51) Int. Cl. C40B 30/04; C12Q 1/68

(52) U.S. Cl. 506/9; 435/6.12

(57) ABSTRACT

The disclosure includes the use of gene expression profiles, or patterns, with clinical relevance to breast cancer. In particular, the disclosure provides the identities of genes that are expressed in correlation with the presence of breast cancer, the grade of breast cancer, and the type of breast cancer. The disclosed methods assist in the detection and identification of breast cancer in a patient and so helps determine treatment and clinical outcome, and so prognosis, for the patient. The gene expression levels, whether embodied in nucleic acid expression, protein expression, or other expression formats, may be used detect the presence of in situ or invasive breast cancer.

### RELATED APPLICATIONS

This application claims benefit of priority from U.S. Provisional Patent Application 61/149,012, filed Feb. 2, 2009, and from Patent Cooperation Treaty Application PCT/US10/22929, filed Feb. 2, 2010, which are hereby incorporated by reference as if fully set forth herein.

### FIELD OF THE DISCLOSURE

The disclosure relates to the use of gene expression profiles, or patterns, with clinical relevance to breast cancer. In particular, the disclosure is based in part on the identities of genes that are expressed at higher, or lower, levels in correlation with the presence of breast cancer, the grade of breast cancer, and the type of breast cancer. The levels of gene expression form a molecular index that assists in the detection and identification of breast cancer in a patient and so help determine treatment and clinical outcome, and so prognosis, for the patient. The gene expression levels, whether embodied in nucleic acid expression, protein expression, or other expression formats, may be used detect the presence of in situ or invasive breast cancer.

The gene expression levels may also be used in the study and/or diagnosis of cancer cells and tissue as well as for the study of a subject's prognosis. When used for diagnosis or prognosis, the profiles are used to determine the treatment of cancer based upon the type of cancer present, or likely to occur, in a patient.

### BACKGROUND OF THE DISCLOSURE

While the role of tumor microenvironment in breast cancer has been of interest and study, the critical molecular changes in the tumor stroma accompanying cancer progression remains unclear. The tumor microenvironment or the stroma “hosting” the malignant breast epithelial cells is comprised of multiple cell types, including fibroblasts, myoepithelial cells, endothelial cells and various immune cells (Bissell et al., Nab. Rev. Cancer 1(1):46-54 (2001); de Visser et al. Contrib. Microbiol. 3:118-137 (2006); Liotta et al. Nature, 411(6835):375-379 (2001); Egeblad et al. Cold Spring Harbor Symp. Quant. Biol., 70:383-388 (2005)).

One prevailing view is that the tumor-associated stroma is “activated” by the malignant epithelial cells to foster tumor growth, for example, by secreting growth factors, increasing angiogenesis, and facilitating cell migration, ultimately resulting in metastasis to remote organ sites (Liotta, supra.). For example, two chemokines (CXCL12 and CXCL14), which bind to tumor epithelial cells to promote proliferation, migration and invasion, have been reported to be over-expressed by the activated tumor fibroblasts and myoepithelial cells (Muller et al., Nature, 410(6824):50-56 (2001); Orimo et al., Cell, 121(3):335-348 (2005); and Allinen et al., Cancer Cell, 6(1):17-32 (2004)).

Using the serial analysis of gene expression (SAGE) technique, Allinen et al. performed the first systematic profiling of the various stromal cell types isolated via cell type-specific cell surface markers and magnetic beads (Allienen id.) Using serial analysis of gene expression (SAGE) coupled with antibody-based ex vivo tissue fractionation, Allinen id reported a limited set of 417 cell type-specific genes among the most prominent cell types in breast cancer (epithelial, myoepithelial, and endothelial cells, fibroblasts, and leukocytes). They demonstrated gene expression alterations in all cell types within the tumor microenvironment accompanying progression from normal breast tissue to ductal carcinoma in situ (DCIS) to invasive ducal carcinoma (IDC) (Allred et al., Endocr. Relat. Cancer, 8(1):47-61 (2001)), consistent with the possibility that these cell types participate in tumorigenesis. Finak et al., (Finak et al., Breast Cancer Res., 8(5):R58 (2006)) reported gene expression profiles of both epithelial and stromal compartments from the same tumor biopsy via laser capture microdissection (LCM). However, these workers only analyzed the morphologically “normal” epithelium and “normal” stroma, leaving the gene expression changes in the tumor-activated stroma unexplored.

Using LCM, previous gene expression analysis of the epithelial compartment of malignant lesions during breast cancer progression revealed that most of the gene expression changes take place prior to local invasion (even in atypical ductal hyperplasia (ADH)) and there are no major changes in gene expression accompanying the in situ to invasive growth transition (Ma et al., Proc. Natl. Acad. Sci. USA, 100(10):5974-5979 (2003)).

The citation of documents herein is not to be construed as reflecting an admission that any is relevant prior art. Moreover, their citation is not an indication of a search for relevant disclosures. All statements regarding the dates or contents of the documents is based on available information and is not an admission as to their accuracy or correctness.

### BRIEF SUMMARY OF THE DISCLOSURE

The instant disclosure advances the field based in part on a comparative analysis of global gene expression changes in the stromal and epithelial compartments during breast cancer progression from normal to pre-invasive to invasive carcinoma, including both ductal and lobular forms. Thus, the disclosure is relevant to ductal carcinoma in situ (DCIS), invasive (or infiltrating) ductal carcinoma (IDC), lobular carcinoma in situ (LCIS), and invasive (or infiltrating) lobular carcinoma (ILC) The extension of analysis to the tumor stroma microenvironment demonstrates that, like the tumor epithelium, the tumor stroma microenvironment undergoes extensive gene expression alterations even at the pre-invasive stage of DCIS. This supports the view that cell-cell communication via paracrine mechanisms between the two compartments is involved in tumor progression in both ductal and lobular carcinomas.

The disclosure relates to compositions and methods for the use of cells from the stromal and epithelial compartments of breast tissue to provide diagnostic, prognostic, and predictive information regarding ductal or lobular breast cancer. In many embodiments, the disclosure relates to ductal carcinoma. The compositions of the disclosure include nucleic acid molecules, such as probes and primers, for the detection of known genes as well as the detection of the expression levels of those genes. These compositions are used in the methods of the disclosure, including those for the detection of breast cancer, detection of disease progression, and determination of prognosis for a subject with breast cancer. Additionally, the disclosure includes methods for the treatment or palliative care of a subject with breast cancer.

In a first aspect, the disclosure includes compositions and methods for detecting the presence or occurrence of breast cancer in a subject by analysis of gene expression in an epithelial or stromal cell from the subject. In many cases, the breast cancer is ductal carcinoma. In other cases, the breast cancer is lobular carcinoma. The disclosure thus includes the identities of individual genes and their expression levels in an epithelial or stromal cell from the breast of a subject with breast cancer relative to a normal epithelial or stromal cell of the breast. The disclosure is not based upon the first identification of any gene but rather based upon the identification of increased or decreased gene expression in an epithelial or stromal cell from the breast of a subject with breast cancer relative to a normal epithelial or stromal cell.

So one method of the disclosure includes detecting, in an epithelial cell from a subject, an increased or decreased expression level of one or more genes relative to a normal epithelial cell where the increased or decreased expression has been identified as indicative of breast cancer as disclosed herein. In some embodiments, the method is used to identify the presence or occurrence of breast cancer in a subject based upon the expression levels detected in an epithelial cell from the subject. And because the disclosure includes the identities of genes with expression levels that discriminate between subjects with and without carcinoma in situ (DCIS) and/or invasive/infiltrating carcinoma (IDC) as well as gene expression levels that discriminate the presence or occurrence of one but not the other, the disclosure includes methods to identify DCIS and/or IDC (or LCIS and/or ILC) based upon gene expression levels in an epithelial cell from a subject.

The disclosure also includes a method of detecting, in a stromal cell from a subject, an increased or decreased expression level of one or more genes relative to a normal stromal cell where the increased or decreased expression has been identified as indicative of breast cancer as disclosed herein. In some embodiments, the method is used to identify the presence or occurrence of breast cancer in a subject based upon the expression level(s) detected in a stromal cell from the subject. And like the methods based on an epithelial cell, the disclosure includes methods to identify DCIS and/or IDC (or LCIS and/or ILC) based upon gene expression levels of disclosed genes in a stromal cell from a subject.

The increase or decrease in expression of a disclosed gene, relative to a normal cell, is of at least a factor of 0.05 on a log₂ scale.

And while the method may be practiced with only the use of an epithelial cell or a stromal cell, embodiments of the disclosure include methods using both cell types with detection of the gene expression level of at least one of the disclosed genes in each cell type.

In a related aspect, the disclosure includes a method of detecting the progress of breast cancer treatment in a subject based upon expression levels of genes in a stromal cell. The method may include detecting, in a stromal cell from a subject undergoing a breast cancer treatment, an increased or decreased expression level of one or more genes relative to a normal stromal cell where the increased or decreased expression has been identified as indicative of breast cancer as disclosed herein. The method may be performed over time, such that a reversal of observed expression level(s) that are indicative of breast cancer, indicates that the treatment has been effective in part or in whole. In contrast, the continuation of observed expression level(s) that are indicative of breast cancer indicates that the treatment has been at least in part ineffective.

Similarly, the disclosure includes a method of detecting the progress of breast cancer treatment in a subject based upon expression levels of genes in an epithelial cell. The method may include detecting, in an epithelial cell from a subject undergoing a breast cancer treatment, an increased or decreased expression level of one or more genes relative to a normal epithelial cell where the increased or decreased expression has been identified as indicative of breast cancer as disclosed herein. The method may be performed over time, such that a reversal of observed expression level(s) that are indicative of breast cancer, indicates that the treatment has been effective in part or in whole. In contrast, the continuation of observed expression level(s) that are indicative of breast cancer indicates that the treatment has been at least in part ineffective.

In another aspect, the disclosure includes a method of identifying the type of ductal (or lobular) carcinoma and/or breast cancer grade in a subject. The method may include detecting, in a stromal cell from a subject with breast cancer, an increase or decreased expression of one or more genes disclosed herein, where the expression level discriminates between the presence and absence of DCIS or IDC (or LCIS or ILC) in a subject and/or discriminates between grades of DCIS and IDC (or LCIS and ILC). The disclosure thus includes a method of distinguishing in situ breast cancer from invasive breast cancer in a subject based upon assessment of expression levels of genes disclosed herein. The disclosure also includes a method of grading in situ and/or invasive breast cancer based upon the expression levels of genes disclosed herein.

In an additional aspect, the disclosure includes a method for determining the likelihood of breast cancer recurrence in a subject treated for breast cancer. The method may include detecting, in an epithelial or stromal cell from the treated subject, an increase or decreased expression of one or more genes, relative to expression in a normal epithelial or stromal cell, as disclosed herein. In many embodiments, a stromal cell is used, and detection is of expression levels for stroma cells in tumor-associated stroma as described herein.

In the methods of the disclosure, the identification of gene expression levels as relevant to in stromal and epithelial cells in breast cancer is independent of the form of the assay or detection means used to determine the actual level of expression. An assay or detection means may utilize any identifying feature of a disclosed gene as long as the assay reflects, quantitatively or qualitatively, expression of the gene. Identifying features include, but are not limited to, unique nucleic acid sequences used to encode (DNA), or express (RNA), of a disclosed gene or epitopes specific to, or activities of, a polypeptide encoded by the gene. Additionally, all or part of a consensus sequence that may be readily identified by comparison of available sequences for a single gene may be used for the detection of nucleic acid expression. All that is required is the identity of the gene(s) necessary to discriminate between two or more possibilities in a stromal or epithelial cell and an appropriate sample for use in an expression assay.

Thus, the disclosure includes the preparation of RNA from a cell for use in detecting gene expression levels. In some embodiments, the RNA is amplified before use in detection, such as by conversion to a cDNA before linear amplification or exponential amplification, such as by polymerase chain reaction (PCR). In further embodiments, the RNA or cDNA may be detected via the use of reverse transcription-PCR and/or quantitative real time-PCR (RT-PCR). In other embodiments, the RNA or cDNA may be used detected via use of an array, such as a microarray, capable of detecting one or more disclosed gene.

In methods with the use of cells, the cell may be isolated from a cell containing sample prior to use. In some embodiments, the sample may be tissue removed from a subject. Non-limiting examples include biopsied material, including a needle biopsy; or a sample obtained by less invasive means, such as a needle aspirate or ductal lavage. The sample may be fresh, frozen, or fixed, such as the case of a formalin fixed paraffin embedded (FFPE) sample as a non-limiting example.

In another aspect, the disclosure includes a method of detecting the presence or occurrence of breast cancer in a subject by analysis of a biological fluid from said subject. The method may include detecting, in a biological fluid from a subject, an increase or decreased expression of a polypeptide encoded by a gene disclosed herein as expressed at levels that can discriminate between the presence and absence of breast cancer. In some embodiments, the polypeptide is an extracellular matrix constituent, a matrix metalloprotease, or a chemokine encoded by a disclosed gene. Non-limiting examples include MMP2, MMP11, MMP14, inhibin, and Gremlin1.

In a further aspect, the disclosure includes a method to determine therapeutic treatment for a cancer patient. The method may include first identifying the presence or occurrence of breast cancer in a subject as disclosed herein and then selecting treatment for a patient with the type of breast cancer identified.

While the present disclosure has been described mainly in the context of human breast cancer and human patients, it may be practiced in the context of breast cancer of any animal known to be potentially afflicted by breast cancer. Non-limiting examples of animals include mammals, particularly those important to agricultural applications (such as, but not limited to, cattle, sheep, horses, and other “farm animals”) and for human companionship (such as, but not limited to, dogs and cats).

### BRIEF DESCRIPTION OF THE FIGURES

FIG. 1 illustrates the LCM experimental design. An example of the tumor microenvironment compartments targeted by LCM are shown: the epithelial (white asterisk) and stromal (black outlined areas with black asterisk) compartments of the normal terminal ductal lobular unit, of ductal carcinoma in situ (DCIS) and invasive ductal carcinoma (IDC).

FIG. 2 illustrates a comparative analysis of gene expression changes in tumor and stroma. Up arrows indicate up-regulated genes, and down arrows indicate down-regulated genes.

FIG. 3A is a heatmap of 849 genes with >3-fold differential expression in either DCIS vs. N or IDC vs. N in the epithelium. FIG. 3B shows a heatmap of 557 genes with >3-fold differential expression in either DCIS-S vs. N-S or IDC-S vs. N-S. Data shown were log₂ (fold change) relative to the average expression in normal controls (N or N-S). In each heatmap, genes (rows) are hierarchically clustered using 1-Pearson correlation as distance metric.

FIG. 4 is a heatmap of differential expression of ribosomal protein genes in the malignant epithelium and tumor stroma. Data shown were log₂ (fold change) relative to the average expression level in the normal controls (N or N-S). The expression measurements for multiple probesets representing the same gene were collapsed to the single representative probeset with the largest differential gene expression. All genes shown were significant at adjusted p<0.05. The horizontal line denoted with the asterisk indicates the division between genes with increased expression (above the line) and genes with decreased expression (below the line).

FIG. 5 is a heatmap of gene expression signature correlated with tumor grade in the stroma. Comparison of grade III tumors with grade I tumors identified 526 up-regulated and 94 down-regulated genes in grade III-stroma. Data shown were log₂ (fold change) relative to the median expression level across all samples. Genes in rows were hierarchically clustered and samples in columns were arranged by sample type.

FIG. 6 illustrates the validation of selected genes. Parts A-D are boxplots of relative gene expression by QRT-PCR. Y-axis, cycling threshold (Ct) values relative to the median value for the entire series. Asterisks denote statistically significant differences by Wilcoxon rank sum test (*, p<0.05; **, p<0.01; ***, p<0.001, ****, p<0.0001). In Parts A and B, the reference groups were the normal components (N for epithelium and N-S for stroma); in Parts C and D, the reference groups were grade I (E-I for epithelium and S-I for stroma). Part E shows immunostaining of an estrogen receptor-positive breast cancer. Arrows point to positive staining in stromal fibroblasts.

FIG. 7 contains Table 8, listing differentially expressed genes in the epithelium in 3 comparisons: DCIS vs. N, INV vs N, DCIS+INV vs N.

FIG. 8 contains Table 9, listing differentially expressed genes in the stroma in 3 comparisons: DCIS vs. N-S, INV vs N-S, DCIS+INV vs N-S.

FIG. 9 contains Table 10, listing genes associated with the in situ (DCIS) to invasive (INV) transition as observed in the stroma. Fold change is INV vs DCIS.

FIG. 10 contains Table 11, listing grade-associated genes in the stroma. Fold change is G3 (grade III) vs G1 (grade I) genes.

### ABBREVIATIONS USED IN THE DISCLOSURE

N, normal breast epithelium; ADH, atypical ductal hyperplasia; DCIS, ductal carcinoma in situ; IDC, invasive ductal carcinoma; LCM, laser capture microdissection; N-S, normal stromal compartment: DCIS-S, DCIS-associated stroma; IDC-S, IDC-associated stroma; WIF1, WNT inhibitory factor 1; SFRP1, secreted frizzled-related protein 1; GREM1, gremlin 1; INHBA, inhibin beta A; MMP, matrix metalloproteinase; CXCL, chemokine (C-X-C motif) ligand; ER, estrogen receptor; PR, progesterone receptor; pos, positive; neg, negative; ND, not determined; N/A, not available; NES, normalized enrichment score; FDR, false discovery rate.

### DETAILED DESCRIPTION OF MODES OF PRACTICING THE DISCLOSURE

### General

The disclosure is based on the first comprehensive comparative analysis of in vivo gene expression changes in the tumor epithelium and its stromal microenvironment during breast cancer progression from normal to DCIS to IDC. So the disclosure provides the first comparative analysis of the in situ gene expression profiles of patient-matched normal and neoplastic breast epithelial and stromal compartments of both pre-invasive and invasive stages of human breast cancer progression. Without being bound by theory, and offered to improve the understanding of the disclosure, the disclosed results of the breast cancer microenvironment at the transcriptome level, and previous studies at the genomic (Fukino et al., Cancer Res., 64(20):7231-7236 (2004); Patocs et al., N. Engl. J. Med., 357(25):2543-2551 (2007)) and epigenetic (Fiegl et al., Cancer Res., 66(1):29-33 (2006); Hu et al., Nat. Genet, 37(8):899-905 (2005)), levels support the view that tumor microenvironment is an important co-conspirator rather than a passive bystander during tumorigenesis. Molecular alterations within the stroma offer novel avenues for disease prognosis (Final(et al., Nat. Med., 14(5):518-527 (2008)). The disclosed gene expression datasets of carefully procured in situ tumor epithelium and stroma should be a valuable addition to the resources for breast cancer diagnosis, treatment, and research.

### Cells and Samples

The discovery of the disclosed data of expression levels of genes in stromal and epithelial cells is based in part on laser capture microdissection and gene expression microarrays to analyze 14 patient-matched normal epithelium, normal stroma, tumor epithelium and tumor-associated stroma as described herein. Differential gene expression and gene ontology analyses were also performed.

The compositions of the disclosure include cells from the stromal and epithelial compartments of breast tissue. The cells may be prepared as highly enriched populations of normal or malignant epithelial cells, normal stromal cells, or tumor-associated stromal cells, and used as disclosed herein. In some embodiments, the cells are isolated from a larger cell containing breast tissue sample or breast ductal tissue sample from a subject. In some cases, the sample includes breast tissue isolated from an individual suspected of being afflicted with, or at risk of developing, breast cancer. Such a sample is a primary isolate (in contrast to cultured cells) and may be collected by an invasive or non-invasive means. Non-limiting examples include a surgical biopsy, needle biopsy, ductal lavage, needle aspiration, fine needle aspiration. a sample prepared by the devices and methods described in U.S. Pat. No. 6,328,709, or any other suitable means recognized by the skilled person.

In some embodiments, the expression level of one or more disclosed genes is detected or determined in a stromal or epithelial cell. The disclosure thus includes the use of known techniques for the isolation of a stromal or epithelial cell from a breast tissue sample. As known to the skilled person, the stroma contains multiple cell types, including fibroblasts, myoepithelial cells, endothelial cells and various immune cells. Non-limiting examples of methods to isolate stromal, or epithelial, cells include microdissection, such as laser capture microdissection (LCM) using a PixCell® IIe system (Molecular Devices, Mountain View, Calif.) as previously described (Ma, supra.). The isolation or capture of a stromal or epithelial cell may be performed with use of an appropriate staining technique to identify the cells to be isolated or captured. Non-limiting examples include the use of hematoxylin and eosin (H&E) stained or immunohistochemically stained sections of cell containing breast tissue from a subject. The sections may of course be of an appropriate thickness for microdissection as known to the skilled person.

In some embodiments, a microdissected normal stromal compartment (N-S) is prepared and used. The cells may be those of the intralobular, rather than the extralobular, stromal compartment of normal breast tissue. In some cases, the cells are at least 0.3 cm or more from any lesion that appears pre-malignant or malignant. Of course in cases of no lesions, then a skilled person may use appropriate discretion in selecting a stomal cell for isolation or capture. In other cases, the cells are at least 0.4, at least 0.5, at least 0.6, at least 0.7, at least 0.8, at least 0.9, or at least 1.0 cm from any lesion that appears pre-malignant or malignant.

In other embodiments, a cell containing sample may contain lesions suspected of being DCIS or IDC but the exact nature of the lesion is uncertain or is deemed to require confirmation or clarification. In such cases, cells from the lesion-associated stroma may be isolated or captured for use in a method of the disclosure. Non-limiting examples include stromal cells isolated or captured from a rim of about 25 microns or less surrounding the lesion, and stromal cells from within the lesion. As additional examples, the stromal cells may be from a rim of about 50 microns or less, about 100 microns or less, about 150 microns or less, about 250 microns or less, about 350 microns or less, about 450 microns or less, about 550 microns or less, about 650 microns or less, about 750 microns or less, about 850 microns or less, about 950 microns or less, or about 1 mm or less surrounding the lesion.

The isolated cells may be used for the detection of gene expression by any appropriate means known to the skilled person. In some embodiments, the detection is by measuring nucleic acid expression, such as the expression of mRNA molecule(s) from one or more of the disclosed genes. In other embodiments, the detection may be by measuring the expression of polypeptide molecule(s) encoded by one or more of the disclosed genes. In further embodiments where measuring a decrease in gene expression is used, the detection may be for the down-regulation of expression from one or more disclosed genes at the DNA or genomic level, such as, but not limited to, detection of gene methylation.

Exemplary genes for use in such a method of the disclosure include those in Tables 4-6 and 11. Non-limiting examples include INHBA (inhibin, beta A), GREM1 (gremlin1, cysteine knot superfamily, homolog), NOX4 (NADPH oxidase 4), and WIF1 (WNT inhibitory factor 1). In other embodiments, a method of the disclosure includes detection of GREM 1 (or WIF1) expression only in combination with one or more other disclosed gene. In yet additional embodiments, a method of the disclosure includes detection of one or more gene disclosed herein with the exception of GREM1 and WIF1. In many embodiments including use of a stromal cell, detection of increased expression by a mitochondrial ribosomal protein encoding gene is used, optionally in combination with a cytoplasmic ribosomal protein gene that is decreased in expression. In some cases, the combination may be used to calculate a ratio of mitochondrial ribosomal protein expression to cytoplasmic ribosomal protein expression and the ratio is used, optionally without the need for comparison of the expression levels to a control gene.

In other embodiments, a sample of a biological fluid from a subject suspected of being afflicted with, or at risk of developing, breast cancer may be used. In these embodiments, the detection of gene expression may be based upon the level of a polypeptide, or fragment thereof, encoded by a disclosed gene and present in the fluid. Of course the detection of more than one polypeptide, or more than one fragment, encoded by more than one disclosed gene may be used in the practice of the disclosure. In some cases, the presence of the polypeptide or polypeptide fragment in the fluid may be due to secretion from a stromal or epithelial cell. The identification of secreted products by the disclosed genes may be readily made by a review of the knowledge regarding the disclosed genes or by a routine assay for presence of a gene encoded polypeptide in the fluid from a subject. Non-limiting examples of a biological fluid from a subject include blood, serum, plasma, and urine.

Exemplary gene encoded polypeptides to be detected in such a method of the disclosure include those that are an extracellular matrix constituent, a matrix metalloprotease, or a chemokine encoded by a disclosed gene. Non-limiting examples include a collagen polypeptide encoded by COL10A1 (collagen, type X, alpha 1), COL11A1 (collagen, type XI, alpha 1), COL10A1 | collagen, type X, alpha 1 (Schmid metaphyseal chondrodysplasia), COL8A1 (collagen, type VIII, alpha 1), COL11A1 (collagen, type XI, alpha 1), COL12A1 (collagen, type XII, alpha 1), or CTHRC1 (collagen triple helix repeat containing 1); a fibronectin polypeptide encoded by FNDC1 (fibronectin type III domain containing 1) or FN1 (fibronectin 1); a polypeptide encoded by CILP (cartilage intermediate layer protein, nucleotide pyrophosphohydrolase); a metalloprotease encoded by MMP2, MMP 11, or MMP 14; a polypeptide encoded by XCL9 (chemokine (C-X-C motif) ligand 9); a polypeptide encoded by INHBA (inhibin, beta A); and a polypeptide encoded by GREM1 (gremlin1, cysteine knot superfamily, homolog). Of course the disclosure includes detecting a combination of more than one of the above gene products in a disclosed method.

In other embodiments, a method of the disclosure includes detection of a polypeptide encoded by the GREM1 (or WIF1) gene only in combination with a polypeptide encoded by one or more other disclosed gene. In yet additional embodiments, a method of the disclosure includes detection of one or more gene encoded polypeptides disclosed herein with the exception of a polypeptide encoded by GREM1 or WIF1.

Detection of expressed polypeptides may be by any suitable means known to the skilled person. One non-limiting example is the use of an antibody, optionally a monoclonal antibody, specific for the polypeptide in the context of the biological fluid being assayed. The antibody is used to form a detectable complex with the polypeptide to be detected. In some cases, detection of the complex may be by use of a labeled antibody or by use of a labeled antibody that detects the complex. In other embodiments, a ligand specific for a polypeptide may be used to form a detectable complex. Again, the complex may be detected by use of a labeled ligand or by use of a labeled antibody that detects the complex.

### Genes and Gene Expression Datasets

The disclosed gene expression datasets are the result of an analysis of RNA expression levels in isolated, enriched cells as disclosed herein. The datasets also provide the identities of human genes, the expression levels of which have the power to discriminate between different breast cancer states in a subject as disclosed herein. The disclosed datasets include identifiers for specific sets of oligonucleotide sequences (“probesets”) used to detect expression of each disclosed gene. Each gene is also identified in the tables of the disclosure by both a publicly recognized accession number at the start of the “probeset” identifier and by a “Gene Description” or “Gene Name”. This information may be used to readily and quickly identify publicly available sequences recognized by a skilled person, and publicly identified, as being those of a disclosed gene. The skilled person is also able to readily and quickly identify other relevant information, such as the coding region of the gene, the 5′-untranslated region, the 3′-untranslated region, and consensus sequences for each gene. Each publicly available sequence for a disclosed gene may thus be considered a representative “species” of that gene, where the plurality of the “species” supports the range of sequences encompassed by each gene identified herein.

The means used in the identification of the genes may also be used to detect expression levels in methods of the disclosure. As a non-limiting example, total RNA may be isolated from captured cells by any suitable means known to the skilled person. In some embodiments, the Picopure™ RNA isolation kit (Molecular Devices) is used, with amplification by T7 RNA amplification (RiboAmp™, Molecular Devices), followed by labeling and hybridized to an array or microarray with probes able to detect one or more genes disclosed herein. In some cases, the probe may be hybridize to the 3′-end of a disclosed gene. The hybridization may be to the translated and/or untranslated region of the gene. A hybridized array or microarray is then washed, stained and scanned according to protocols known to the skilled person.

Alternatively, gene expression may be performed by analysis of messenger RNA (mRNA) encoded and expressed by the disclosed genes. In some embodiments, polyadenylated RNA is used as a template to produce a complementary cDNA molecule that is then amplified and detected. In some cases, the amplification is by use of the polymerase chain reaction (PCR), optionally quantitative or RT-PCR, by methods known to the skilled person. Methods for amplifying mRNA are generally known to the skilled person, with reverse transcription PCR (RT-PCR) as a non-limiting example. And because sequences of the disclosed genes are publicly available to the skilled person, the preparation and use of appropriate probes and primers for the detection of RNA expressed from the genes is routine and requires no more than repetitive reactions. In many cases, the disclosed datasets include identifiers for specific sets of oligonucleotide sequences (“probesets”) used to detect expression of each disclosed gene.

As a non-limiting example, real-time PCR is performed on amplified RNA (aRNA) prepared for microarray analysis as described above and in reference (Ma, supra.). Briefly, aRNA is converted to double-stranded cDNA, and the cDNA is quantitated with PicoGreen® (Molecular Probes) using a spectrofluorometer (Molecular Devices). Each gene is analyzed in triplicate in a 96-well plate using and ABI 7900HT (Applied Biosystem). For the specific genes exemplified in the examples section below, the sequences of representative PCR primer pairs and a representative fluorogenic MGB probe (5′ to 3′) are provided.

The disclosure includes Tables 4 and 8, which contain gene expression data for genes that are expressed at significantly higher and lower levels in tumor epithelium (compared to normal epithelium). Fold changes are indicated in those tables, where positive values indicate increased expression relative to normal epithelium and negative values indicated decreased expression relative to normal epithelium. Decreased expression of cytoplasmic ribosomal proteins and increased expression of mitochondrial ribosomal proteins are found in tumor epithelium.

The disclosure also includes Tables 5 and 9, which contain gene expression data for genes that are expressed at significantly higher and lower levels in tumor-associated stroma (compared to normal stroma). Fold changes are indicated in those tables, where positive values indicate increased expression relative to normal stroma and negative values indicated decreased expression relative to normal stroma. Furthermore, and within the stroma, extensive gene expression changes associated with DCIS and IDC were observed. This is consistent with tumor-adjacent stroma co-evolving (or being altered in phenotype as defined by gene expression levels) with the tumor epithelium. This appears to be the case even before tumor invasion occurs. Highly up-regulated genes in the tumor-associated stroma include constituents of the extracellular matrix and matrix metalloproteases, and cell cycle-related genes. And like tumor epithelium, decreased expression of cytoplasmic ribosomal proteins and increased expression of mitochondrial ribosomal proteins are found in tumor-associated stroma.

Therefore, and in embodiments of the disclosure where a sample of stromal and/or epithelial cells are used, detection of decreased expression of cytoplasmic ribosomal proteins, relative to normal expression levels in stroma and/or epithelium, is indicative of the presence or occurrence of breast cancer. Similarly, detection of increased expression of mitochondrial ribosomal proteins in a sample of stromal and/or epithelial cells is also indicative of the presence or occurrence of breast cancer.

Additionally, Tables 8 and 9 include gene expression datasets of DCIS and IDC as a combination in comparison to normal epithelial or stroma cells. This data may be used in a method of the disclosure to detect the presence of breast cancer without specificity for the stage of breast cancer that is present. In other embodiments, the stage specific gene datasets in Tables 8 and 9 may be used to detect the presence of stage specific breast cancer as disclosed herein.

As pointed out above, the alterations in gene expression include many components of the ECM and ECM-remodeling matrix metalloproteases. Increased mitotic gene expression occurs both in malignant epithelium and adjacent stroma. Without being bound by theory, and offered only to improve the understanding of the disclosure, this may reflect the often observed desmoplastic reaction around tumor cells. And while the general decrease in expression of cytoplasmic ribosomal proteins in stromal (and epithelial) cells of breast (ductal) tissue during cancer progression appears contrary to the expectation that increased protein synthesis is considered a hallmark of cancer, it is nevertheless a discovery of the disclosure.

The mechanism by which ribosomal proteins contribute to tumorigenesis is unknown. Without being bound by theory, and offered only to improve the understanding of the disclosure, decreased expression of ribosomal proteins in breast cancer may reflect a qualitative change in ribosomal structure that allows differential translation of gene products required for rapid tumor growth. Alternatively, it may reflect some unknown non-ribosomal functions by these proteins. In contrast to the decreased expression of cytoplasmic ribosomal protein genes, is the discovery of increased expression of a number of mitochondrial ribosomal protein genes in both the tumor epithelium and the tumor-associated stroma. The human mitochondrial ribosomes are responsible for the production of several key proteins in bioenergetics including subunits of the ATP synthase.

The top differentially expressed genes between tumor-associated stroma and normal stroma included several signaling molecules identified for the first time as important for tumorigenesis in breast cancer. Two antagonists of WNT receptor signaling, WIF1 and SFRP1, are consistently down-regulated both in the tumor epithelium and stroma. Two TGFα superfamily members (GREM1 and INHBA) are strongly induced in the tumor-associated stroma. GREM1 (gremlin 1) is a bone morphogenetic protein (BMP) antagonist, and it has not been reported as over-expressed in cancer-associated stromal cells of the breast. Without being bound by theory, and offered only to improve the understanding of the disclosure, it is possible that the significant down-regulation of WNT antagonists (WIF1 and SFRP1) and up-regulation of GREM1 in the stroma (Klapholz-Brown et al., PLoS ONE, 2(9):e945 (2007)) as disclosed herein may be functionally linked. INHBA is the gene for the beta A subunit of inhibin and activin, which are pleiotropic growth factors regulating growth and differentiation of many cell types via autocrine and paracrine mechanisms (Reis et al., Mol. Cell. Endocrinol., 225(1-2):77-82 (2004)). Its role in breast cancer is unclear. Without being bound by theory, and offered only to improve the understanding of the disclosure, these signaling molecules may serve as key messengers between a tumor and its microenvironment in the breast. This has been reported in other contexts for CXCL12 and CXCL14 (Orimo, supra., Allinen, supra., Burger and Kipps, Blood, 107(5):1761-1767 (2006)). But in this disclosure, CXCL12 and CXCL14 were expressed in normal stroma.

### Gene Expression in Cancer Invasiveness and Grade

A watershed event in breast cancer progression is the invasion of tumor cells into the stromal compartment. The only morphological diagnostic criterion distinguishing DCIS from IDC is the association of DCIS with a complete basement membrane. This disclosure advantageously provides information regarding the molecular events that drive the DCIS-IDC transition. It has been previously shown (Ma, supra.) and confirmed herein that the malignant epithelium of DCIS and IDC are very similar without significant differences at the transcriptome level. This conclusion is supported by the recent demonstration that MCFDCIS cells, a cell line model for DCIS, makes the DCIS-IDC transition spontaneously without further molecular changes in the malignant epithelial cells themselves (Hu et al, Cancer Cell, 13(5):394-406 (2008)). Instead, this transition is driven by fibroblasts and blocked by myoepithelial cells.

The present disclosure describes the stromal compartment's association with a relatively small number of significant changes accompanying the DCIS to IDC transition. The genes identified as able to discriminate between in situ stroma and invasive stroma are disclosed in Tables 6 and 10. In particular, several matrix metalloproteases (MMP2, MMP11 and MMP14) showed significantly increased expression in IDC-associated (invasive) stroma. MMP14, a membrane-type MMP, can activate MMP2 protease activity, which degrades type IV collagen, the major structural component of the basement membrane (Rozanov et al., Cancer Res., 68(10:4086-4096 (2008); Egeblad and Werb, Nat. Rev. Cancer, 2(3):161-174 (2002)).

MMP11 has recently been reported to exhibit protease activity towards type VI collagen and promote tumor progression (Motrescu et al., Oncogene, 27:6347-6355 (2008)). But the instant disclosure is the first to describe increased expression of MMP11 in the IDC-associated stroma but not in the epithelium. This is in contrast to previous work by Schuetz et al. who conducted a study profiling the epithelium of patient-matched DCIS and IDC (Schuetz et al., Cancer Res., 66(10):5278-5286 (2006)) and by Hannemann et al. who profiled mixtures of tumor epithelium and stroma (Hannemann et al., Breast Cancer Res. 8(5):R61 (2006)). The disclosed results support use of stroma-produced MMPs as an indicator of the DCIS to IDC transition.

The disclosure also includes the discovery that like the epithelial compartment (Ma, supra.) tumor-associated stroma also exhibits a robust gene expression signature correlated with histological tumor grade. The genes identified as able to discriminate between Grade I and Grade III in both in situ stroma and invasive stroma are disclosed in Table 11. The genes identified with this correlation are primarily involved in immune response and cell cycle progression. The association of an immune response signature with the more aggressive high grade tumors appears unexpected. Without being bound by theory, and offered only to improve the understanding of the disclosure, the immune response signature associated with high grade tumors may identify the “escape” phase (Strausberg, Genome Biol., 6(3):211 (2005)), when breast cancer cells become resistant to immune attack and are able to utilize or neutralize the abundant cytokines and chemokines produced by immune cells.

### Diagnostic Methods

The compositions and methods of the disclosure may be used in the detection of breast cancer in a subject by assessment of stromal and/or epithelial cells from the breast of the subject. In some embodiments, the detection of expression levels of identified genes disclosed herein is used to diagnose the presence or occurrence of breast cancer in the subject. In other embodiments, the detection of the absence of the expression levels of identified genes disclosed herein is used to diagnose the absence of breast cancer in the subject.

In many embodiments of the disclosed methods, detection of expression of more than one disclosed gene is used in combination. Non-limiting examples include the determination of expression levels of about 2, about 3, about 4, about 5, about 6, about 7, about 8, about 9, about 10 or more disclosed genes. The number of genes may be influenced by the methodology used, such as multiplex PCR or a microarray. Additional embodiments include the use of genes with expression levels that discriminate in stromal cells as well as genes with expression levels that discriminate in epithelial cells. The number of genes assayed for each cell type may be independently determined. Also, the presence, or exclusion, of overlapping genes between the two cell types may also be used.

In some embodiments, a diagnostic method of the disclosure is used for early detection of breast cancer. Some of these cases are where visualization of a breast tissue sample from a subject shows no presence of cancer cells or reveals unclear situations such as with atypical hyperplasia. Other non-limiting examples of such cases include those where indeterminate, pre-cancerous, or suspicious lesions are present. So molecular assessment of A stromal and/or epithelial cells, by a disclosed method is used to identify the presence or occurrence of breast cancer in a subject. In a related manner, a method of the disclosure may be used to help rule out the presence of breast cancer because the gene expression levels do not correspond to those disclosed herein for the presence of breast cancer. And in additional embodiments, a diagnostic method of the disclosure may be used to confirm the presence of breast cancer in a breast tissue sample from a subject. So a method of the disclosure may be used to discriminate between the presence of benign and malignant breast cancer. This is particularly advantageous in that the disclosed methods provide an objective molecular basis for determining the presence of breast cancer, which can be used to complement subjective methods such as histological staining and assessment by a pathologist.

The increase or decrease in expression of a disclosed gene, relative to a normal cell, can be determined either quantitatively or qualitatively. In some embodiments, the assessment is performed on a log, scale, where a change by a factor of at least 0.05 is used to determine an increase or decrease. In other embodiments, a factor of at least about 0.05, at least about 0.1, at least about 0.15, at least about 0.2, at least about 0.25, at least about 0.3, at least about 0.35, at least about 0.4, at least about 0.45, at least about 0.5, at least about 0.6, at least about 0.7, at least about 0.8, at least about 0.9, at least about 1, at least about 1.5, at least about 2, at least about 2.5, at least about 3, at least about 3.5, at least about 4, at least about 4.5, at least about 5, at least about 5.5, at least about 6, at least about 6.5, at least about 7 or more is used to determine an increase or decrease. Of course the factor used will vary with the gene being assessed as described herein, and optionally as necessitated by the nature of the detection method.

In further embodiments, a disclosed method may be used to assess the recurrence of breast cancer in a subject. To detect recurrence, a sample from a subject treated for breast cancer, such as via surgical intervention, may be obtained and used to detect gene expression levels as disclosed herein. Stromal and/or epithelial cells may be obtained and used as described herein to detect expression levels indicative of the presence or recurrence of breast cancer in the subject.

In related embodiments, a disclosed method may be used to monitor breast cancer treatment, such as in a breast cancer patient undergoing chemotherapy or radiation therapy as non-limiting examples. In some cases, the chemotherapy is treatment with tamoxifen, an SERM (selective estrogen receptor modulator), an SERD (selective estrogen receptor down-regulator), or an aromatase inhibitor. In one embodiment, the method may be performed with a sample from a subject undergoing treatment for breast cancer to detect gene expression levels as disclosed herein. Stromal and/or epithelial cells may be obtained and used as described herein to detect expression levels indicative of successful treatment (indicated by the absence of expression levels that identify the presence of breast cancer) or indicative of unsuccessful treatment (indicated by the presence of expression levels that identify the presence of breast cancer). The method may be performed once or repeatedly over time, such as at intervals of about 3 months, about 6 months, or about a year or longer.

In another embodiment, the method may be performed with a sample of stromal cells from a subject undergoing treatment for breast cancer to detect gene expression levels as disclosed herein (Tables 6 and 10) that are indicative of an environment suitable for the occurrence of invasive breast cancer. The stromal cells may be obtained and used as described herein to detect expression levels indicative of an environment that discriminates invasive stroma from in situ stroma. The method may be performed once or repeatedly over time, such as at intervals of about 3 months, about 6 months, or about a year or longer.

In some embodiments of identifying invasive from in situ stroma, the expression levels of more than one disclosed gene are combined to form a single index that serves as a strong determination factor. The index is a summation of the expression levels of the genes used and uses coefficients determined from principle component analysis to combine cases of more than one disclosed gene into a single index. The coefficients are determined by factors such as the standard deviation of each gene's expression levels across a representative dataset, and the expression value for each gene in each sample. The representative dataset is quality controlled based upon the average expression values for reference gene(s) as disclosed herein.

In some cases, normalized expression levels for a set of genes from experimental data may be standardized to mean of 0 and standard deviation of 1 across samples within each dataset and then combined into a single index per sample via principle component analysis (PCA) using the first principle component. As a result, and following scaling parameters, a formula for the summation of expression values that defines the index is generated. The precision of the scaling parameters is then be tested based on the means, standard errors, and standard deviations (with confidence intervals) of the expression levels of the genes across the data set. Therefore, generation of the formula for the index is dependent upon the dataset, reference gene, and genes used to discriminate between the two stroma types.

For embodiments of the index using real-time PCR, obviously abnormal raw C_{T} values are removed prior to averaging the values over duplicates for each gene and each sample. The averaged raw C_{T} value for each gene is then normalized by the averaged C_{T} value of reference genes used. The normalized expression levels (ΔC_{T}) for the disclosed genes used are combined into a single index per sample, which can be compared to a pre-determined cutoff value, such as 0, where a high index is above the cutoff and a low index is below the cutoff. The calculation and the cutpoint for such an index are defined without using any clinical outcome data and instead was a natural cutpoint. Such an index can provide good discrimination of invasive/infiltrating stroma versus in situ stroma tumors using a determined cutpoint. Model-based clustering of the index that indicates a natural bimodal distribution about the cutpoint used further supports a selected cutpoint. A cutpoint can be further supported by receiver operating characteristic (ROC) analysis.

In further embodiments, the above described methodology is applied to a stromal cell from a subject to determine the grade of breast cancer in the subject based on gene expression levels as disclosed herein (Table 11) that discriminate between Grade I and Grade III cancer. In many embodiments, stromal cells are obtained and used as described herein to detect expression levels indicative of cancer of Grade I, or Grade III, breast cancer. In some cases, the expression levels of more than one disclosed gene are combined to form a single index, as described above, to serve as a strong determination factor. The index is a summation of the expression levels of the genes used and uses coefficients determined from principle component analysis to combine cases of more than one disclosed gene into a single index.

In additional embodiments, a disclosed method may be used to determine the likelihood of breast cancer recurrence in a subject treated for IDC and/or DCIS. The treatment may be any known to the skilled person, including surgical intervention, chemotherapy, and radiotherapy as described herein. The method may be performed with a sample from a subject that has undergone, or is still undergoing, treatment for breast cancer to detect gene expression levels as disclosed herein. Stromal and/or epithelial cells may be obtained and used as described herein to detect expression levels indicative of a likelihood of cancer recurrence.

In further embodiments, a disclosed method may detect expression of a polypeptide, or fragment thereof, expressed by a disclosed gene in a biological fluid from a subject. Detection of an increase or decrease of such a polypeptide, or fragment, is used to indicate or suggest the presence of breast cancer in a subject. As with other disclosed methods, this embodiment may be performed with the use of multiple polypeptide gene products. In many cases, the polypeptide is one that is secreted or sloughed off from a stromal or epithelial cell. In other cases, the polypeptide is released upon lysis of a stromal or epithelial cell.

### Therapeutic Methods

Embodiments of the disclosure include a method to determine therapeutic treatment for a cancer patient. The method may include first identifying the presence or occurrence of breast cancer in a subject as disclosed herein, optionally early detection as described herein. This diagnosis is then followed by selecting treatment for a patient with the breast cancer that has been diagnosed. The treatment may be any that is recognized as suitable, including surgical intervention, chemotherapy, and radiotherapy as non-limiting examples.

Having now generally provided the disclosure, the same will be more readily understood through reference to the following examples which are provided by way of illustration, and are not intended to be limiting of the disclosure, unless specified.

### EXAMPLES

### Example 1: Materials and Methods

### Clinical Specimen

All breast cancer specimens were fresh frozen biopsies obtained from the Massachusetts General Hospital between 1998 and 2001. Diagnostic criteria and tumor grading were described previously (Ma, supra.). Patient and tumor (primary ductal breast cancer) characteristics of the 14 tumor specimens in these examples are listed in Table 1.

TABLE 1
Patient and tumor characteristics of samples in study.

| Case | | | | | | | Nodal | Tumor |
|---|---|---|---|---|---|---|---|---|
| No. | Age | Grade | ER | PR | Her-2 | Size | status | Type |
| 44 | 28 | III | Pos | Pos | Neg | 1 | Neg | Ductal |
| 45 | 36 | I | Pos | Pos | Neg | N/A | Neg | Ductal |
| 79 | 54 | I | Pos | Pos | Neg | 2.1 | Pos | Ductal |
| 96 | 31 | III | Neg | Neg | Neg | 3.7 | Neg | Ductal |
| 102 | 55 | I | Pos | Neg | Neg | 5.2 | Pos | Ductal |
| 121 | 45 | II | Pos | Pos | Pos | 1.5 | Pos | Ductal |
| 131 | 37 | II | Pos | Pos | Pos | 1.5 | Pos | Ductal |
| 133 | 44 | III | Neg | Neg | Pos | 1.5 | Pos | Ductal |
| 148 | 42 | II | Pos | Pos | Neg | 1.9 | Pos | Ductal |
| 153 | 46 | I | Pos | Pos | ND | N/A | Pos | Ductal |
| 169 | 34 | II | Pos | Pos | Neg | 2.6 | Pos | Ductal |
| 178 | 43 | III | Pos | Pos | Pos | 2.8 | Pos | Ductal |
| 179 | 37 | III | Neg | Neg | Pos | 1.5 | Pos | Ductal |
| 180 | 46 | I | Pos | Pos | Neg | 1.9 | Pos | Ductal |

Abbreviations:
Pos, positive;
neg, negative;
ND, not determined;
N/A, not available.

Patients were selected in which patient matched normal and tumor samples were available and the normal breast lobules did not show fibrocystic change.

This study was approved by the Massachusetts General Hospital human research committee in accordance with National Institutes of Health human research study guidelines.

### LCM, RNA Extraction and Microarray Analysis

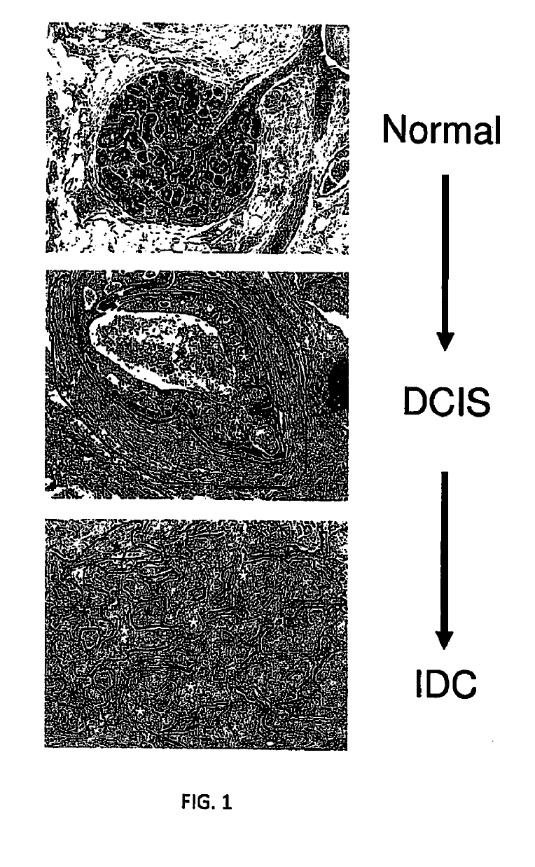

Highly enriched populations of patient-matched normal or malignant epithelial cells, normal stroma or tumor-associated stroma from the different stages of breast cancer progression were procured by laser capture microdissection (LCM) using a PixCell® lie system (Molecular Devices, Mountain View, Calif.) as previously described (Ma, supra.). Enrichment for cells of interest was verified by microscopic examination of the LCM caps after microdissection. The microdissected normal stromal compartment (N-S) consisted of the intralobular, rather than the extralobular, stromal compartment of normal breast tissue that was at a minimum 0.3 cm from any pre-malignant or malignant lesion (FIG. 1). The DCIS-associated stroma (DCIS-S) consisted of a 25 μ rim of cells that surrounded the DCIS; for cases in which metachronous DCIS and invasive ductal carcinoma (IDC) were present, the DCIS-S was obtained from areas of DCIS that were at least 0.3 cm from the invasive component. The IDC-associated stroma (I DC-S) consists of stromal cells predominantly within the invasive tumor mass.

Total RNA was isolated from captured cells using the Picopure™ RNA isolation kit (Molecular Devices), amplified by T7 RNA amplification (RiboAmp™, Molecular Devices), labeled and hybridized to the whole genome array U133X3P (3′-biased design) according to manufacturer's instructions (Affymetrix, Santa Clara, Calif.). The hybridized microarrays were then washed, stained and scanned per manufacture's protocols (Affymetrix).

### Data Analysis

Raw data from U133X3P arrays were processed using the Bioconductor package rma with default parameters for background correction, quantile normalization and signal summation (Gentleman et al., Genome Biol. 5(10):R80 (2004); Bolstad et al., Bioinformatics, 19(2):185-193 (2003)). Differential gene expression analyses were performed using linear regression models in the limma package (Wettenhall et al., Bioinformatics, 20(18):3705-3706 (2004)). For comparing normal and tumor samples, patient id was used as a blocking variable. For tumor grade comparison, tumor stage (in situ or invasive) was used as the blocking variable. Statistical significance was corrected for multiple testing using the Benjamini-Hochberg procedure (Benjamin et al., J. Royal Stat. Soc., Series_{—}B, (57):289-300 (1995)). All procedures were performed in the R statistical environment (available at www.r-project.org). For gene ontology analysis, ranked gene lists were first generated according to the moderated t-statistics from linear models and then examined for enriched ontology terms using the Gene Set Enrichment Analysis (GSEA) software (Subramanian et al., Bioinformatics, 23(23):3251-3253 (2007)).

### Quantitative Real-Time PCR and Immunohistochemistry

TaqMan™ real-time PCR was performed on amplified RNA (aRNA) used for microarray analysis as previously described (Ma, supra.). Briefly, aRNA was converted to double-stranded cDNA, and the cDNA was quantitated with PicoGreen® (Molecular Probes) using a spectrofluorometer (Molecular Devices). Each gene was analyzed in triplicate in a 96-well plate using ABI 7900HT (Applied Biosystem). For the following genes, the sequences of the PCR primer pairs and the fluorogenic MGB probe (5′ to 3′), respectively, are as follows:

ESR1,
(SEQ ID NO: 1)
ATGATCAACTGGGCGAAGA,
(SEQ ID NO: 2)
GGTGGACCTGATCATGGA,
(SEQ ID NO: 3)
VIC-TGCCAGGCTTTGTGGA;
RRM2,
(SEQ ID NO: 4)
CCTTTAACCAGCACAGCCAGTT,
(SEQ ID NO: 5)
TTATTTGTTTGTAAAGTGCCAGGTTT,
(SEQ ID NO: 6)
VIC-TGCAGCCTCACTGCTTCAACGCA;
GREM1,
(SEQ ID NO: 7)
ACGGCAAAGAATTATATAGACTATGAGGTA,
(SEQ ID NO: 8)
TTTTATGAGACTATCAACTCCCCTTTC,
(SEQ ID NO: 9)
VIC-CTTGCTGTGTAGGAGGA;
WIF1,
(SEQ ID NO: 10)
CACTGTGGTAGTGGCATTTAAACAATA,
(SEQ ID NO: 11)
GCCAATGCAAAAAGTTCATACATT,
(SEQ ID NO.: 12)
VIC-TTCTAAACACAATGAAATAGGGA.

ER and PR immunohistochemistry staining was performed as previously described using the rabbit monoclonal antibody (SP1) from Lab Vision for ER (1:50 dilution) and the mouse monoclonal antibody (PgR 636) from Dako (Carpinteria, Calif.) for PR (1:50 dilution) (Ma et al., Cancer Cell, 5(6):607-616 (2004)).

### Example 2: Subject Profiles and Sample Preparation

The patient samples used were primarily estrogen receptor positive (78.6%), lymph node-positive (78.6%), and pre-menopausal (mean age=41). Laser capture microdissection (LCM) was used to isolate epithelial and stromal cells separately from each of the 14 fresh frozen biopsies. In the epithelial compartment, normal (N) and malignant epithelium from ductal carcinoma in situ (DCIS) and/or invasive ductal carcinoma (IDC) were captured. In the stroma compartment, normal stroma (N-S) at least 3mm away from the malignant lesion and the DCIS-associated stroma (DCIS-S) and/or IDC-associated stroma (IDC-S) were captured whenever possible. An example of the microdissected compartments is shown in FIG. 1.

As shown in Table 2, in the epithelial compartment, 4 cases had all three stages (N, DCIS, and IDC) available, 5 cases had N and IDC only, and 5 cases had N and DCIS only; in the stroma, 6 cases had all three stages available, 5 cases had N-S and DCIS-S, and 3 cases had N-S and IDC-S.

TABLE 2
Laser capture microdissection of 14 primary breast cancer

| | Tumor | | | Stroma | | |
|---|---|---|---|---|---|---|
| patient | N | IS | INV | NSS | ISS | INVS |
| 44 | x | x | x | x | x | x |
| 45 | x | | x | x | x | x |
| 79 | x | | x | x | x | x |
| 96 | x | x | x | x | x | x |
| 102 | x | x | x | x | x | x |
| 121 | x | x | x | x | x | x |
| 131 | x | x | | x | x | |
| 133 | x | x | | x | x | |
| 148 | x | | x | x | | x |
| 153 | x | | x | x | | x |
| 169 | x | | x | x | | x |
| 178 | x | x | | x | x | |
| 179 | x | x | | x | x | |
| 180 | x | x | | x | x | |

x denotes component captured.

RNA was isolated from the captured cells and interrogated with the Affymetrix whole-genome array U133X3P.

### Example 3: Gene Expression Changes in the Stromal and Epithelial Compartments During Breast Cancer Progression

The gene expression patterns of the tumor epithelium and stroma at each stage of progression (DCIS or IDC) was compared to their respective normal state using the limma (linear models of microarrays) software package (Wettenhall, supra.). The resulting p values for differential gene expression in each pair-wise comparison were adjusted for multiple testing (Benjamin, supra.), and the genes with a significant adjusted p value (p<0.05) were extracted.

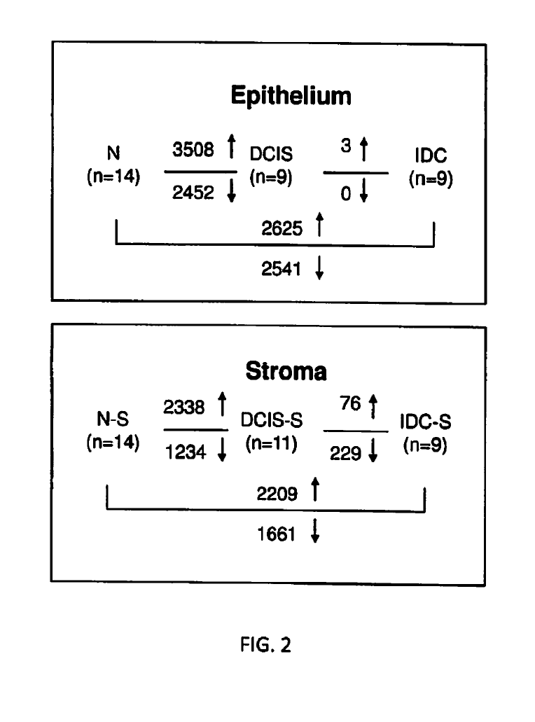

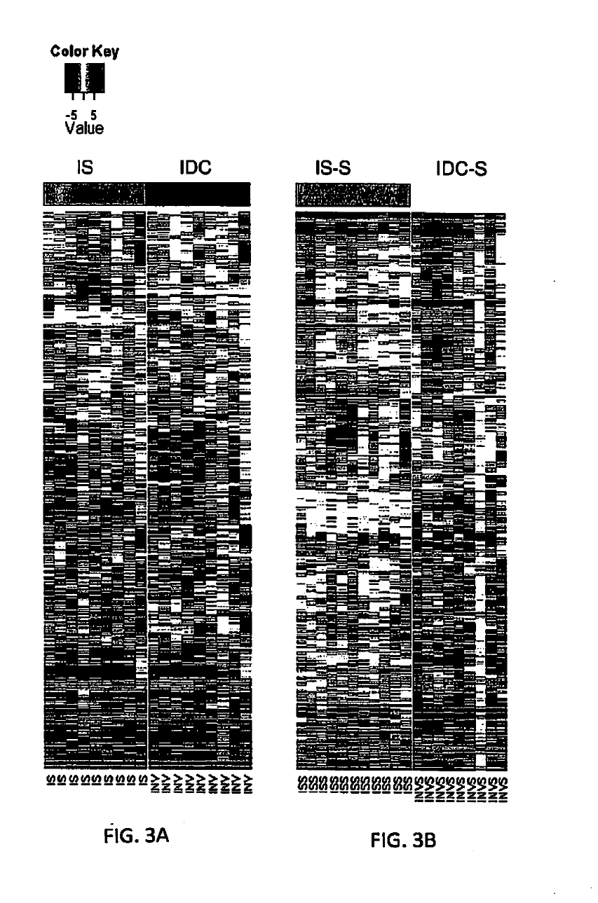

The DCIS and IDC stages were each associated with thousands of gene expression alterations relative their respective normal state in both the tumor epithelium and the stroma (FIG. 2). Furthermore, within each compartment, the expression patterns of DCIS and IDC-associated genes were highly similar to each other (FIG. 3).

To gain an overview of the biological processes in which these differentially expressed genes are involved, gene set enrichment analysis (GSEA) (Subramanian et al., Proc. Natl. Acad. Sci. USA, 102(43):15545-15550 (2005)) was performed using the gene ontology (GO) database (Ashburner et al., The Gene Ontology Consortium, Nat. Genet., 25(1):25-29 (2000)). Table 3 lists the top 20 GO terms significantly enriched within genes up-regulated in the invasive stage in the epithelium and the stroma.

TABLE 3
Top 20 gene ontology terms enriched in tumor epithelium and stroma.

| NAME | SIZE | NES | FDR q-val |
|---|---|---|---|
| Epithelium | | | |
| SPINDLE | 39 | 2.33 | 0 |
| CHROMOSOME_SEGREGATION | 28 | 2.15 | 0 |
| CELL_CYCLE_PROCESS | 180 | 2.12 | 0 |
| MICROTUBULE_CYTOSKELETON_ORGANIZATION_AND_BIOGENESIS | 34 | 2.11 | 0 |
| CHROMOSOME_PERICENTRIC_REGION | 27 | 2.11 | 0 |
| MICROTUBULE_CYTOSKELETON | 142 | 2.11 | 0 |
| PROTEASOME_COMPLEX | 22 | 2.09 | 1.40E−04 |
| CONDENSED_CHROMOSOME | 30 | 2.06 | 2.48E−04 |
| M_PHASE | 105 | 2.06 | 2.20E−04 |
| NUCLEAR_ENVELOPE | 71 | 2.05 | 1.98E−04 |
| CELL_CYCLE_PHASE | 157 | 2.05 | 1.80E−04 |
| M_PHASE_OF_MITOTIC_CELL_CYCLE | 78 | 2.04 | 2.49E−04 |
| CHROMOSOME | 115 | 2.03 | 2.30E−04 |
| CYTOSKELETAL_PART | 221 | 2.03 | 2.14E−04 |
| MITOSIS | 75 | 2.02 | 2.66E−04 |
| MICROTUBULE | 32 | 1.99 | 2.49E−04 |
| MITOTIC_CELL_CYCLE | 139 | 1.99 | 2.35E−04 |
| CELL_CYCLE_CHECKPOINT_GO_0000075 | 45 | 1.98 | 2.21E−04 |
| SPINDLE_MICROTUBULE | 16 | 1.97 | 2.63E−04 |
| DNA_REPAIR | 120 | 1.94 | 6.01E−04 |
| STRUCTURAL_CONSTITUENT_OF_RIBOSOME | 74 | −3.09 | 0 |
| Stroma | | | |
| EXTRACELLULAR_MATRIX_STRUCTURAL_CONSTITUENT | 25 | 2.12 | 0 |
| COLLAGEN | 23 | 2.07 | 0.001566 |
| METALLOENDOPEPTIDASE_ACTIVITY | 26 | 2.06 | 0.001044 |
| EXTRACELLULAR_MATRIX | 94 | 1.99 | 0.001568 |
| PROTEINACEOUS_EXTRACELLULAR_MATRIX | 93 | 1.97 | 0.002923 |
| EXTRACELLULAR_MATRIX_PART | 54 | 1.91 | 0.007826 |
| SPINDLE | 39 | 1.89 | 0.008346 |
| METALLOPEPTIDASE_ACTIVITY | 45 | 1.82 | 0.027006 |
| SKELETAL_DEVELOPMENT | 99 | 1.80 | 0.032482 |
| STRUCTURAL_CONSTITUENT_OF_RIBOSOME | 74 | −3.04 | 0 |

Abbreviations:
NES, normalized enrichment score;
FDR, false discovery rate.

In the epithelium, the genes were dominated by those associated with the cell cycle (mitosis, in particular). In the stroma, the genes prominently featured the components of the extracellular matrix (ECM) and matrix metalloproteases responsible for remodeling ECM.

Additionally, the stromal genes also included those related to the cell cycle, indicating increased proliferation as a common feature in both the tumor epithelium and the stroma.

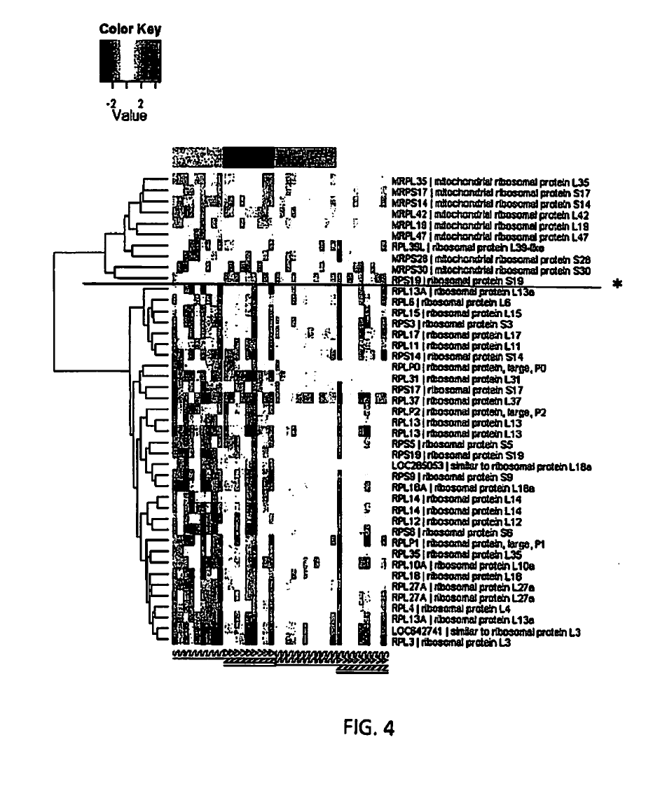

In both compartments, a single GO term “Structural Constituents of Ribosome” was significantly enriched within the down-regulated genes (Table 3). To examine this further, all ribosomal protein-encoding genes that were differentially expressed between DCIS or IDC vs. N in the epithelium were extracted and their expression patterns visualized in both compartments. There was an almost complete bipartite partitioning of these genes (FIG. 4): while the down-regulated genes were all those encoding for the cytoplasmic ribosomal proteins, the up-regulated genes were mostly those encoding for the mitochondrial ribosomal proteins.

In addition to these global patterns, Tables 4 and 5 list the top 50 differentially expressed genes in the epithelium and the stroma, respectively.

TABLE 4
Top 50 genes differentially expressed in tumor epithelium.

| Probeset | DCIS | IDC | Gene Description |
|---|---|---|---|
| g5174662_3p_at | 5.3 | 4.0 | S100P\|S100 calcium binding protein P |
| g11993936_3p_s_at | 4.5 | 3.4 | CYB561\|cytochrome b-561 |
| g7415720_3p_a_at | 4.0 | 3.0 | SCD\|stearoyl-CoA desaturase (delta-9- |
| | | | desaturase) |
| Hs.75319.0.S3_3p_at | 3.0 | 3.8 | RRM2\|ribonucleotide reductase M2 polypeptide |
| Hs.106552.0.S3_3p_s_at | 4.2 | 2.3 | CNTNAP2\|contactin associated protein-like 2 |
| g12803628_3p_at | 3.7 | 2.6 | HIST1H1C\|histone cluster 1, H1c |
| g5031780_3p_at | 4.0 | 2.3 | IFI27\|interferon, alpha-inducible protein 27 |
| Hs.180779.1.S1_3p_at | 3.1 | 3.0 | HIST1H2BD\|histone cluster 1, H2bd |
| Hs.184572.0.S2_3p_at | 2.7 | 3.3 | CDC2\|cell division cycle 2, G1 to S and G2 to M |
| Hs.223025.0.S2_3p_a_at | 2.6 | 3.4 | RAB31\|RAB31, member RAS oncogene family |
| g12804874_3p_a_at | 2.8 | 3.1 | RRM2\|ribonucleotide reductase M2 polypeptide |
| Hs.239884.0.S1_3p_x_at | 3.5 | 2.4 | HIST1H2BC\|histone cluster 1, H2bc |
| g7661973_3p_at | 2.7 | 3.1 | MELK\|maternal embryonic leucine zipper kinase |
| g13259549_3p_at | 3.2 | 2.4 | IFI6\|interferon, alpha-inducible protein 6 |
| g4504584_3p_at | 3.5 | 2.0 | IFIT1\|interferon-induced protein with |
| | | | tetratricopeptide repeats 1 |
| Hs.155956.0.S1_3p_at | 2.6 | 2.8 | NAT1\|N-acetyltransferase 1 (arylamine N- |
| | | | acetyltransferase) |
| Hs.152677.0.S1_3p_at | 3.1 | 2.3 | DHRS2\|dehydrogenase/reductase (SDR family) |
| | | | member 2 |
| Hs.239884.0.S1_3p_at | 3.3 | 2.1 | HIST1H2BC\|histone cluster 1, H2bc |
| g5803130_3p_a_at | 2.2 | 3.2 | RAB31\|RAB31, member RAS oncogene family |
| g13699814_3p_s_at | 2.7 | 2.7 | CYP2B6\|cytochrome P450, family 2, subfamily |
| | | | B, polypeptide 6 |
| g9963780_3p_a_at | 2.2 | 3.2 | RAB31\|RAB31, member RAS oncogene family |
| Hs.72472.0.A1_3p_at | 2.3 | 2.9 | — |
| g13477106_3p_s_at | 3.3 | 1.8 | CEACAM6\|carcinoembryonic antigen-related |
| | | | cell adhesion molecule 6 (non-specific cross |
| | | | reacting antigen) |
| Hs.133342.0.S1_3p_x_at | 2.9 | 2.2 | GPC1\|glypican 1 |
| Hs.325335.1.S1_3p_at | 2.6 | 2.5 | CAPN13\|calpain 13 |
| Hs.34853.0.S2_3p_at | −3.5 | −4.0 | ID4\|inhibitor of DNA binding 4, dominant |
| | | | negative helix-loop-helix protein |
| g3387766_3p_a_at | −3.9 | −3.6 | GPM6B\|glycoprotein M6B |
| Hs.10587.0.S1_3p_at | −3.2 | −4.3 | DMN\|desmuslin |
| Hs2.348883.1.S1_3p_s_at | −3.9 | −3.6 | FOXC1\|forkhead box C1 |
| g4758521_3p_at | −4.9 | −2.7 | SPARCL1\|SPARC-like 1 (mast9, hevin) |
| g11037715_3p_x_at | −3.9 | −3.7 | ROPN1\|ropporin, rhophilin associated protein 1 |
| g4504914_3p_at | −3.9 | −3.8 | KRT15\|keratin 15 |
| Hs.82101.0.S3_3p_at | −3.7 | −4.0 | PHLDA1\|pleckstrin homology-like domain, |
| | | | family A, member 1 |
| Hs.149356.0.S1_3p_at | −3.8 | −3.9 | LOC728264\|hypothetical protein LOC728264 |
| g4506856_3p_s_at | −3.3 | −4.6 | CX3CL1\|chemokine (C—X3—C motif) ligand 1 |
| g4506516_3p_at | −3.9 | −4.1 | RGS2\|regulator of G-protein signaling 2, 24 kDa |
| g7657105_3p_at | −4.2 | −4.0 | GABRP\|gamma-aminobutyric acid (GABA) A |
| | | | receptor, pi |
| Hs.25956.0.S1_3p_at | −4.1 | −4.4 | SOSTDC1\|sclerostin domain containing 1 |
| Hs.153961.2.S2_3p_at | −4.6 | −3.9 | BOC\|Boc homolog (mouse) |
| g11991655_3p_at | −4.5 | −4.2 | C2orf40\|chromosome 2 open reading frame 40 |
| Hs.288850.0.S1_3p_at | −4.1 | −4.7 | PHLDA1\|pleckstrin homology-like domain, |
| | | | family A, member 1 |
| g7662650_3p_at | −5.1 | −3.7 | C13orf15\|chromosome 13 open reading frame 15 |
| g4557694_3p_a_at | −4.5 | −4.5 | KIT\|v-kit Hardy-Zuckerman 4 feline sarcoma |
| | | | viral oncogene homolog |
| Hs.127428.2.S2_3p_a_at | −4.3 | −4.9 | HOXA9\|homeobox A9 |
| g6005949_3p_at | −4.4 | −4.8 | WIF1\|WNT inhibitory factor 1 |
| Hs.34853.0.S3_3p_at | −4.8 | −4.5 | ID4\|inhibitor of DNA binding 4, dominant |
| | | | negative helix-loop-helix protein |
| g4559274_3p_a_at | −4.1 | −5.3 | ELF5\|E74-like factor 5 (ets domain transcription |
| | | | factor) |
| g8400731_3p_a_at | −4.4 | −5.2 | SFRP1\|secreted frizzled-related protein 1 |
| g5032314_3p_a_at | −4.9 | −4.9 | DMD\|dystrophin (muscular dystrophy, Duchenne |
| | | | and Becker types) |
| g6005714_3p_at | −4.7 | −5.6 | SLC6A14\|solute carrier family 6 (amino acid |
| | | | transporter), member 14 |

Shown in the DCIS and IDC columns are log2 (Fold changes) relative to normal epithelium.

TABLE 5
Top 50 genes differentially expressed in tumor-associated stroma.

| Probeset | DCIS | IDC | Gene Description |
|---|---|---|---|
| Hs.179729.0.S1_3p_a_at | 6.5 | 7.0 | COL10A1\|collagen, type X, alpha 1(Schmid |
| | | | metaphyseal chondrodysplasia) |
| Hs.28792.0.S1_3p_at | 5.9 | 6.0 | NA |
| 4876385_3p_at | 4.9 | 6.3 | COL11A1\|collagen, type XI, alpha 1 |
| g7019348_3p_at | 4.9 | 5.7 | GREM1\|gremlin 1, cysteine knot superfamily, |
| | | | homolog (Xenopus laevis) |
| Hs.179729.1.S1_3p_a_at | 4.8 | 5.5 | COL10A1\|collagen, type X, alpha 1(Schmid |
| | | | metaphyseal chondrodysplasia) |
| Hs.297939.3.S1_3p_at | 4.6 | 5.1 | FNDC1\|fibronectin type III domain containing 1 |
| 37892_3p_at | 4.1 | 5.6 | COL11A1\|collagen, type XI, alpha 1 |
| Hs.41271.0.S1_3p_at | 4.7 | 4.9 | COL8A1\|collagen, type VIII, alpha 1 |
| g4502938_3p_s_at | 3.8 | 5.4 | COL11A1\|collagen, type XI, alpha 1 |
| g186414_3p_a_at | 4.5 | 4.4 | INHBA\|inhibin, beta A |
| g8393842_3p_at | 4.4 | 4.4 | NOX4\|NADPH oxidase 4 |
| Hs.288467.0.S1_3p_at | 3.8 | 4.8 | LRRC15\|leucine rich repeat containing 15 |
| Hs.105700.0.S1_3p_a_at | 4.1 | 4.2 | SFRP4\|secreted frizzled-related protein 4 |
| g4481752_3p_at | 3.6 | 4.4 | GJB2\|gap junction protein, beta 2, 26 kDa |
| g8923132_3p_at | 3.6 | 4.3 | ASPN\|asporin |
| Hs.287820.2.A1_3p_s_at | 3.8 | 4.1 | FN1\|fibronectin 1 |
| g8400733_3p_a_at | 3.5 | 3.9 | SFRP4\|secreted frizzled-related protein 4 |
| g10863087_3p_a_at | 3.4 | 3.9 | GREM1\|gremlin 1, cysteine knot superfamily, |
| | | | homolog (Xenopus laevis) |
| g5174662_3p_at | 3.4 | 3.4 | S100P\|S100 calcium binding protein P |
| Hs.283713.0.A1_3p_at | 3.2 | 3.5 | CTHRC1\|collagen triple helix repeat containing 1 |
| g4502844_3p_at | 3.1 | 3.5 | CILP\|cartilage intermediate layer protein, |
| | | | nucleotide pyrophosphohydrolase |
| Hs.76722.2.S1_3p_at | 2.8 | 3.7 | — |
| Hs.70823.0.S3_3p_at | 3.3 | 3.2 | SULF1\|sulfatase 1 |
| g4505186_3p_at | 3.3 | 3.0 | CXCL9\|chemokine (C—X—C motif) ligand 9 |
| Hs.101302.0.S2_3p_s_at | 2.2 | 3.9 | COL12A1\|collagen, type XII, alpha 1 |
| g11415037_3p_at | −3.2 | −3.0 | SLC22A3\|solute carrier family 22 (extraneuronal |
| | | | monoamine transporter), member 3 |
| Hs.325823.0.A1_3p_at | −2.7 | −3.5 | CD36\|CD36 molecule (thrombospondin receptor) |
| g4557418_3p_at | −2.7 | −3.6 | CD36\|CD36 molecule (thrombospondin receptor) |
| g4557544_3p_a_at | −2.9 | −3.5 | EDN3\|endothelin 3 |
| Hs2.147313.1.S1_3p_s_at | −2.9 | −3.5 | CD300LG\|CD300 molecule-like family member g |
| Hs.106283.4.S1_3p_at | −3.0 | −3.4 | KLHL13\|kelch-like 13 (Drosophila) |
| g8400731_3p_a_at | −2.6 | −4.0 | SFRP1\|secreted frizzled-related protein 1 |
| Hs.250692.0.S4_3p_at | −3.0 | −3.7 | HLF\|hepatic leukemia factor |
| g4826977_3p_at | −3.3 | −3.5 | RELN\|reelin |
| Hs.76325.1.A1_3p_x_at | −3.0 | −3.9 | IGJ\|immunoglobulin J polypeptide, linker protein |
| | | | for immunoglobulin alpha and mu polypeptides |
| Hs.76325.1.A1_3p_at | −3.1 | 4.0 | IGJ\|immunoglobulin J polypeptide, linker protein |
| | | | for immunoglobulin alpha and mu polypeptides |
| g10835124_3p_a_at | −4.0 | −3.1 | DCX\|doublecortex; lissencephaly, X-linked |
| | | | (doublecortin) |
| g7657105_3p_at | −3.2 | −4.0 | GABRP\|gamma-aminobutyric acid (GABA) A |
| | | | receptor, pi |
| g4506328_3p_at | −3.4 | −3.9 | PTPRZ1\|protein tyrosine phosphatase, receptor- |
| | | | type, Z polypeptide 1 |
| g4758377_3p_at | −3.9 | −3.4 | FIGF\|c-fos induced growth factor (vascular |
| | | | endothelial growth factor D) |
| g12707575_3p_at | −3.3 | −4.1 | OXTR\|oxytocin receptor |
| g13518036_3p_a_at | −2.5 | −4.9 | MATN2\|matrilin 2 |
| g4559274_3p_a_at | −3.9 | −3.6 | ELF5\|E74-like factor 5 (ets domain transcription |
| | | | factor) |
| Hs.10587.0.S1_3p_at | −2.5 | −5.1 | DMN\|desmuslin |
| Hs.49696.0.A1_3p_at | −3.7 | −4.0 | SCARA5\|scavenger receptor class A, member 5 |
| | | | (putative) |
| g4557578_3p_at | −3.4 | −4.4 | FABP4\|fatty acid binding protein 4, adipocyte |
| g13186315_3p_a_at | −3.5 | −4.3 | CAPN6\|calpain 6 |
| g11991655_3p_at | −3.2 | −5.3 | C2orf40\|chromosome 2 open reading frame 40 |
| g562105_3p_a_at | −4.9 | −4.5 | DLK1\|delta-like 1 homolog (Drosophila) |
| g6005949_3p_at | −5.0 | −4.8 | WIF1\|WNT inhibitory factor 1 |

Shown in the DCIS and IDC columns are log2 (Fold changes) relative to normal stroma.

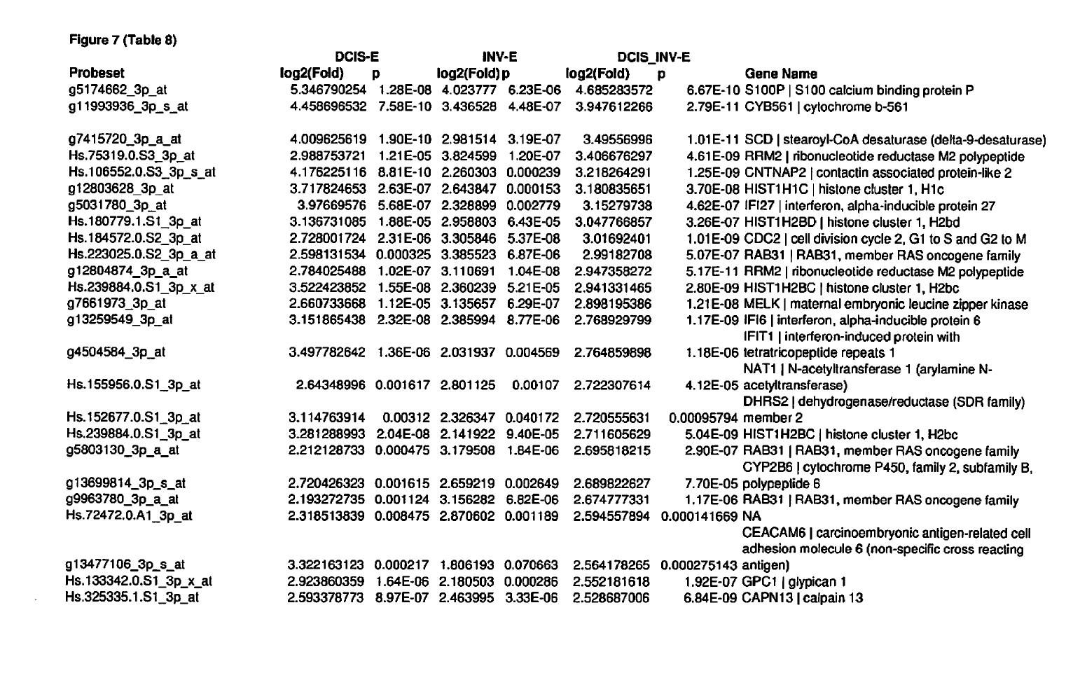

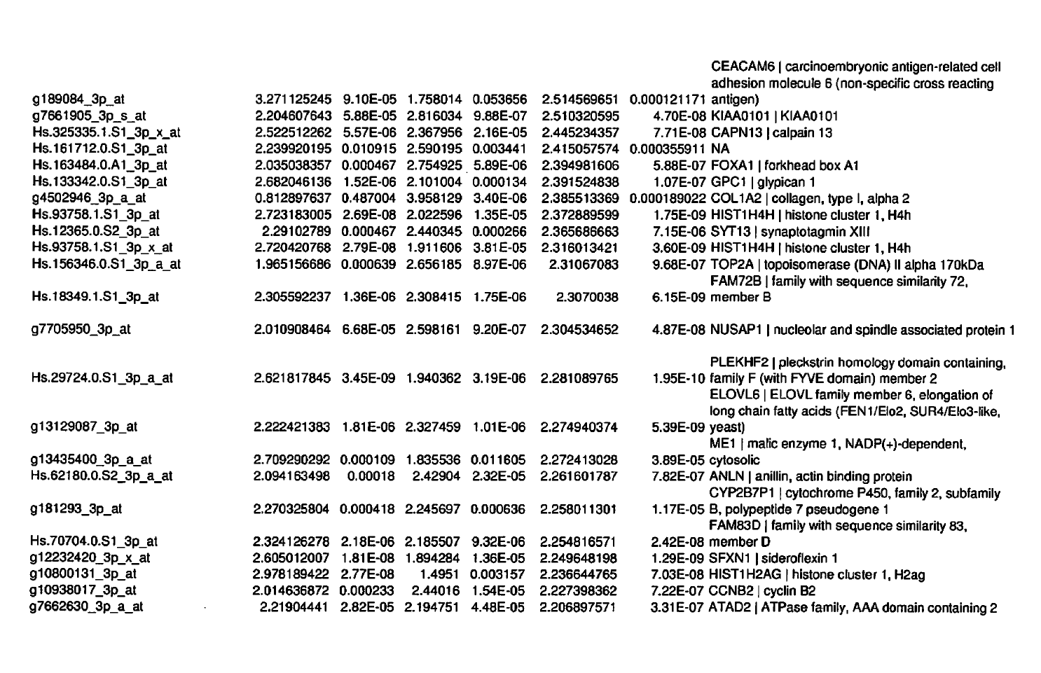

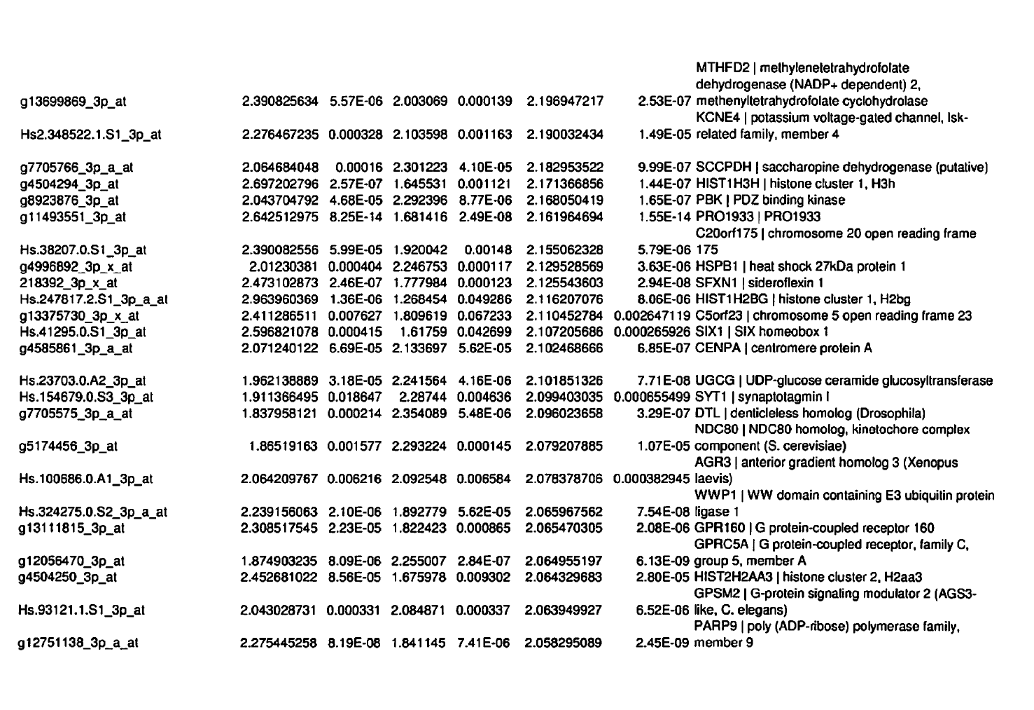

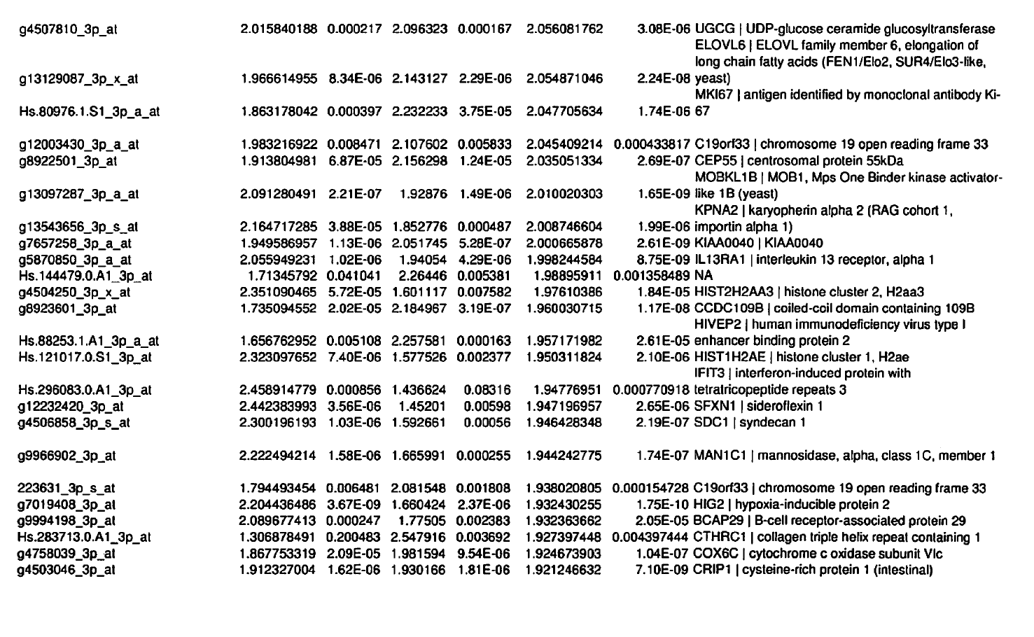

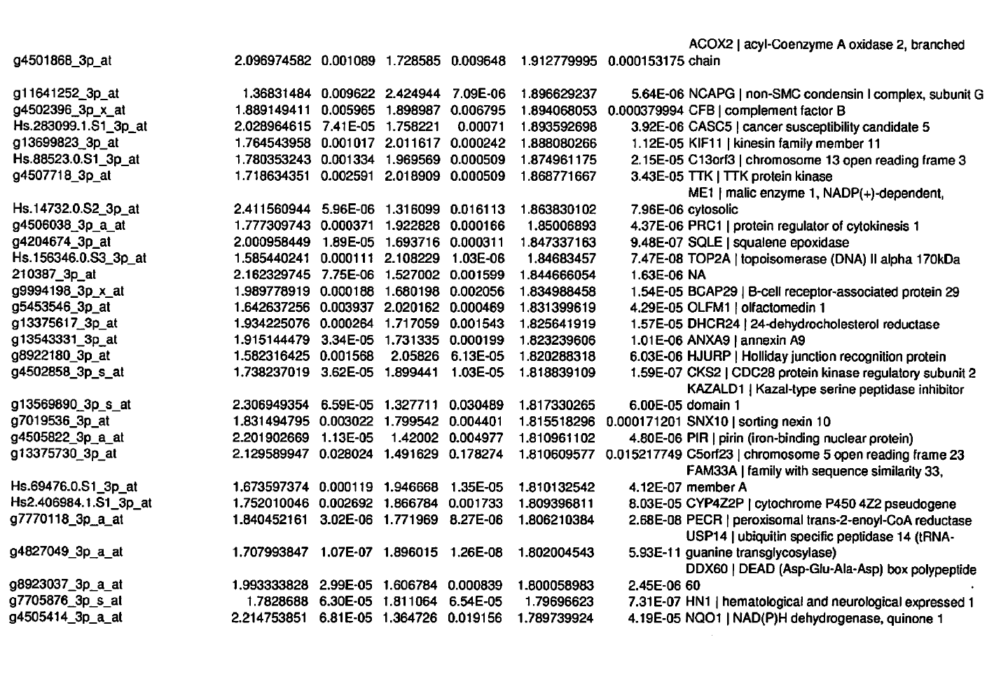

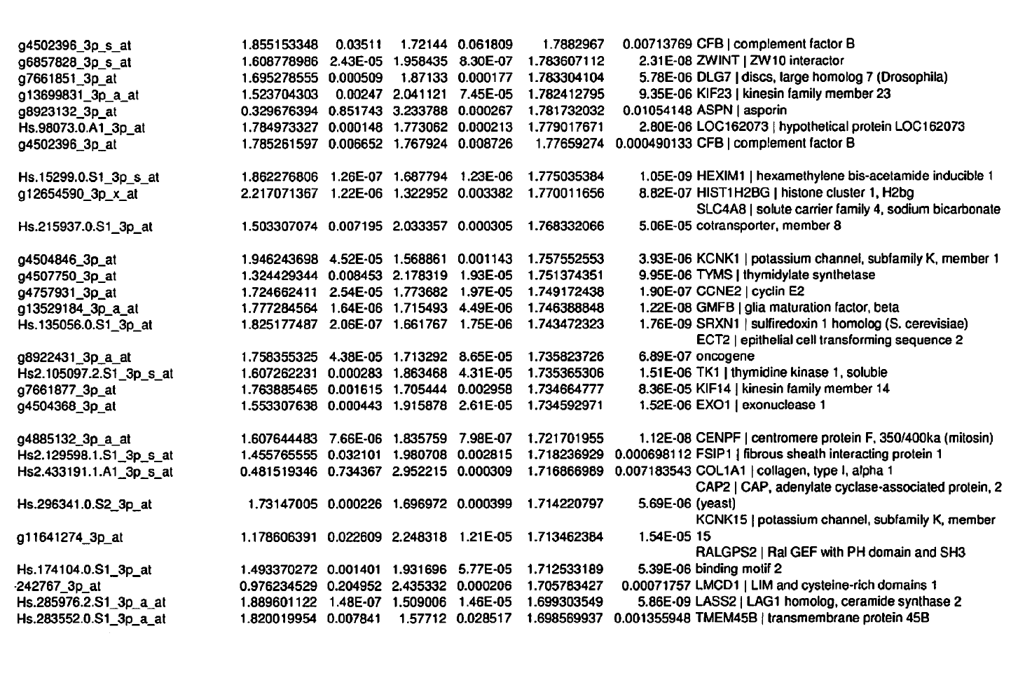

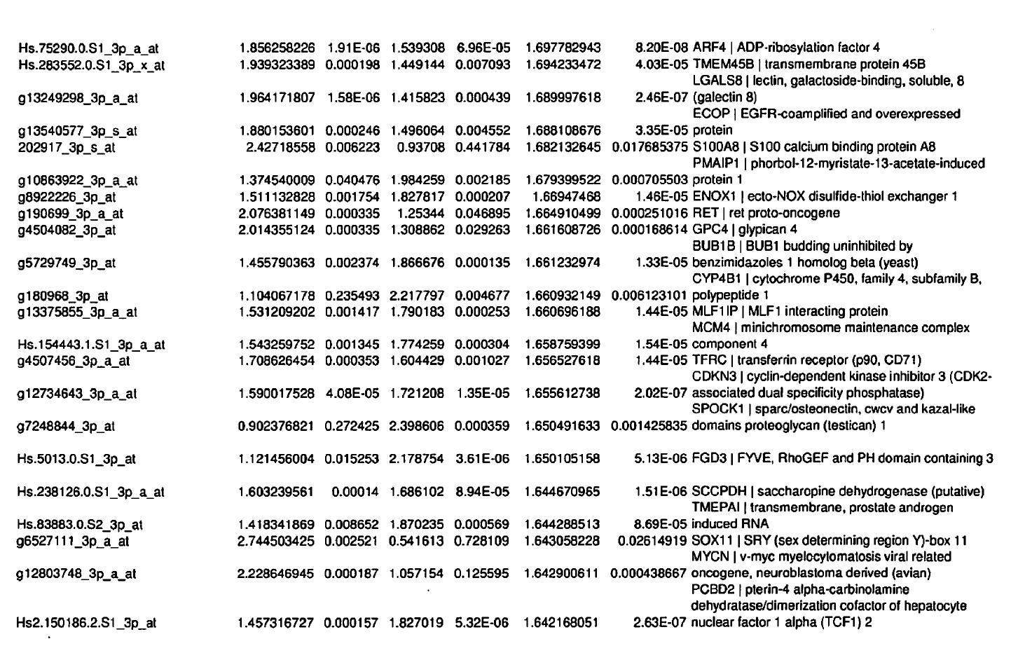

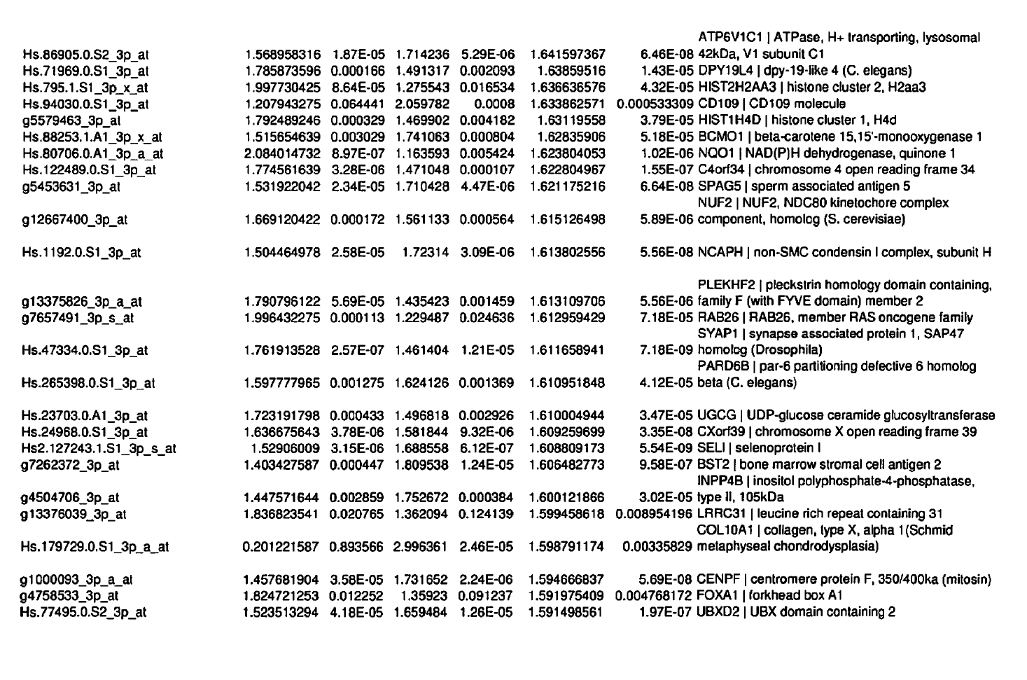

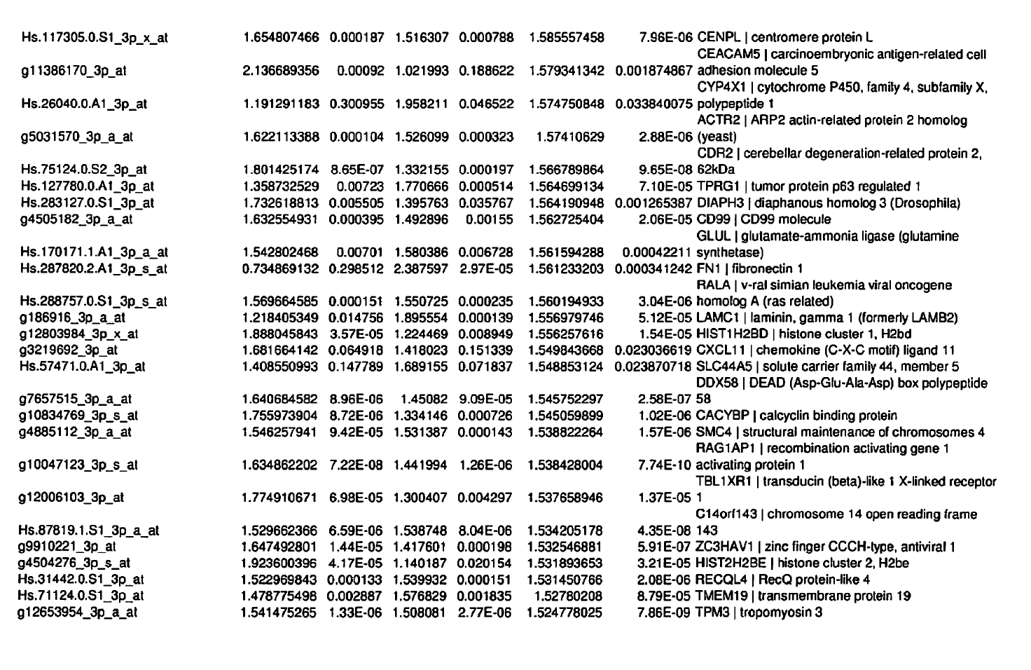

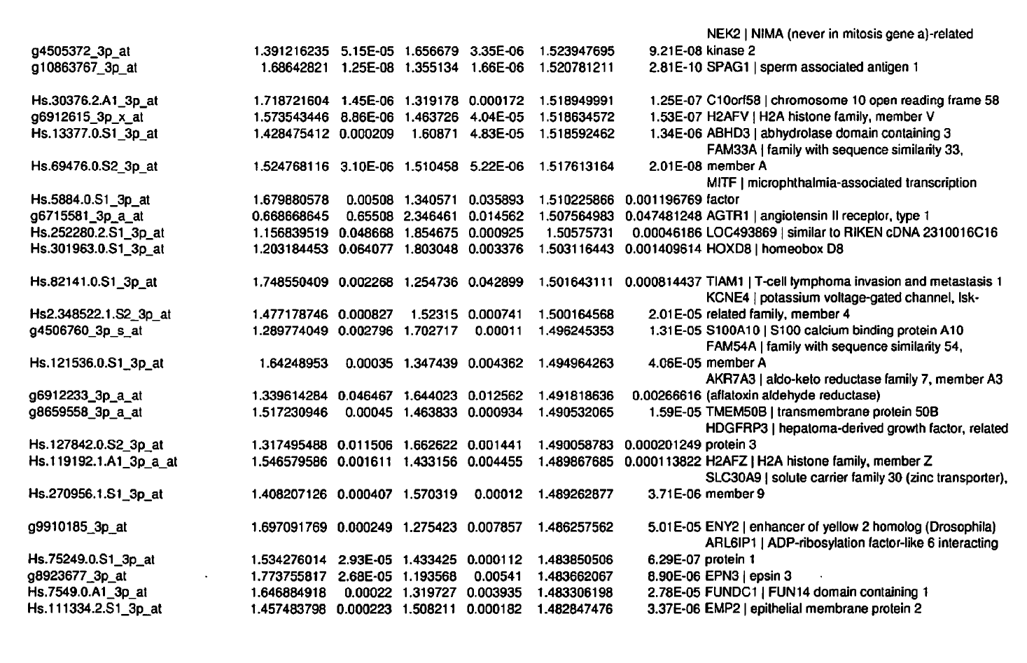

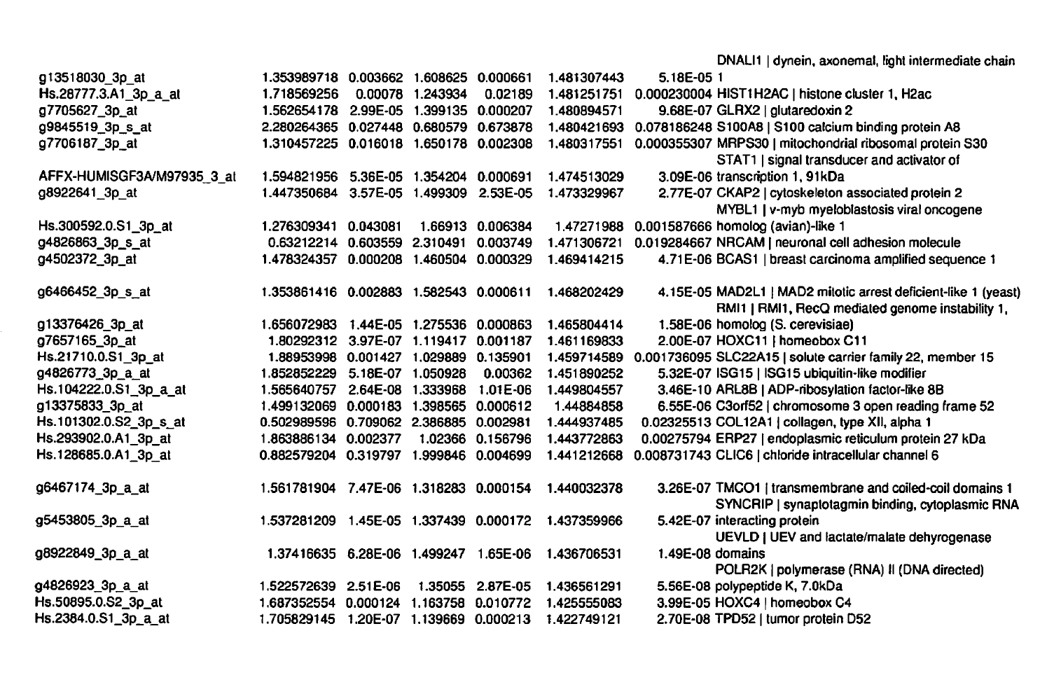

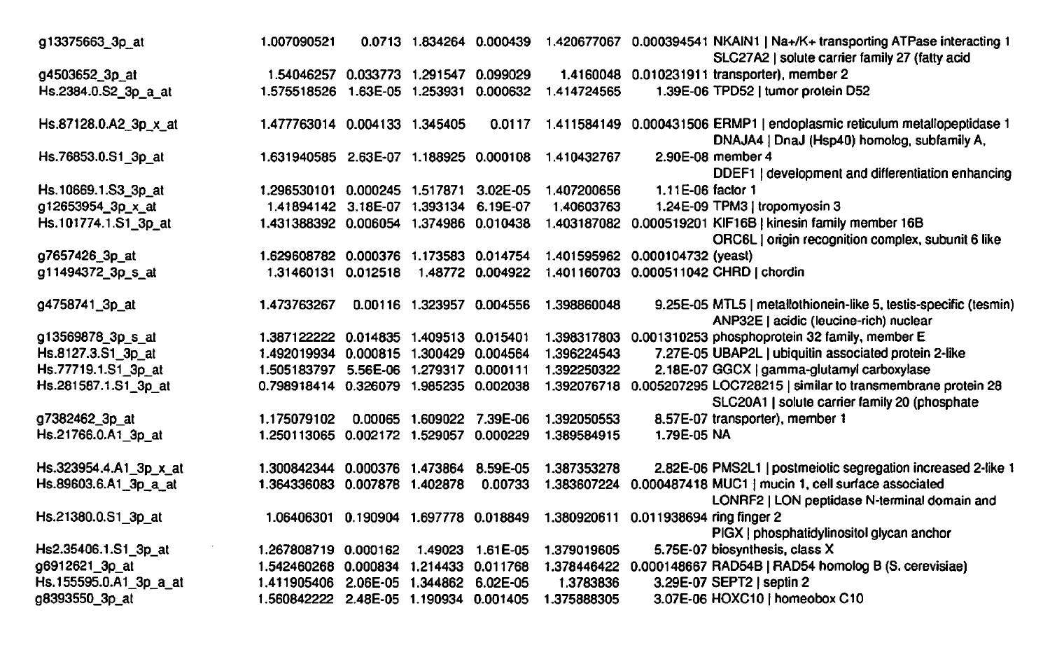

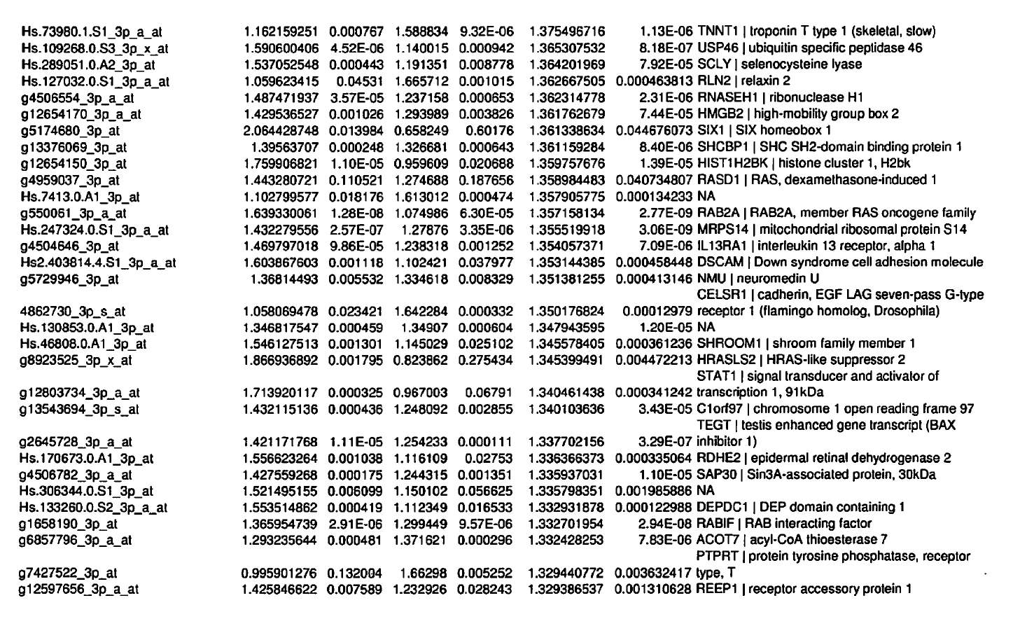

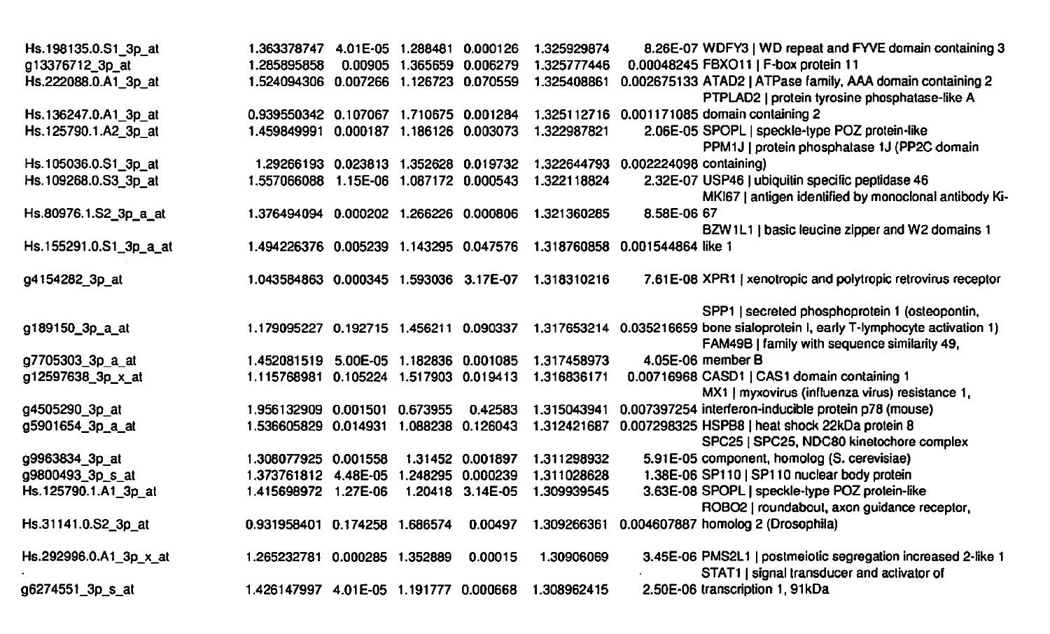

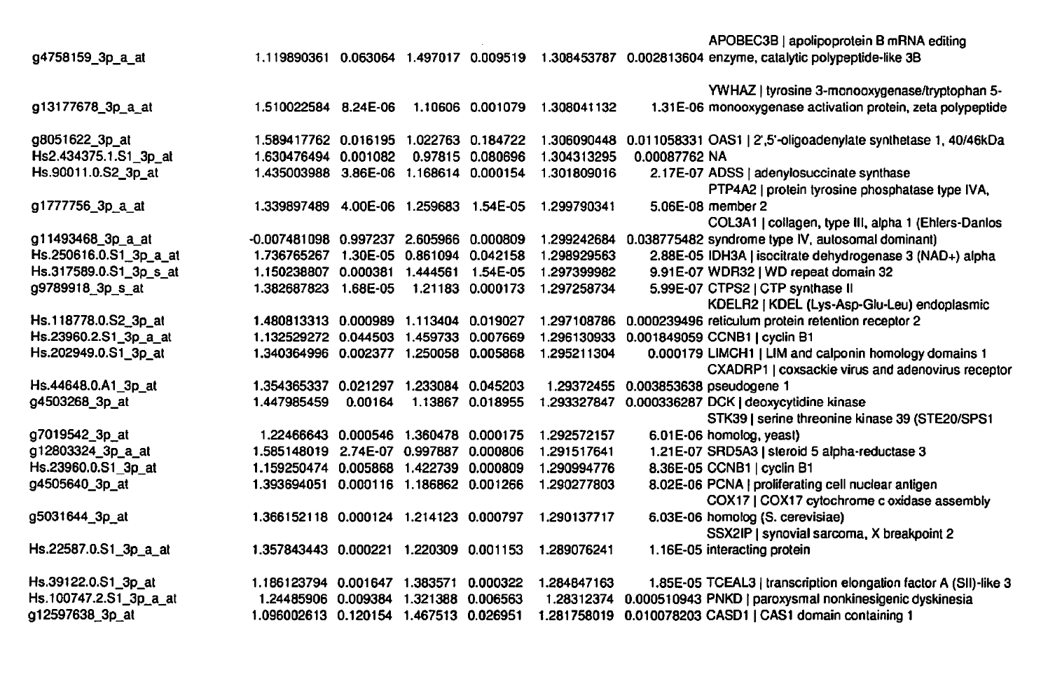

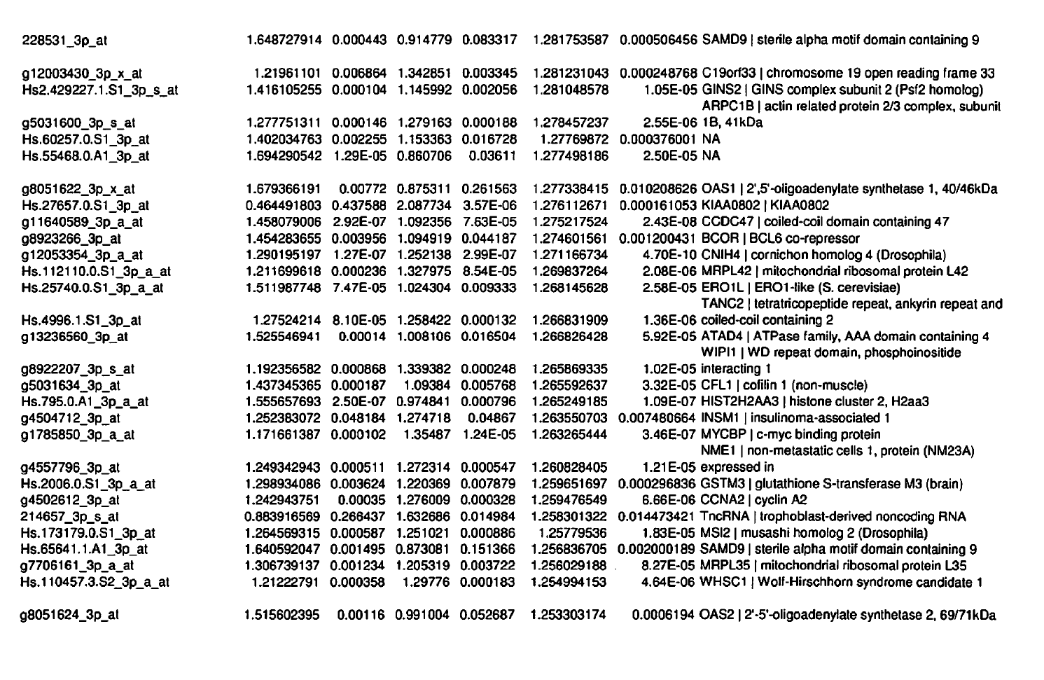

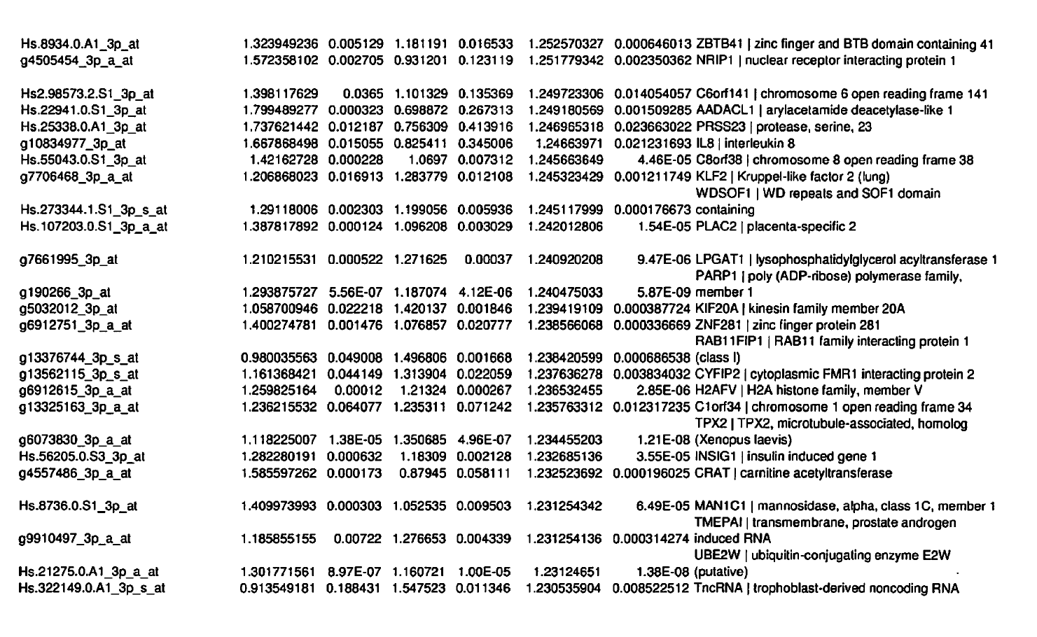

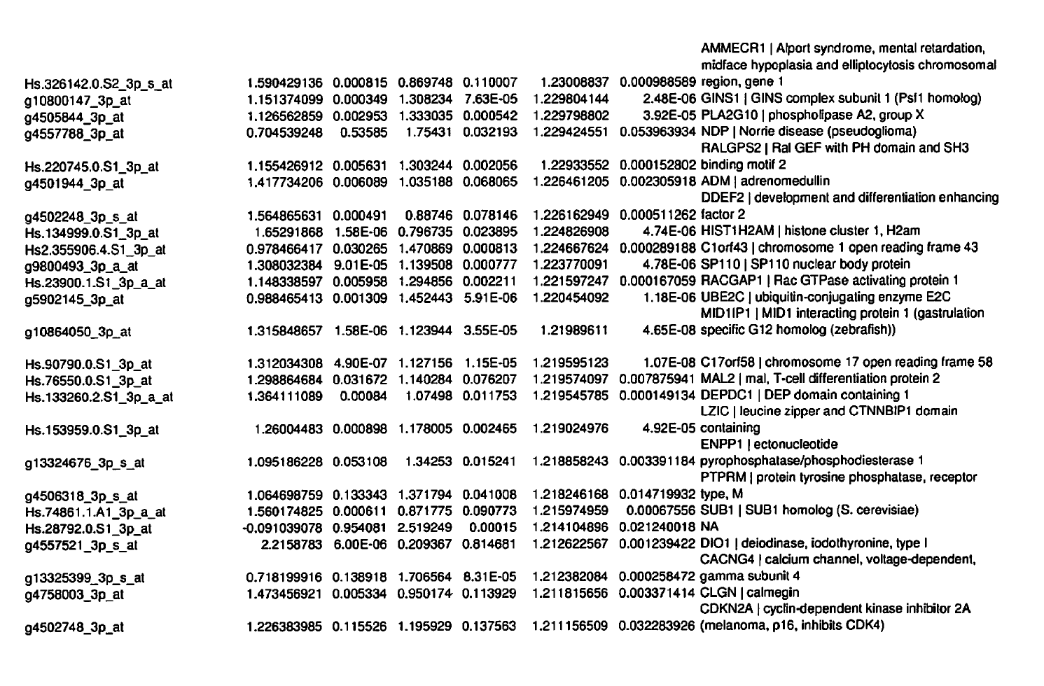

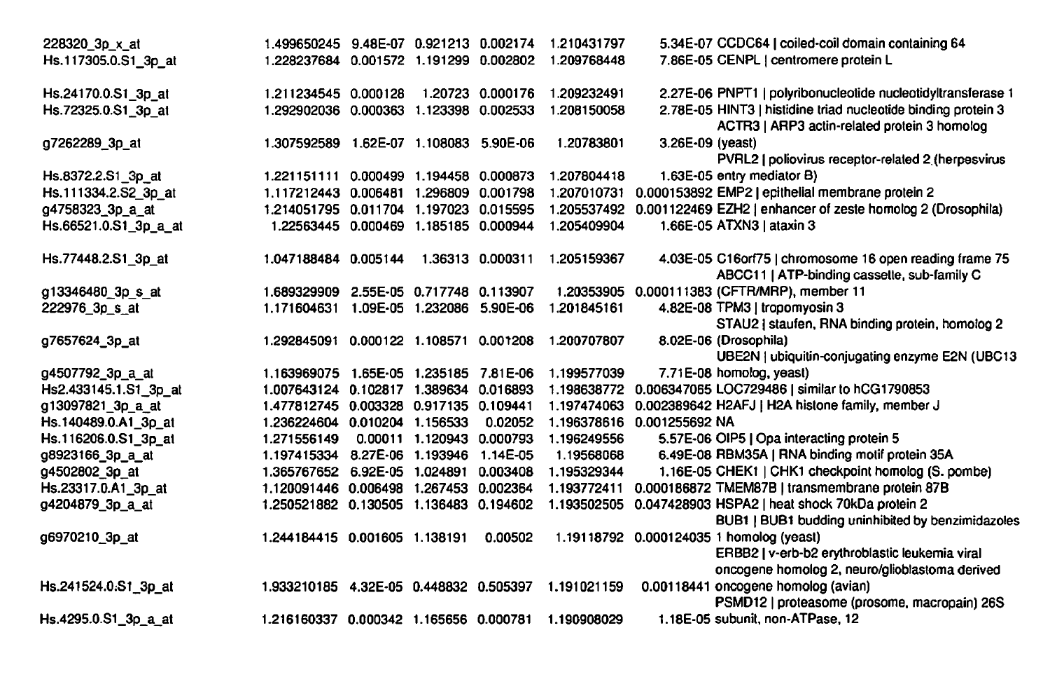

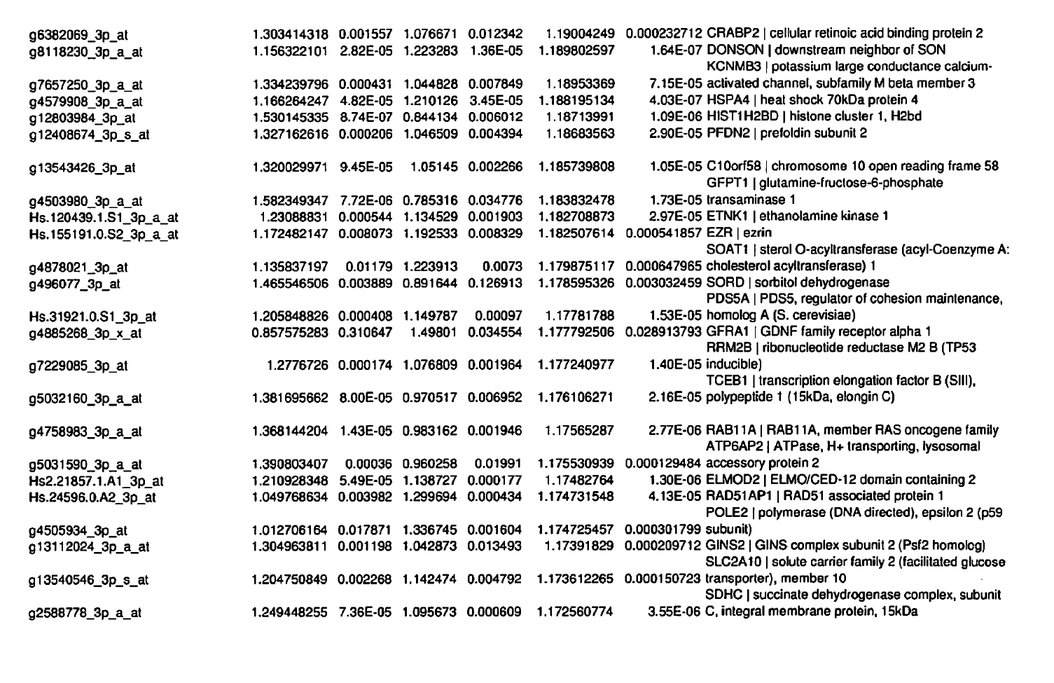

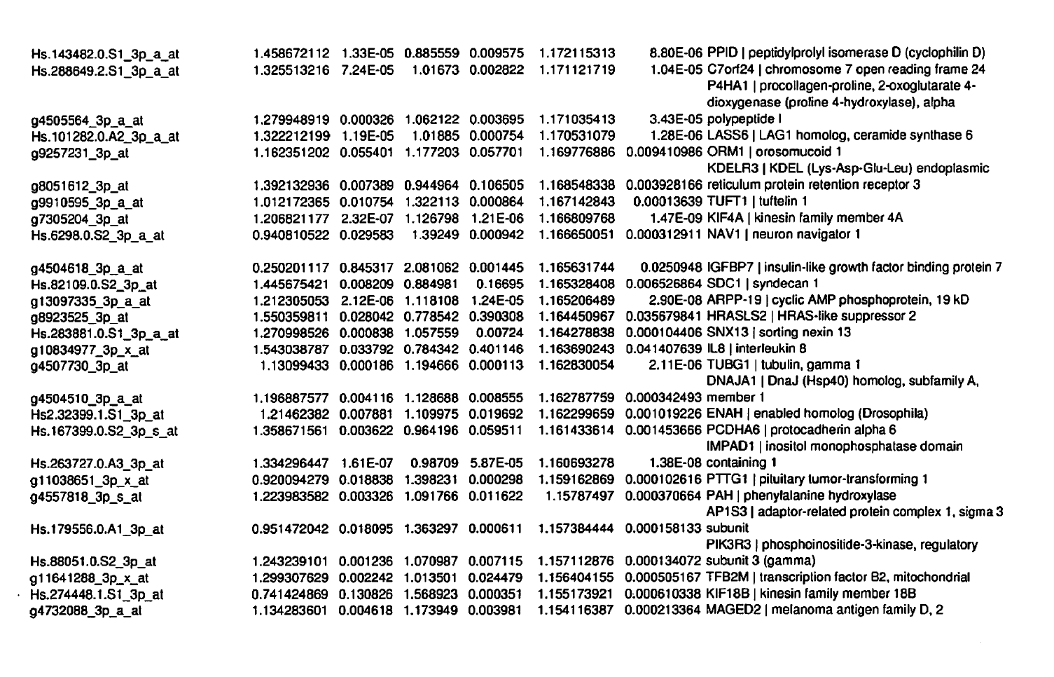

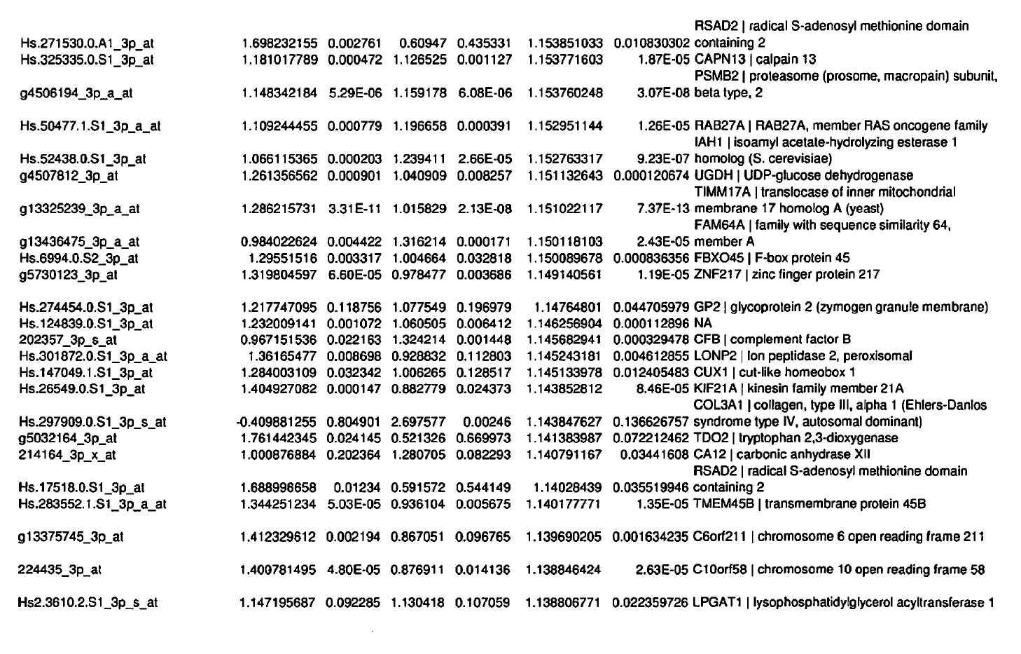

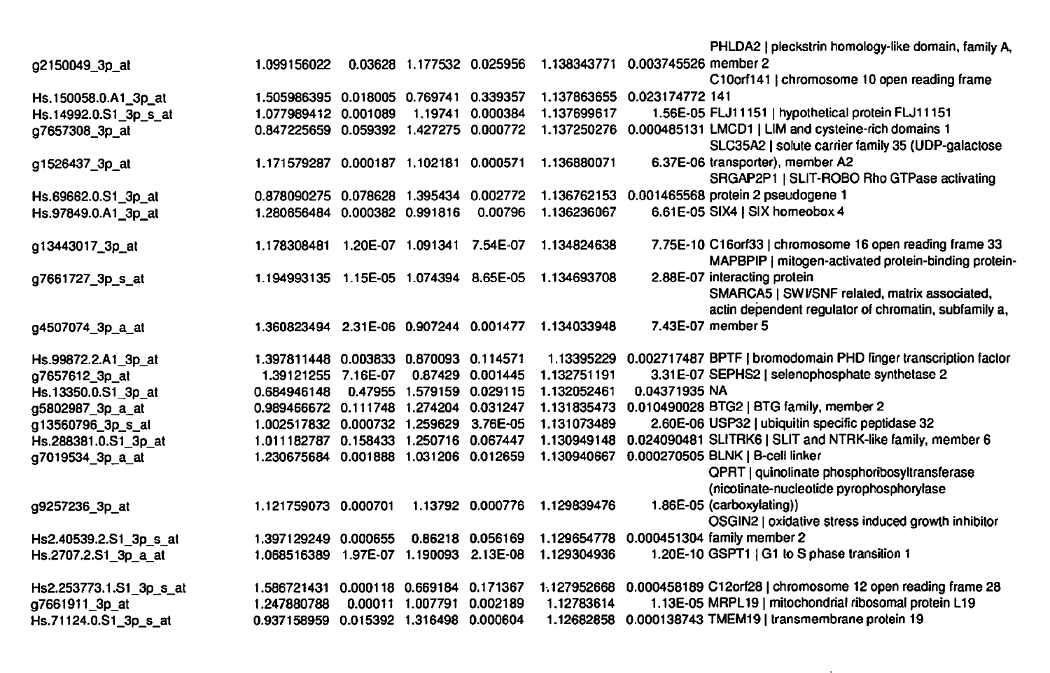

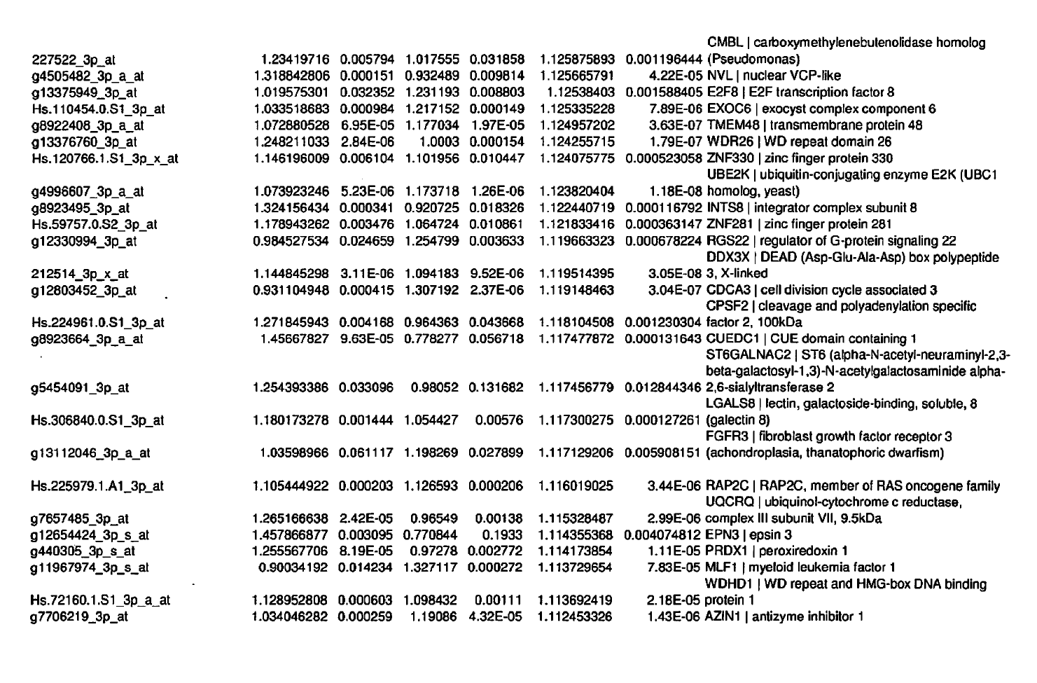

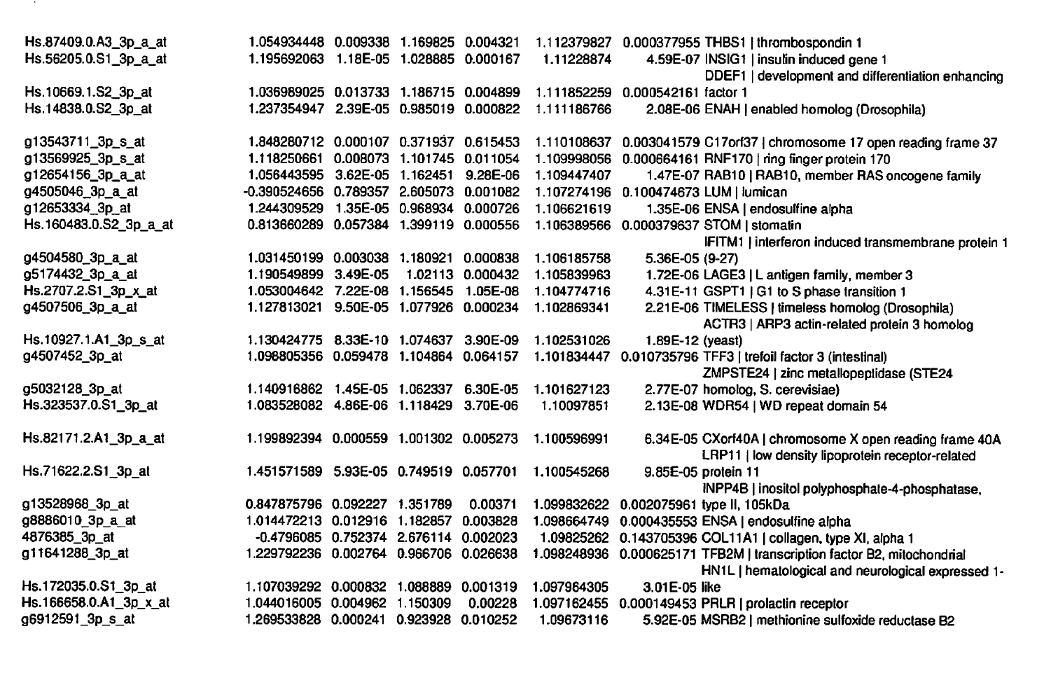

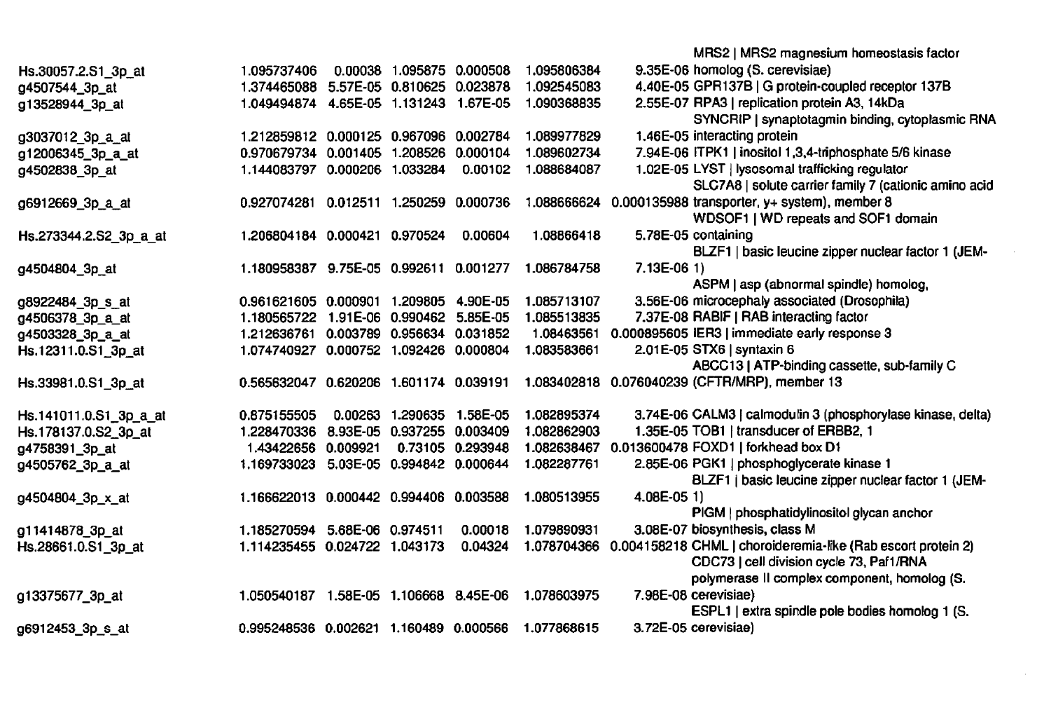

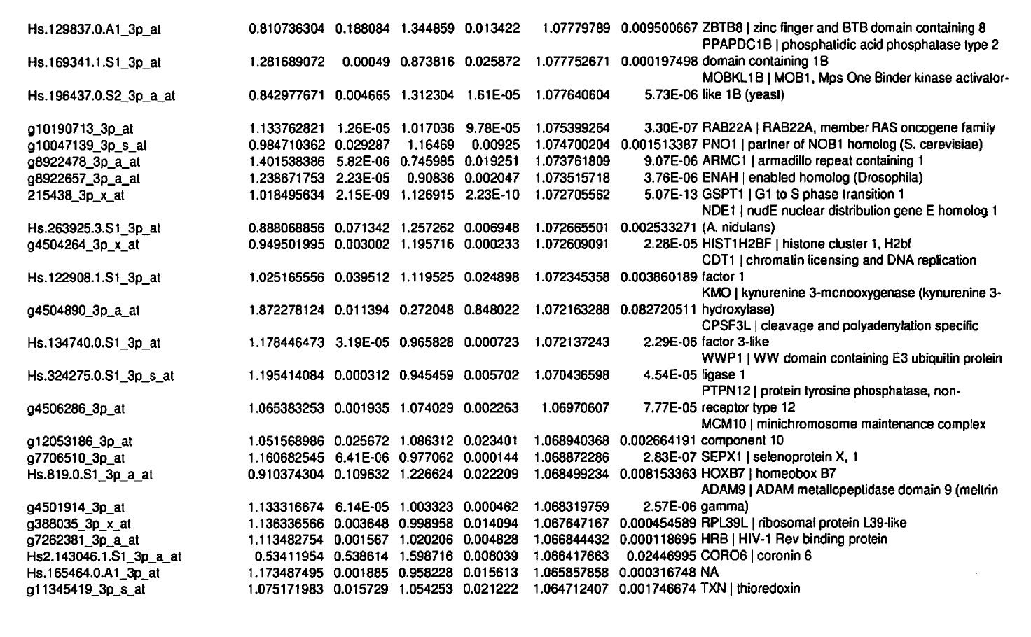

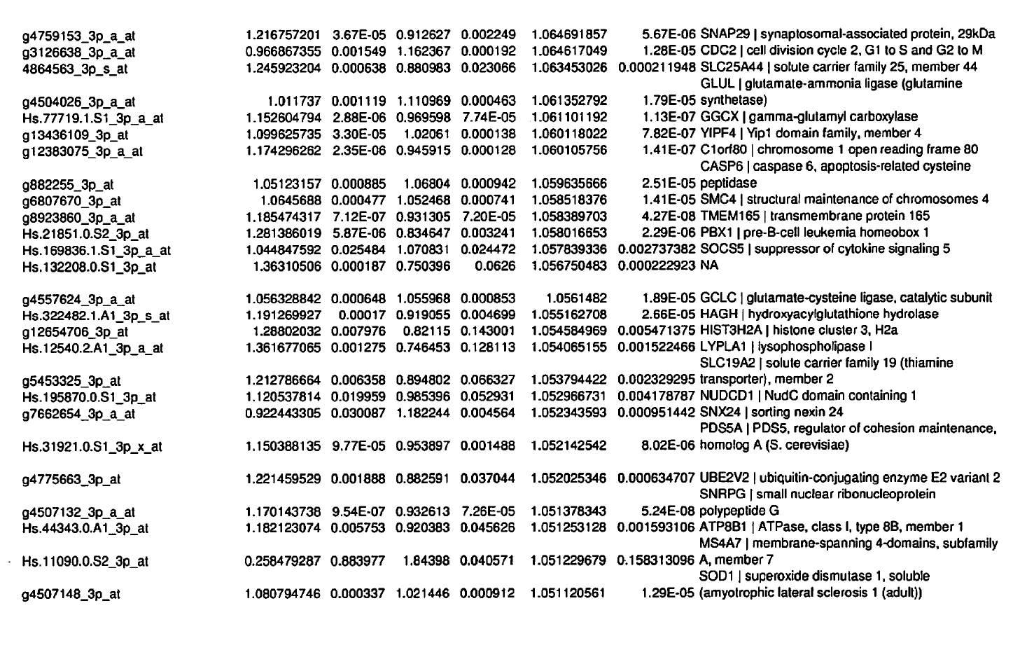

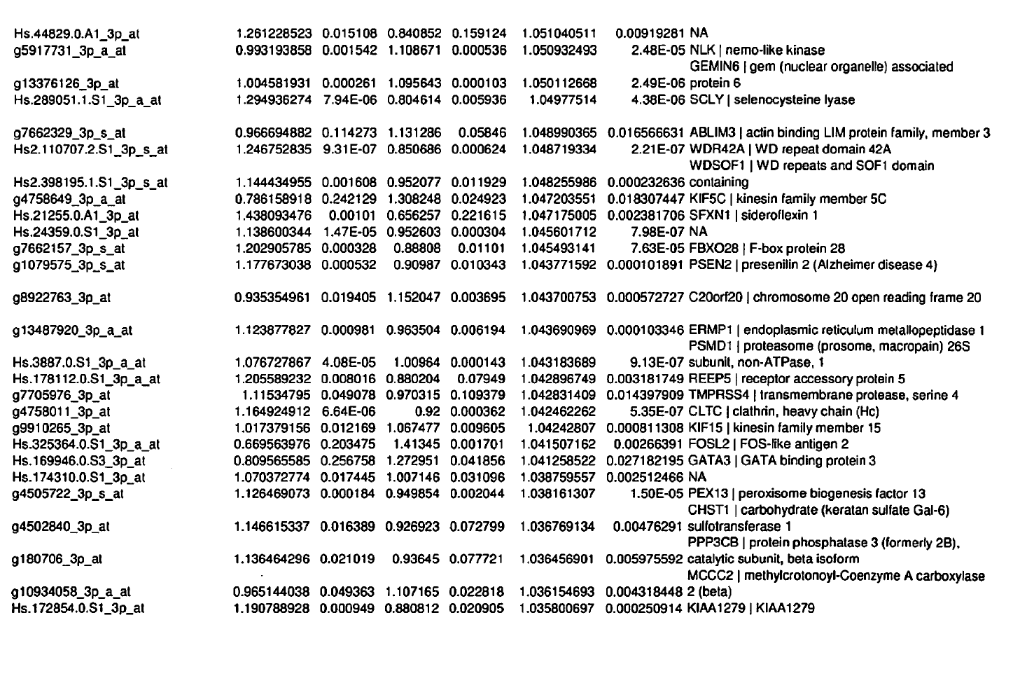

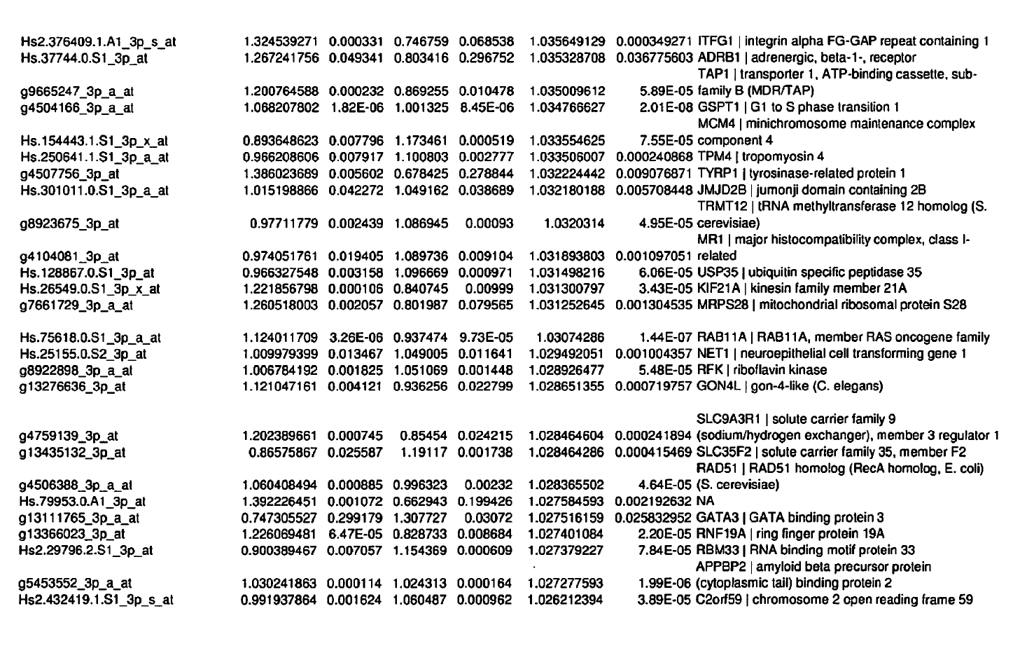

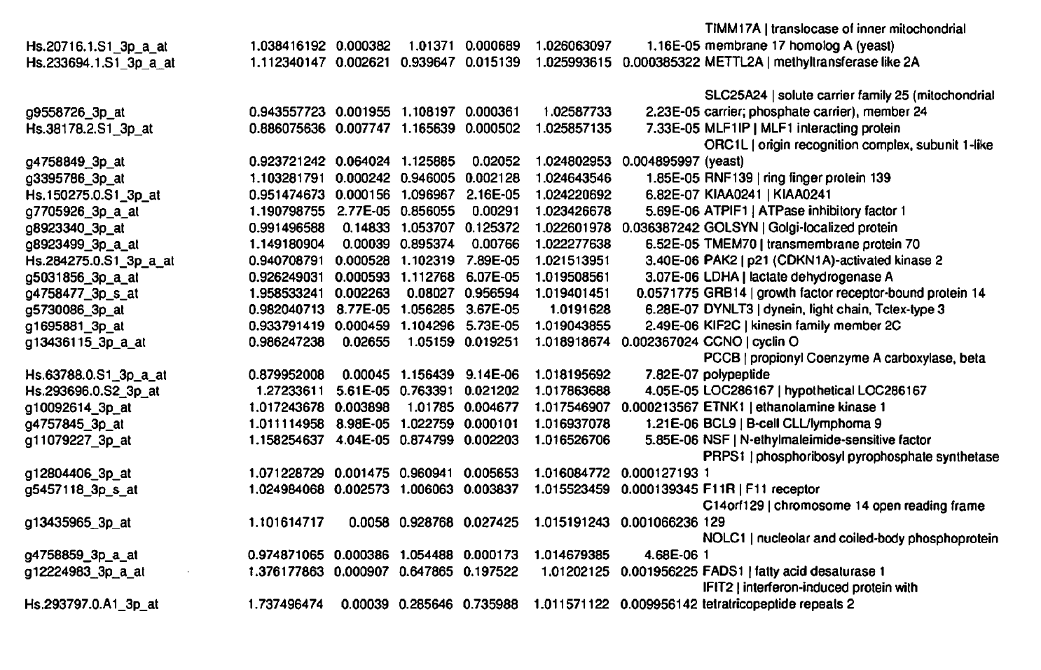

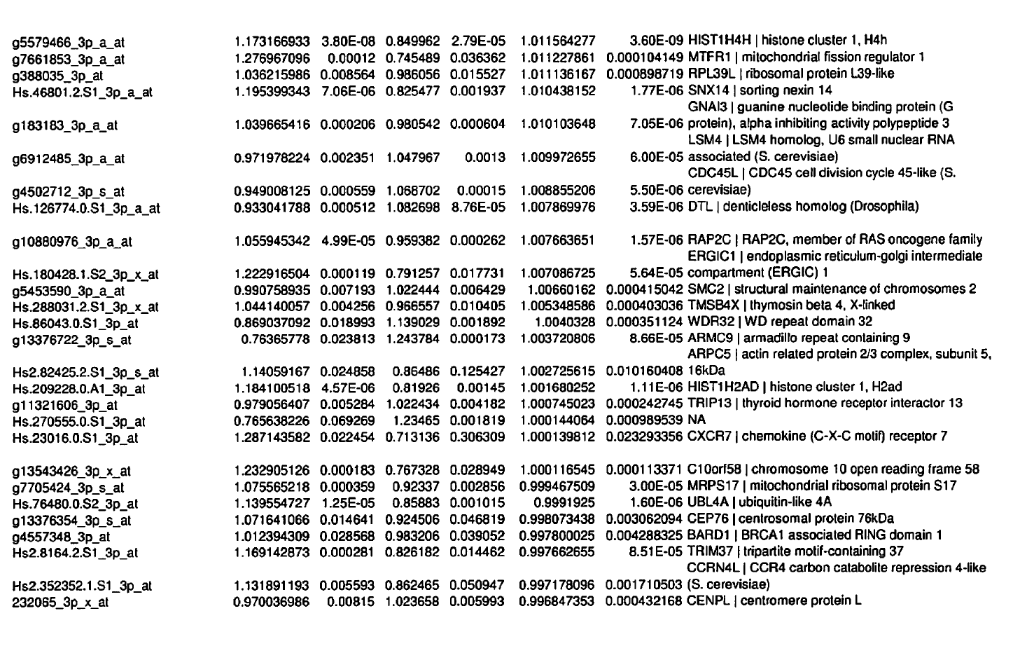

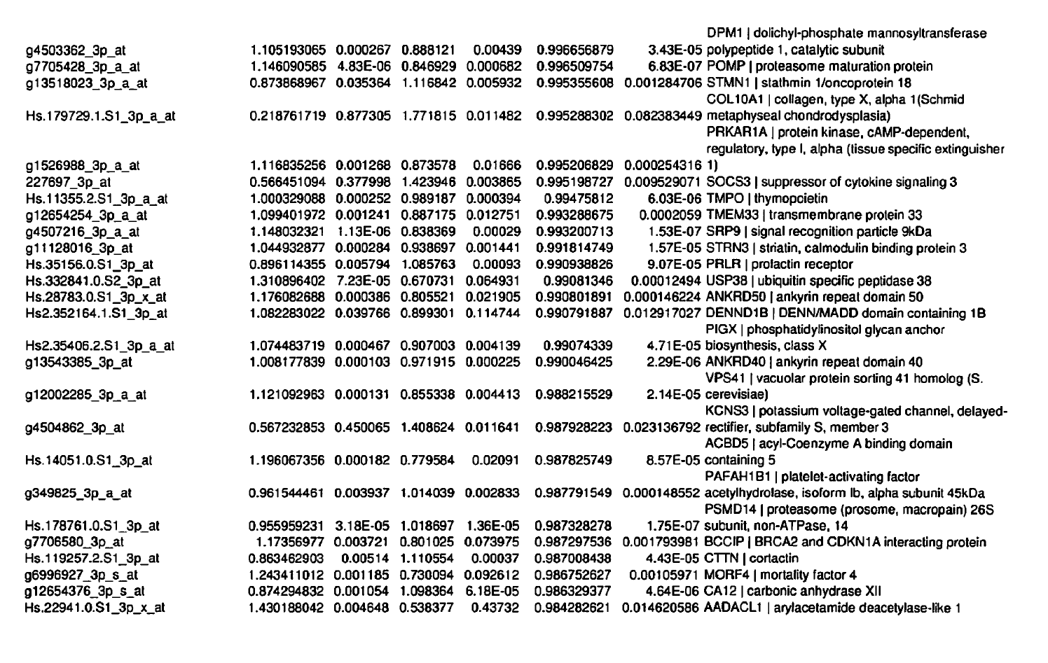

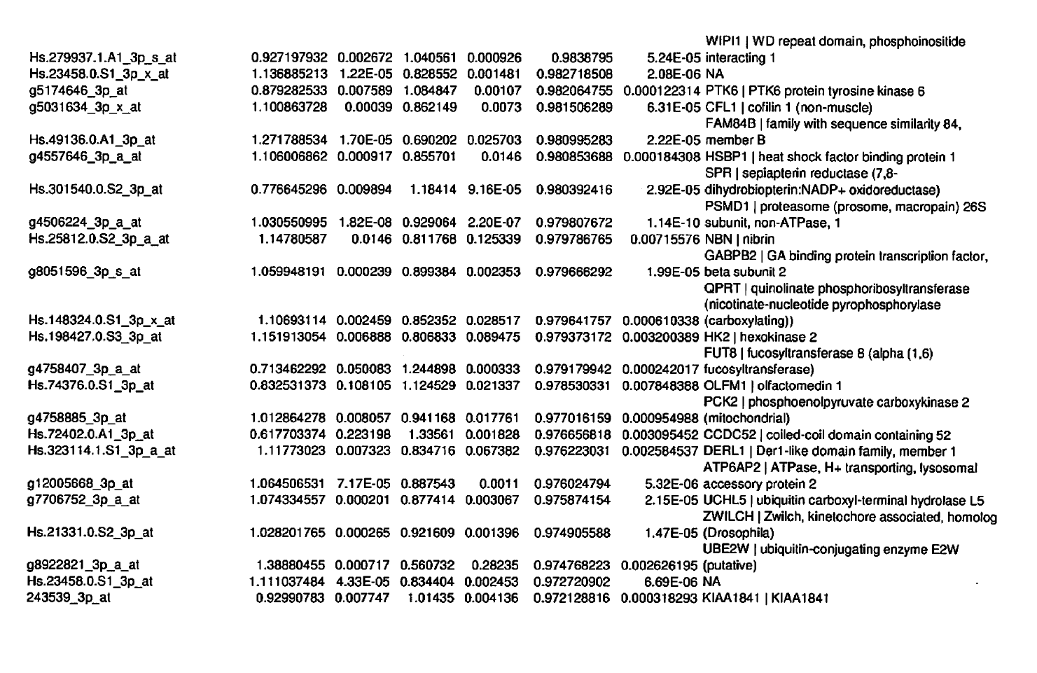

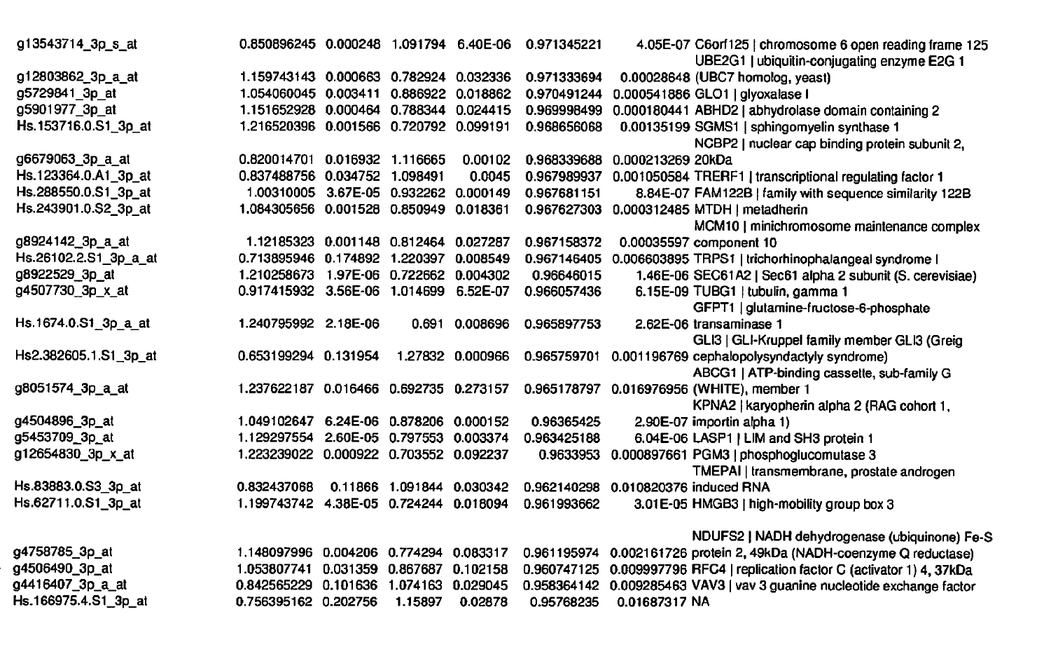

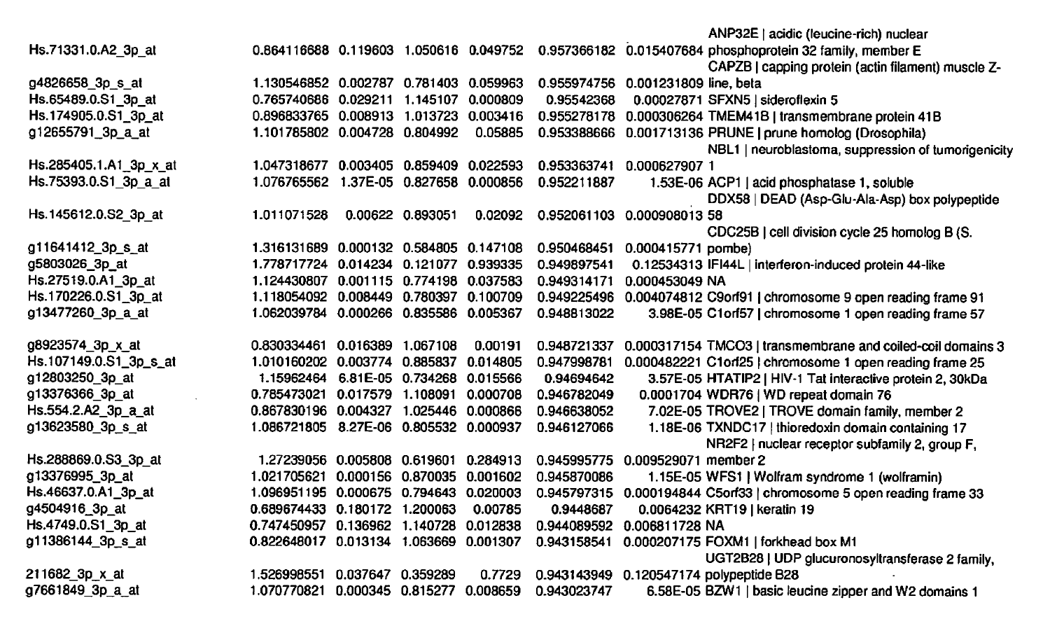

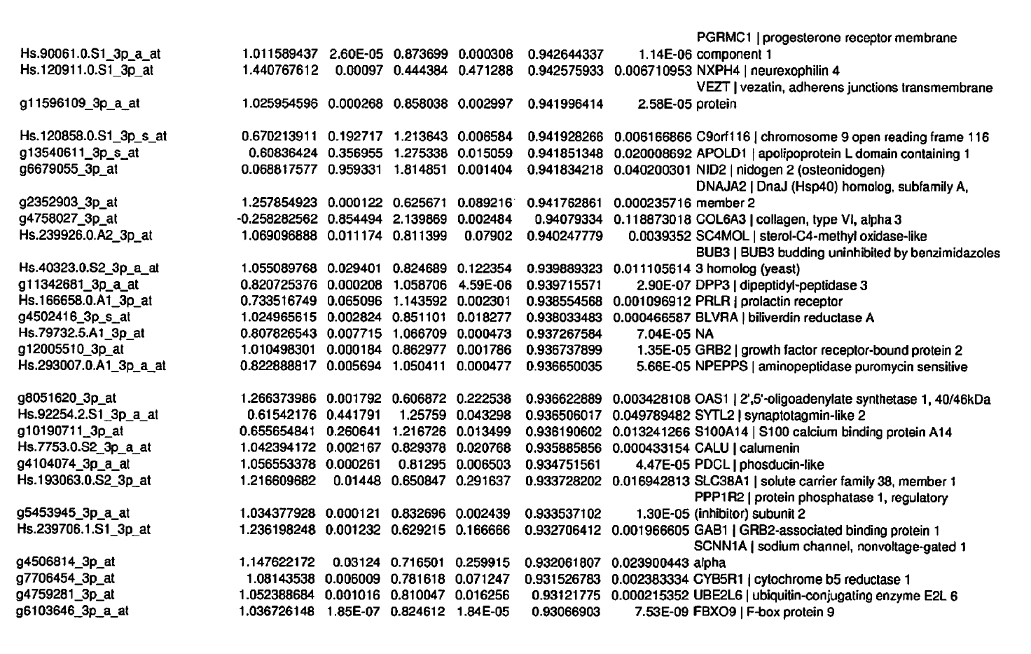

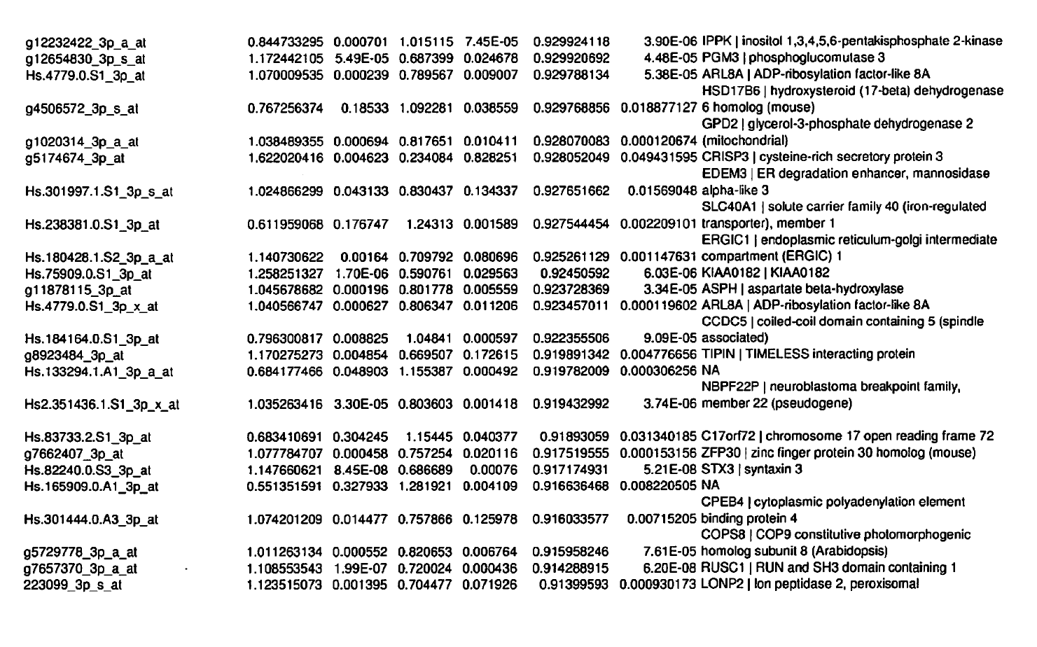

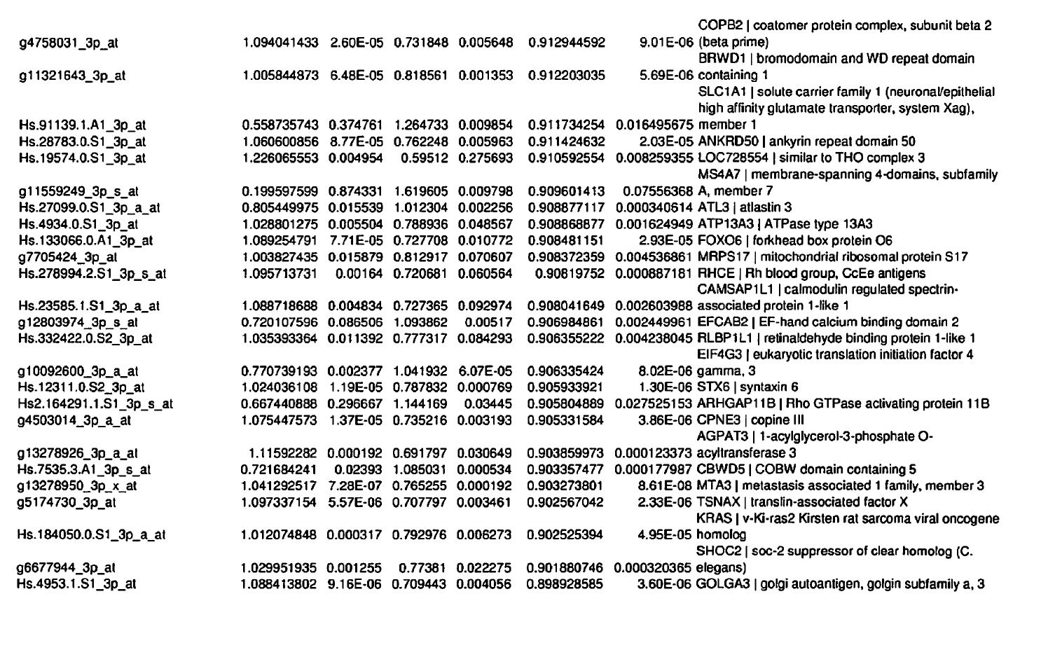

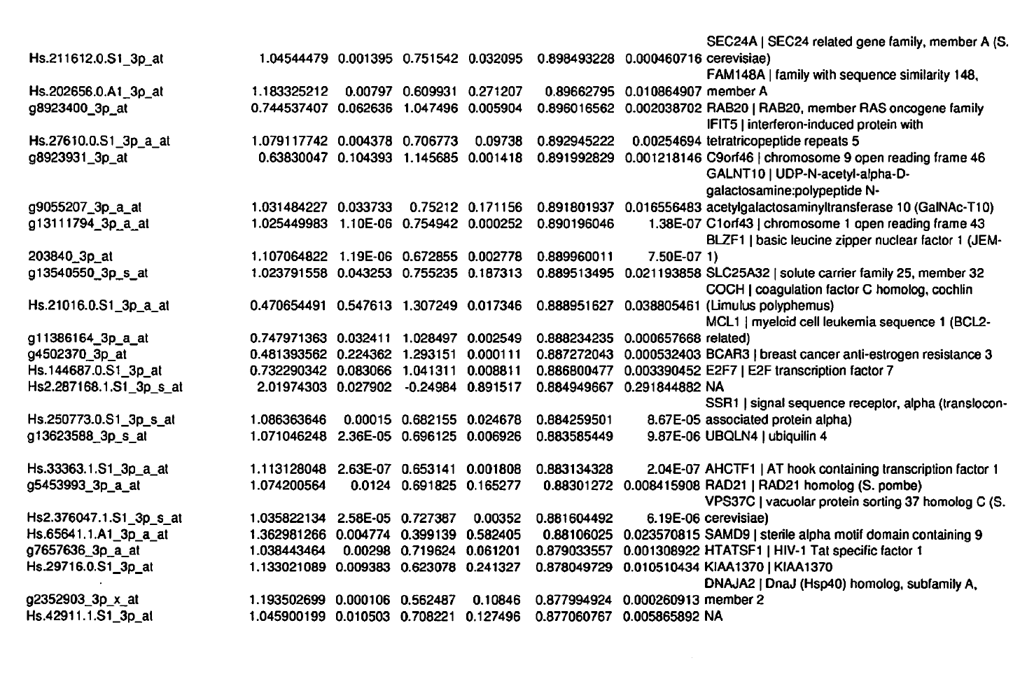

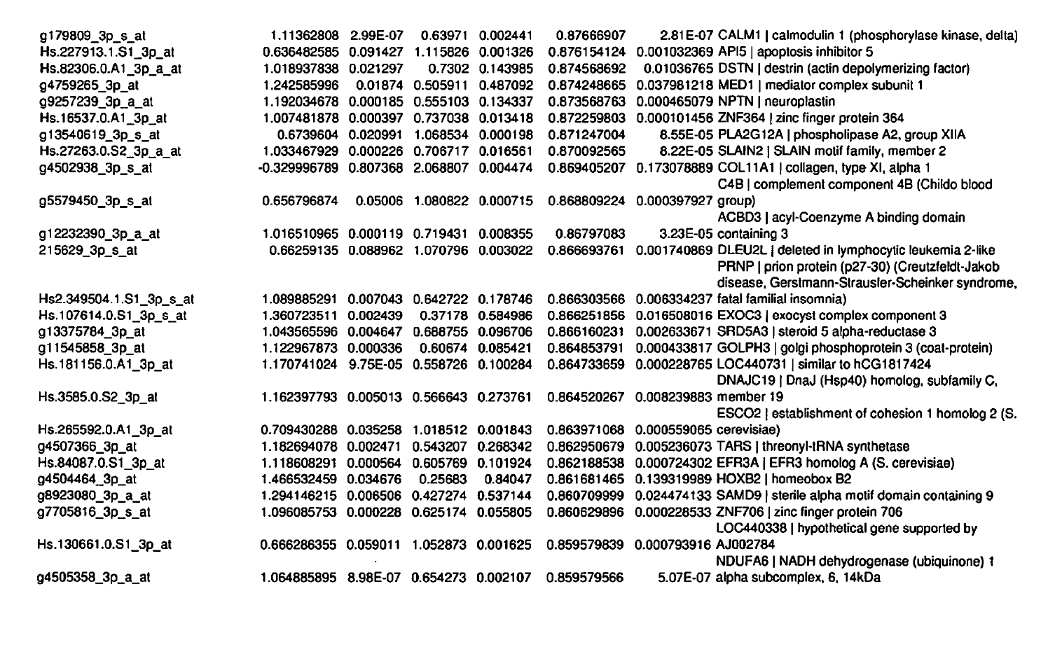

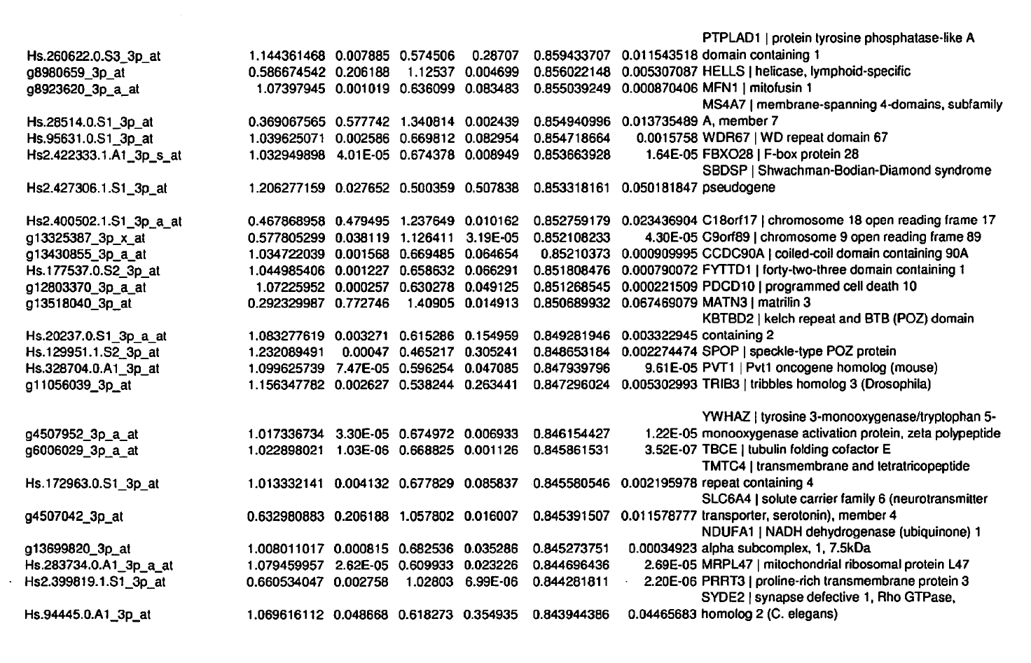

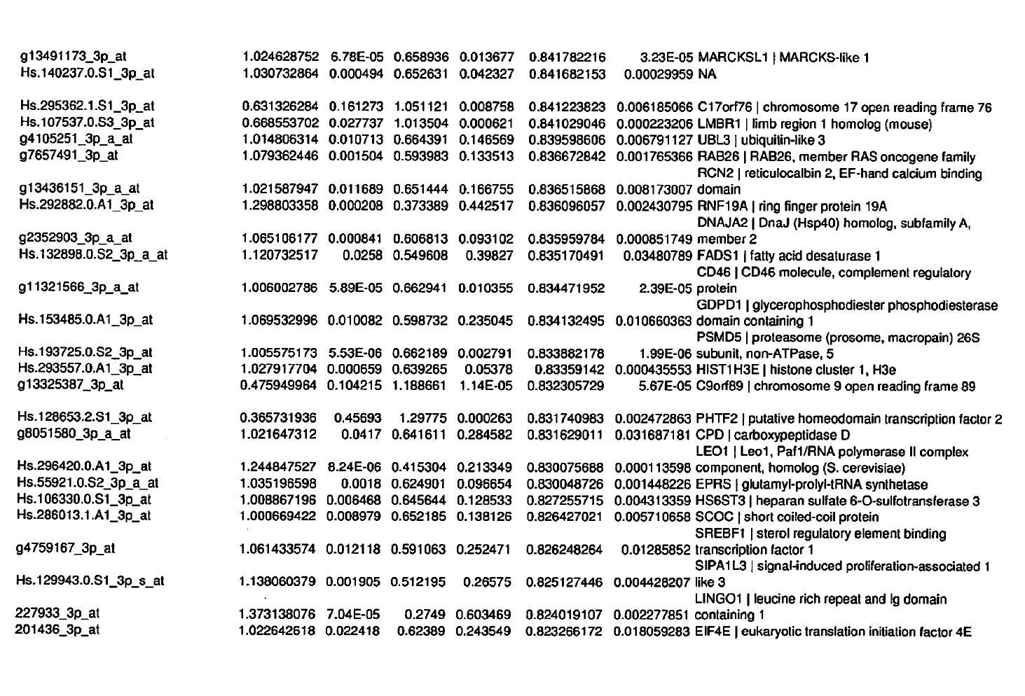

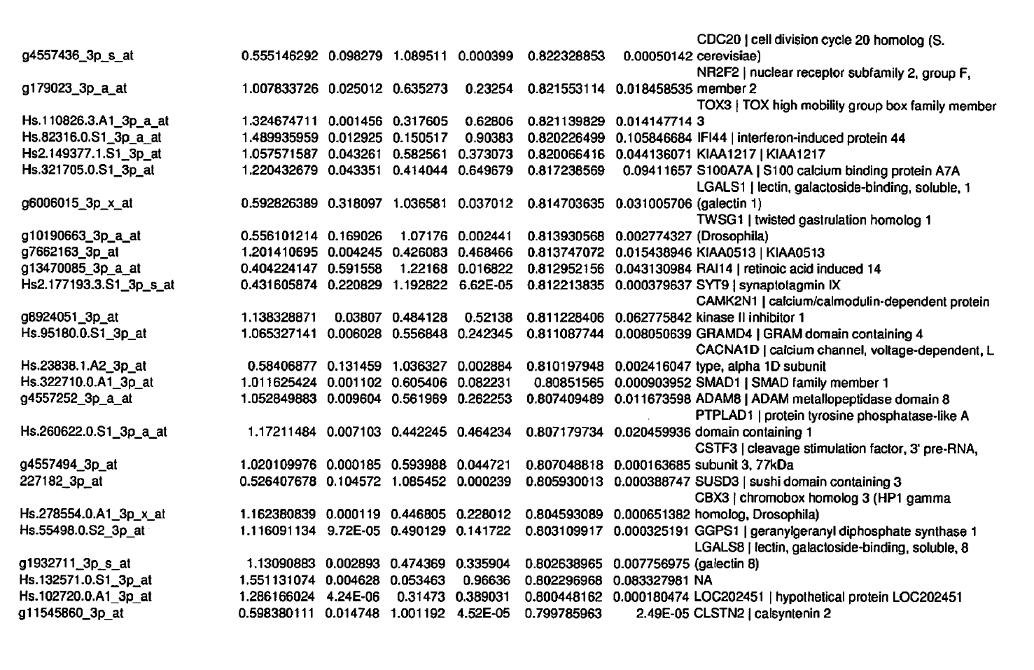

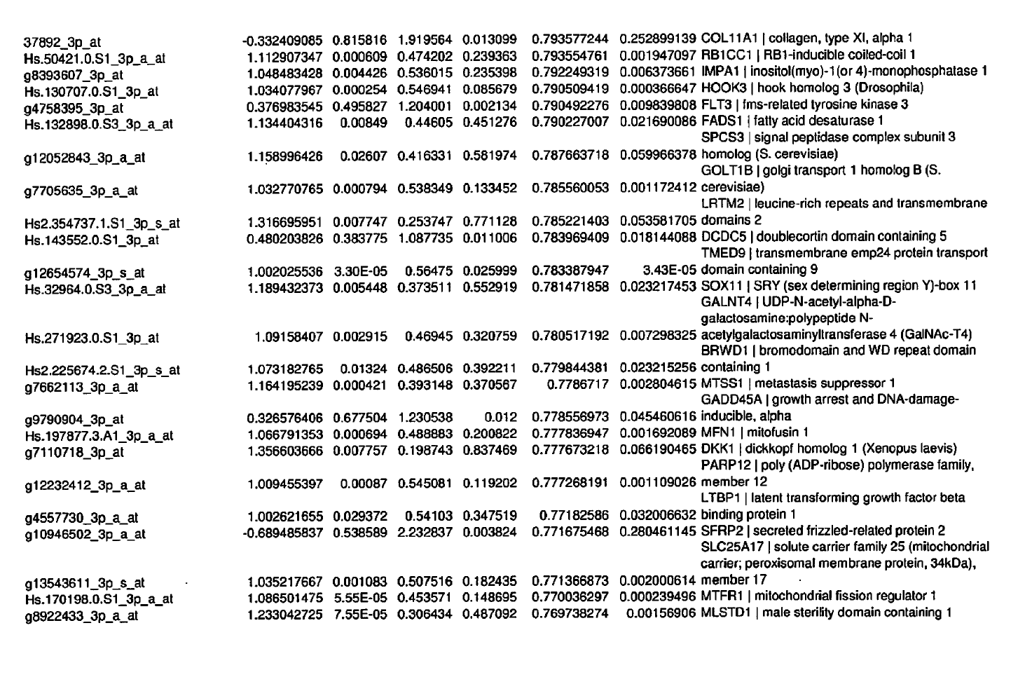

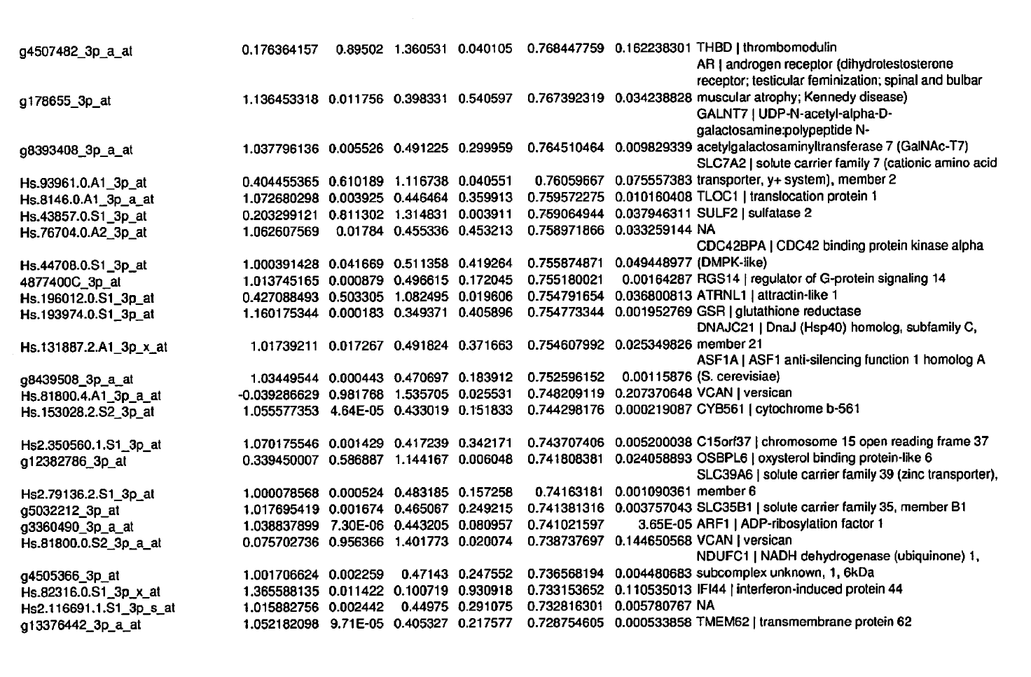

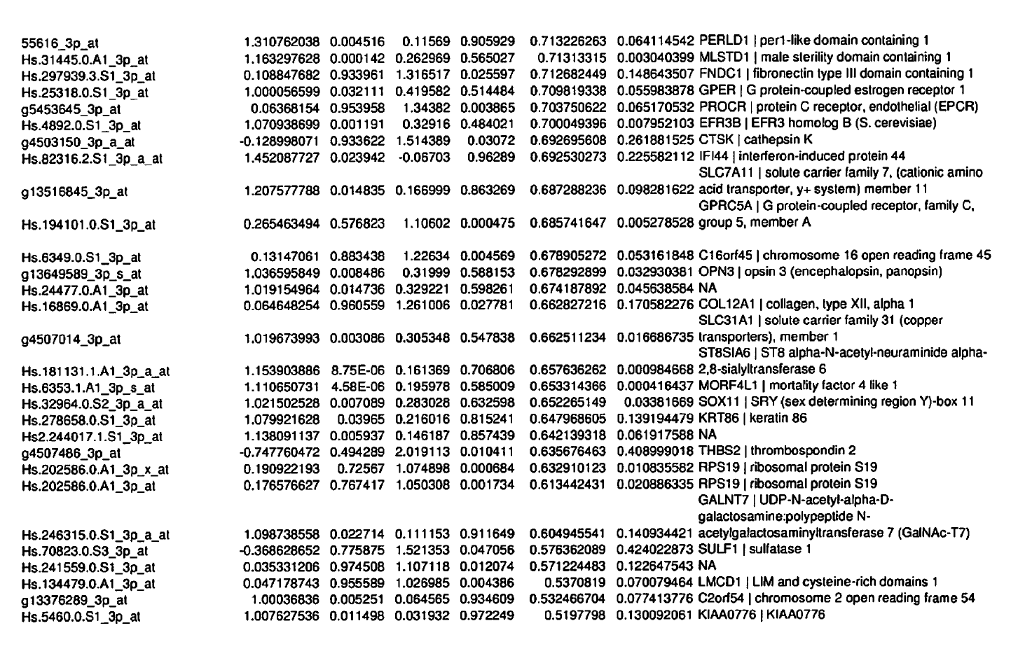

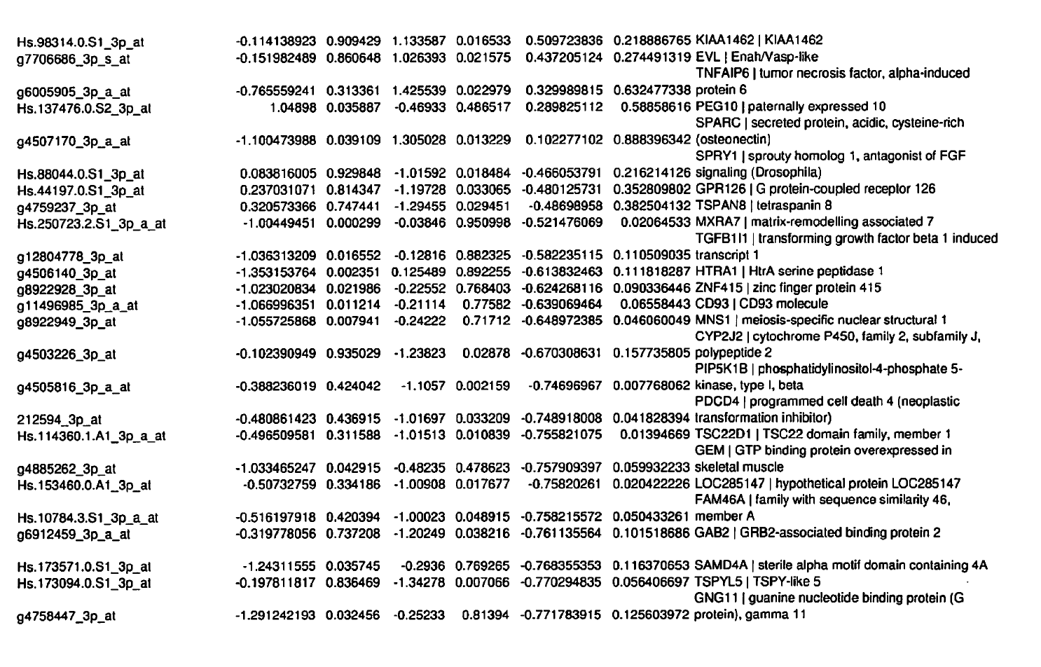

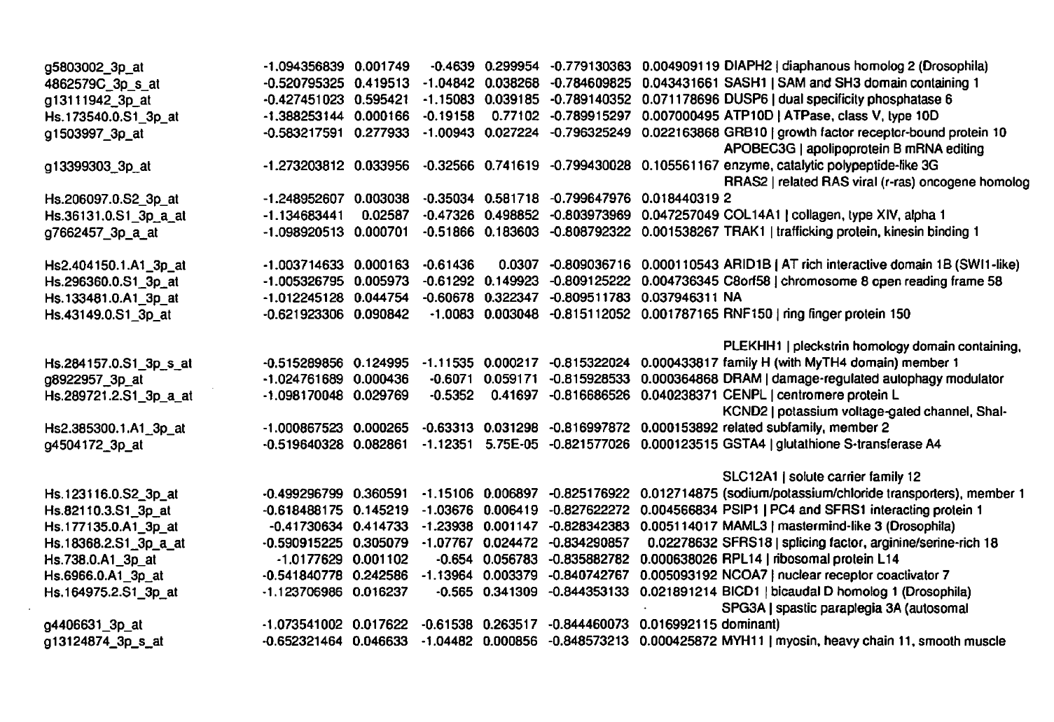

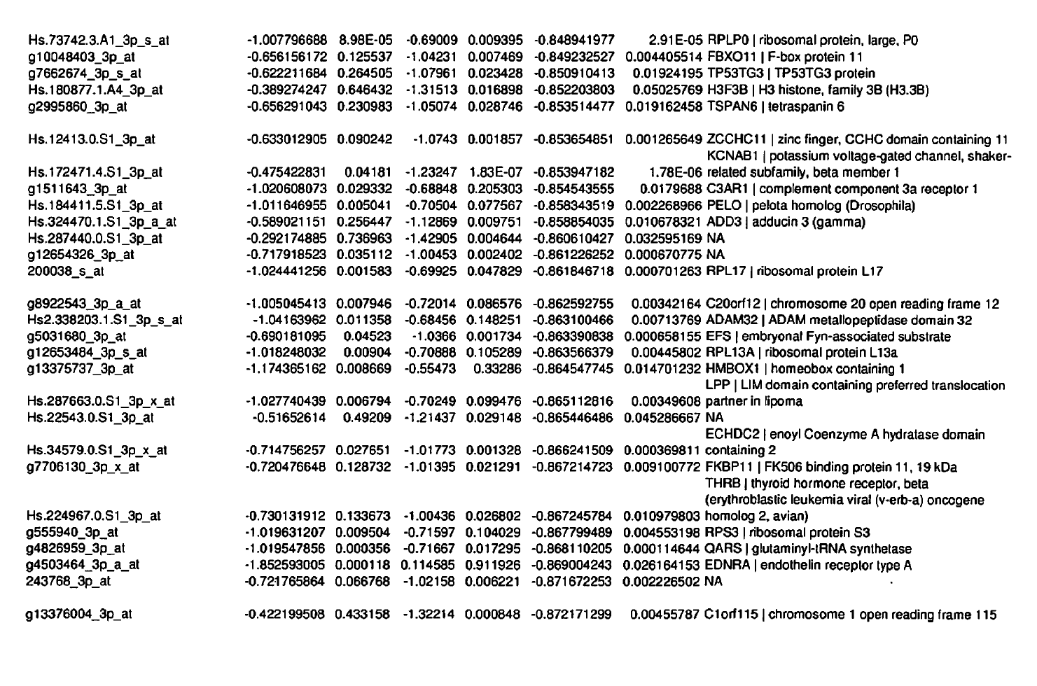

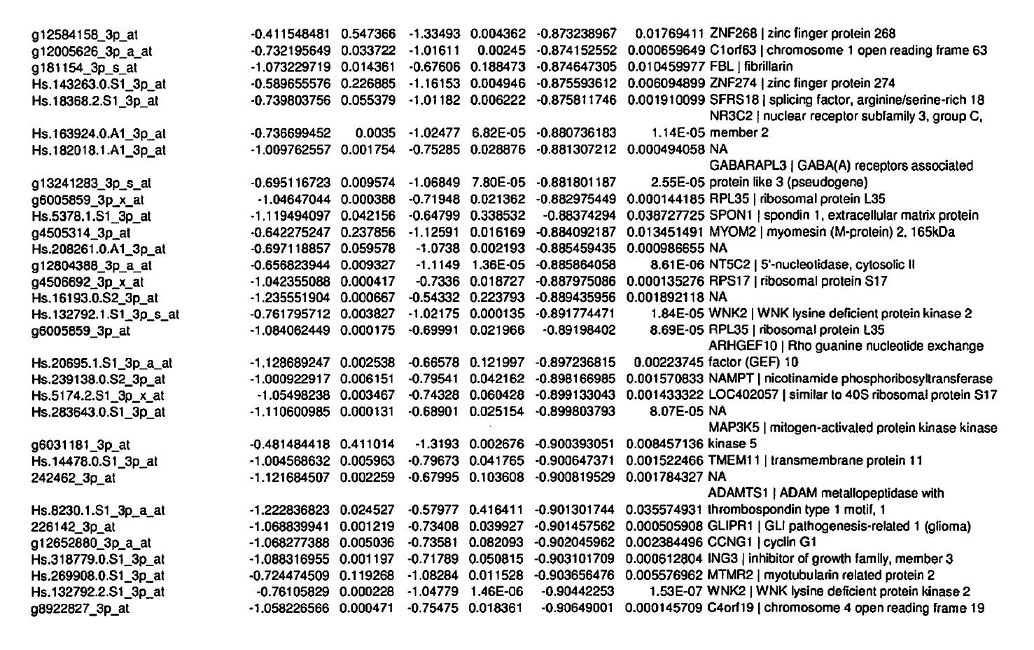

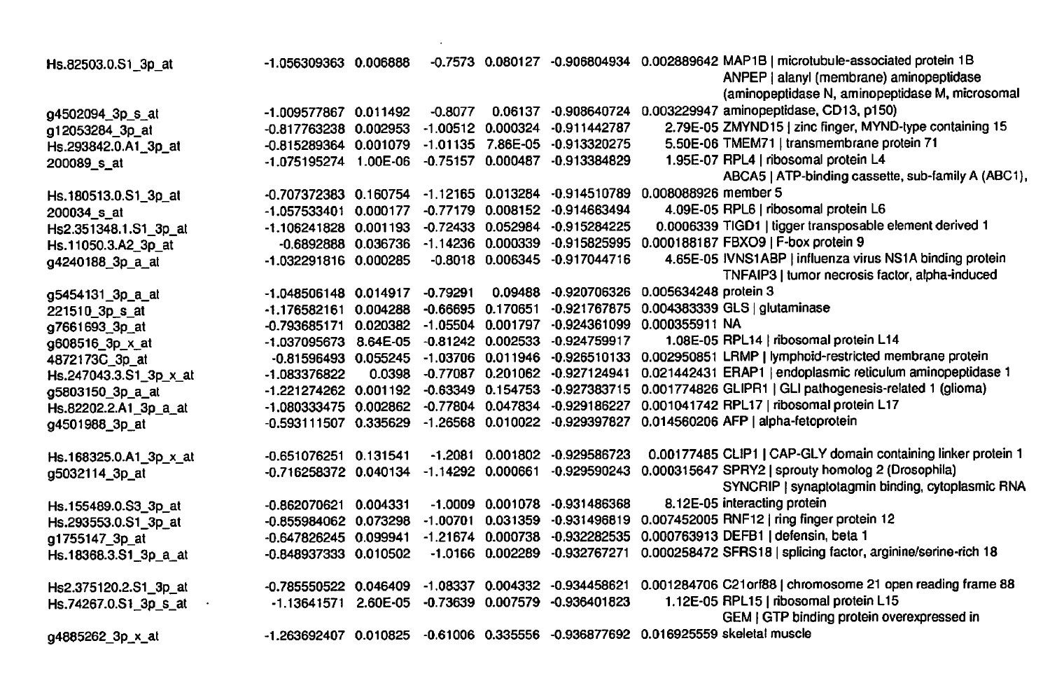

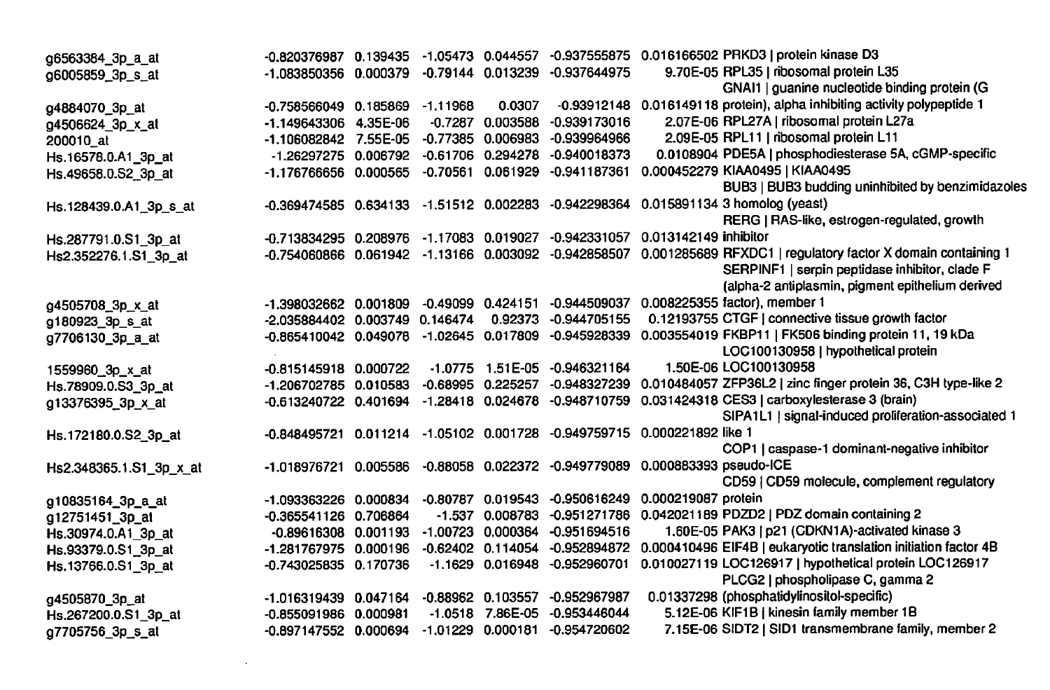

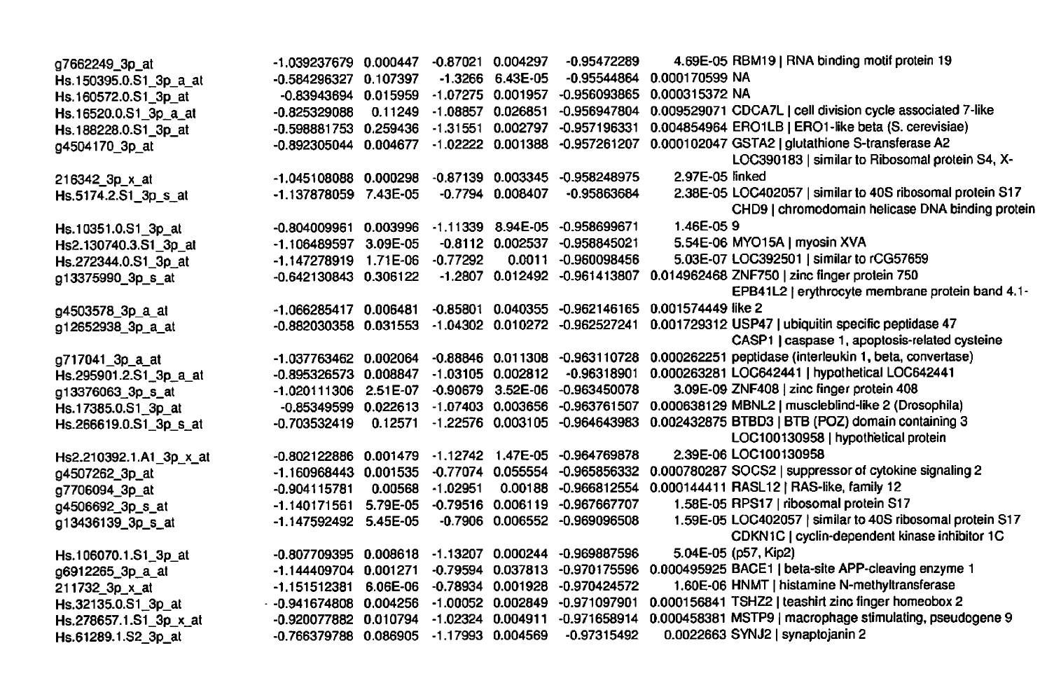

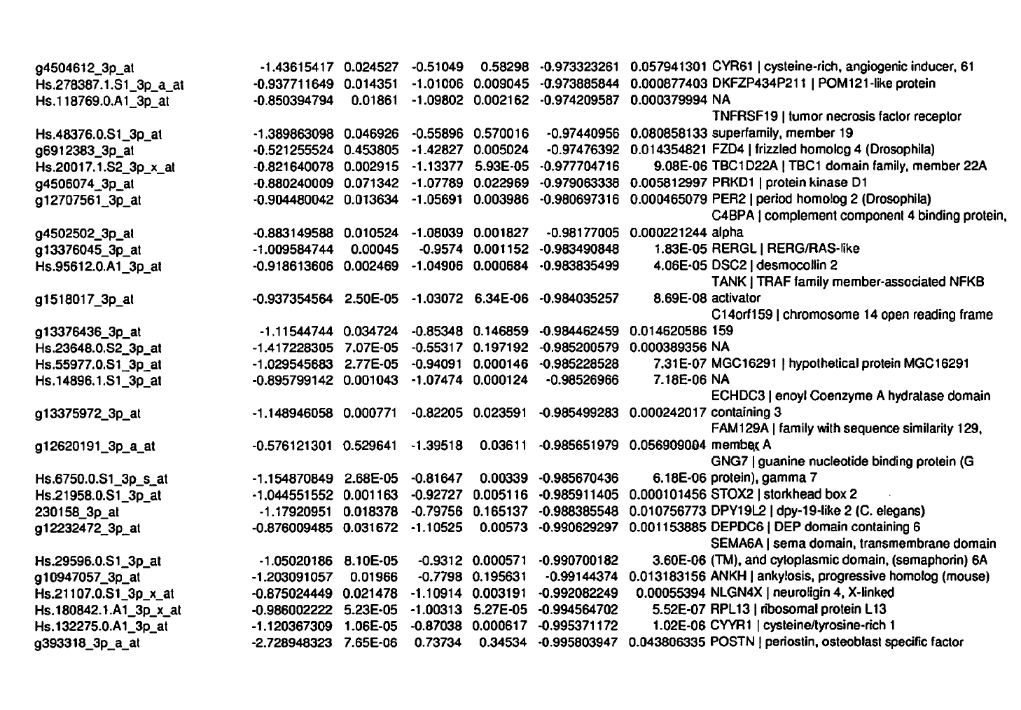

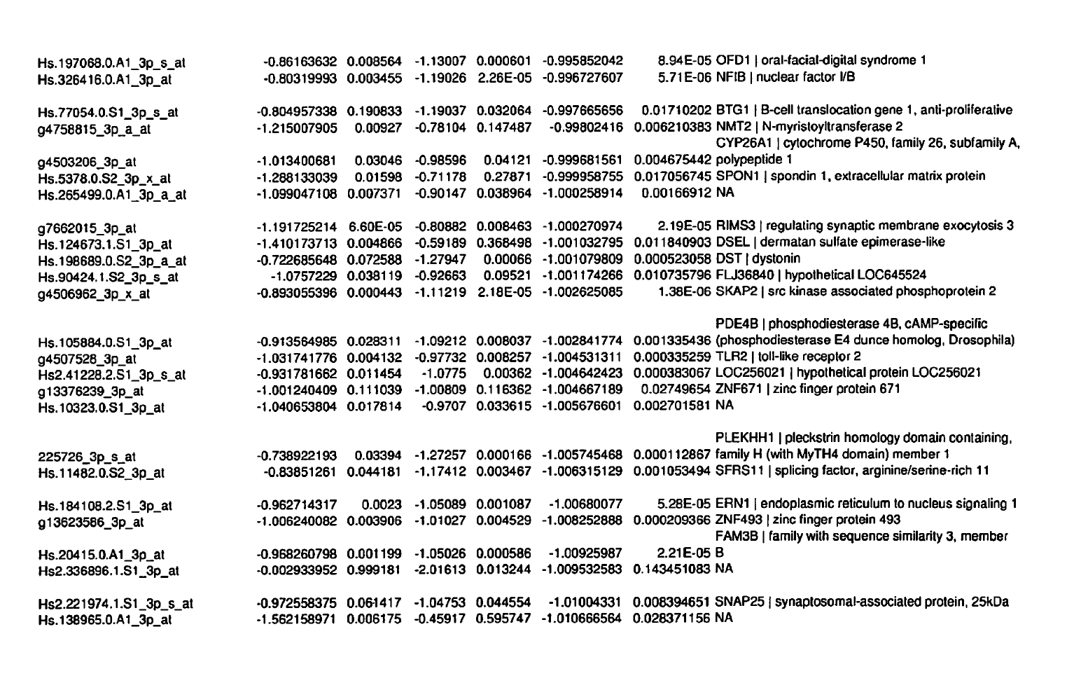

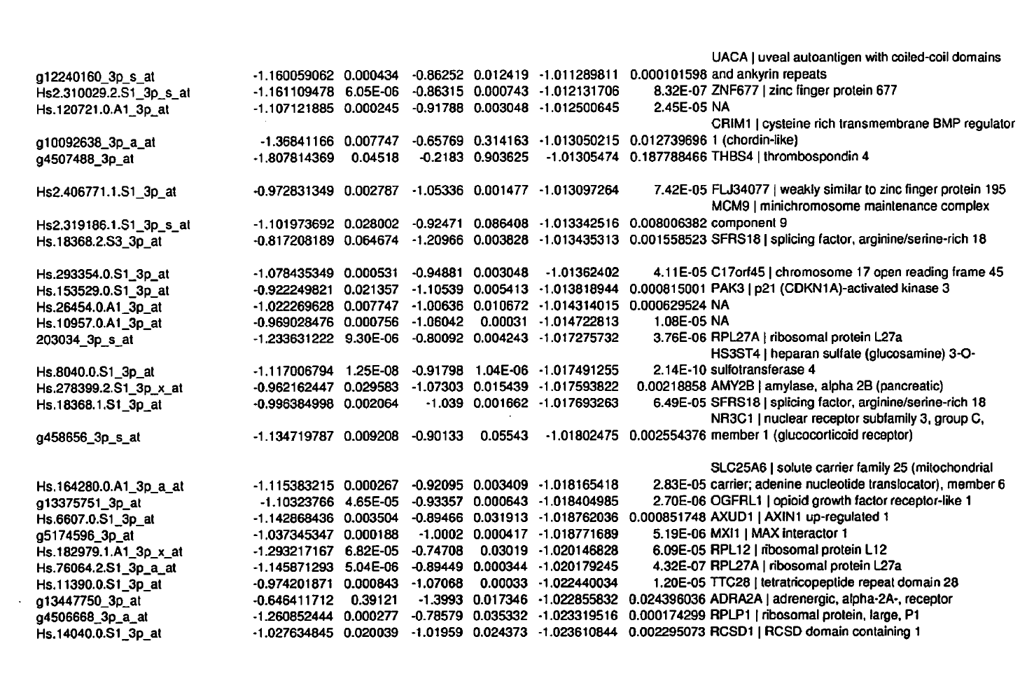

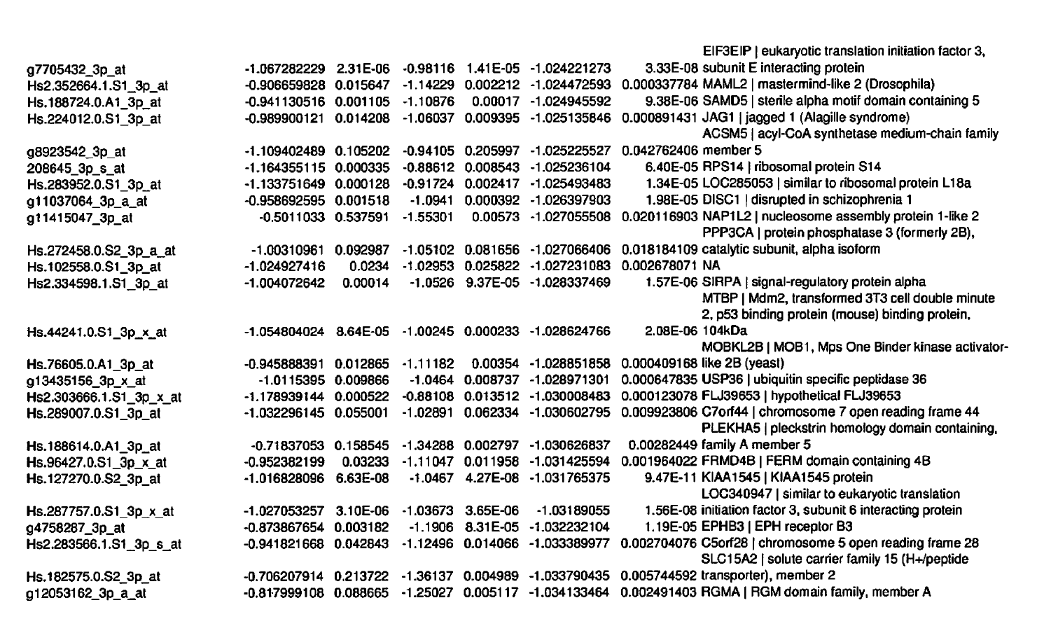

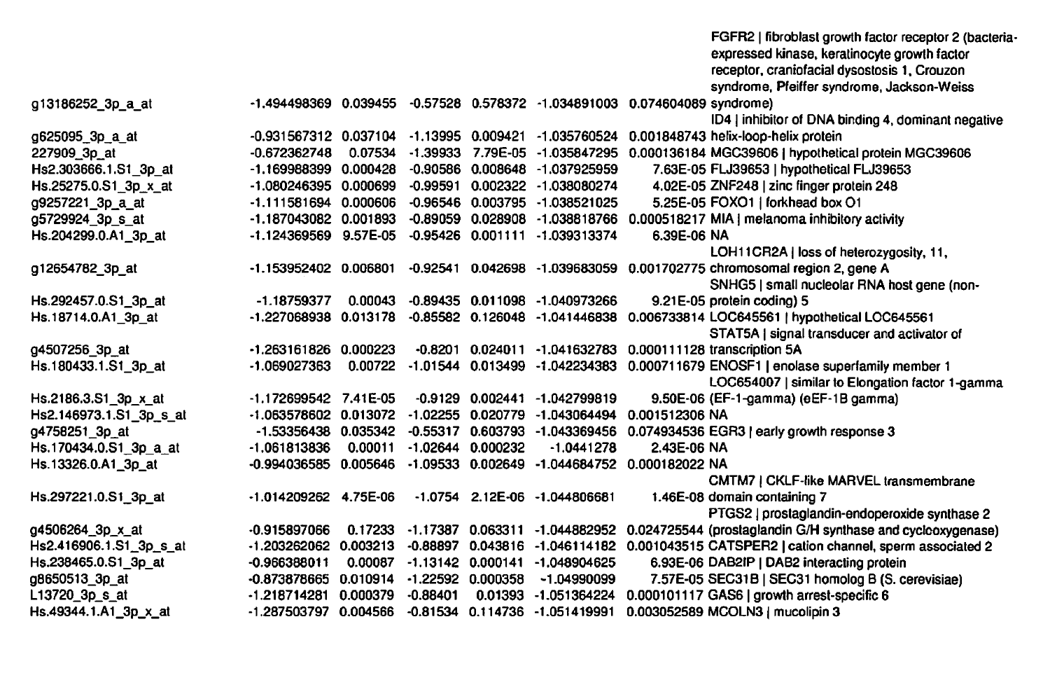

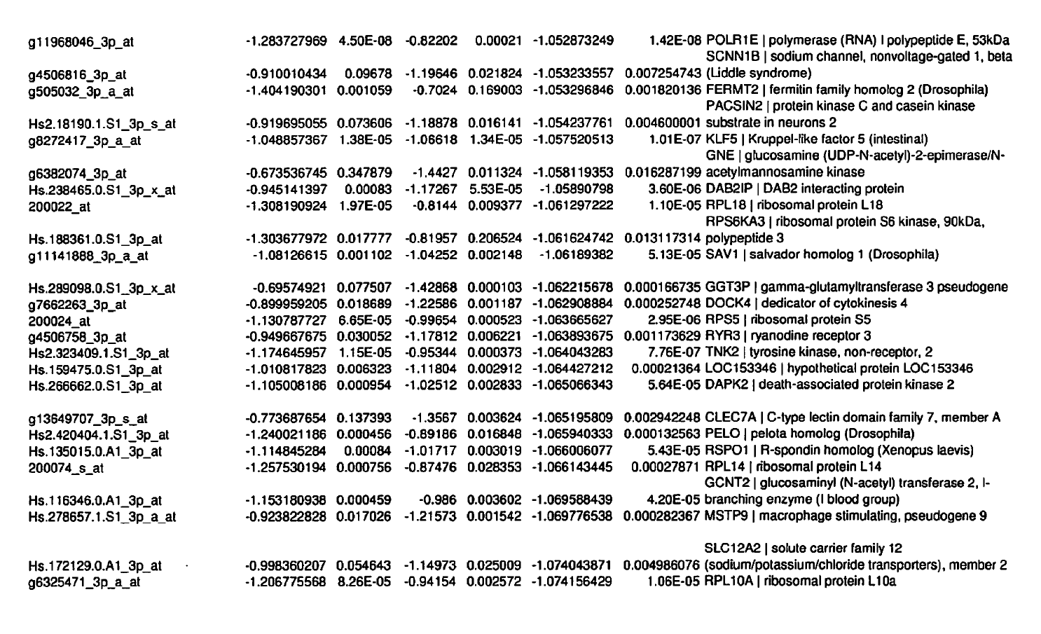

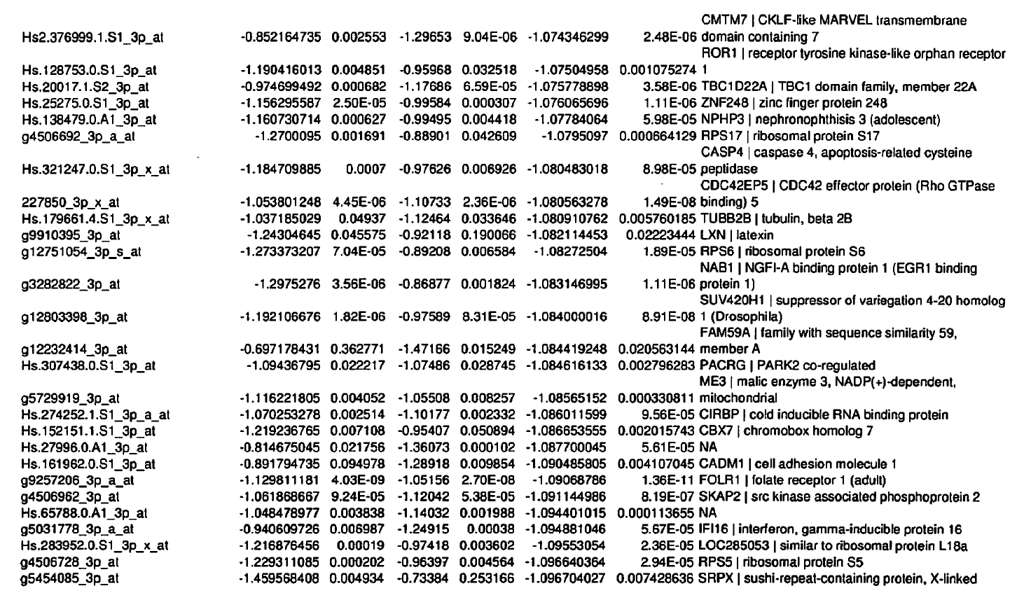

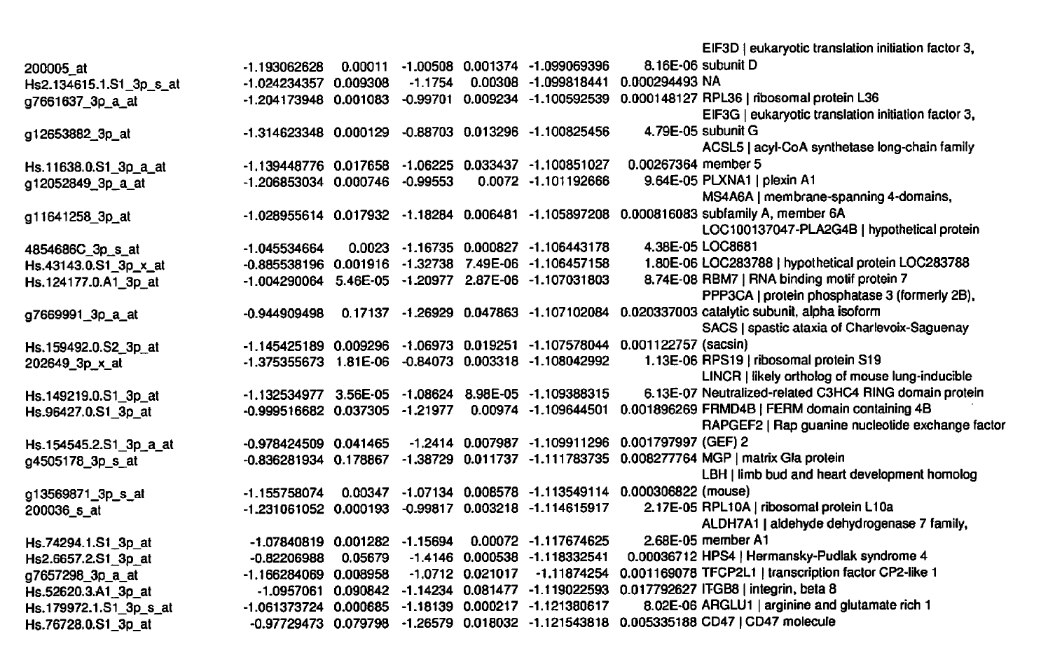

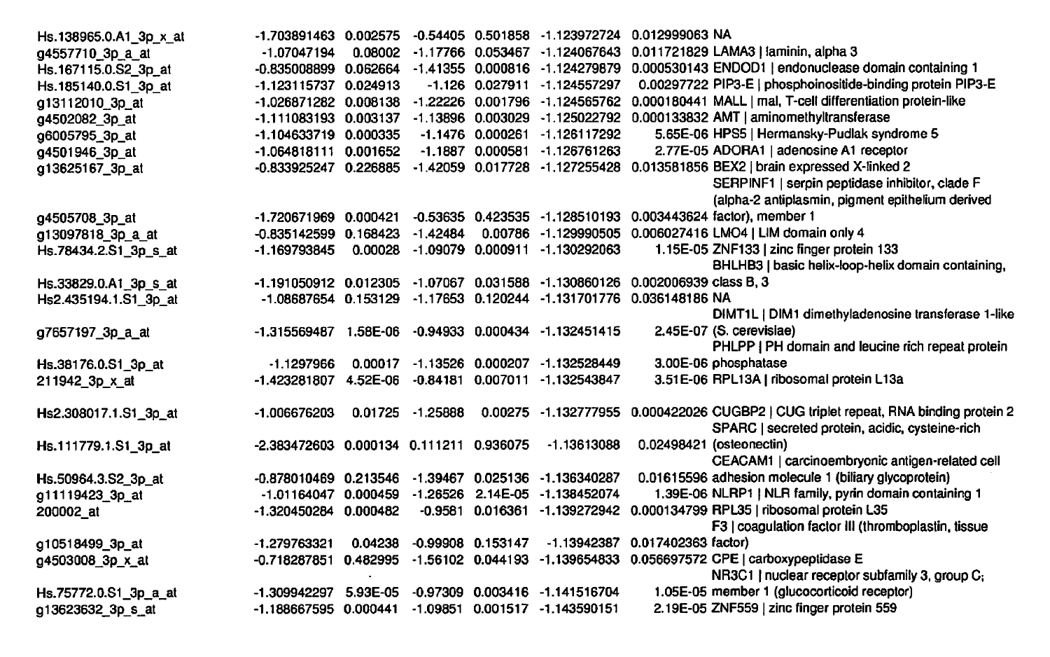

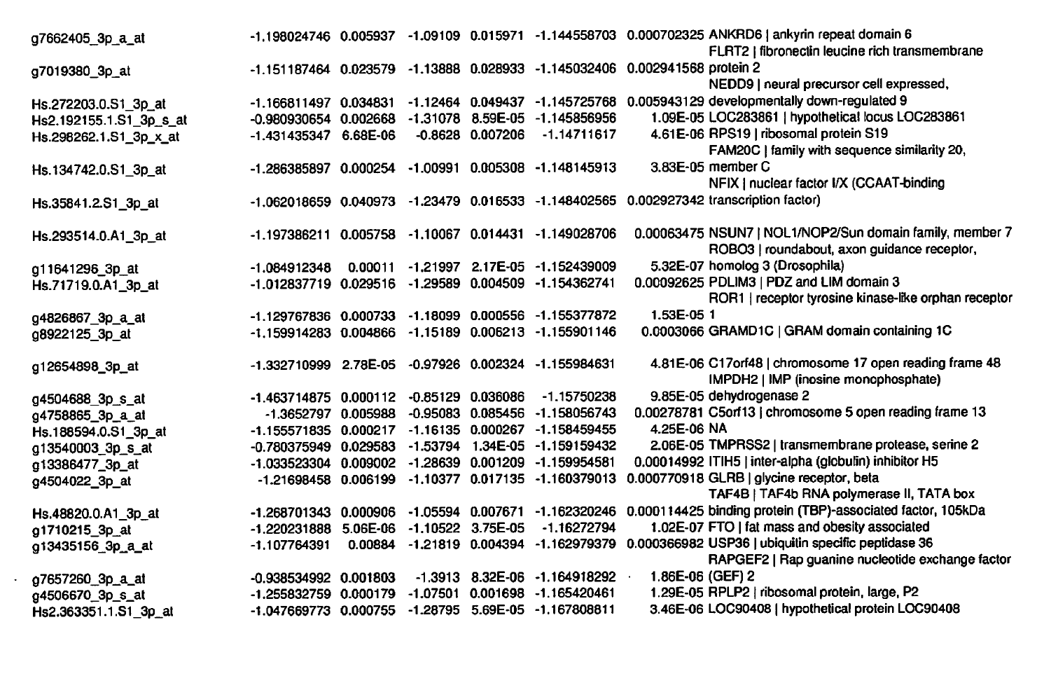

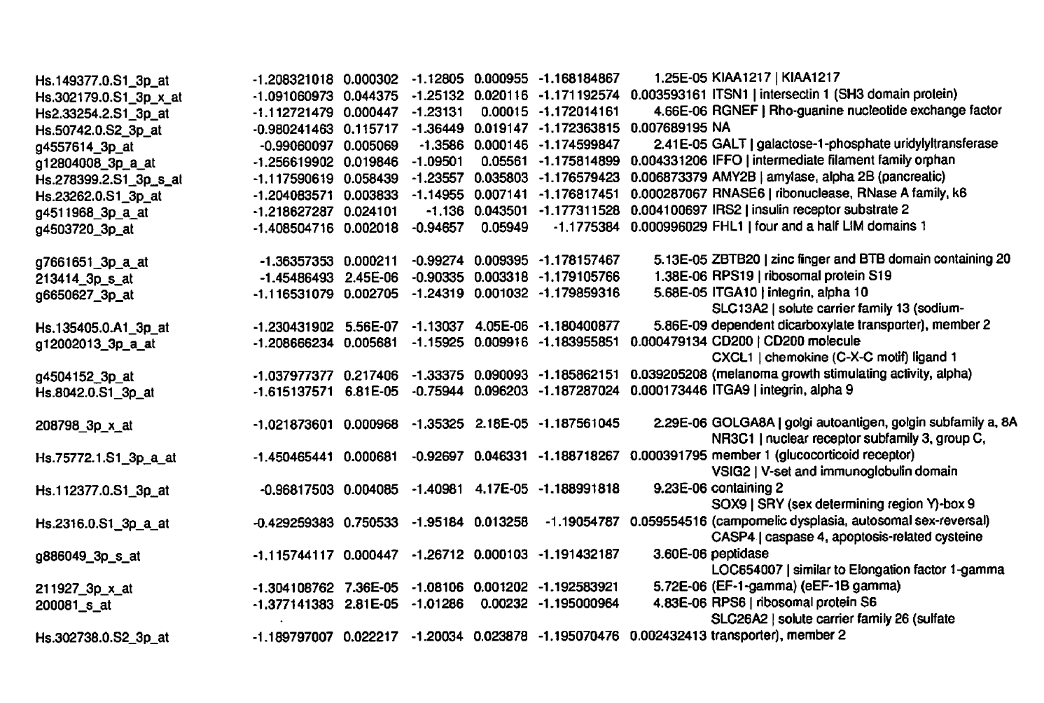

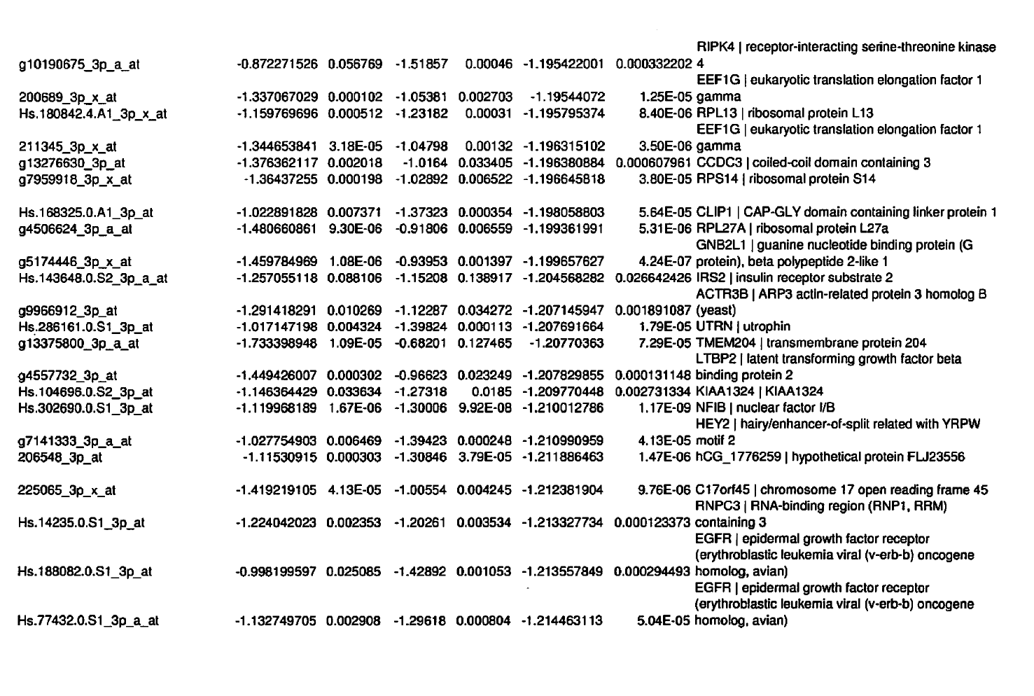

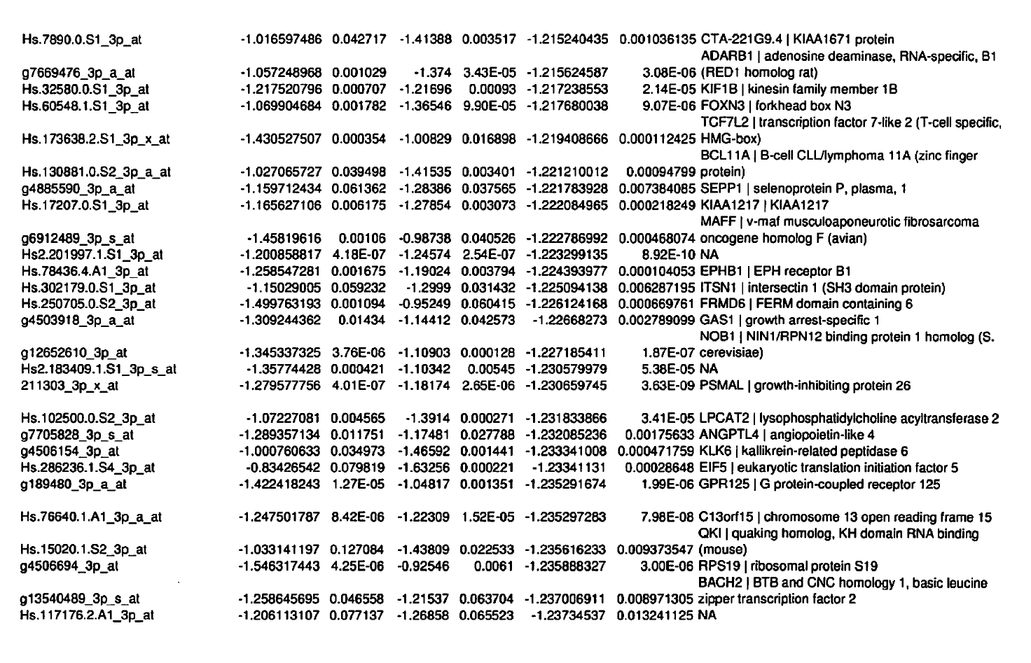

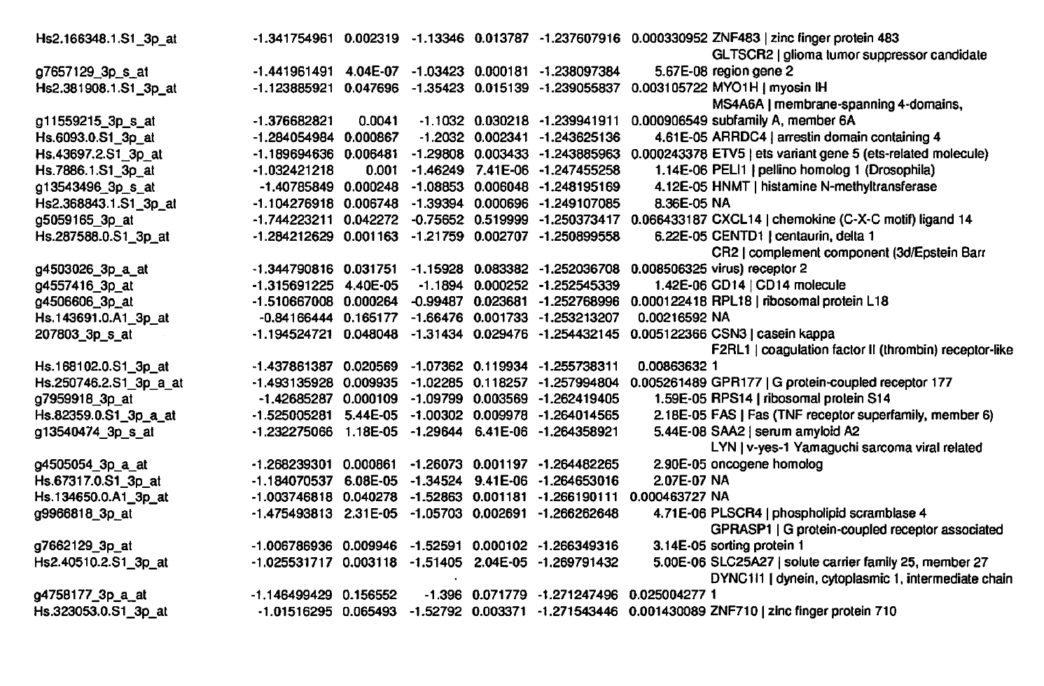

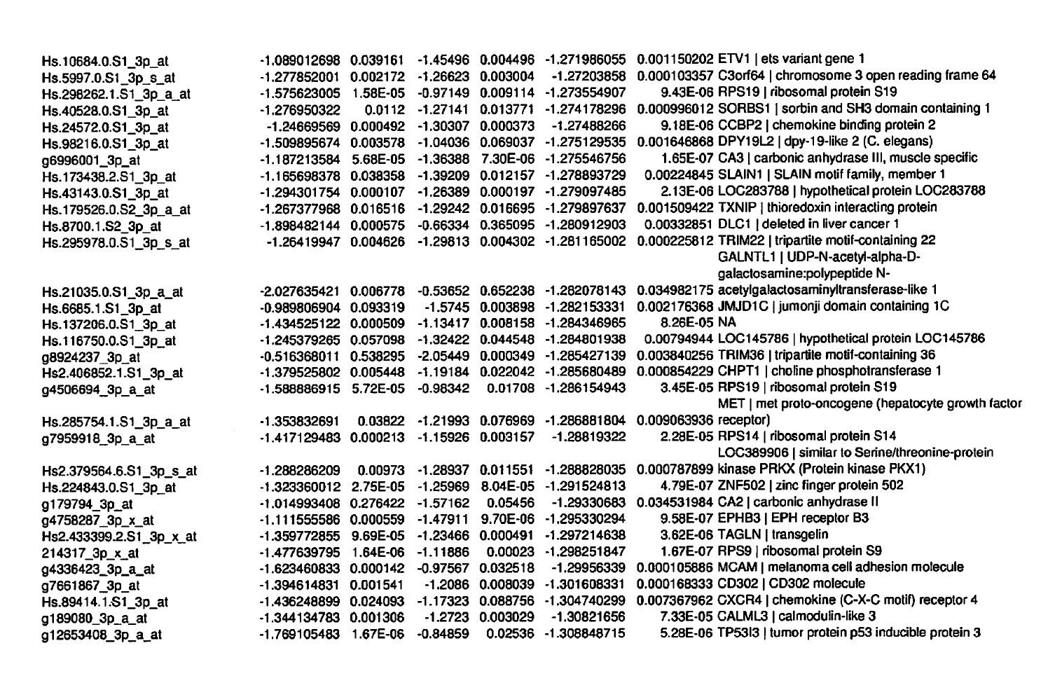

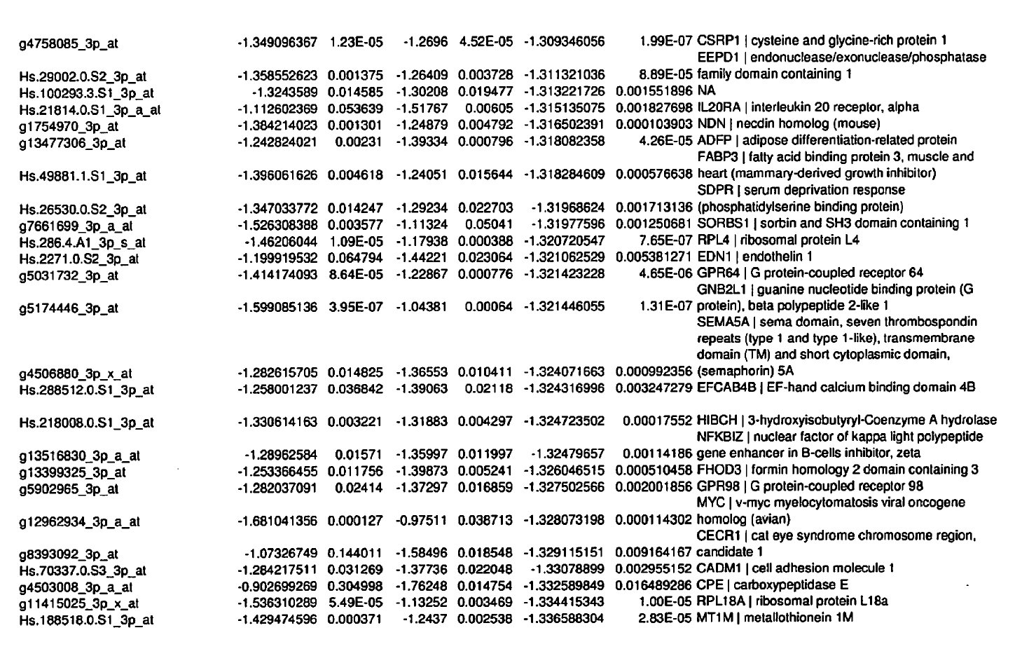

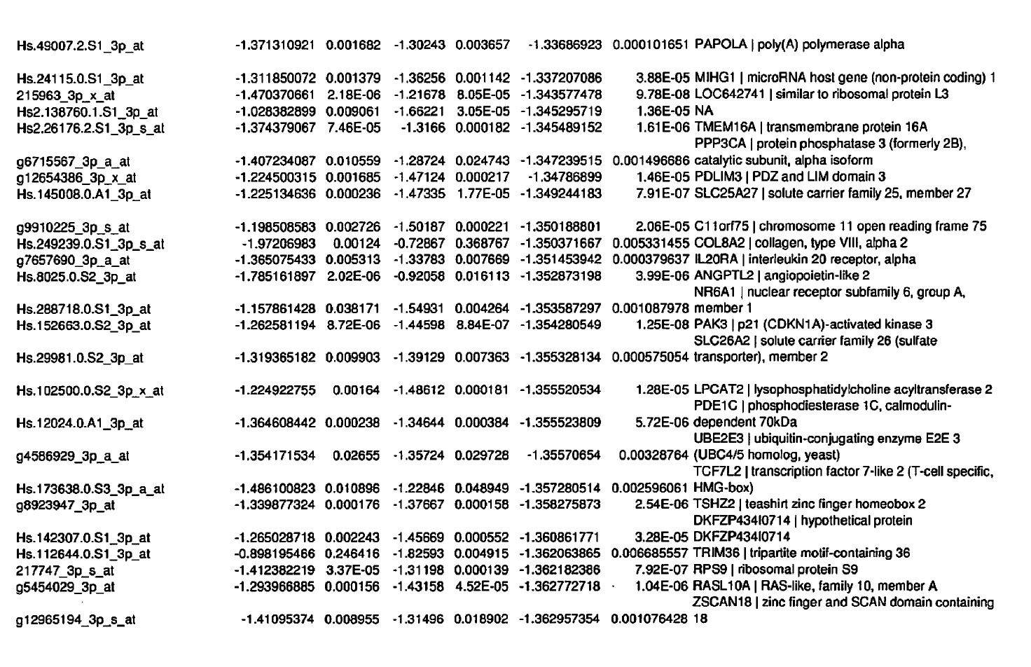

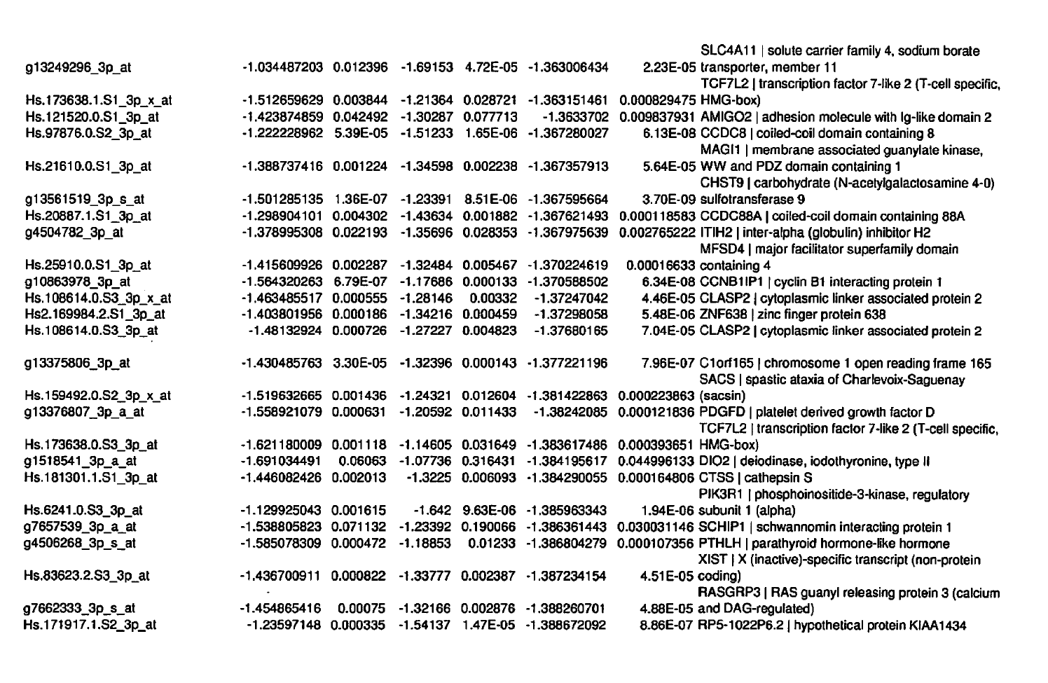

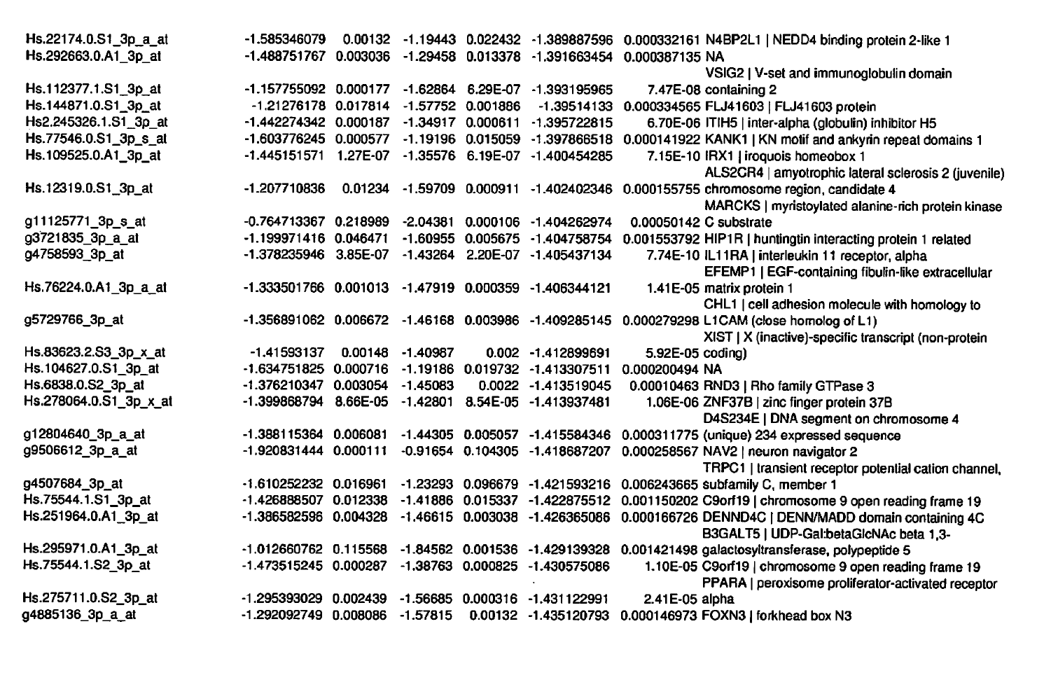

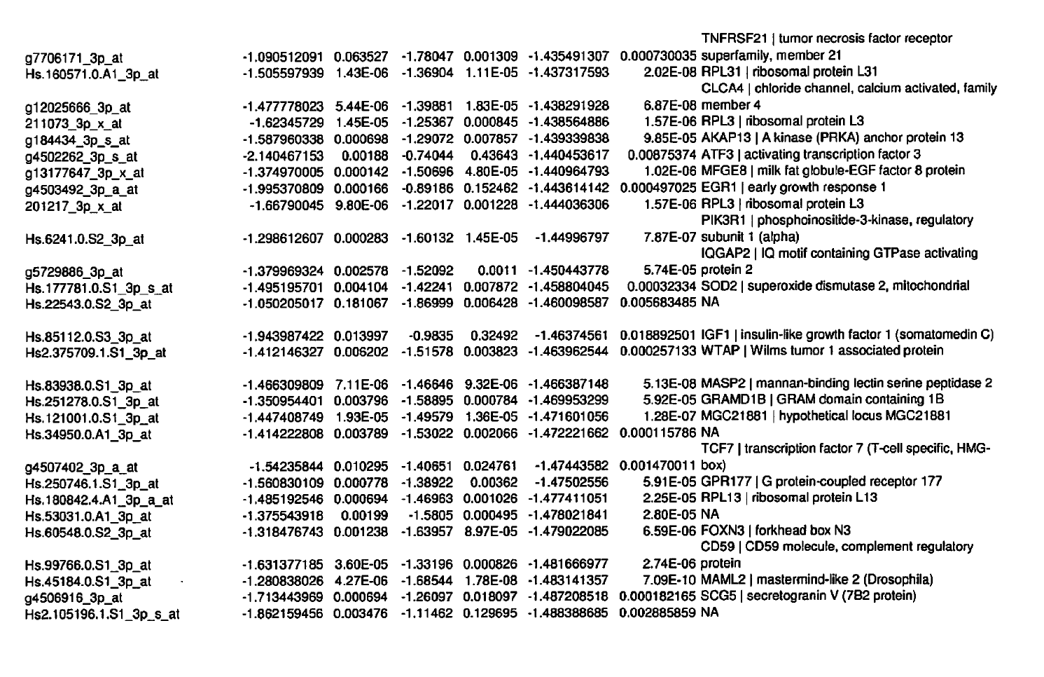

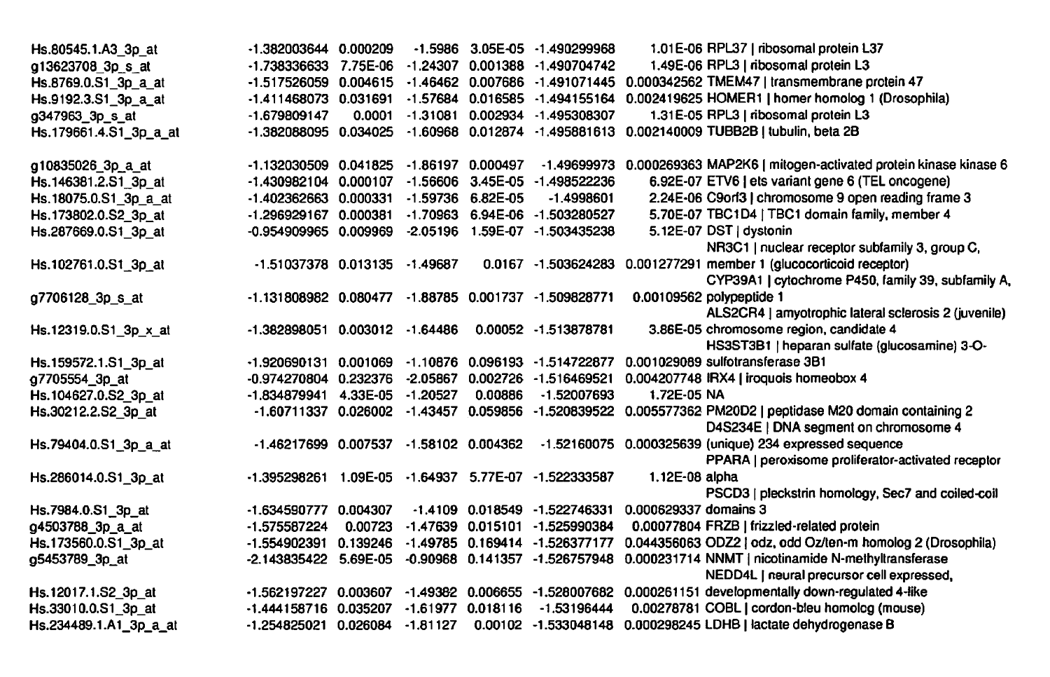

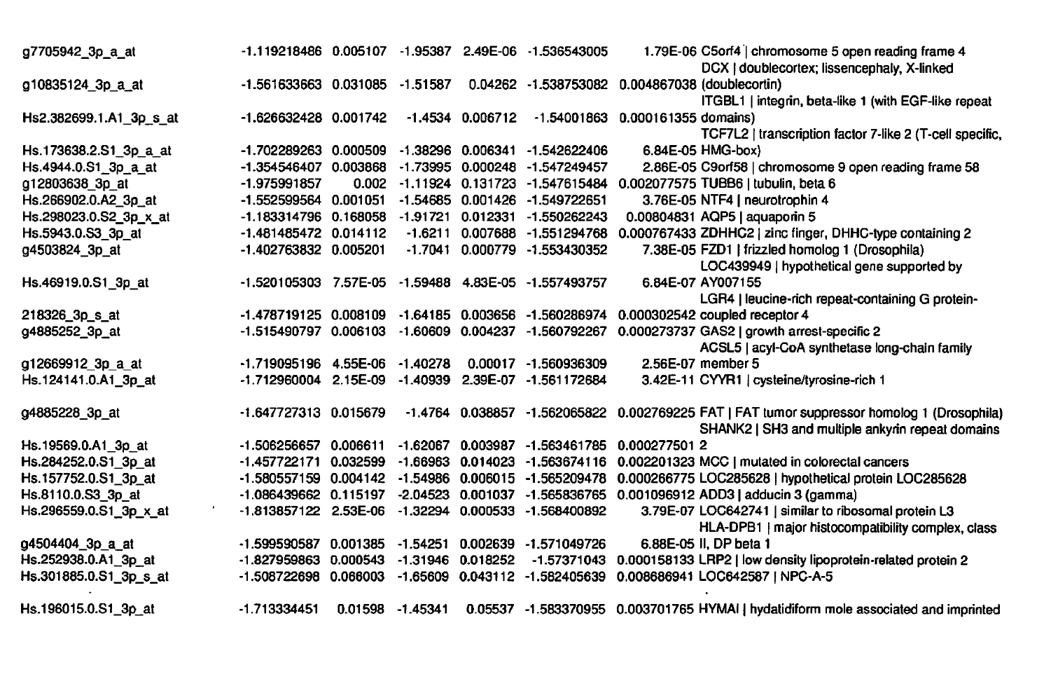

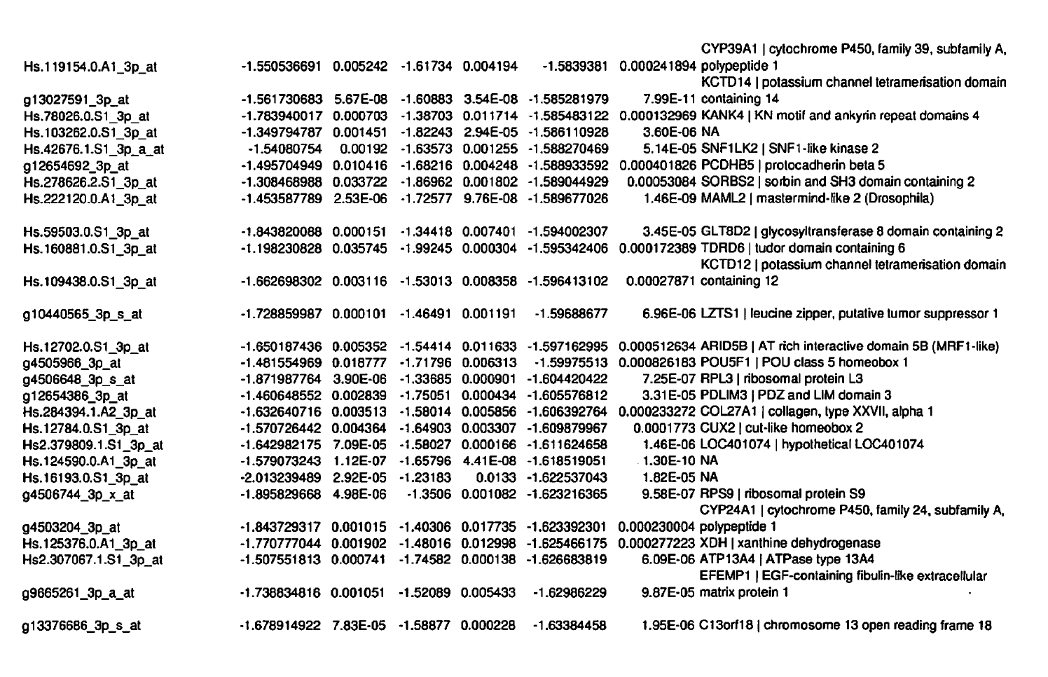

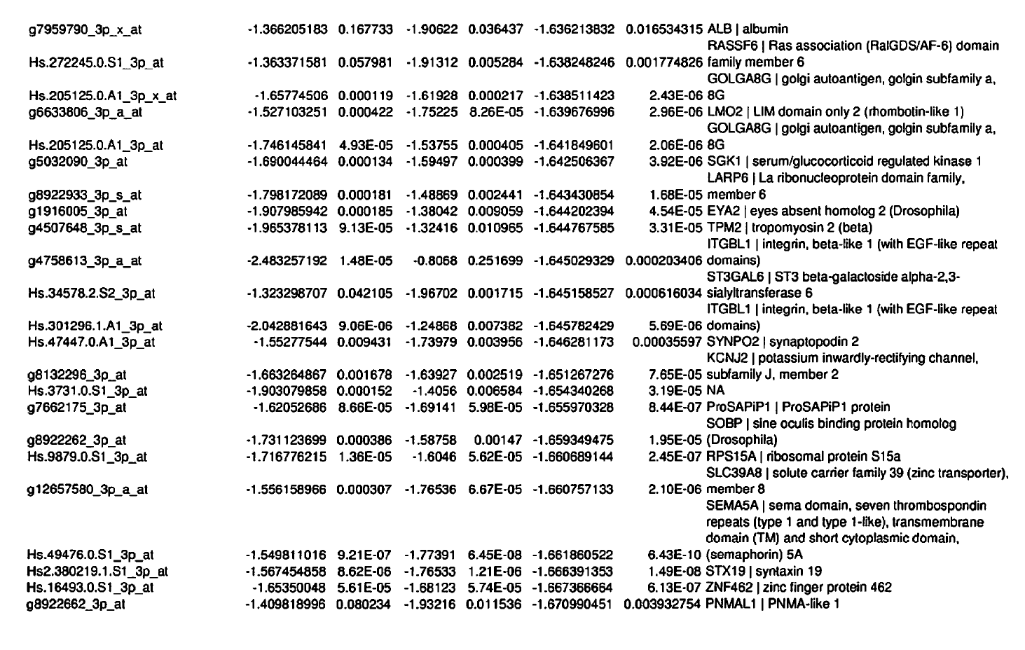

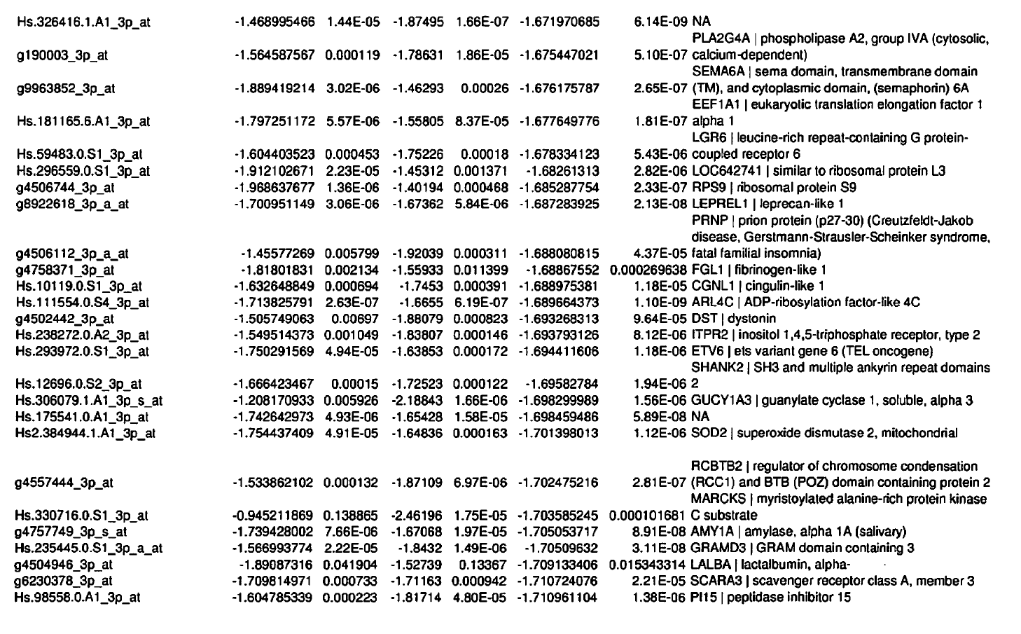

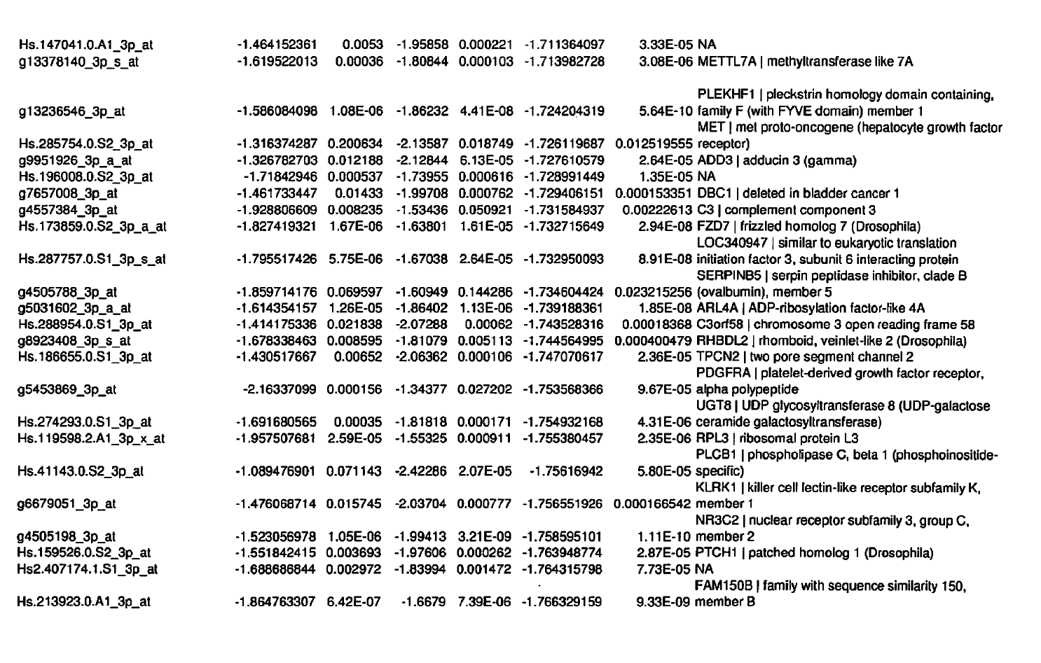

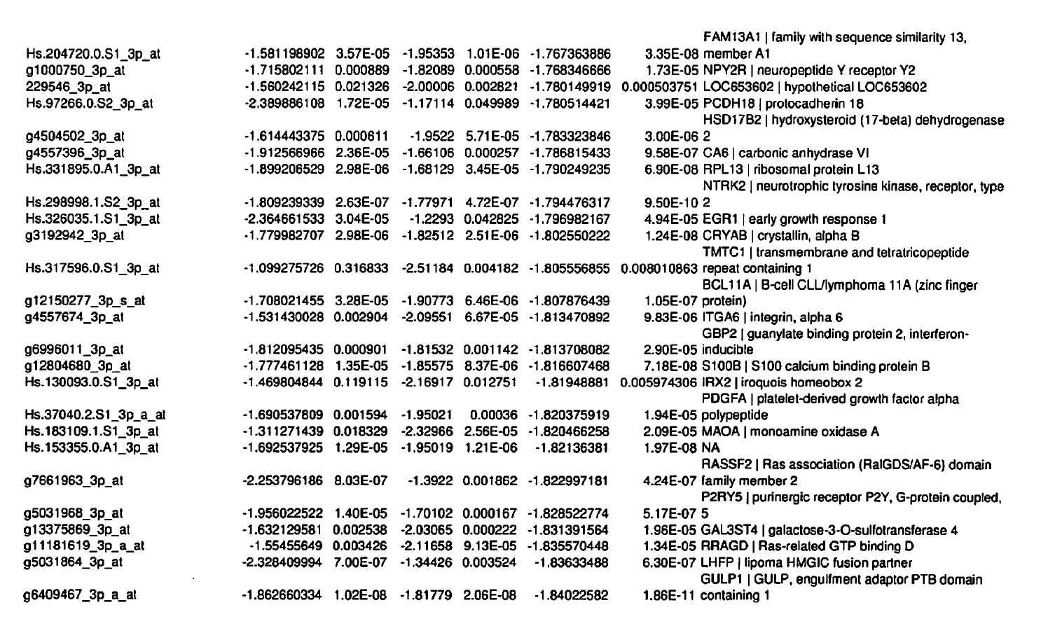

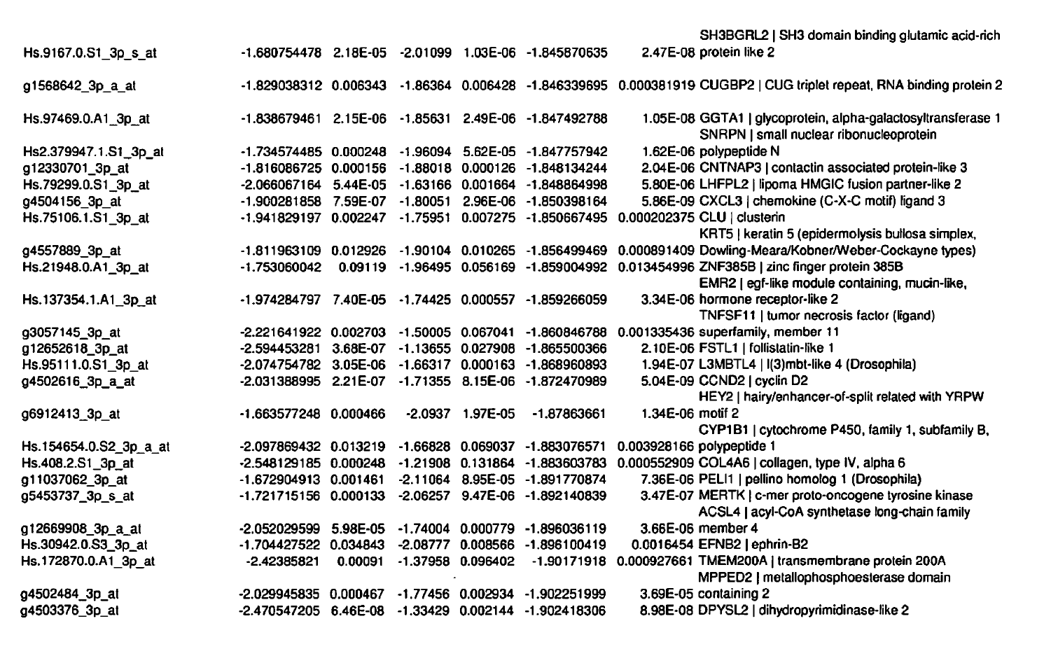

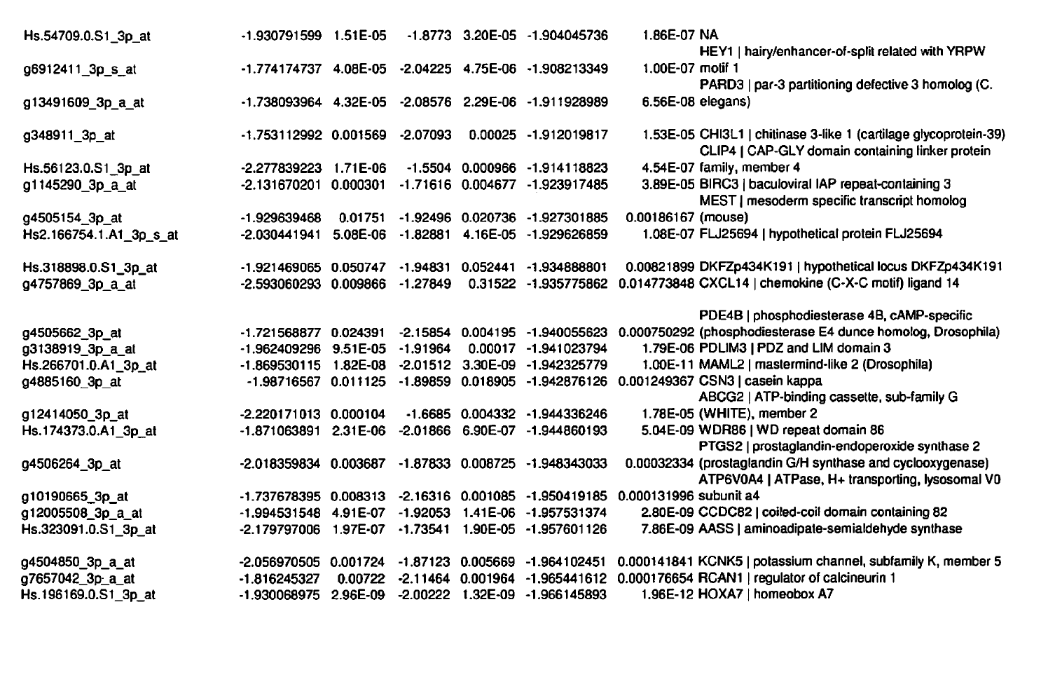

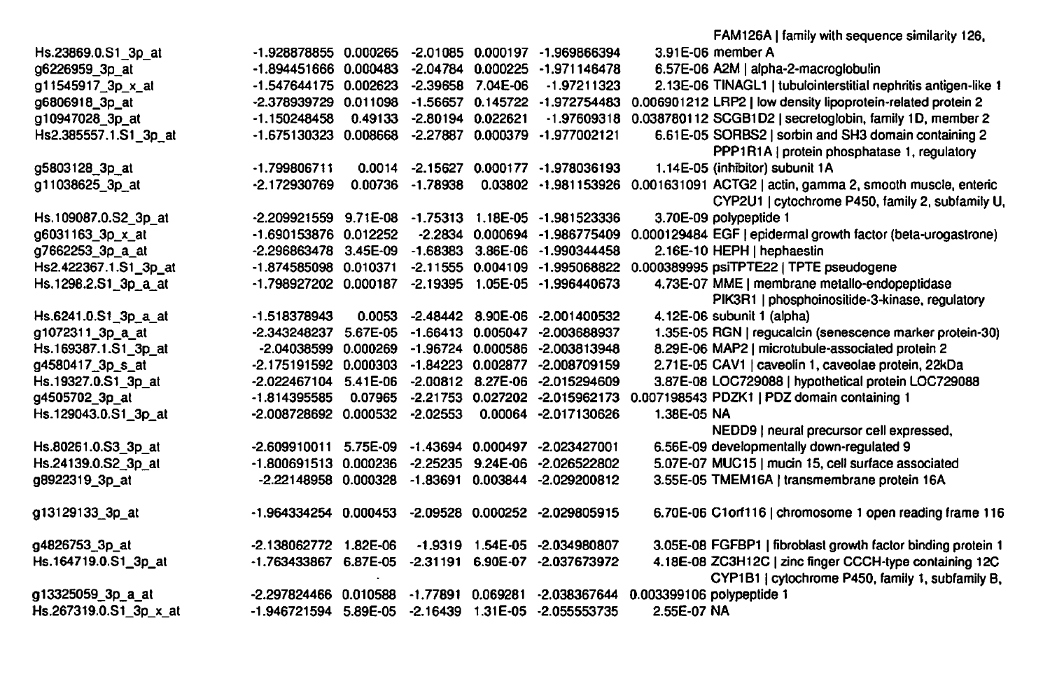

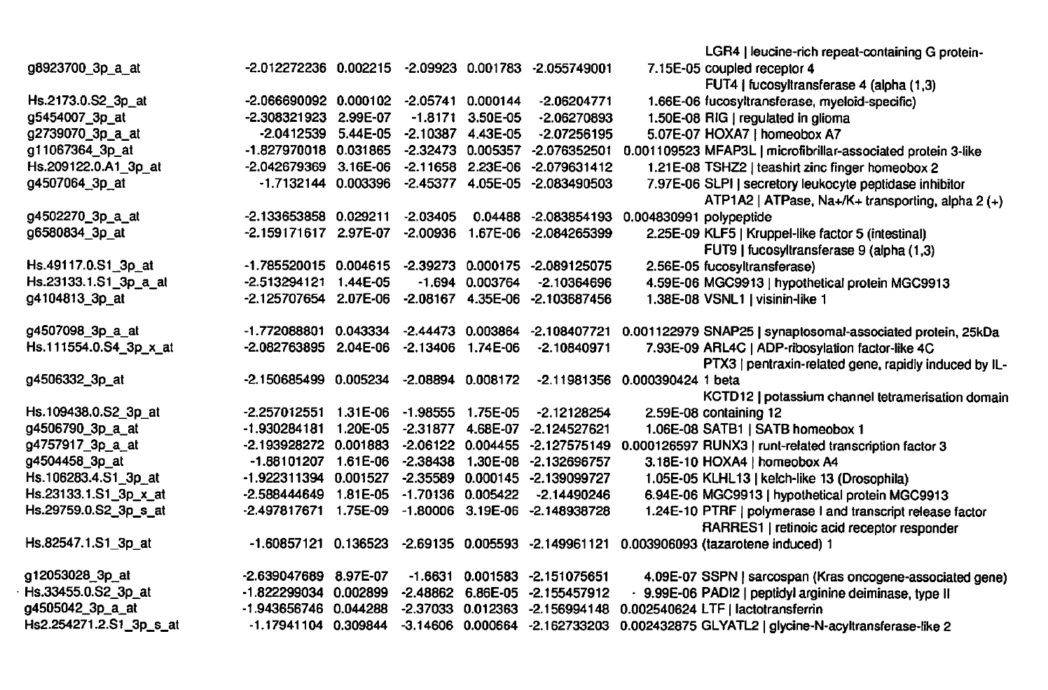

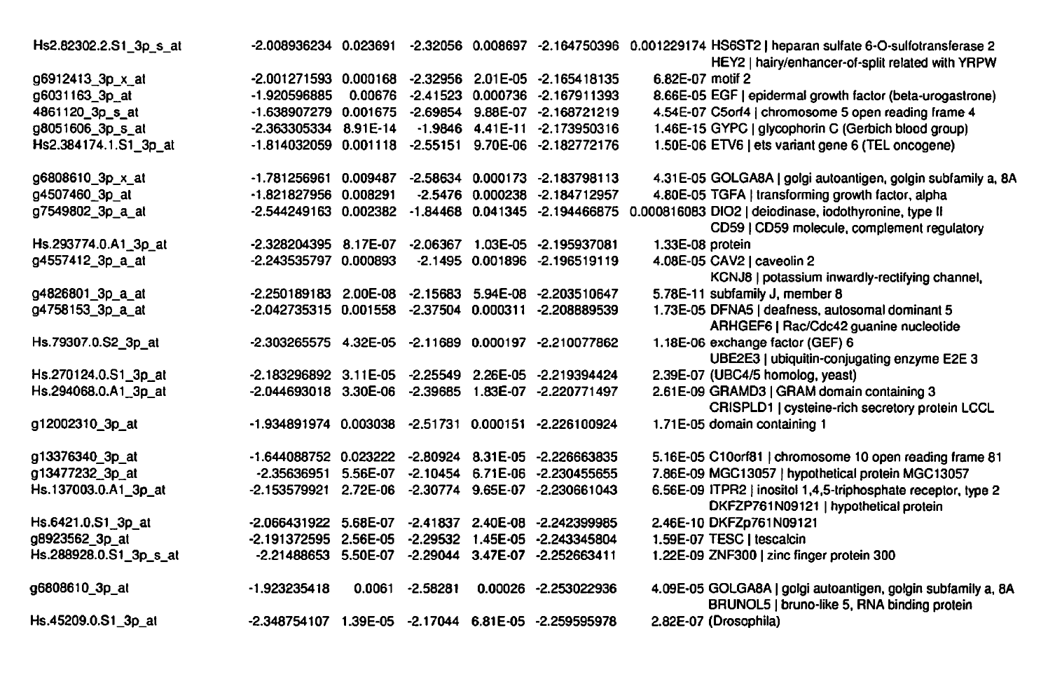

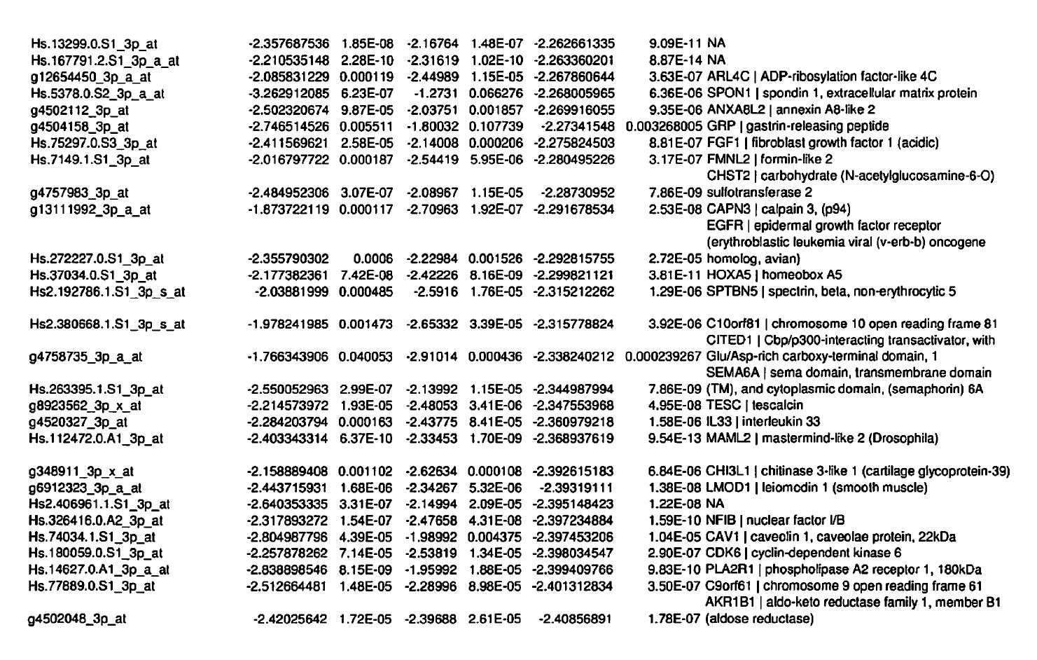

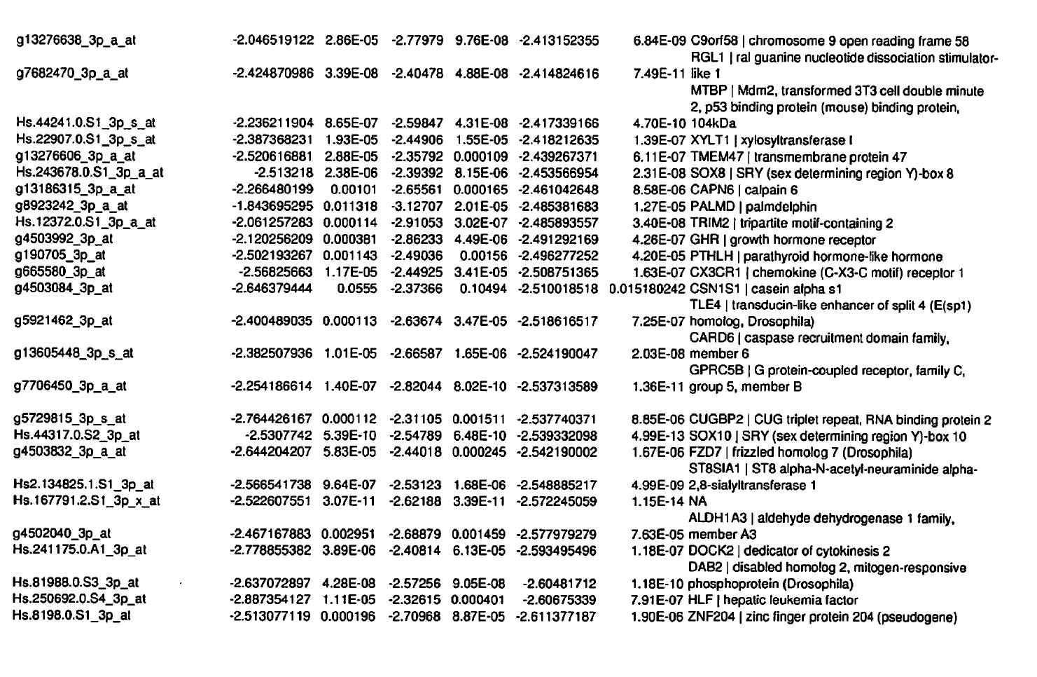

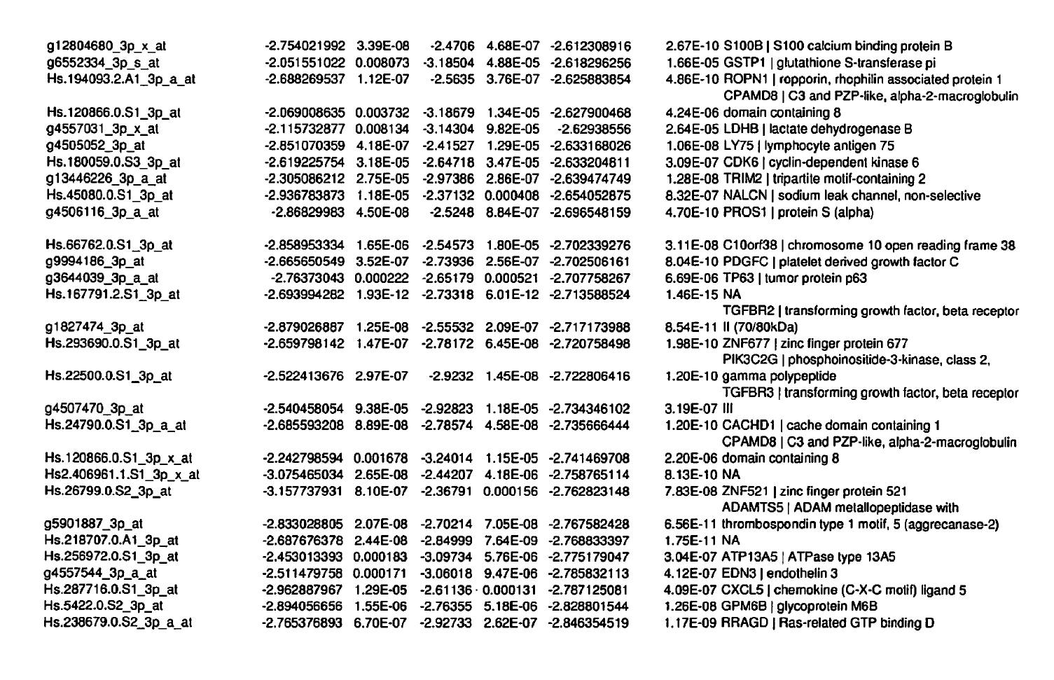

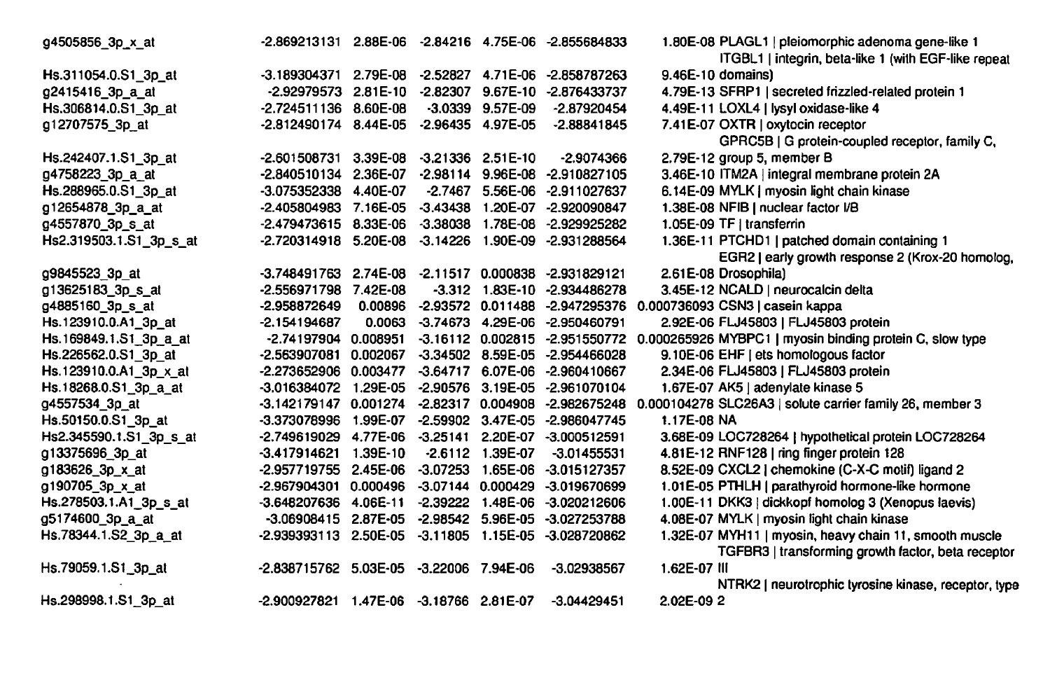

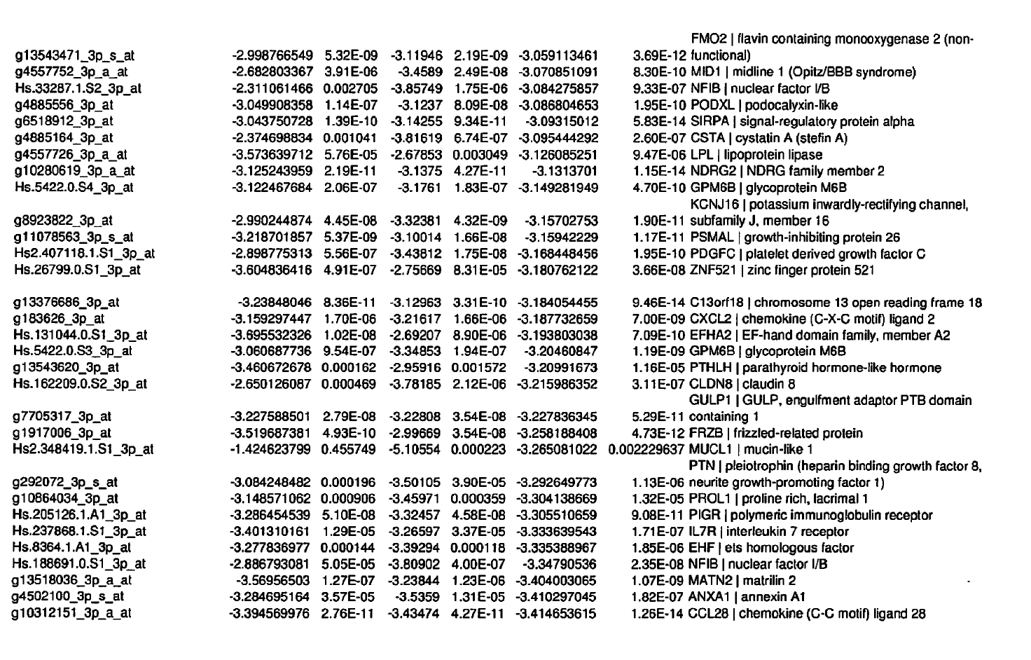

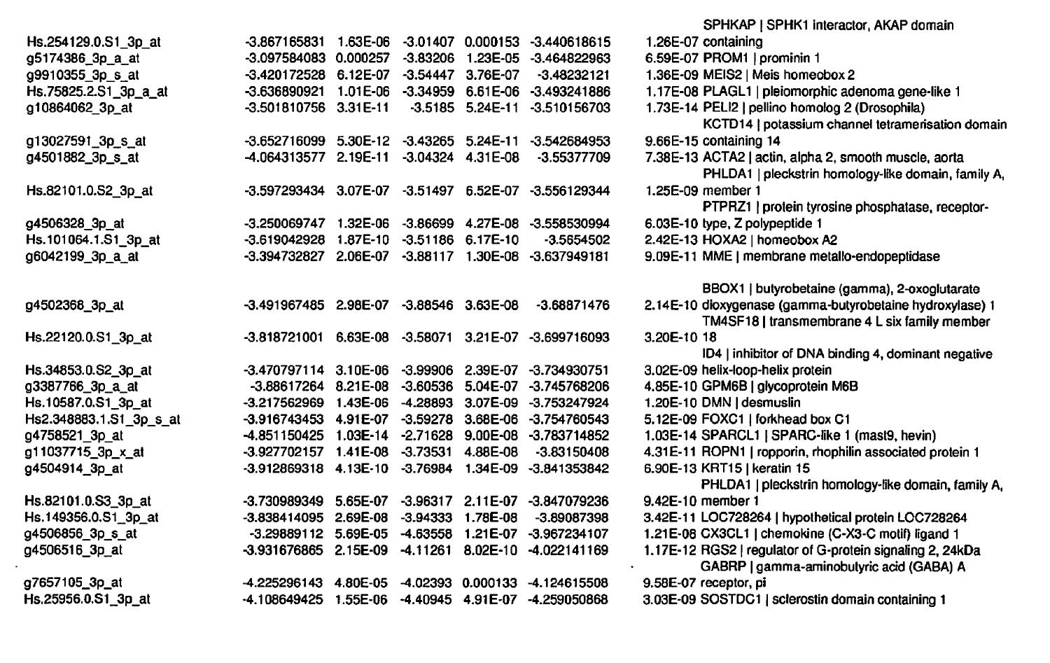

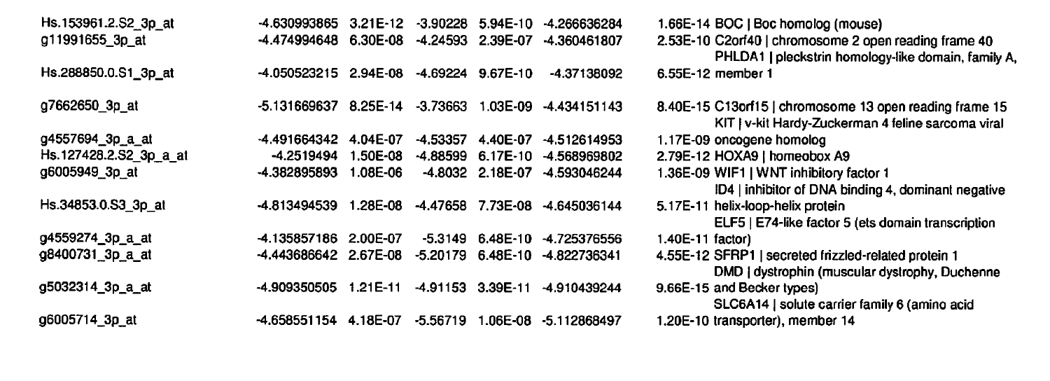

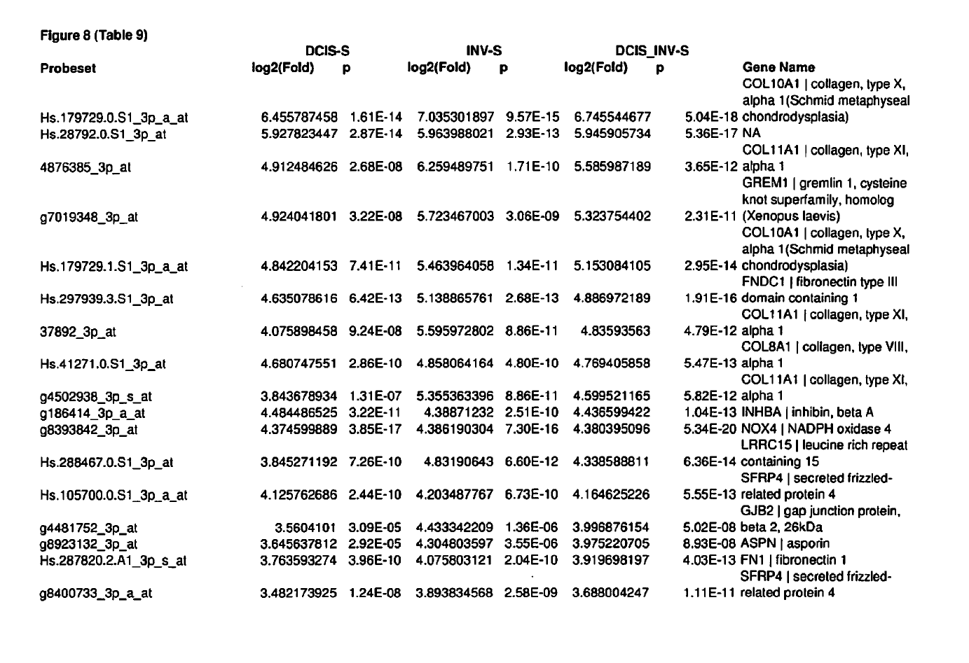

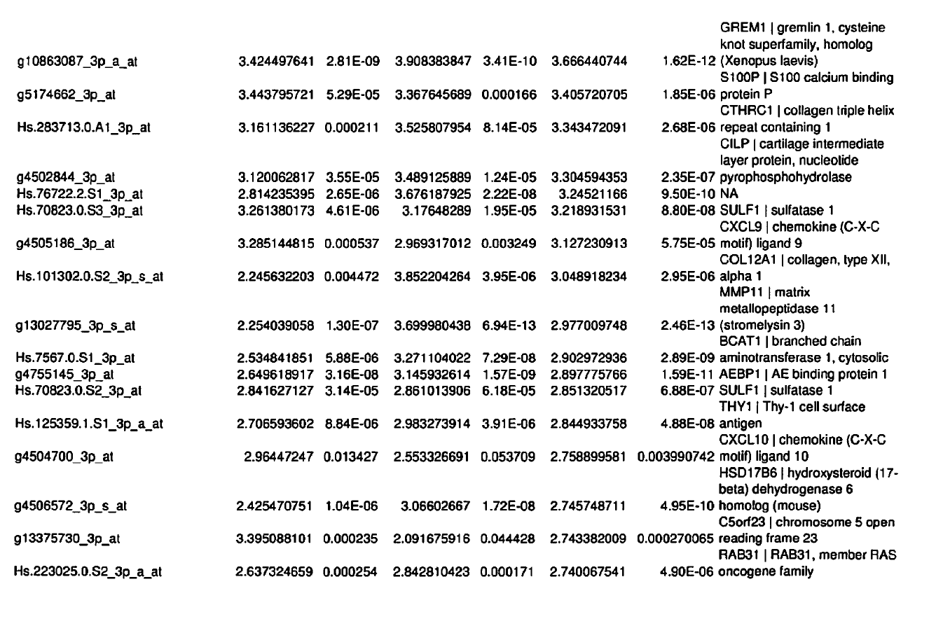

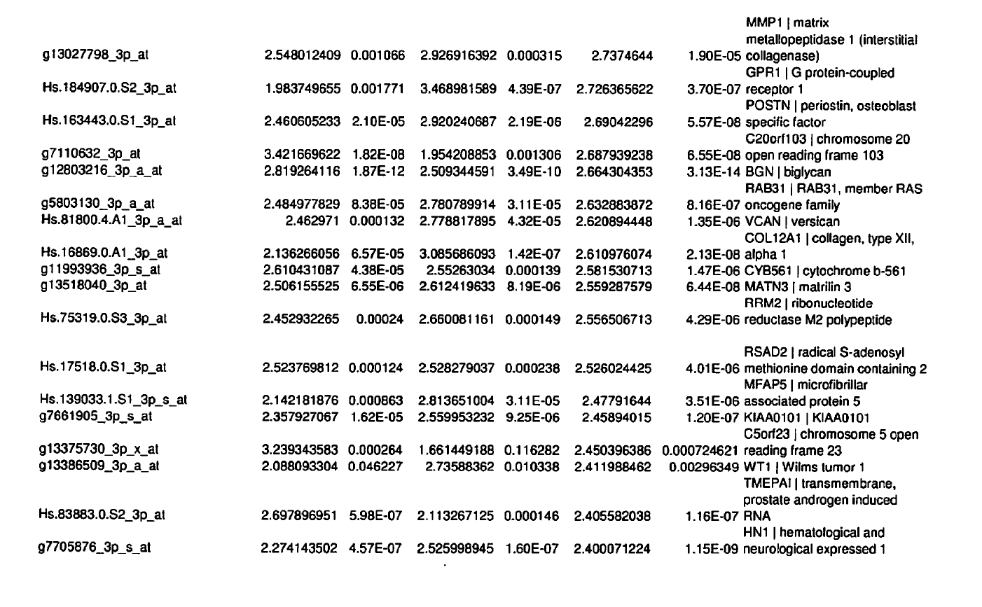

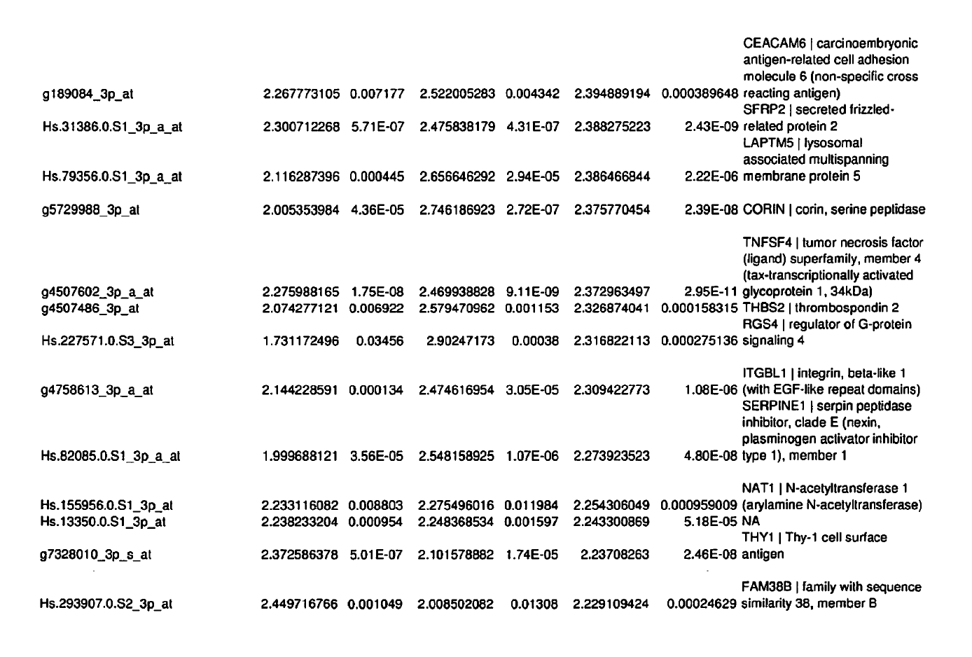

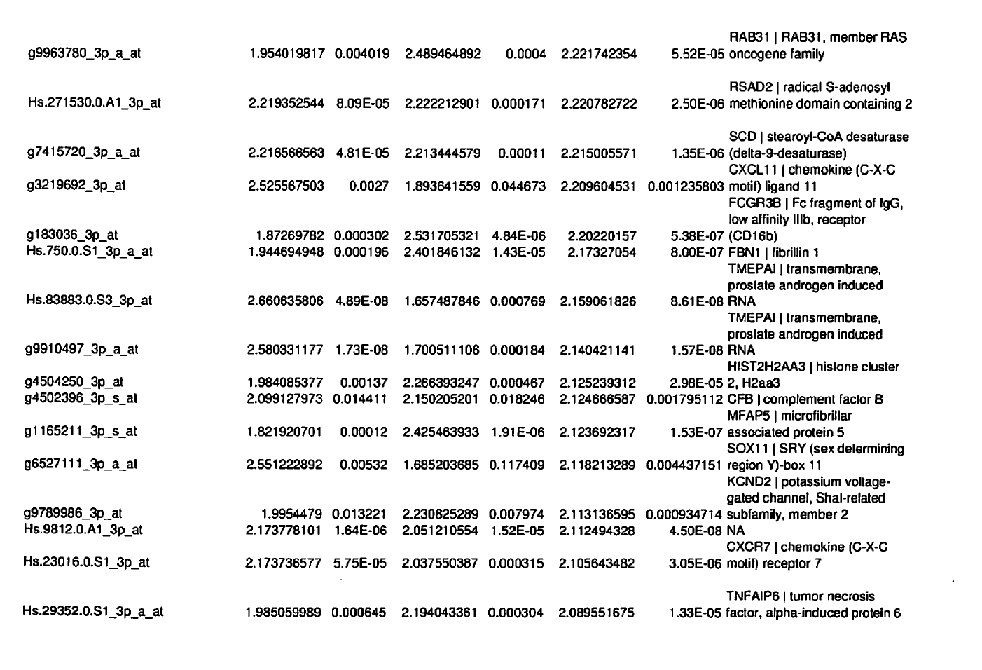

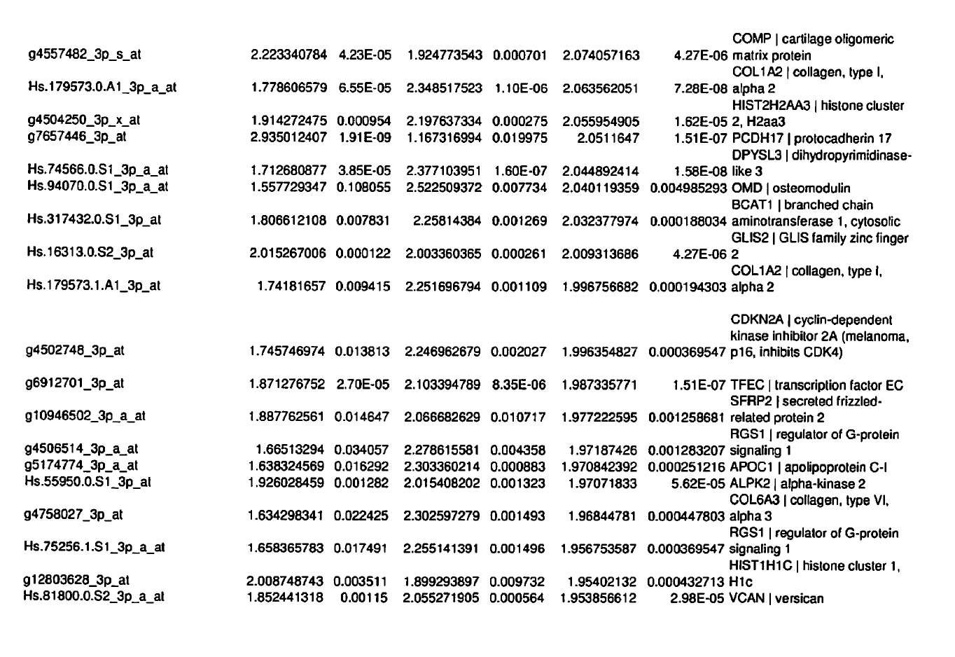

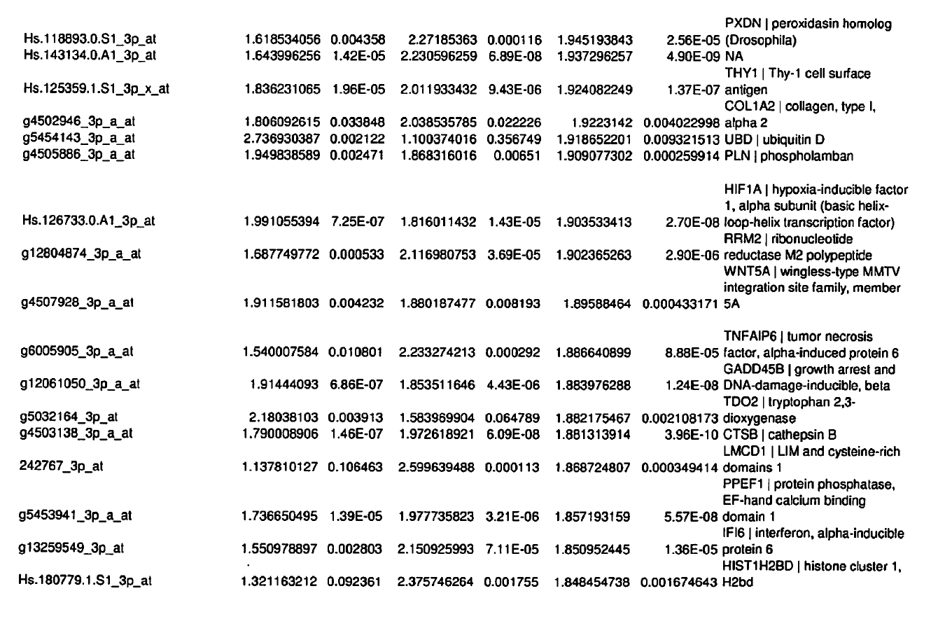

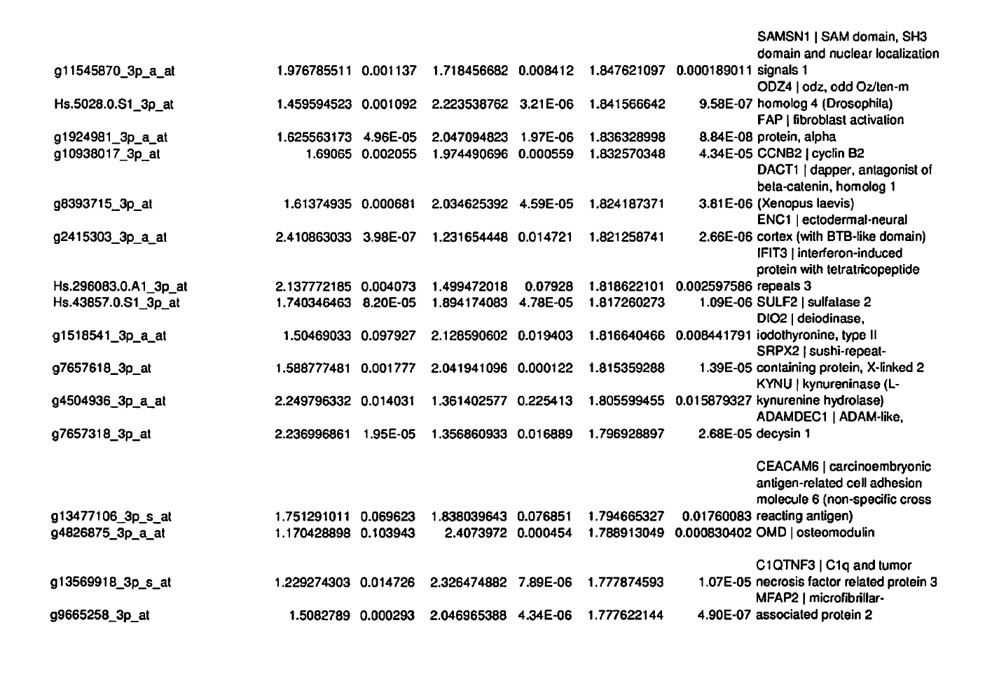

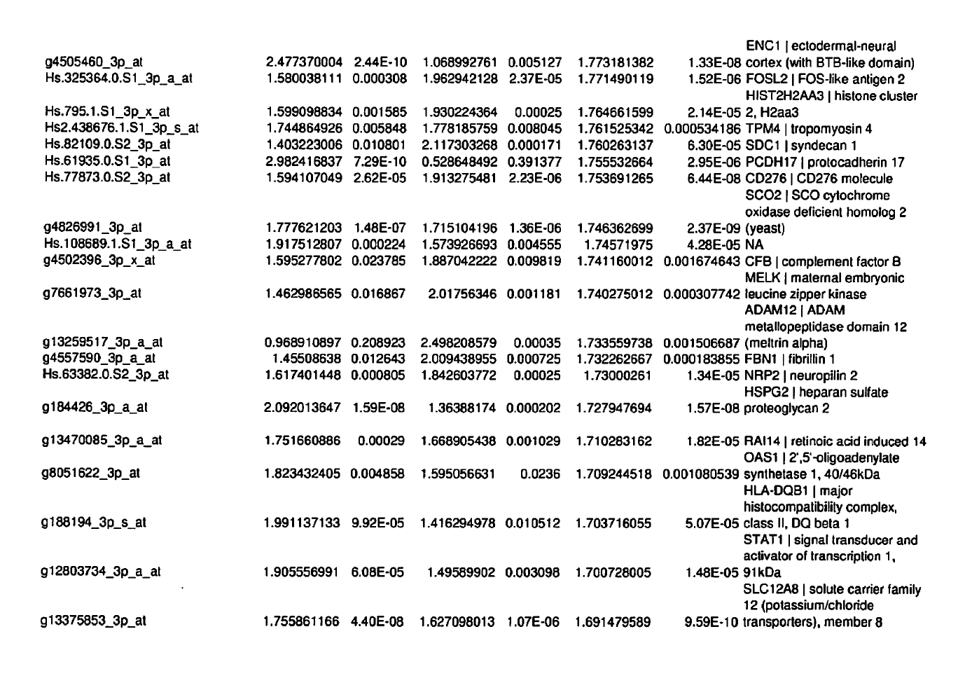

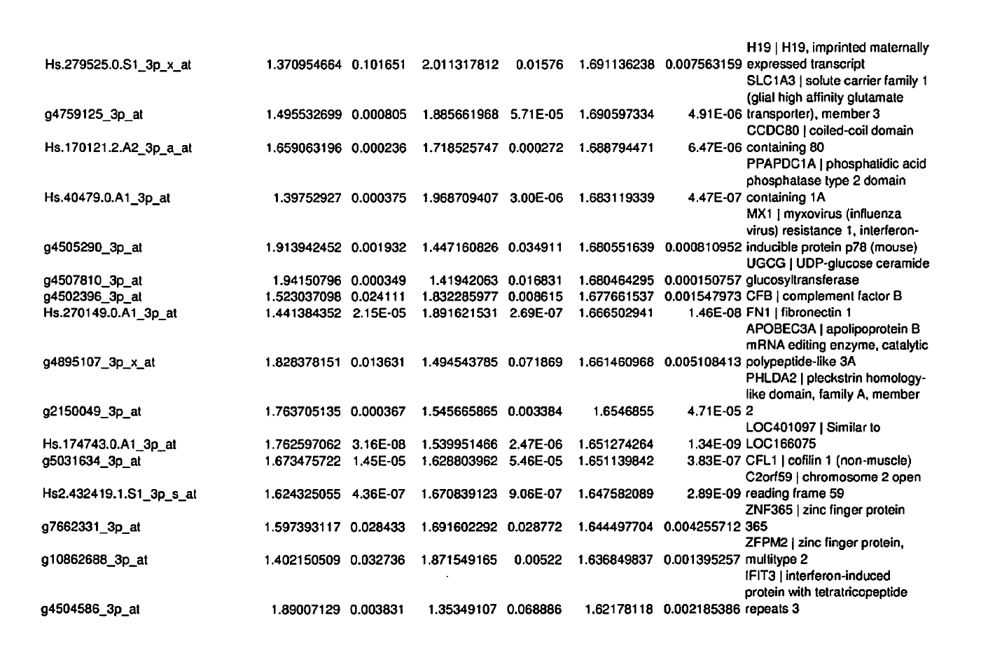

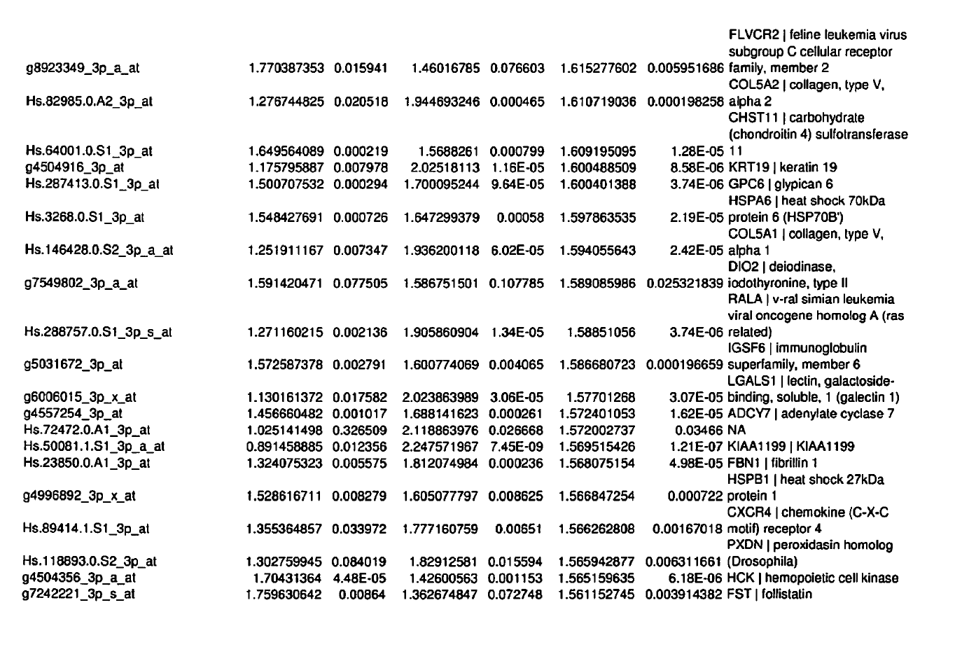

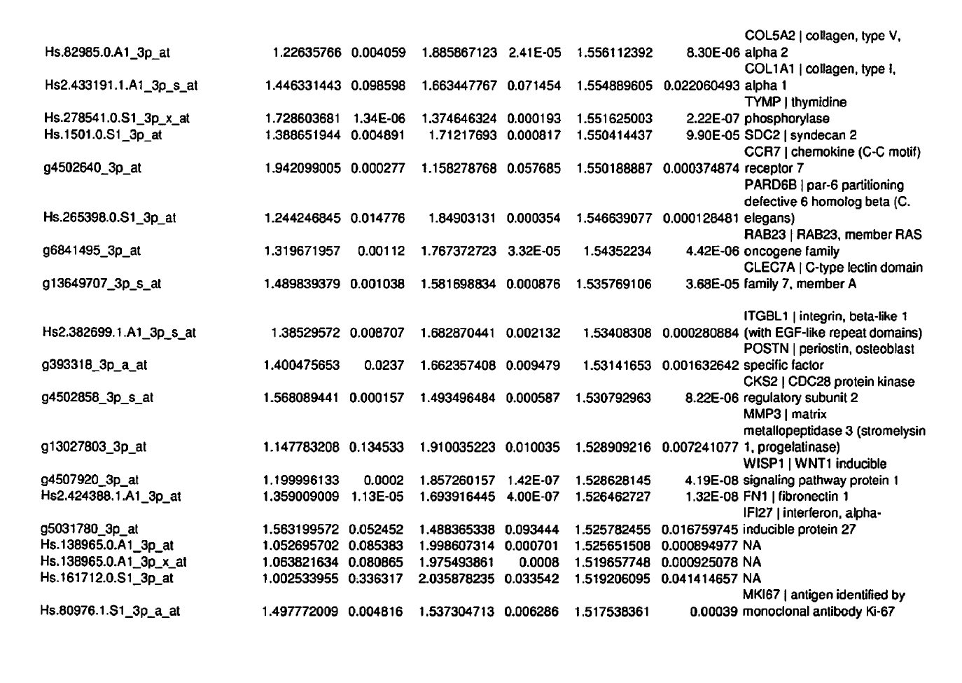

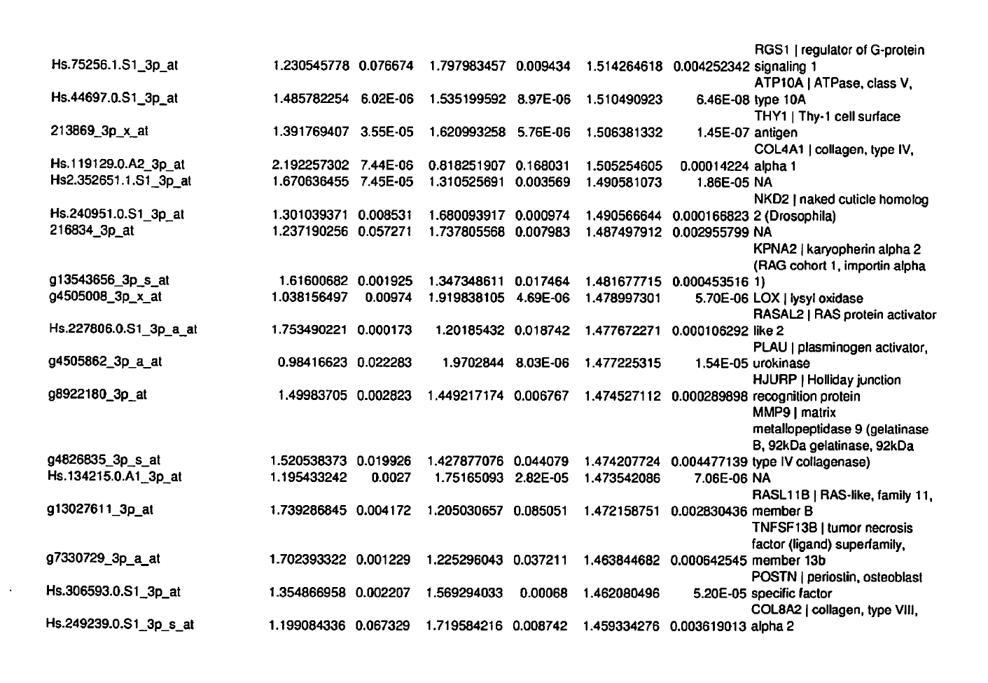

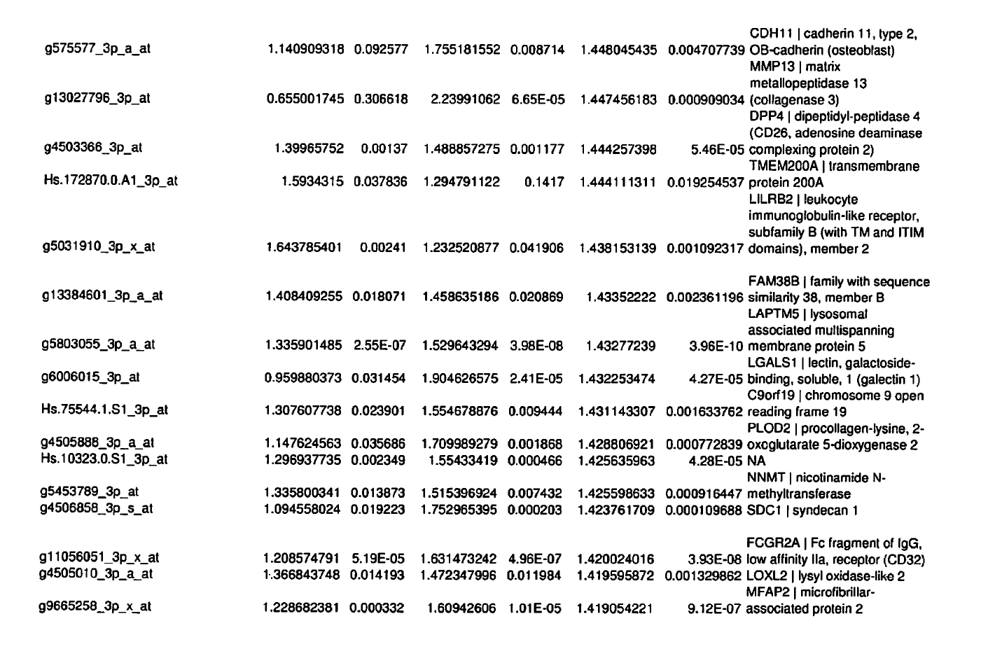

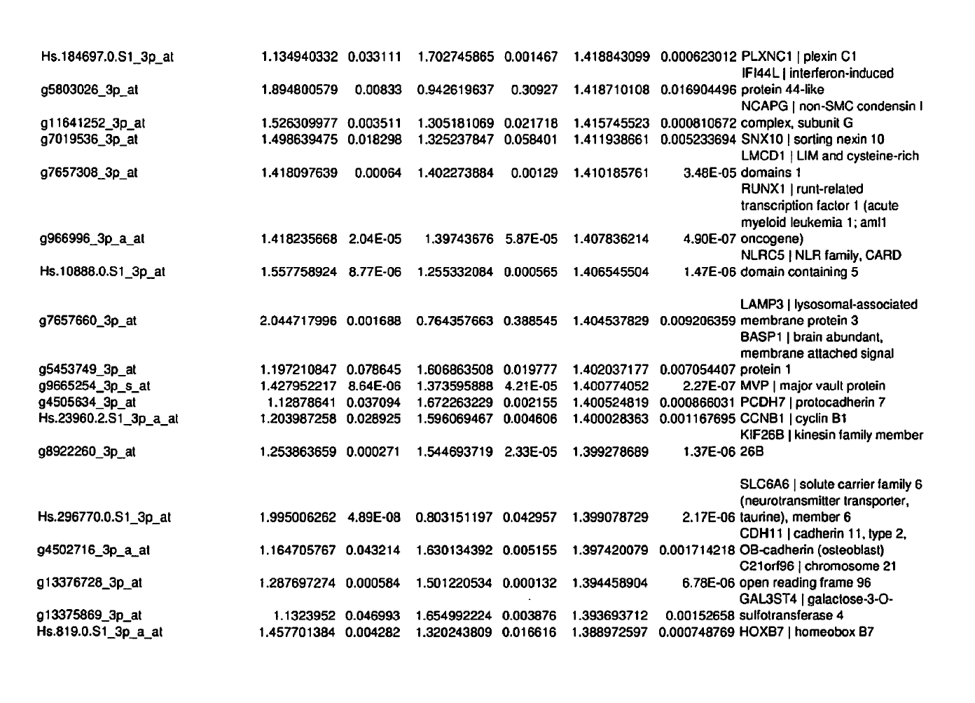

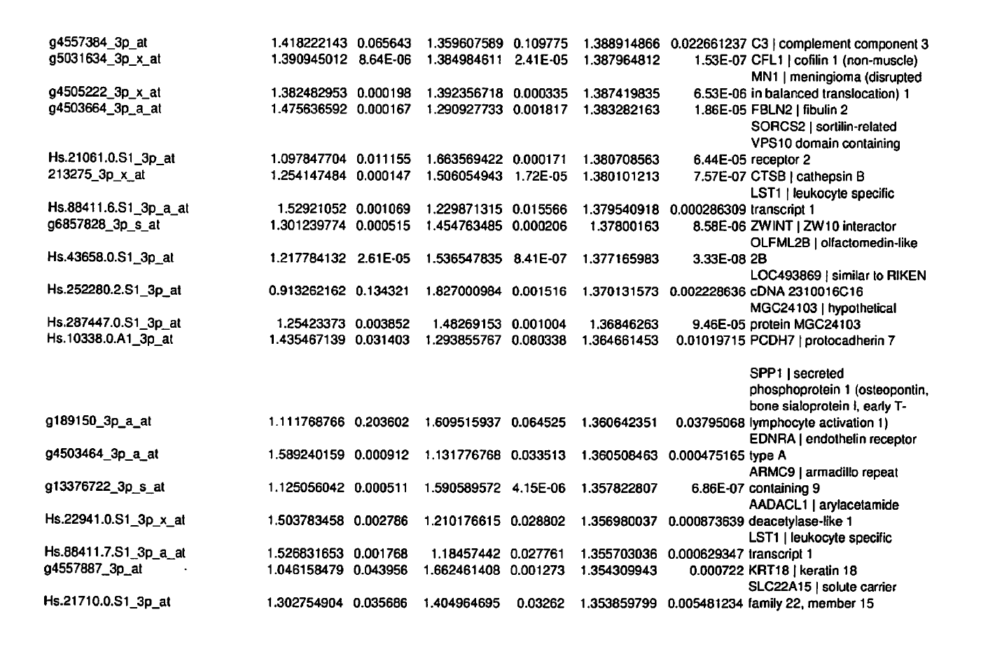

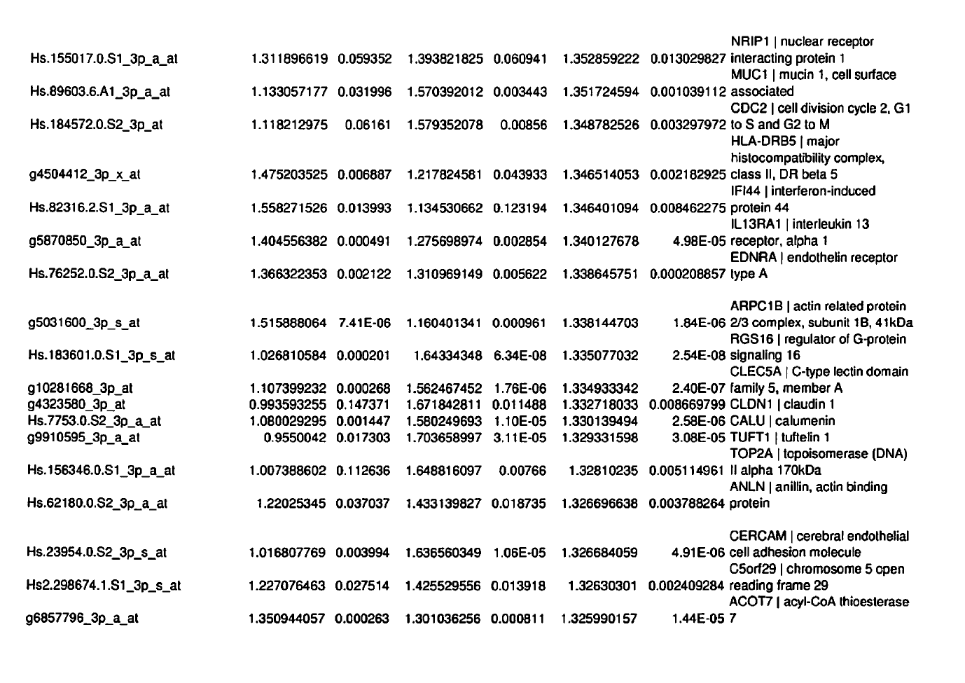

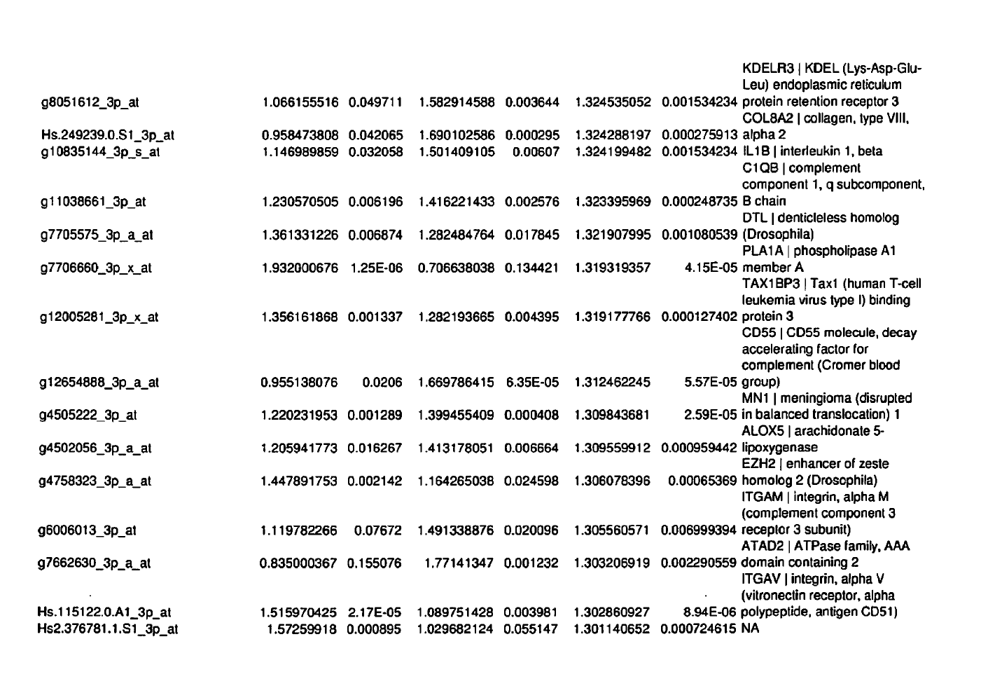

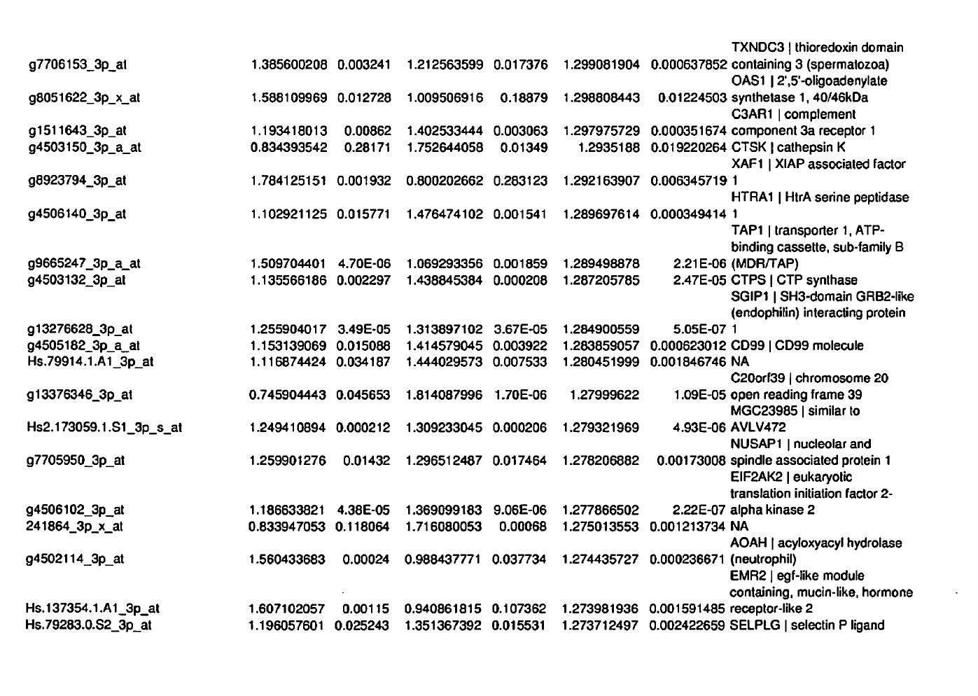

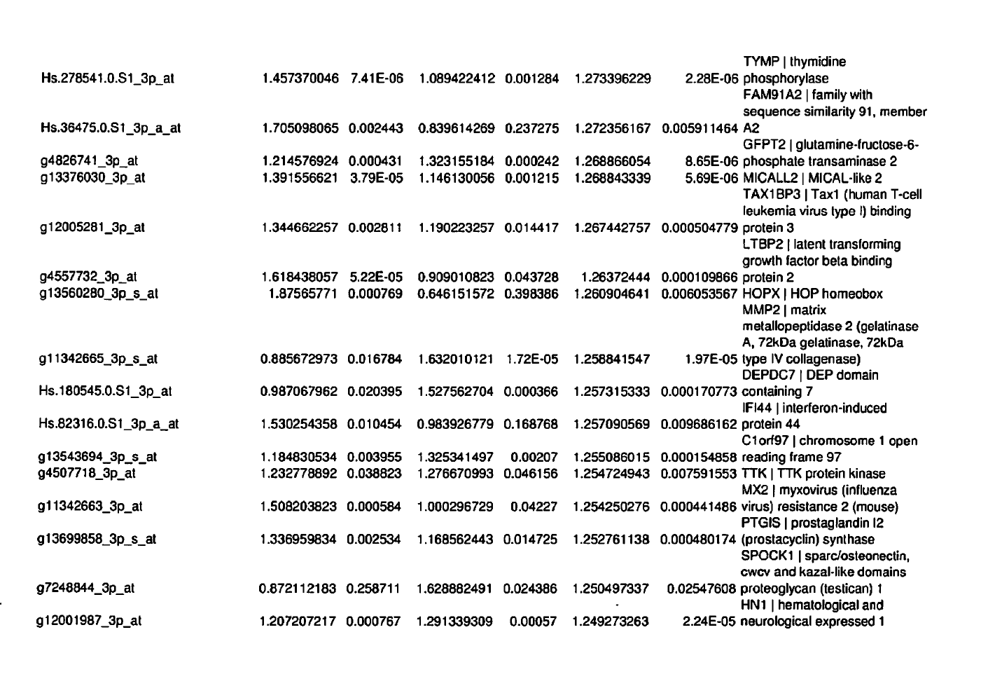

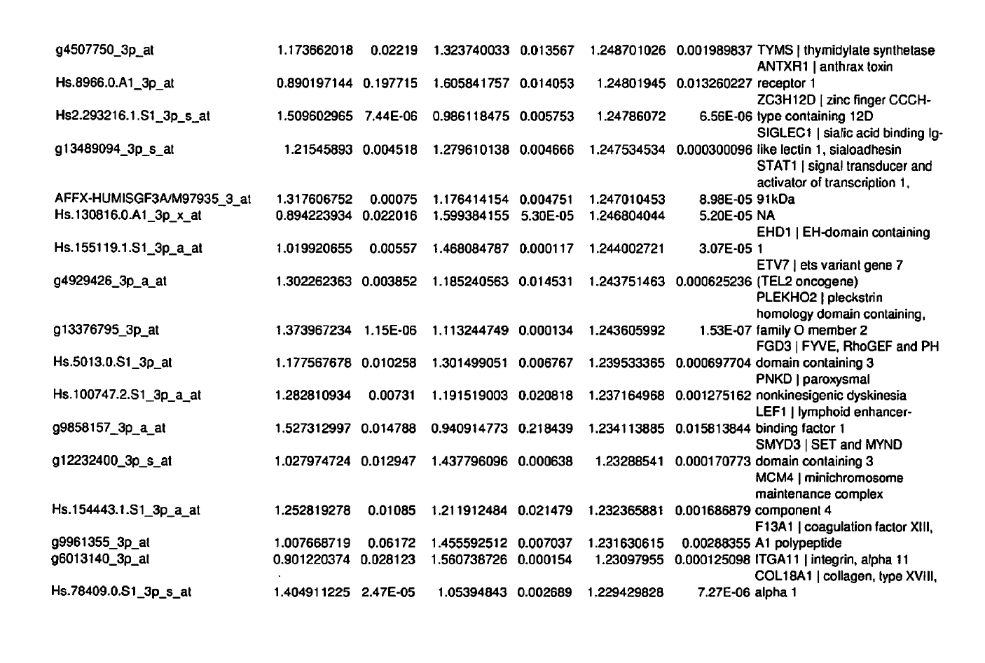

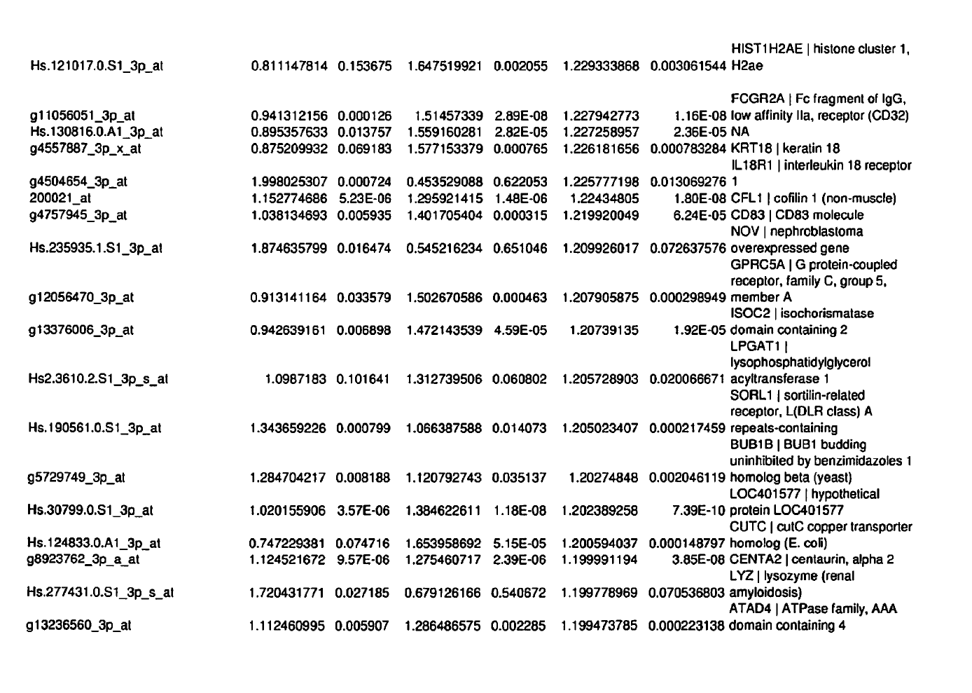

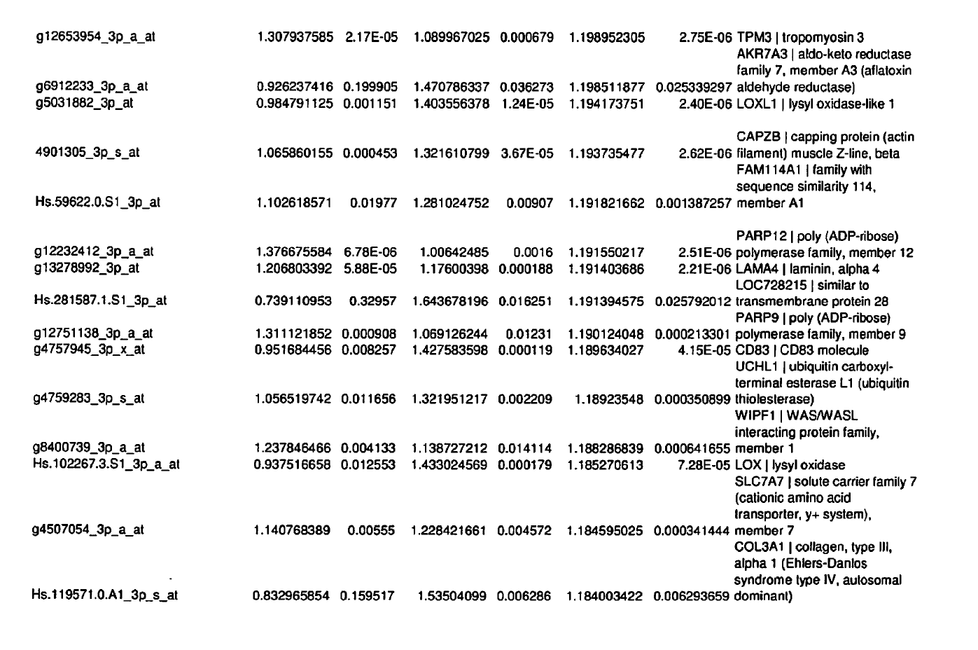

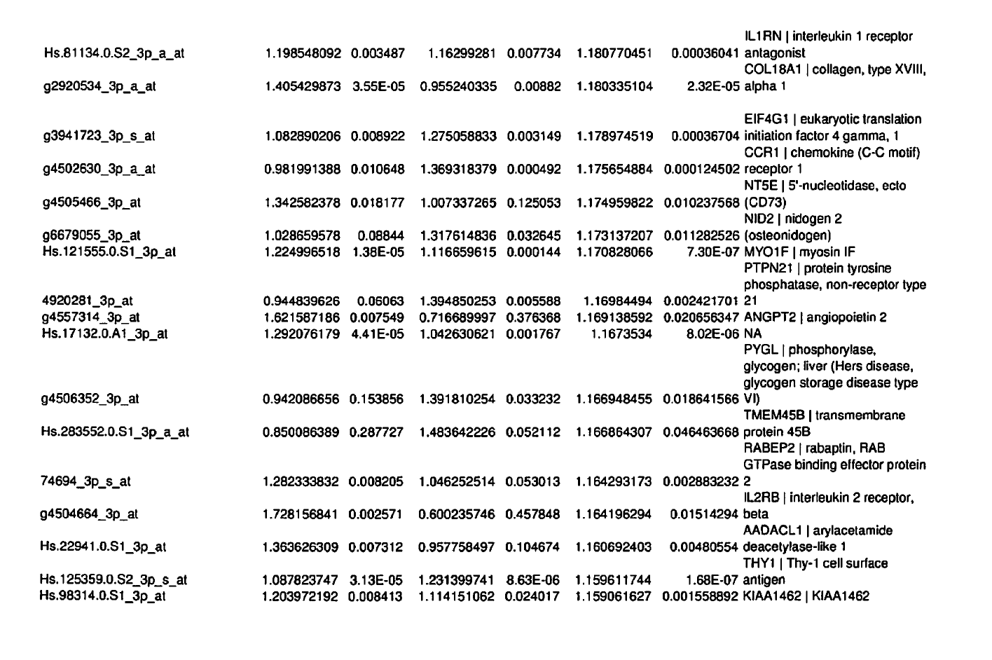

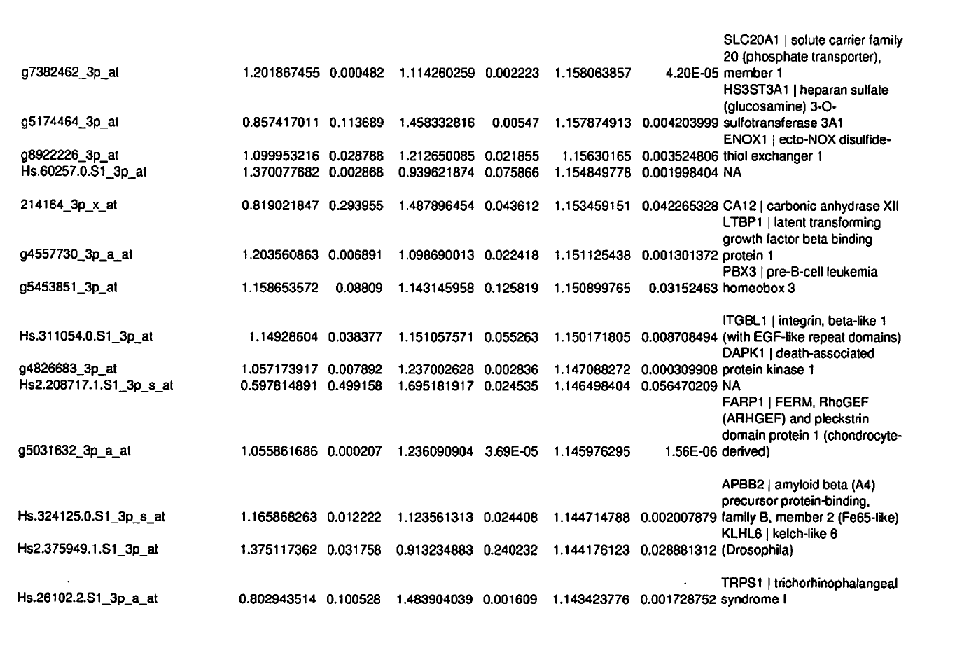

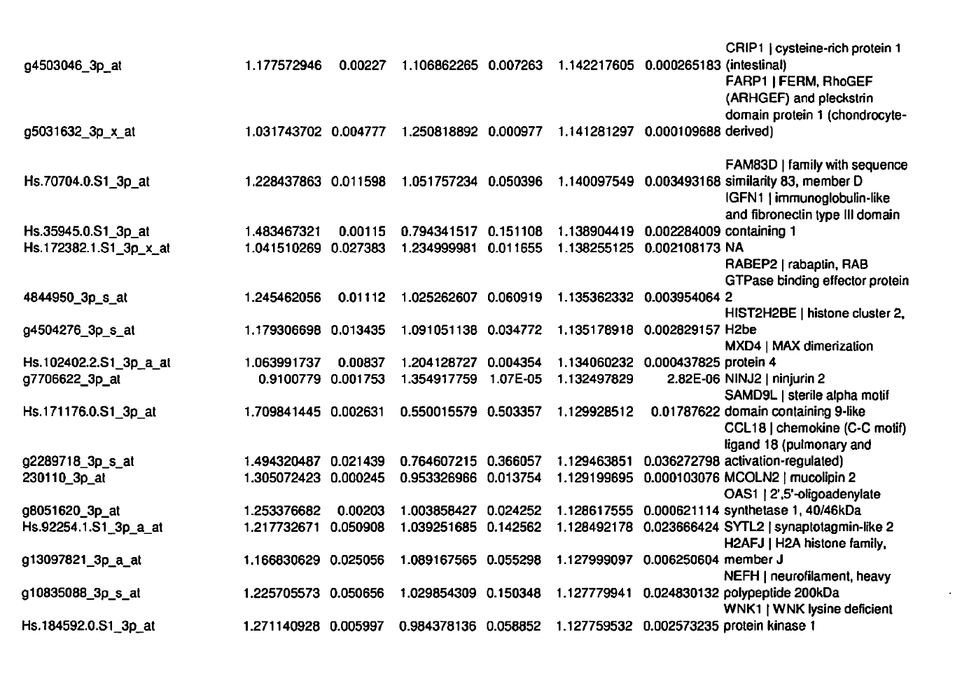

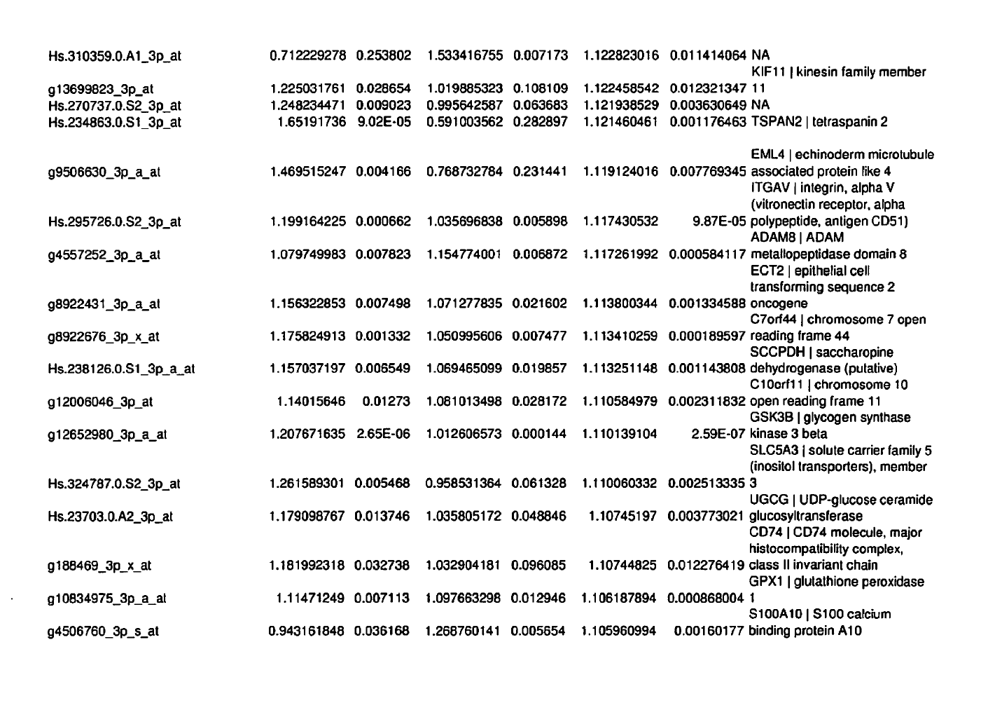

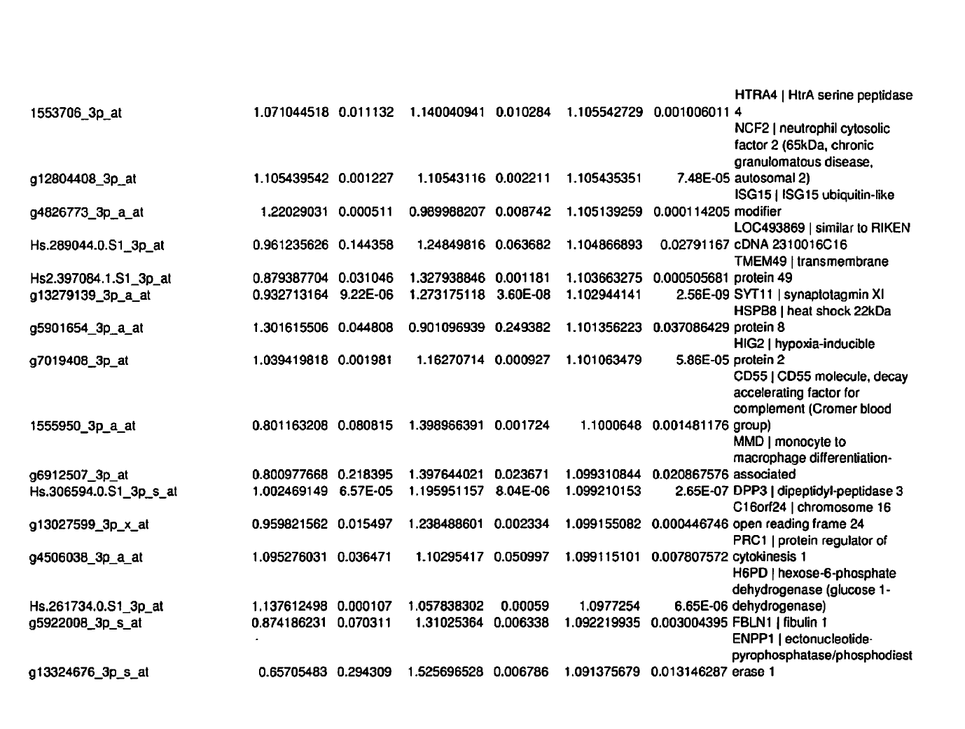

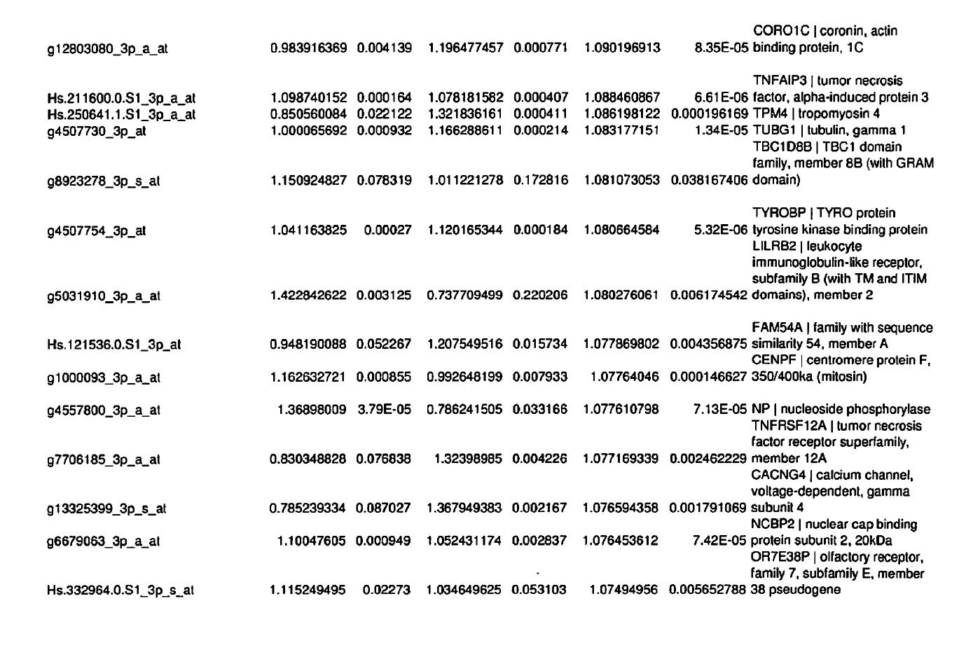

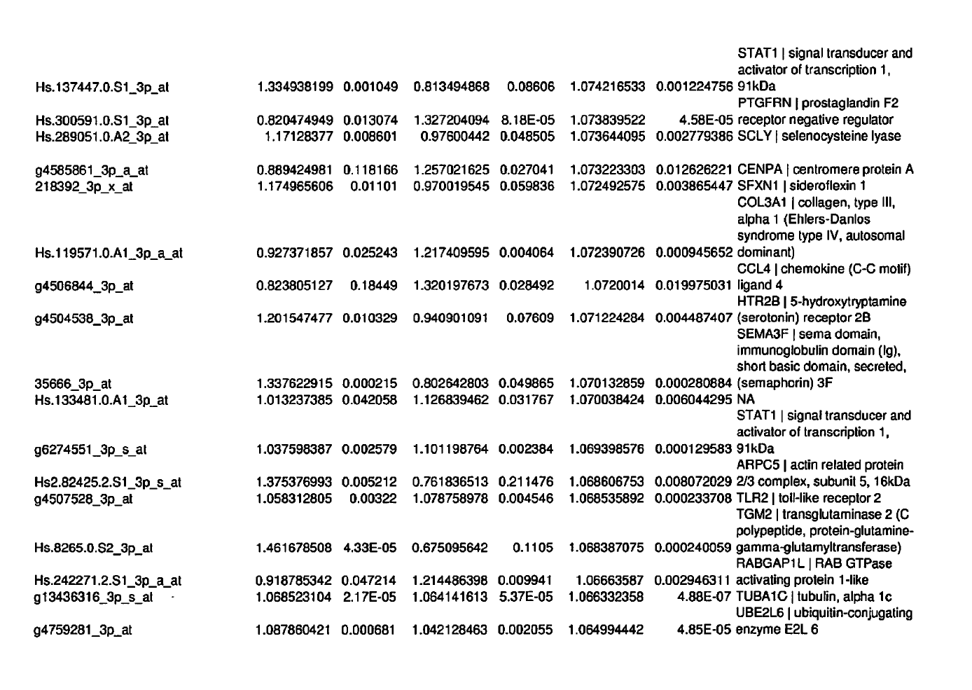

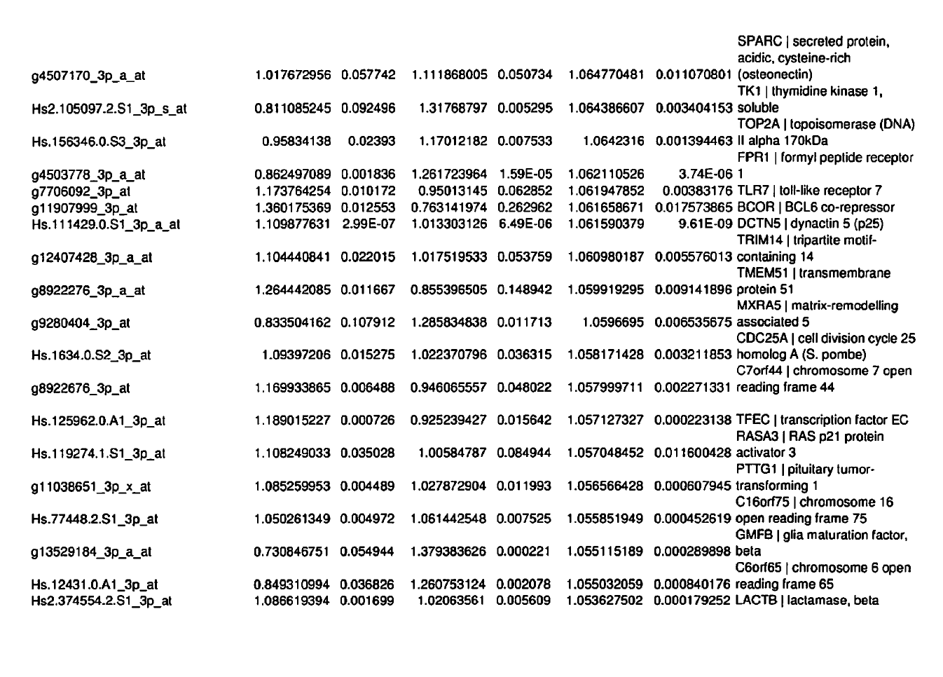

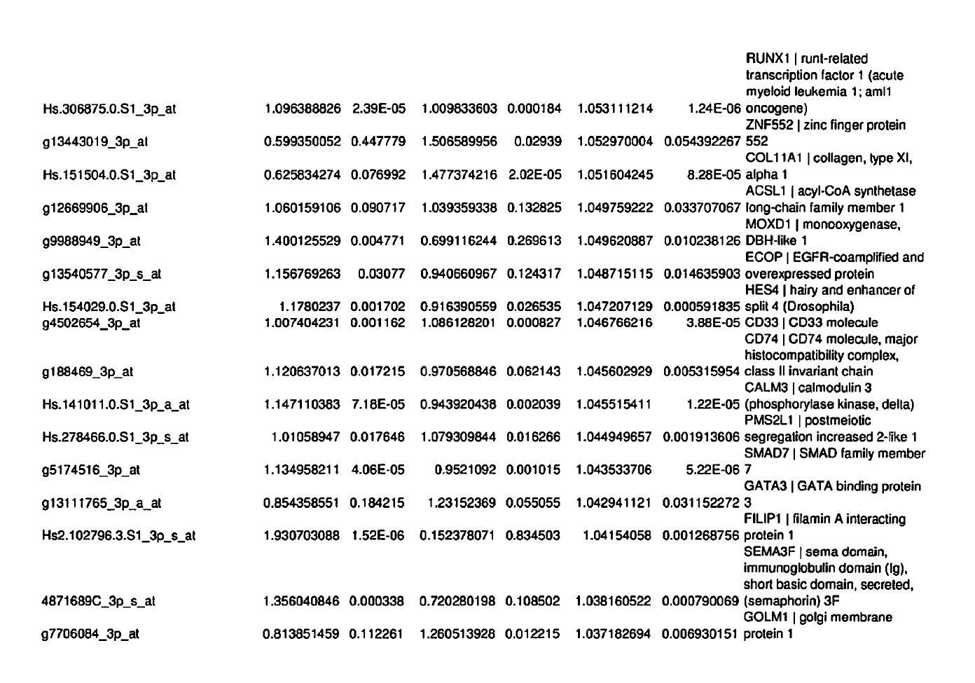

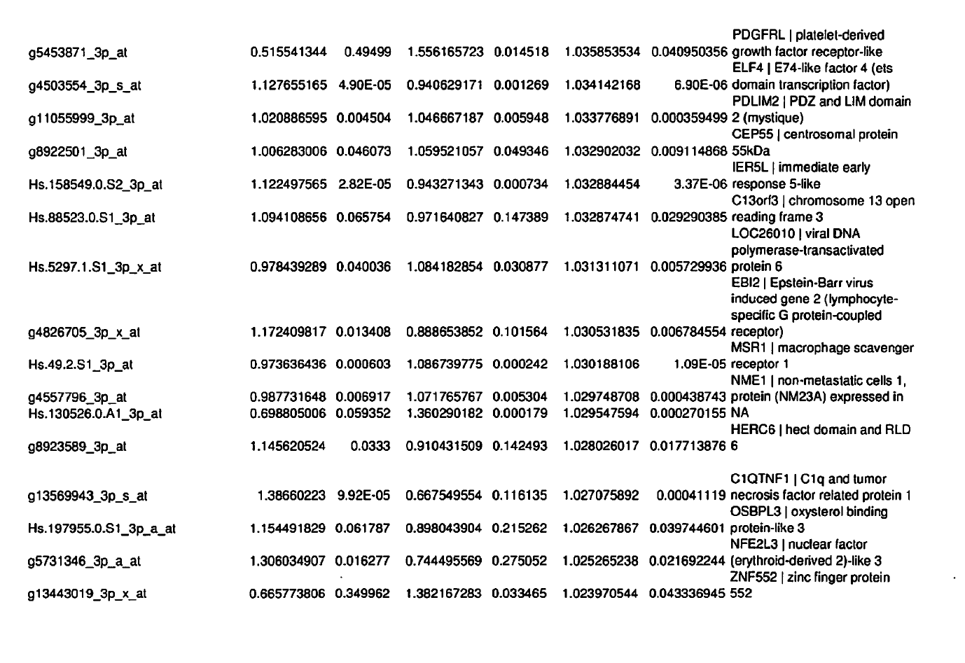

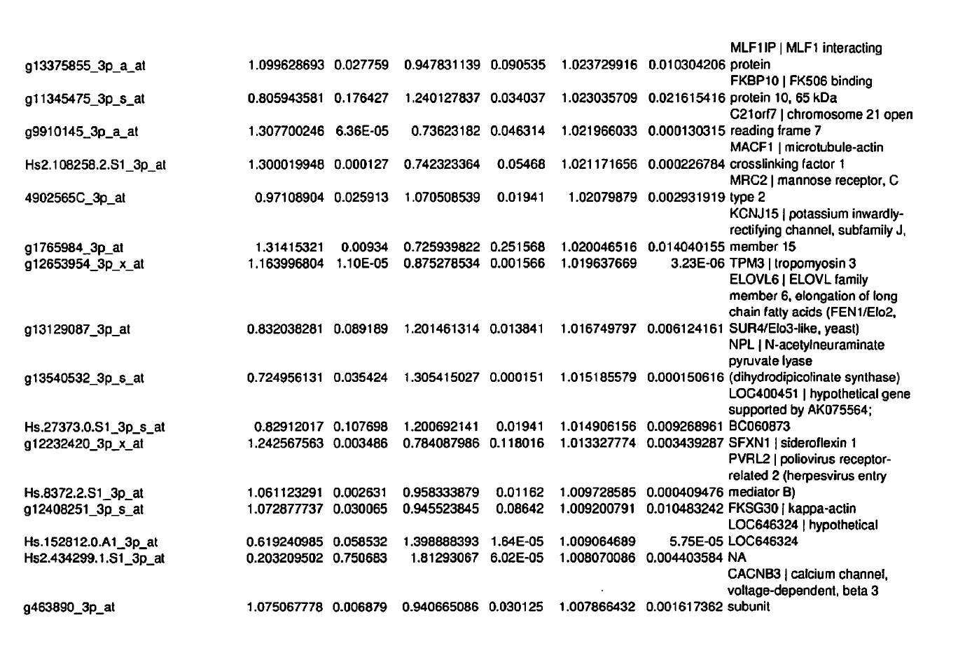

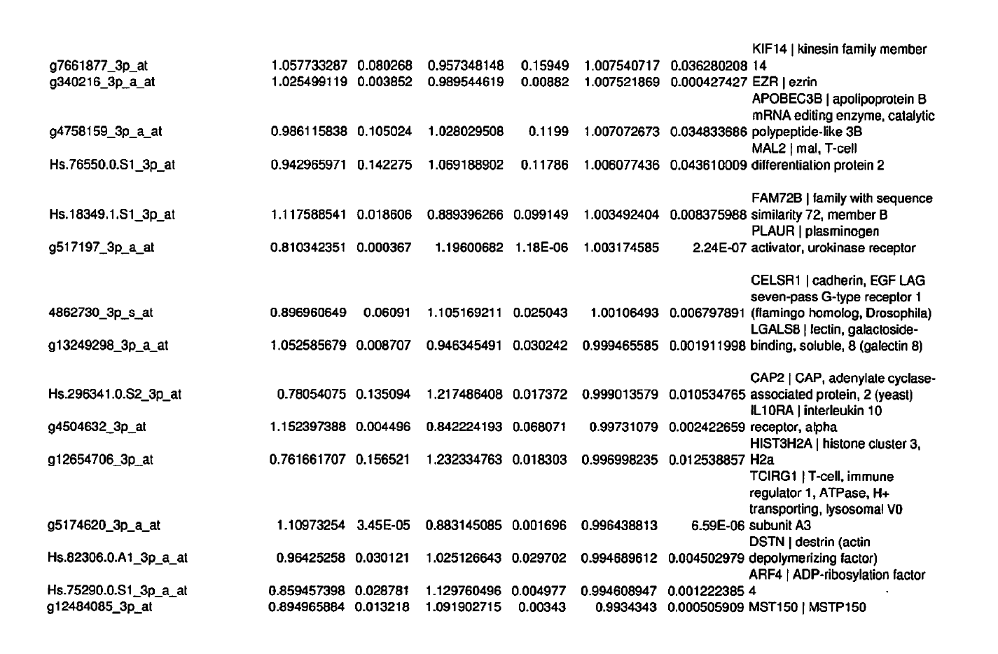

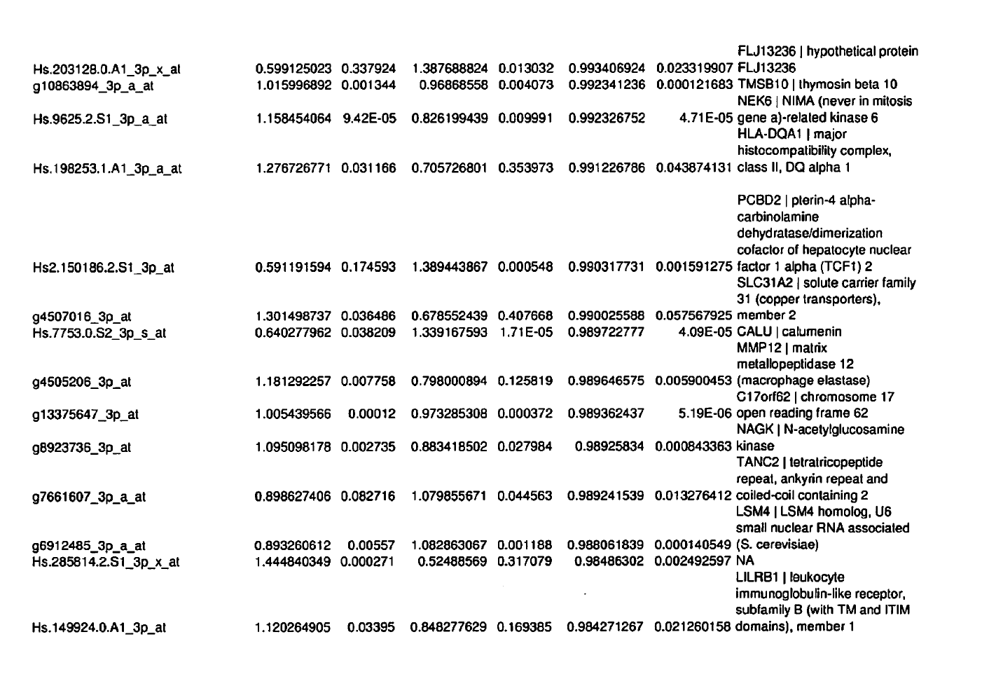

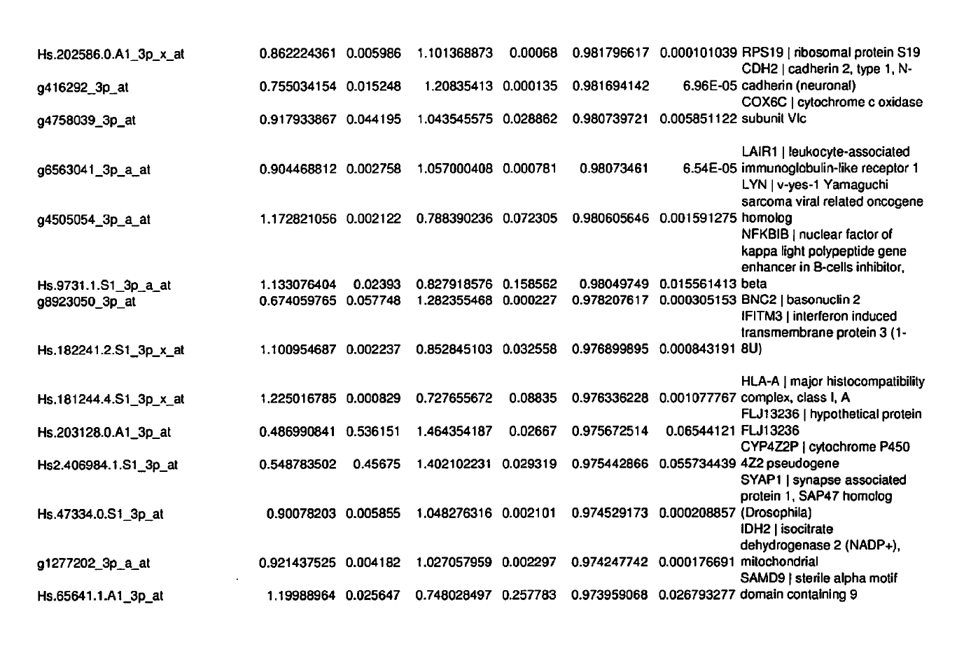

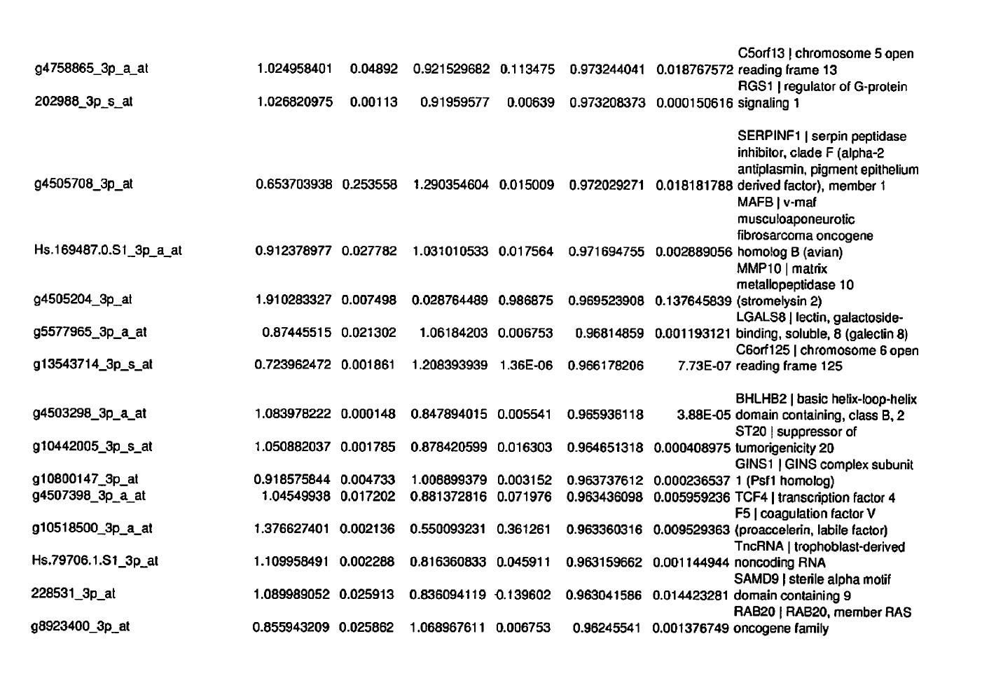

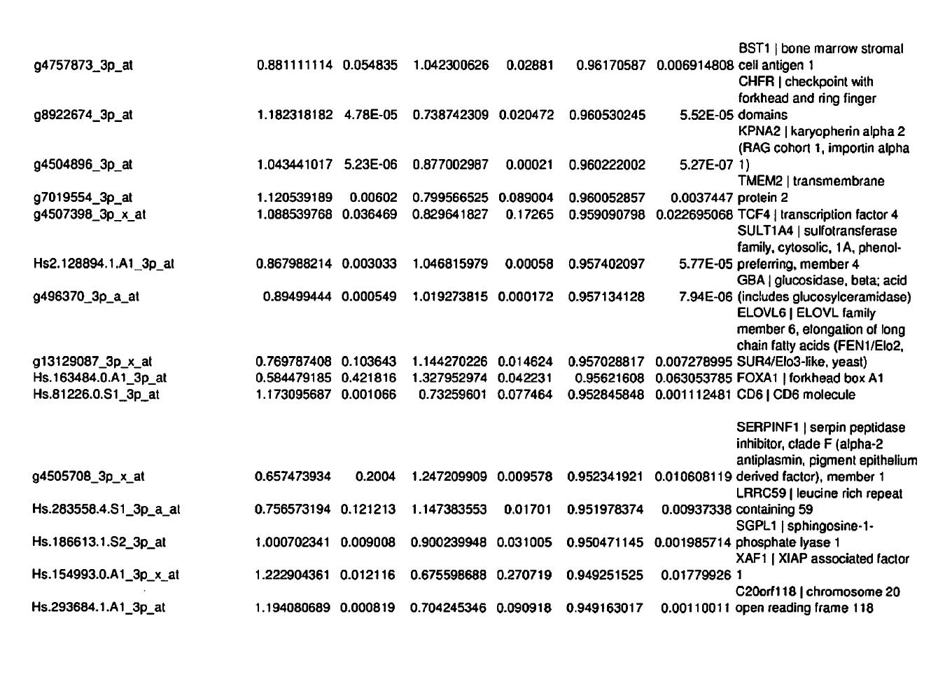

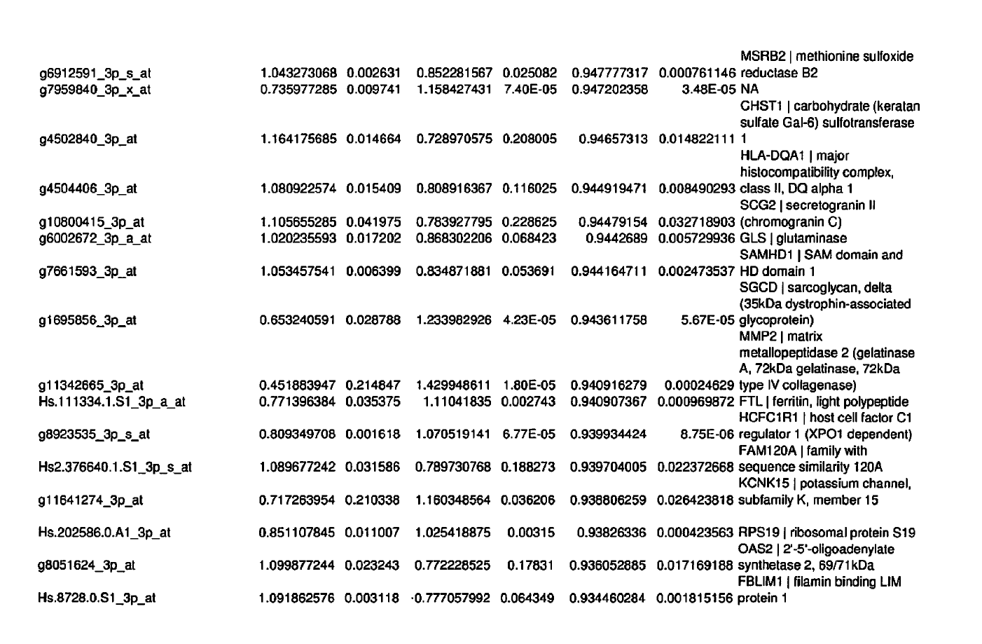

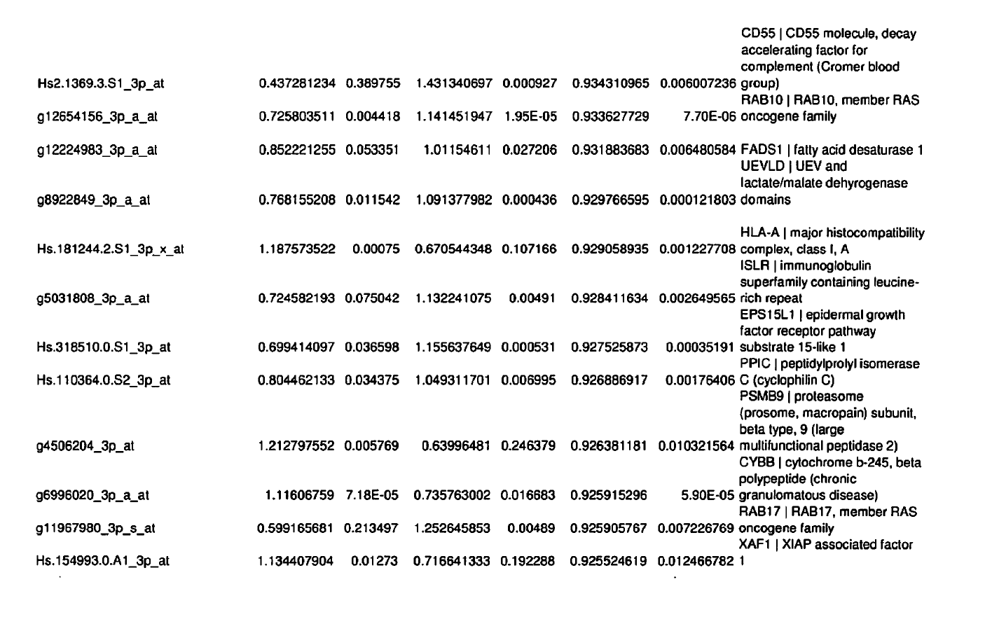

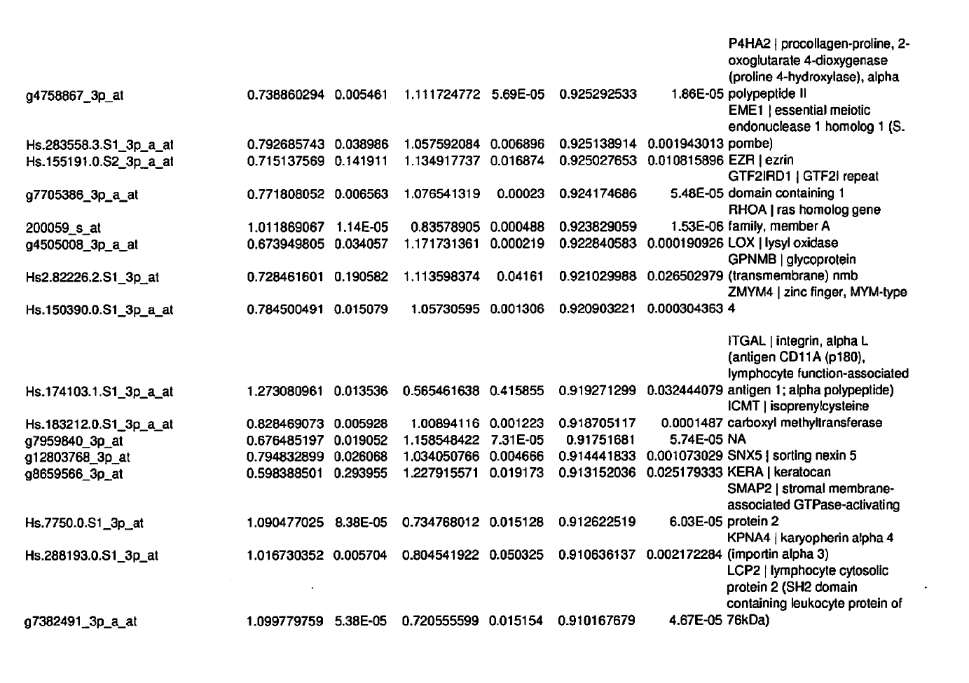

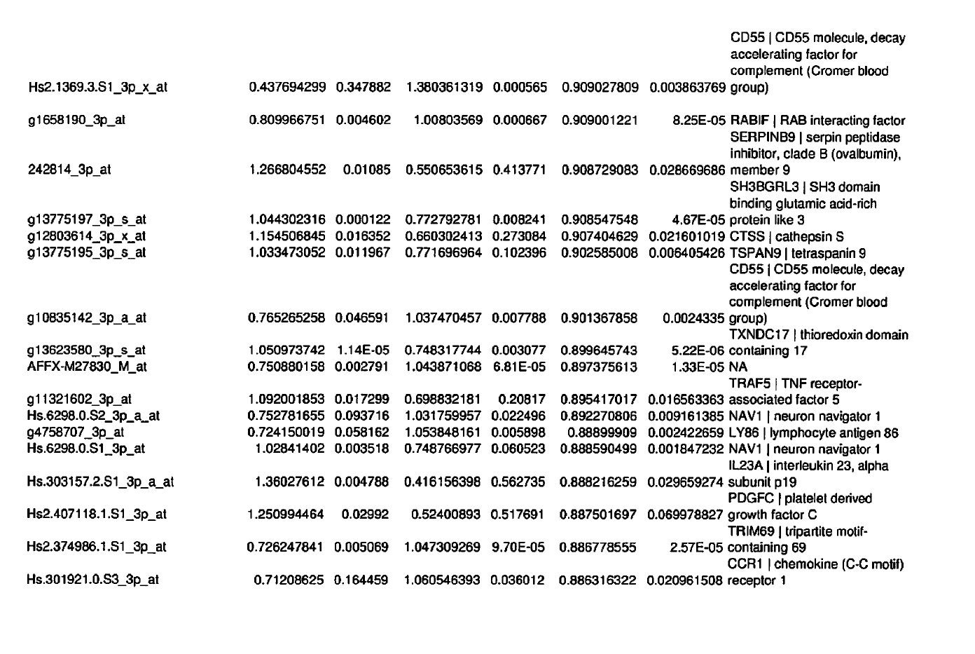

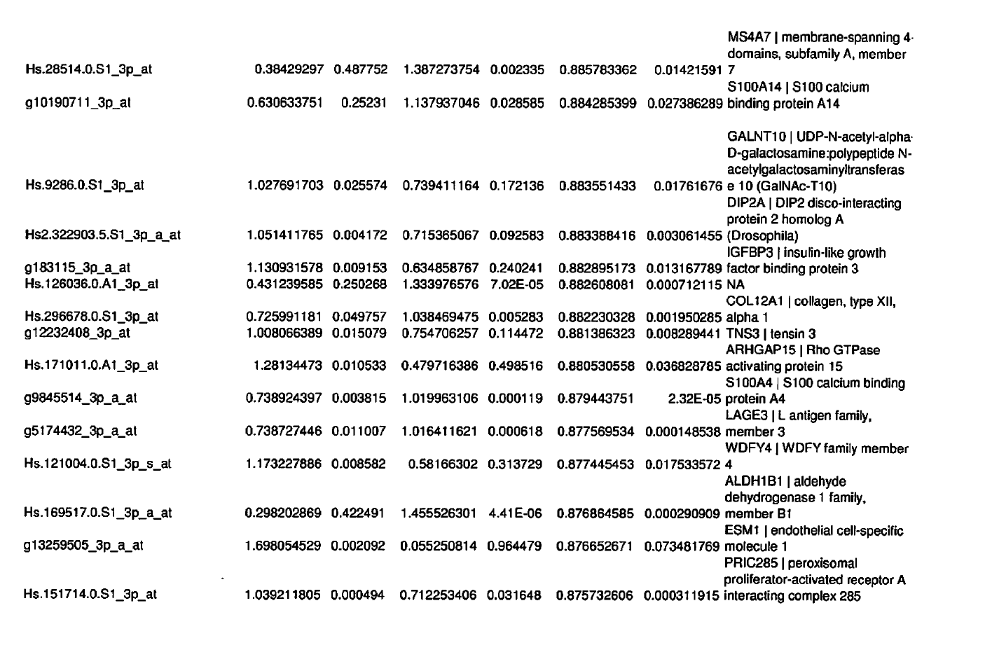

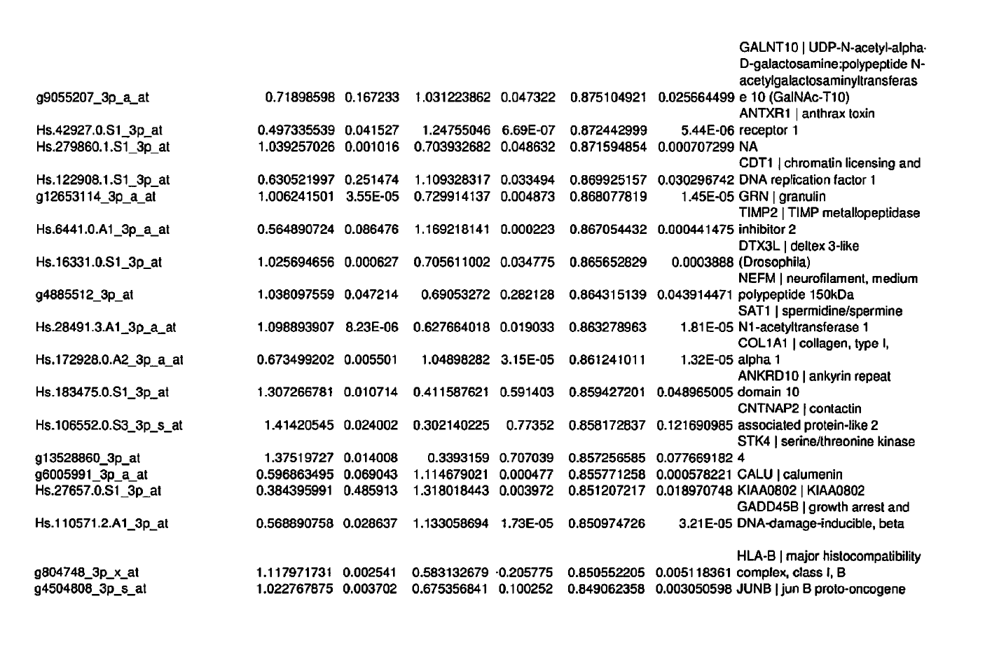

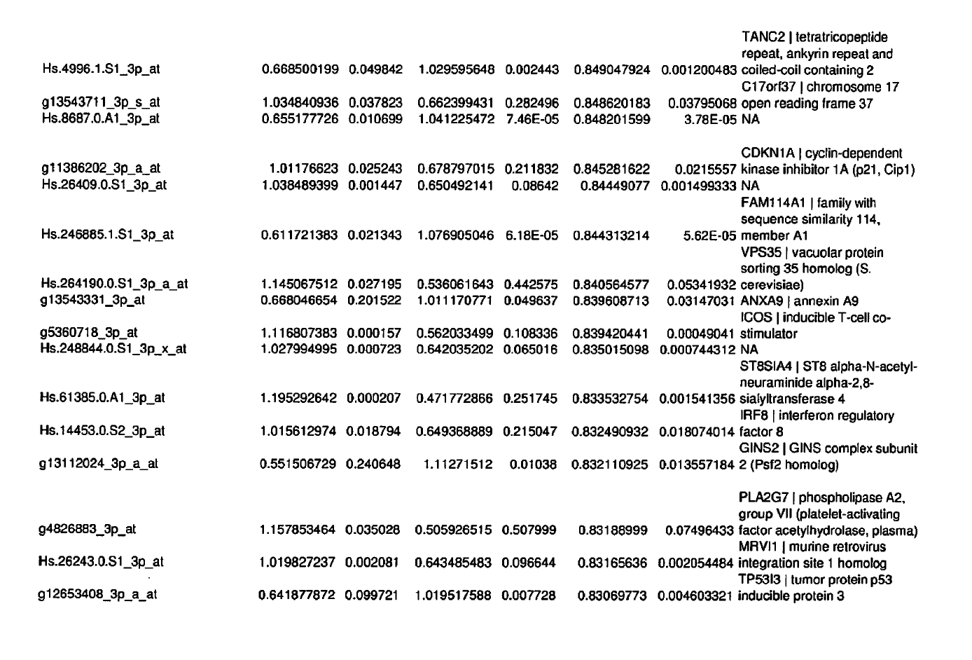

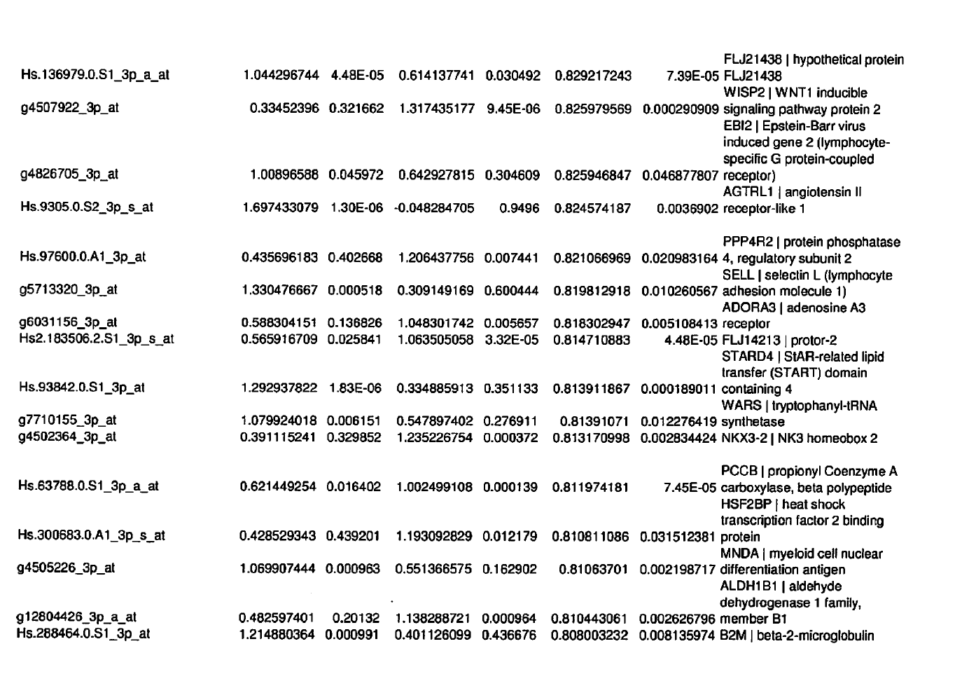

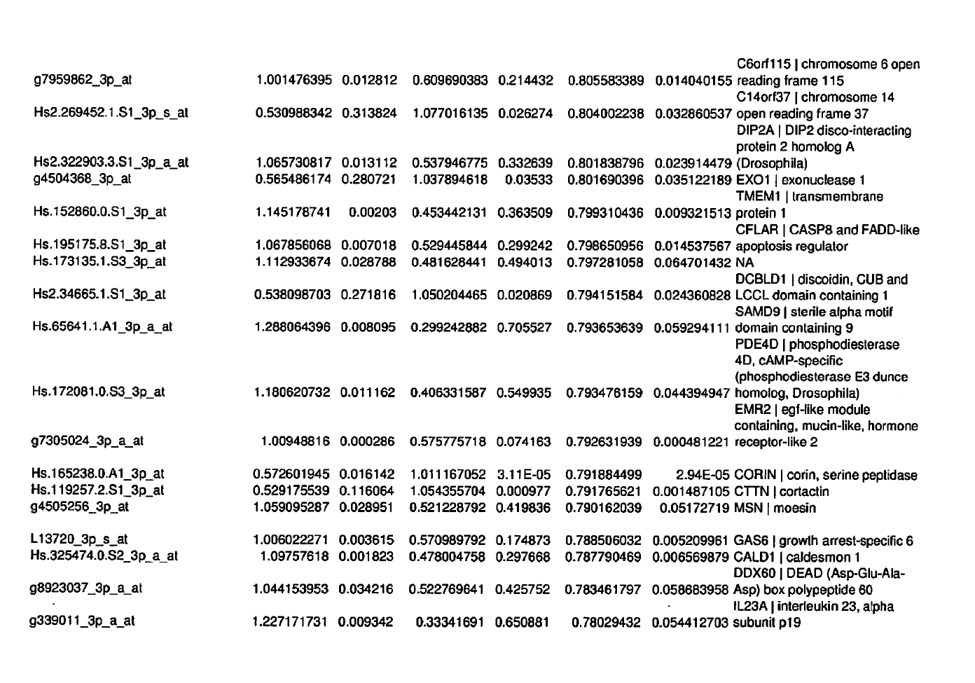

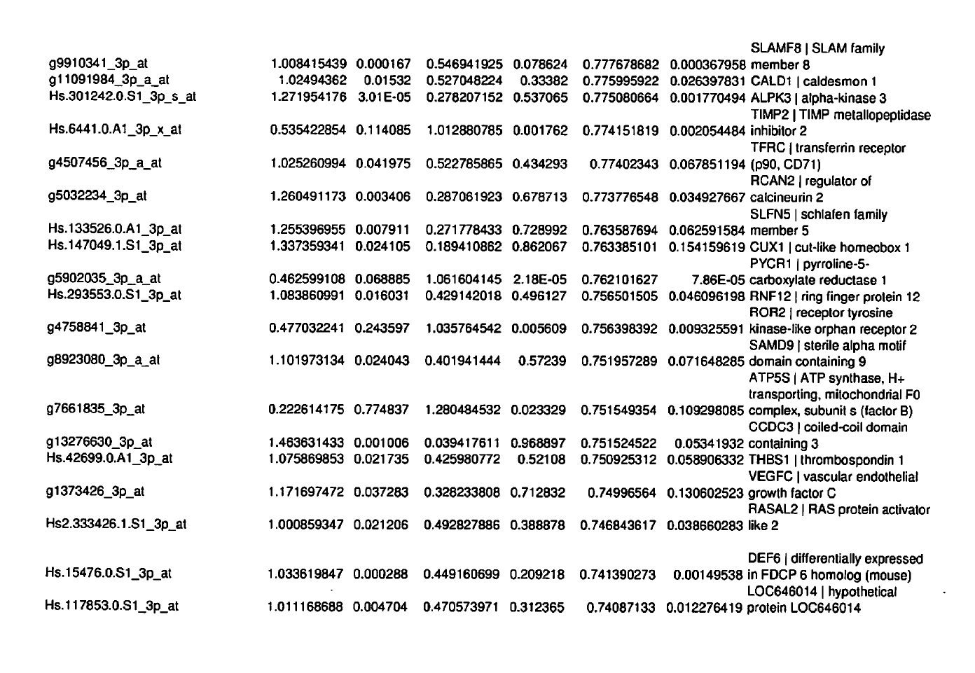

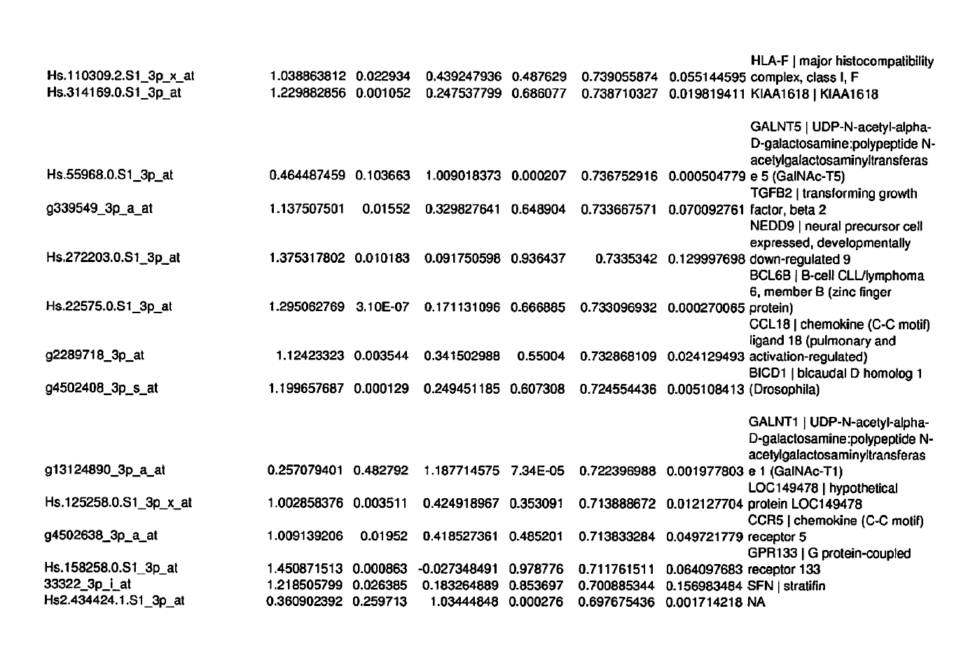

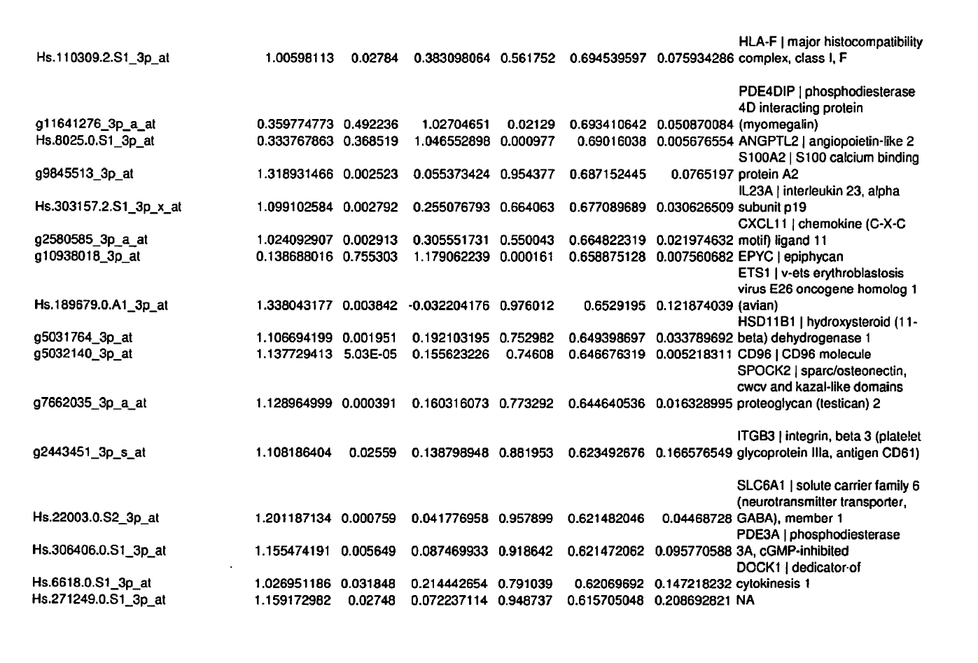

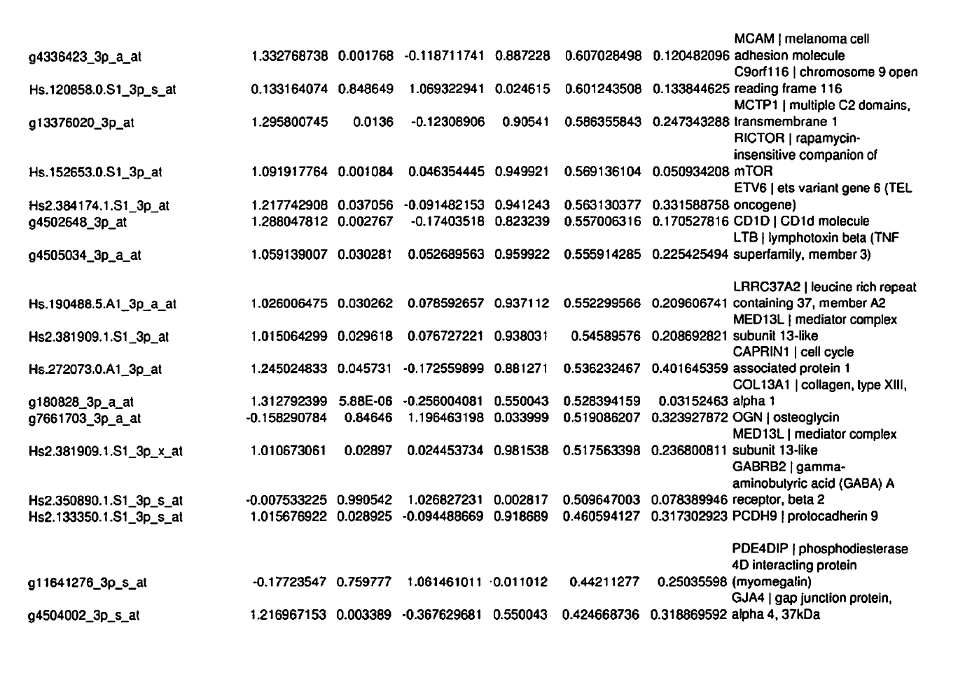

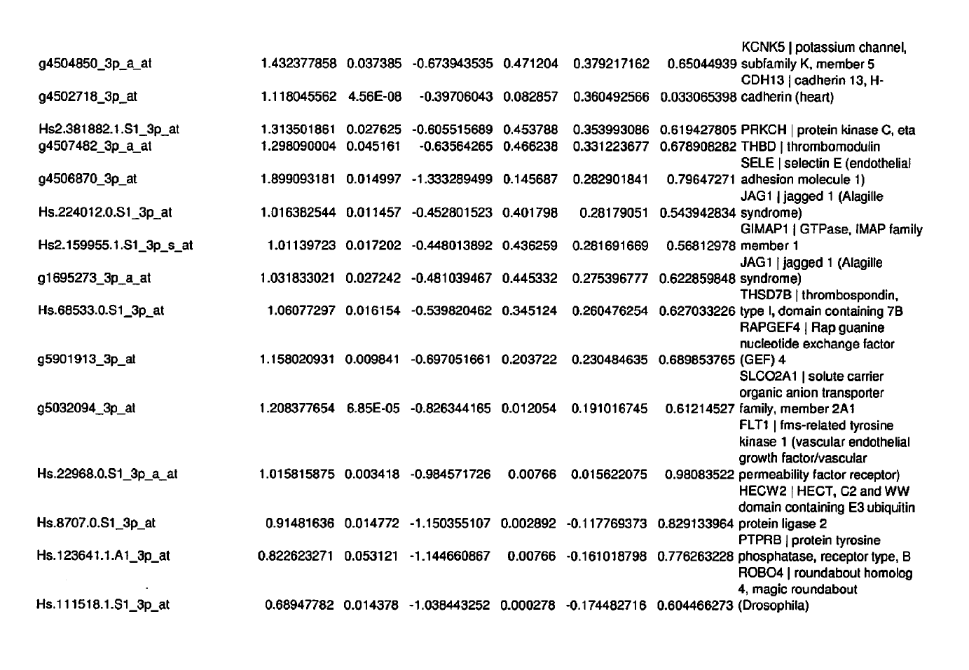

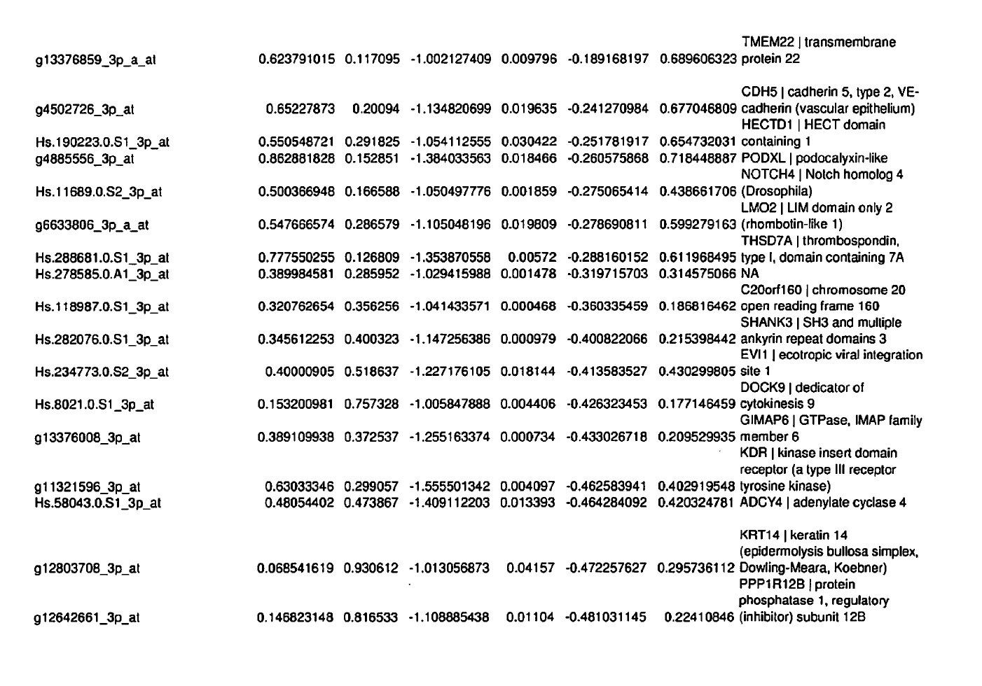

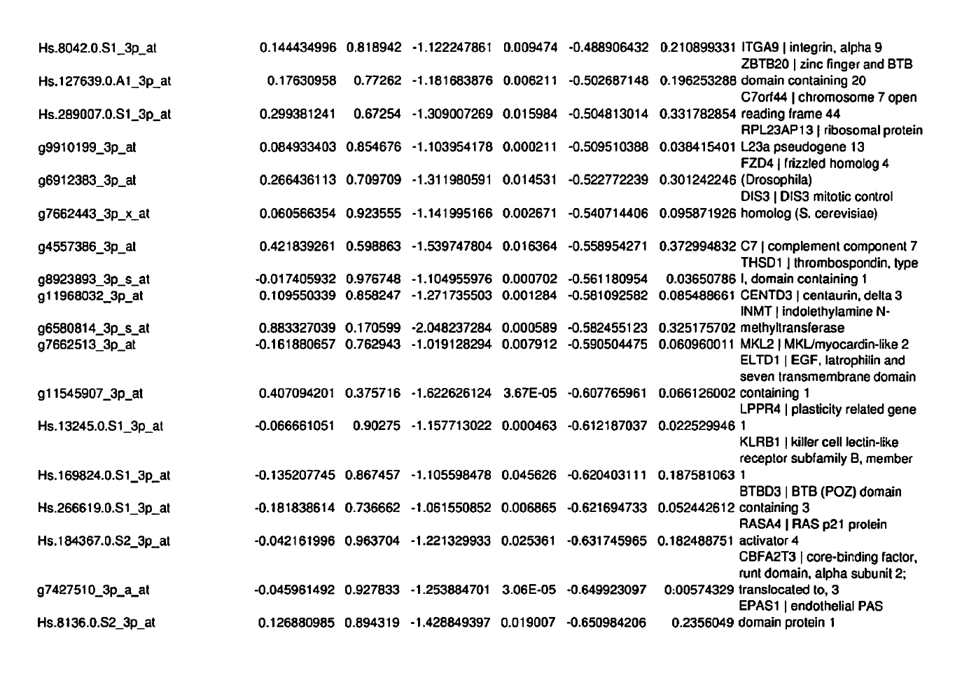

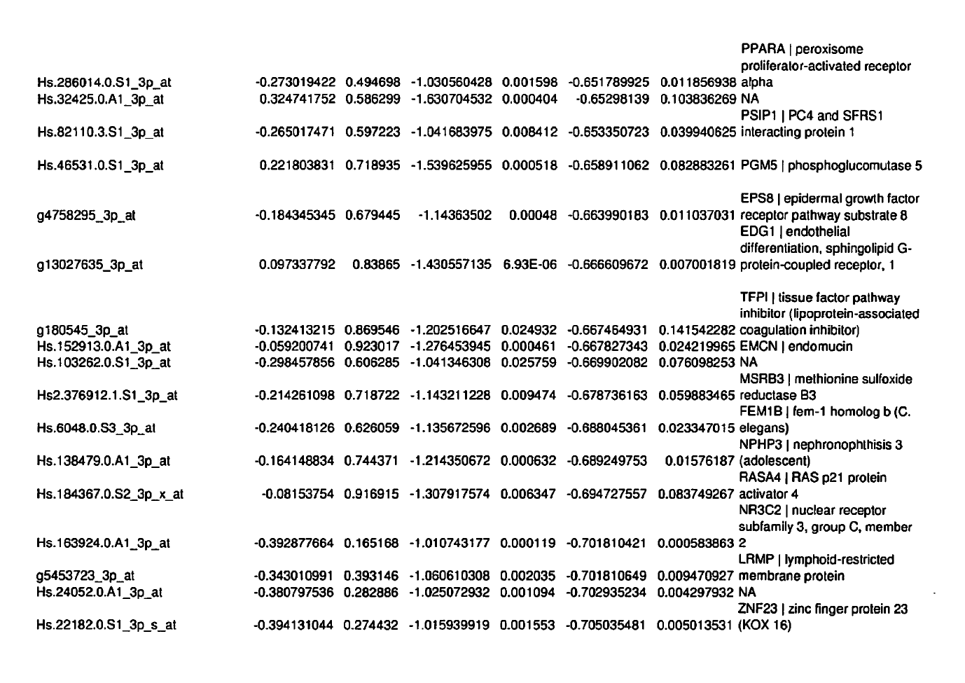

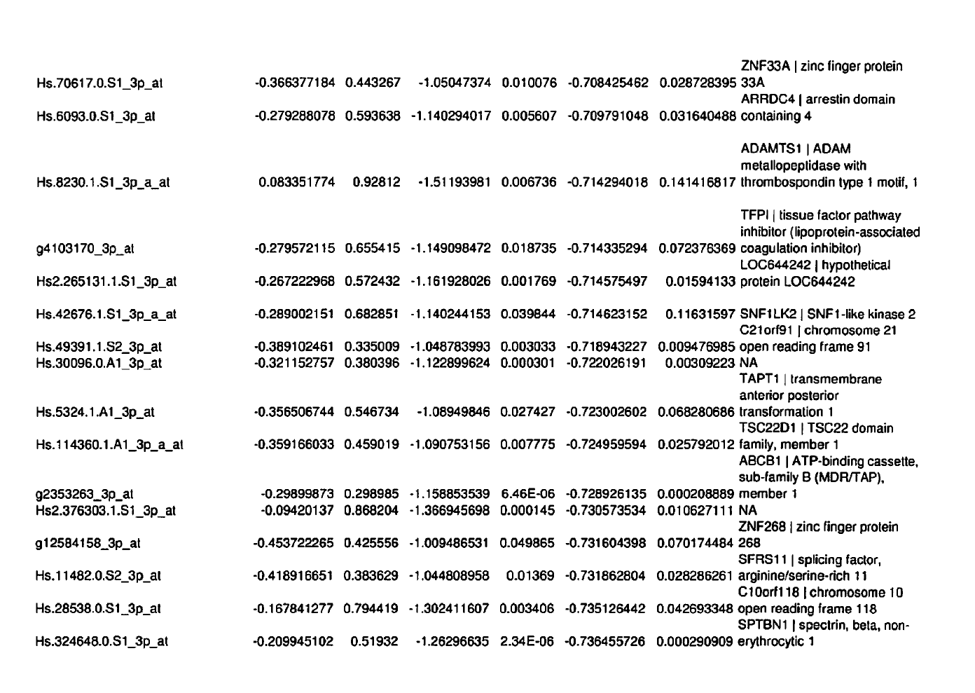

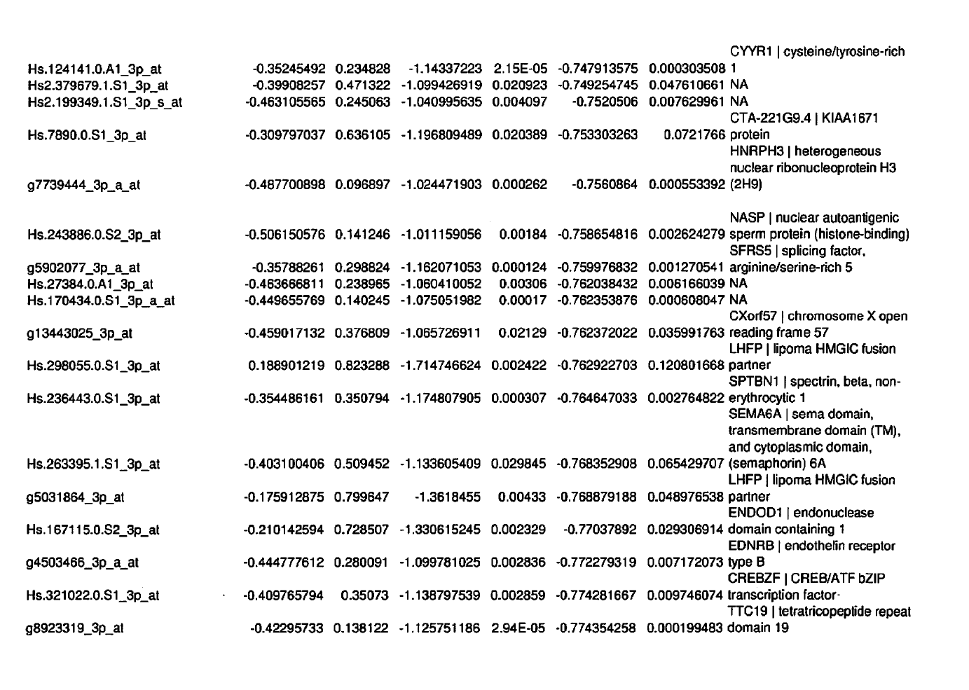

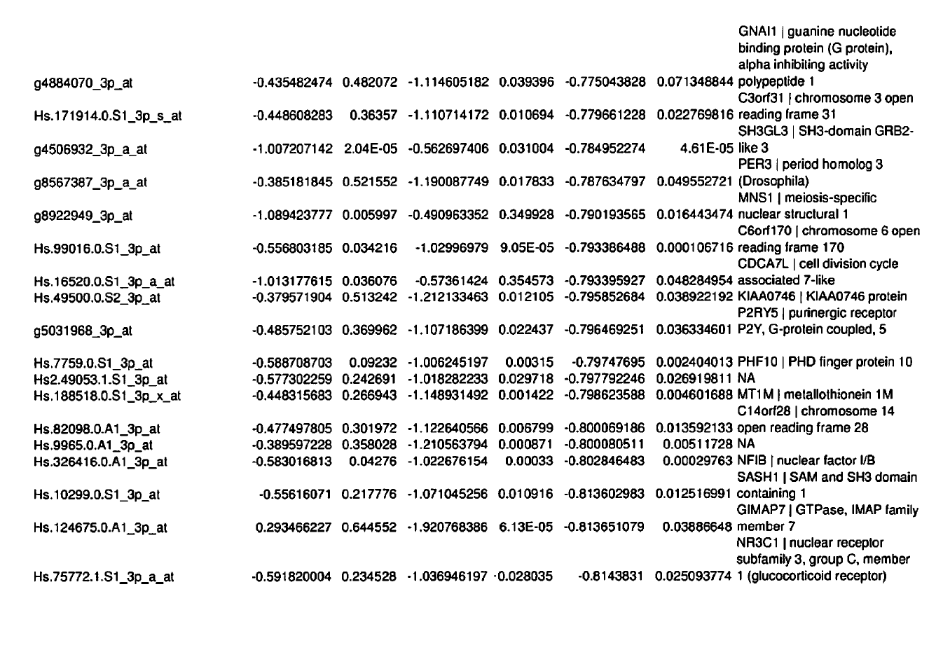

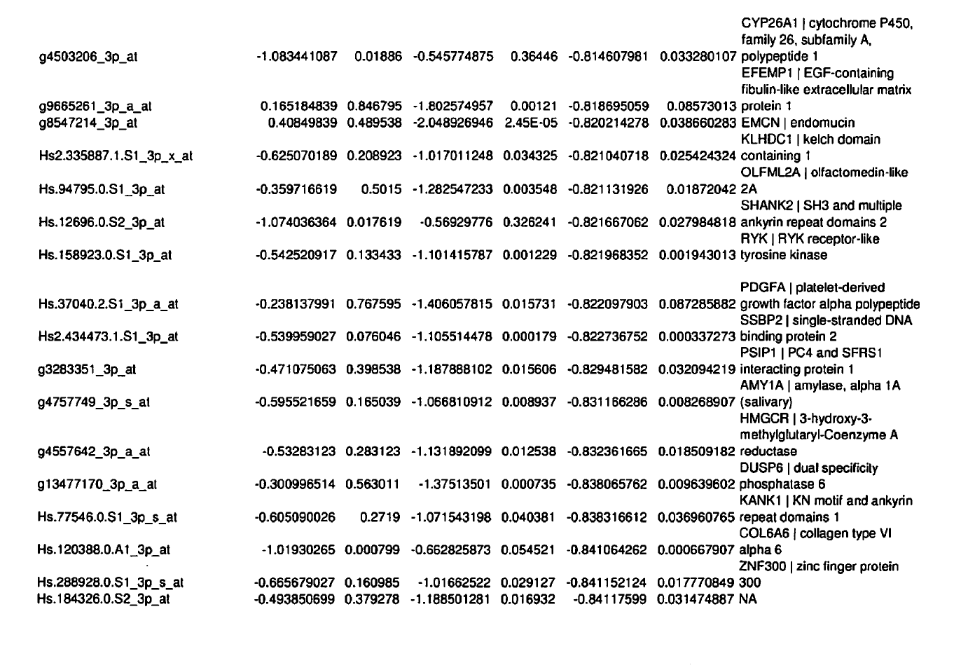

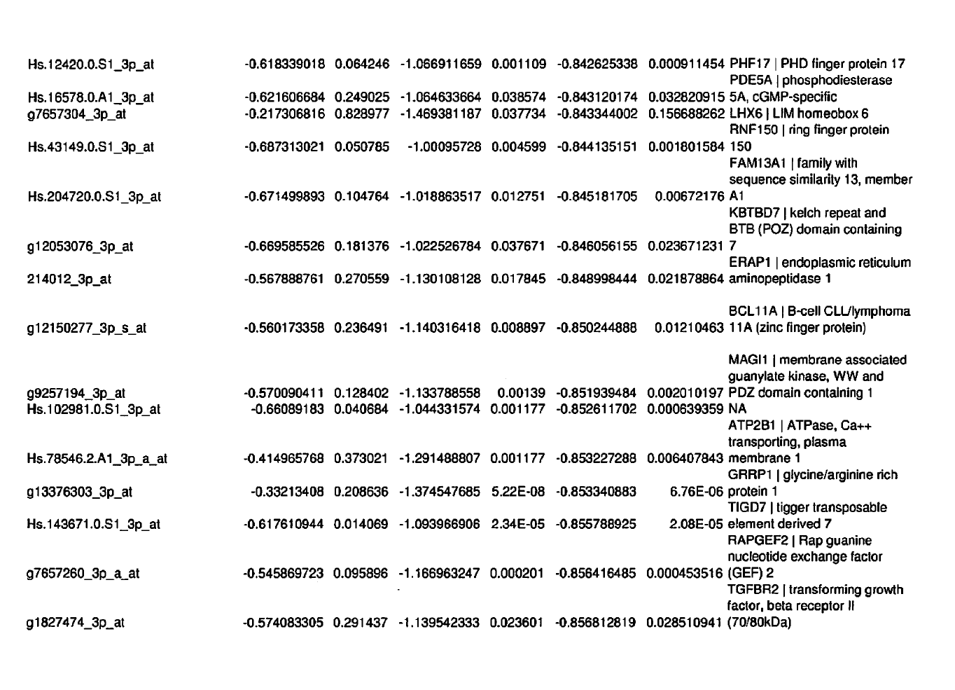

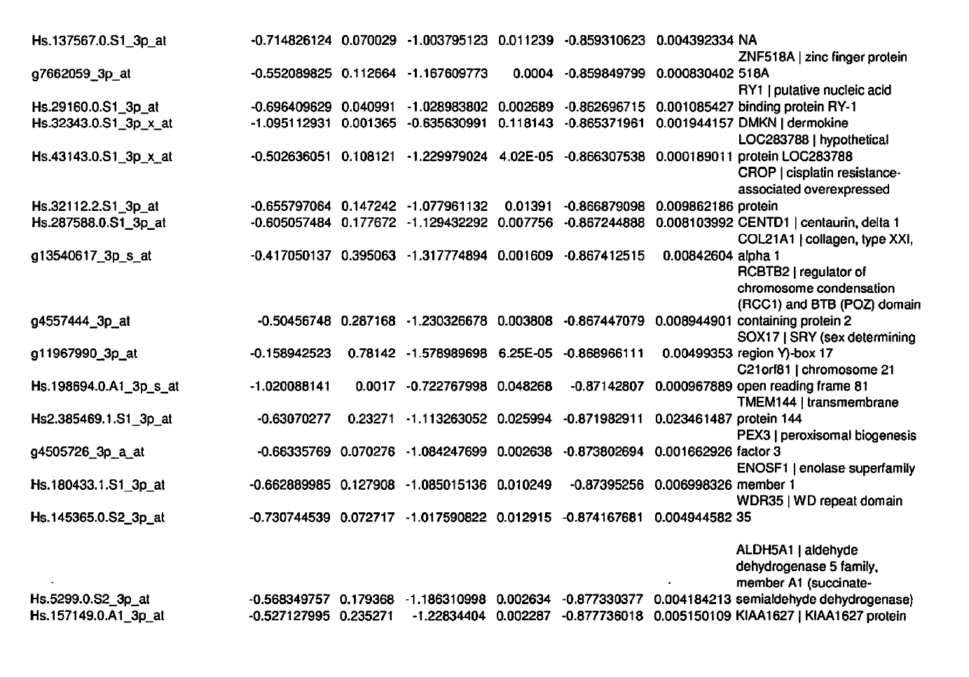

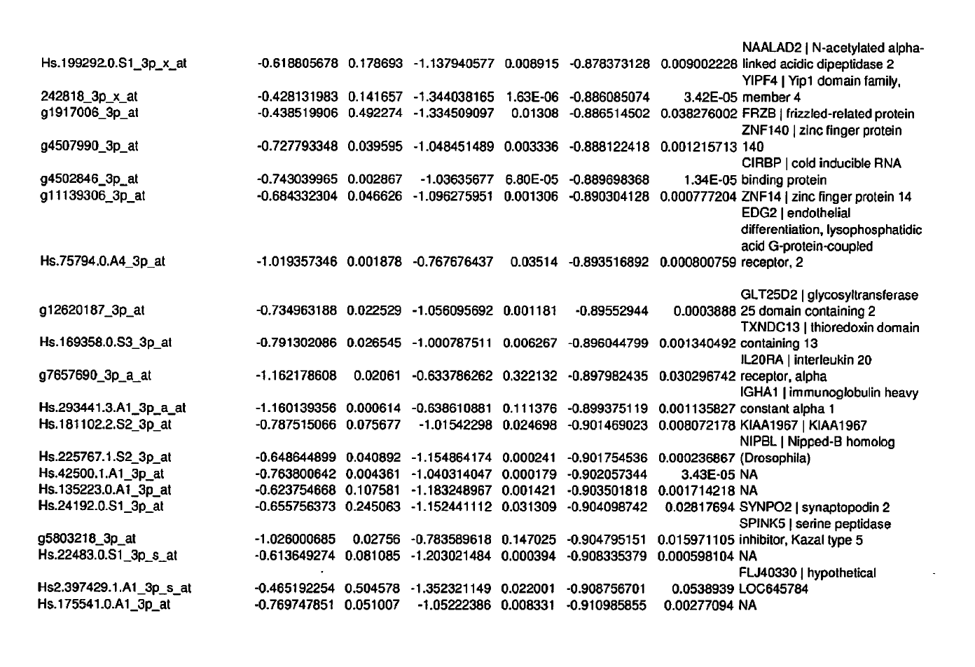

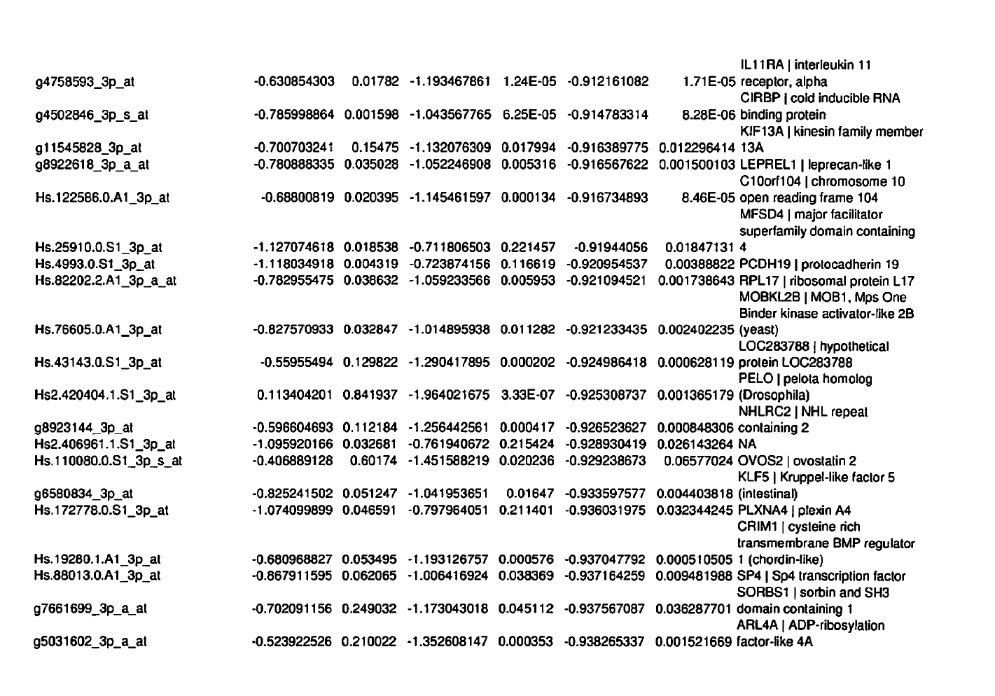

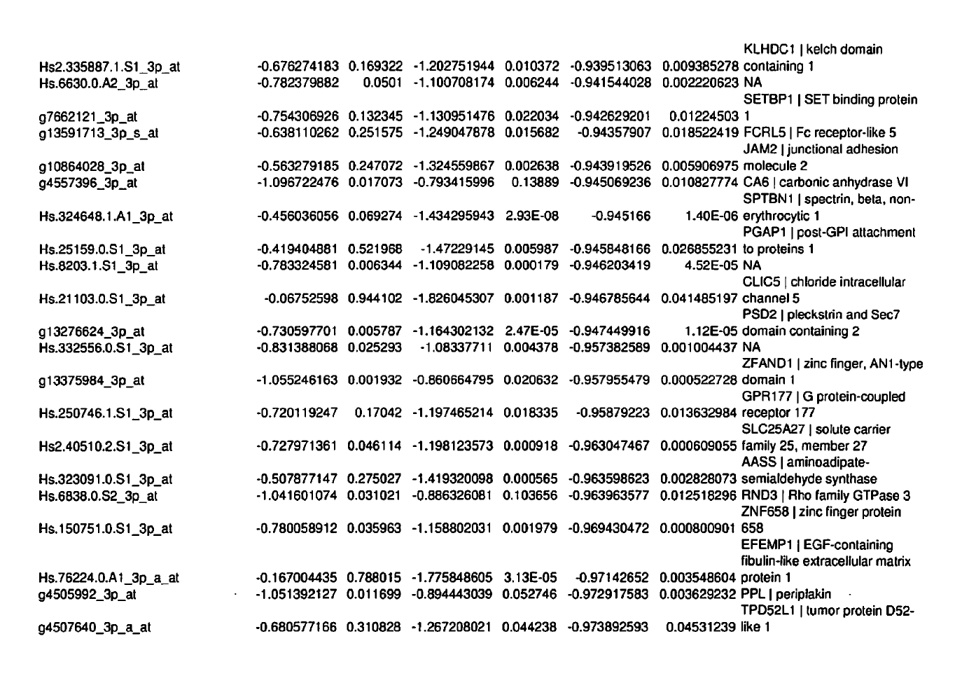

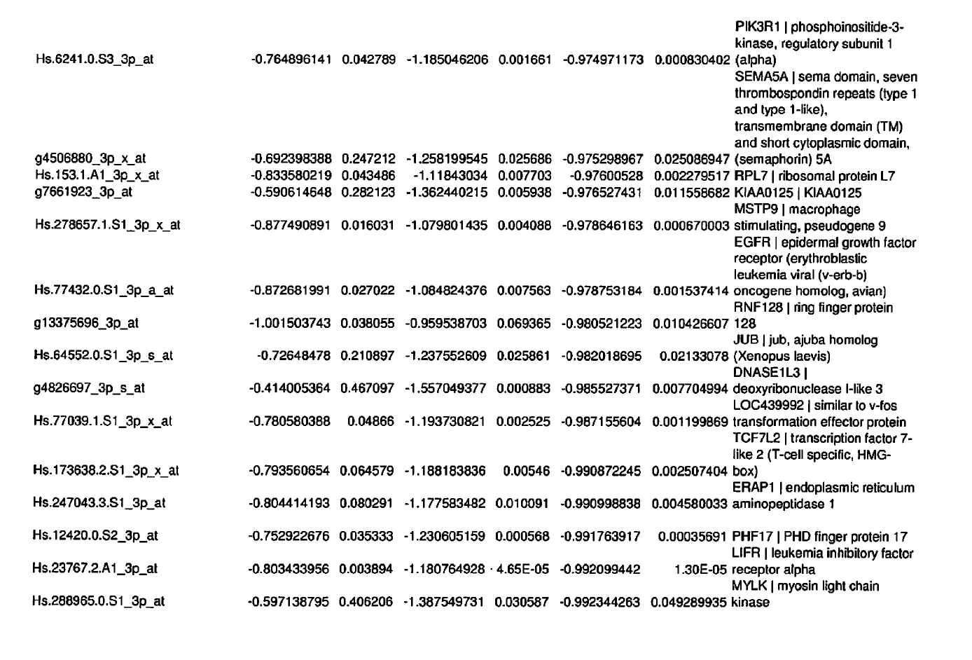

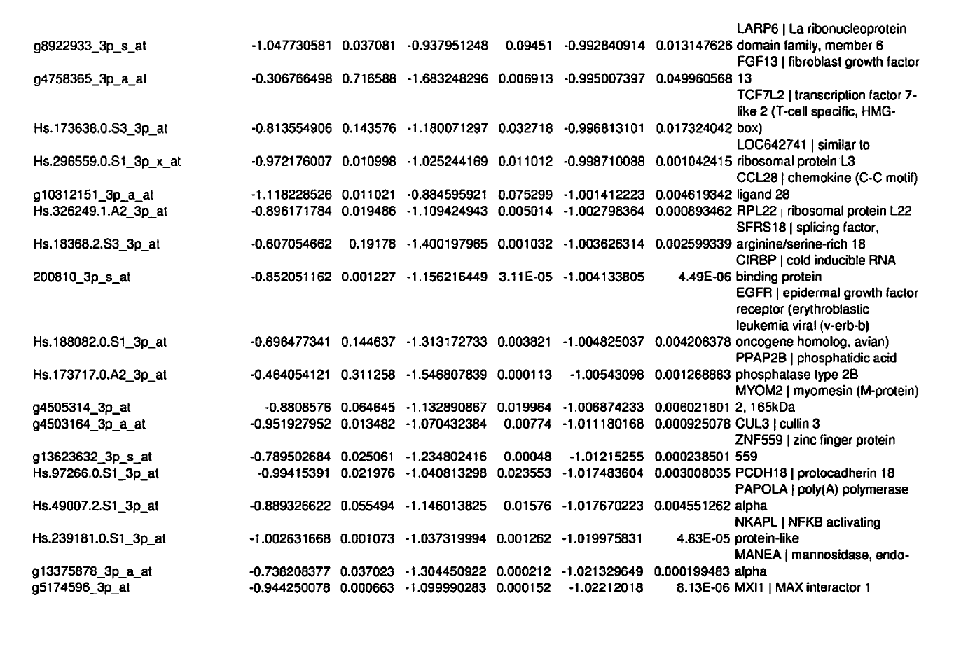

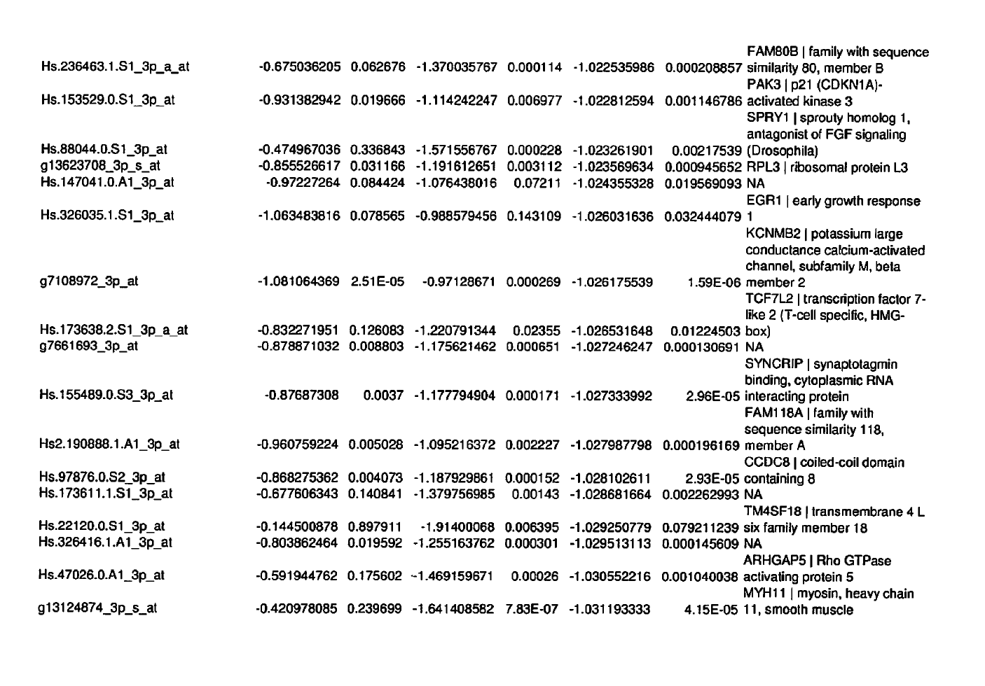

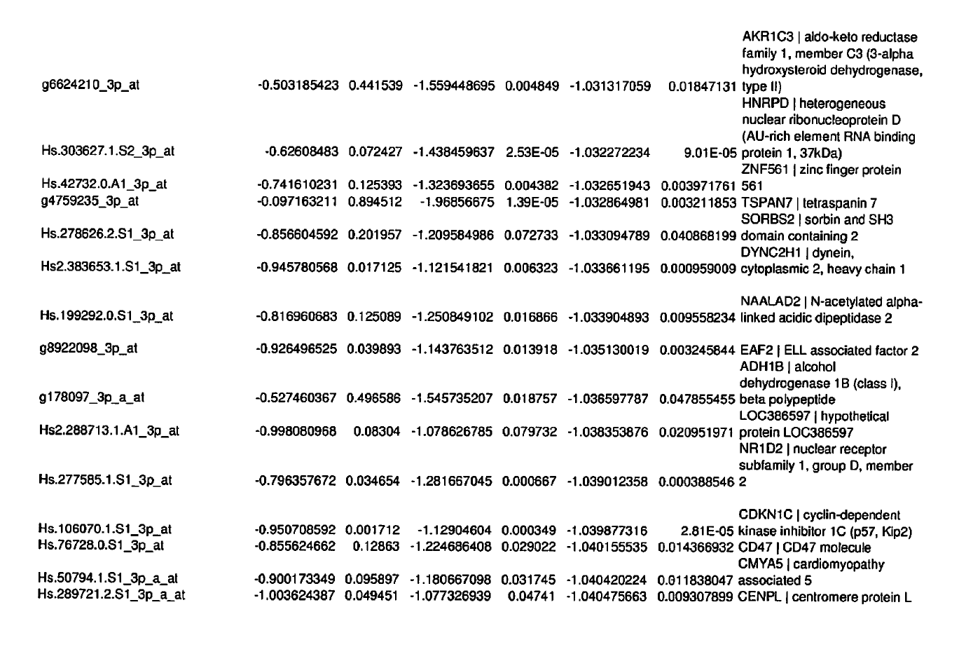

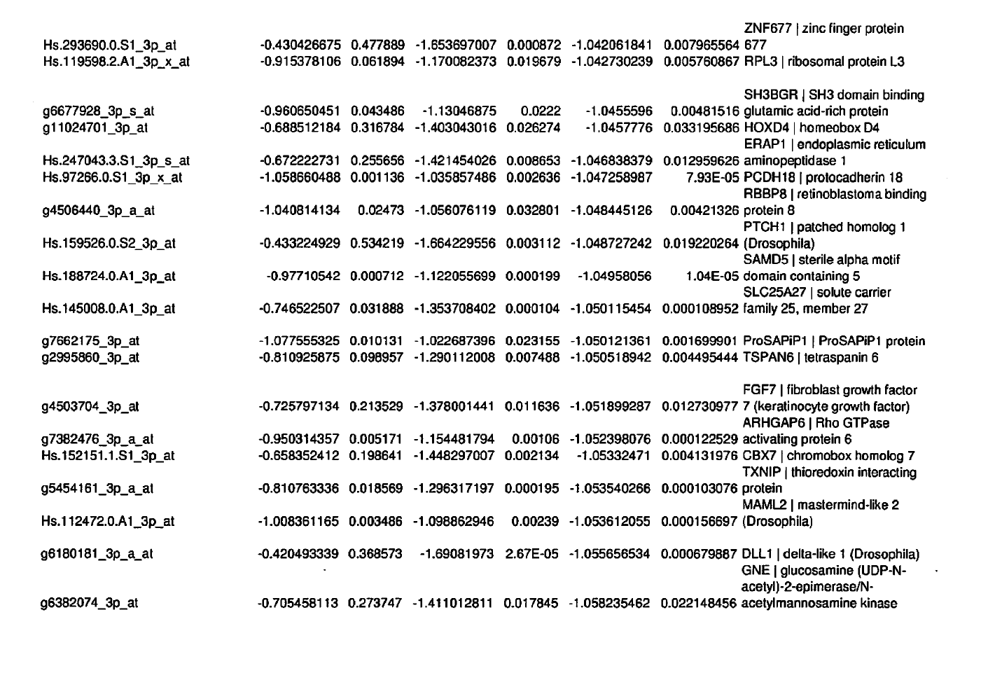

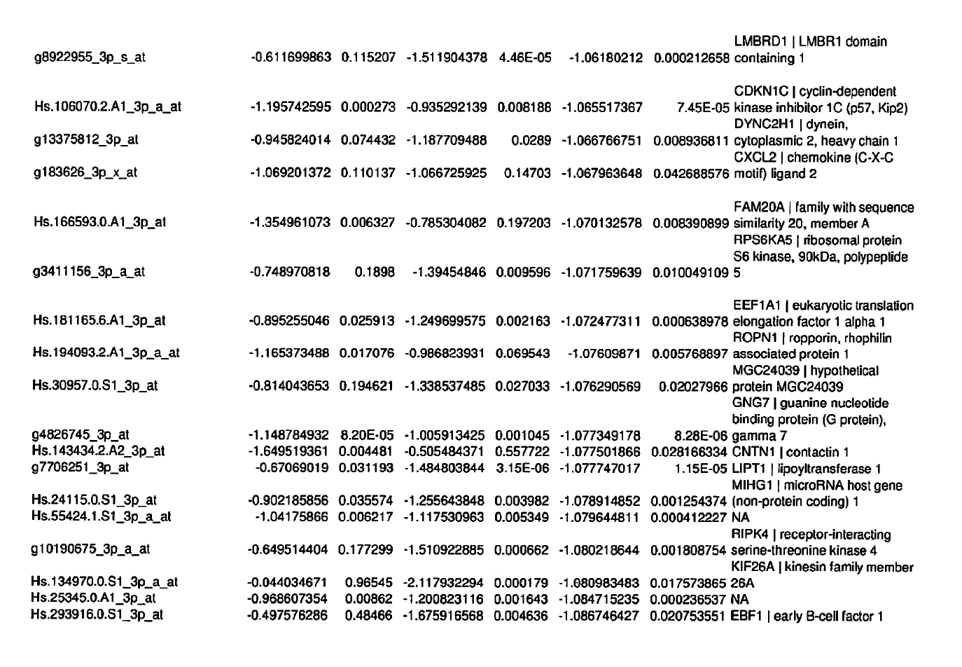

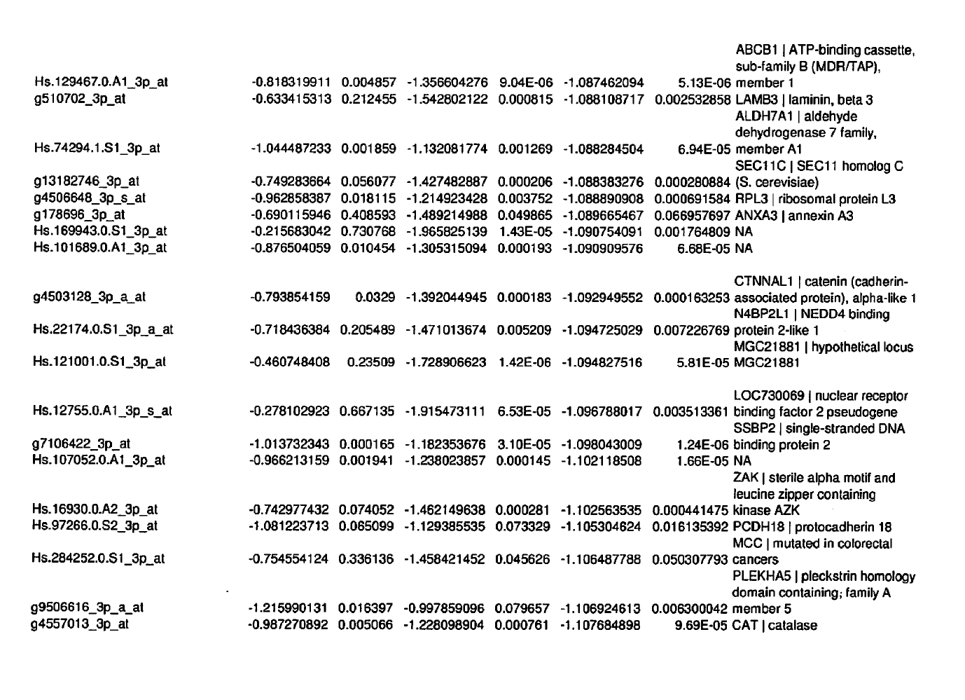

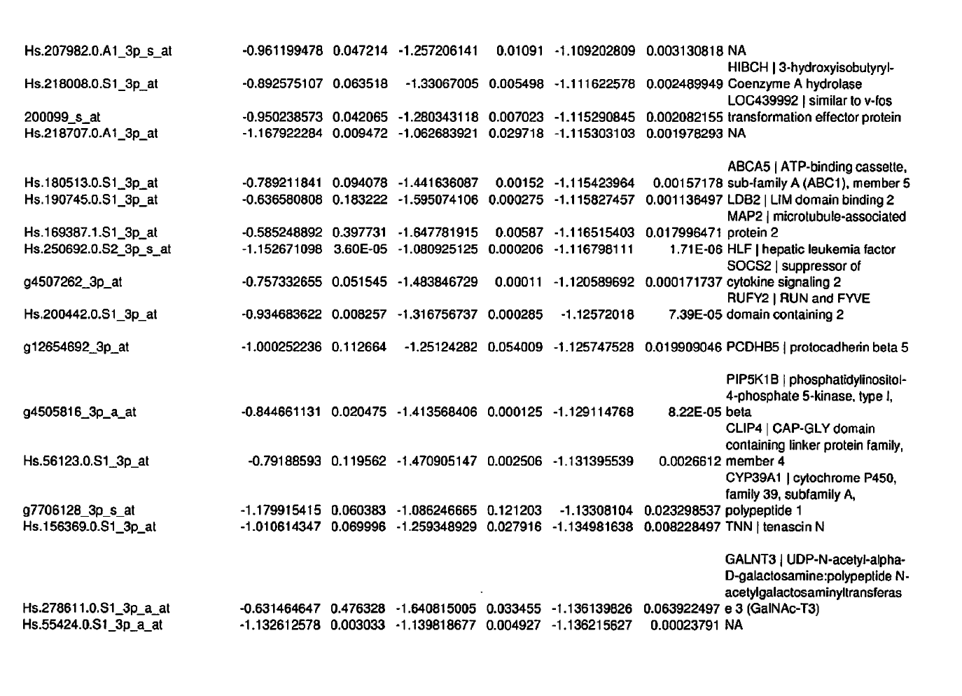

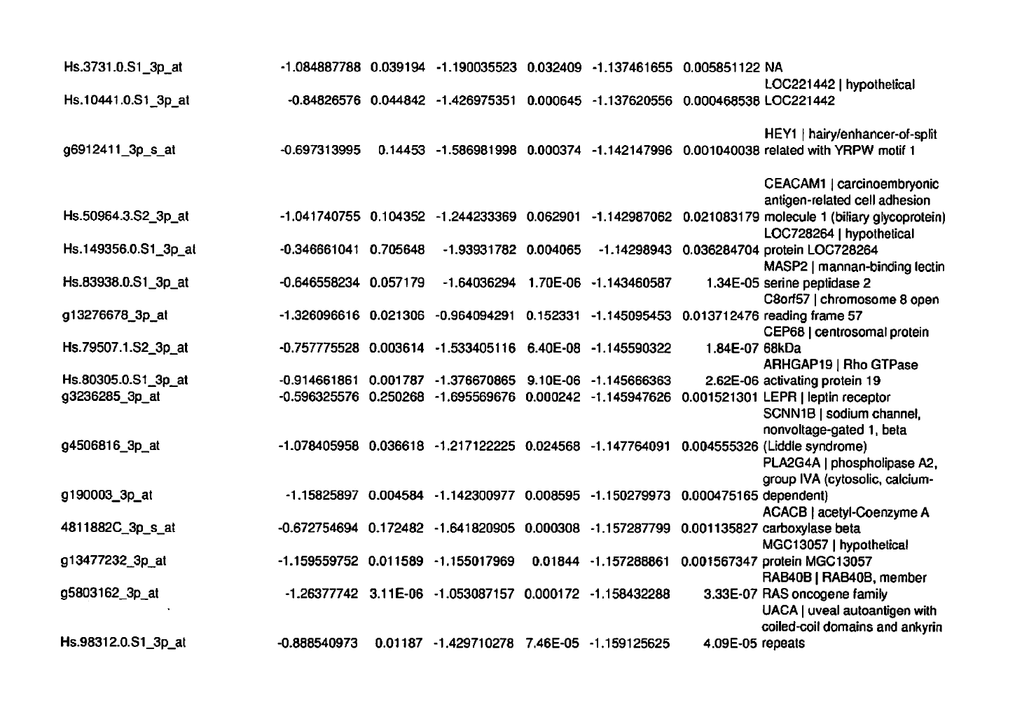

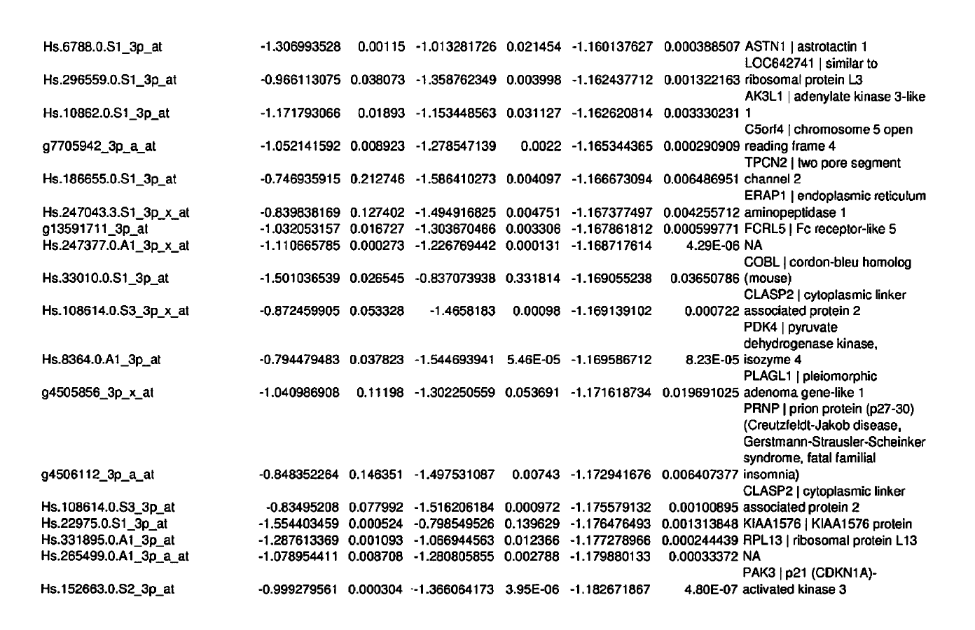

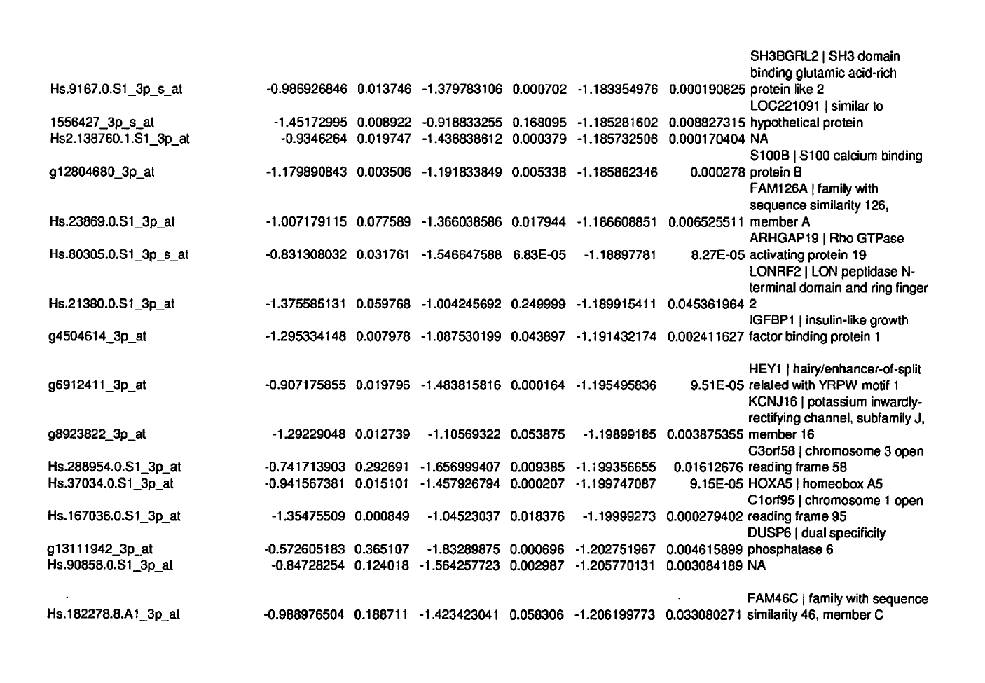

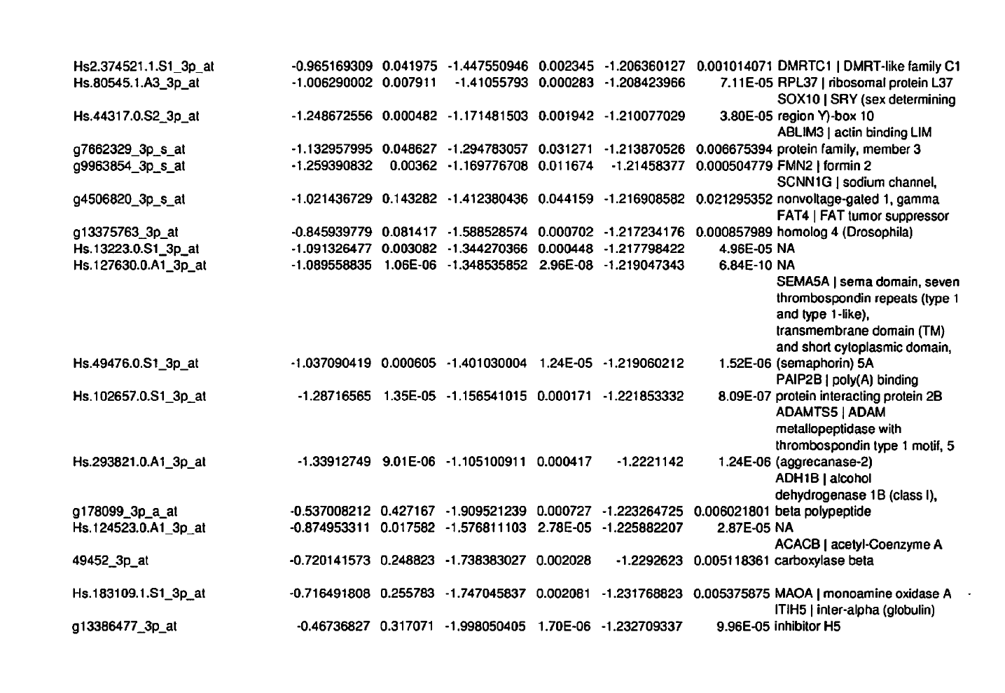

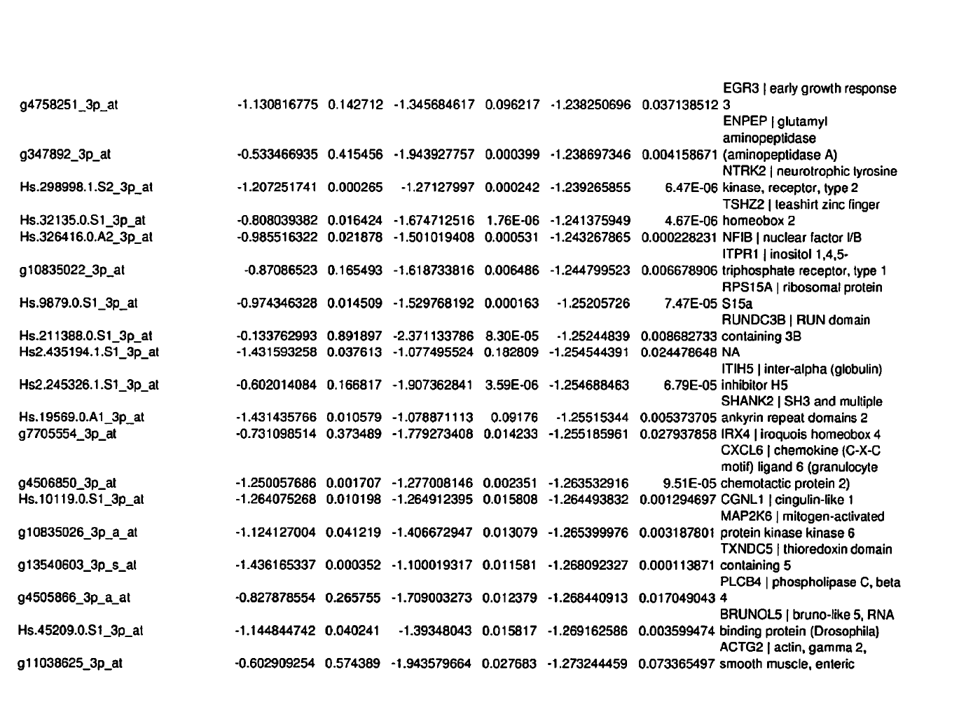

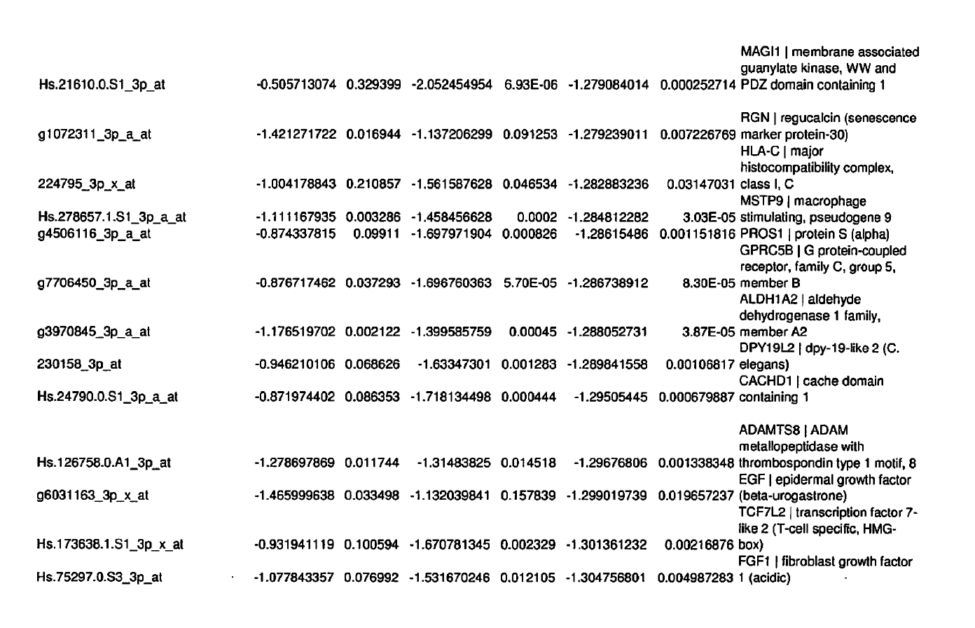

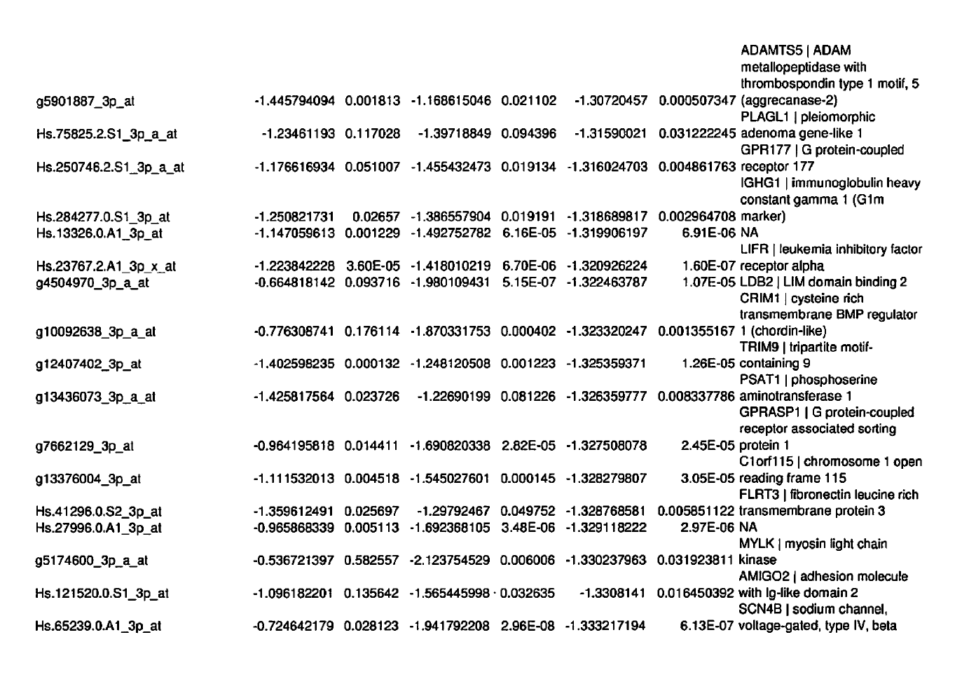

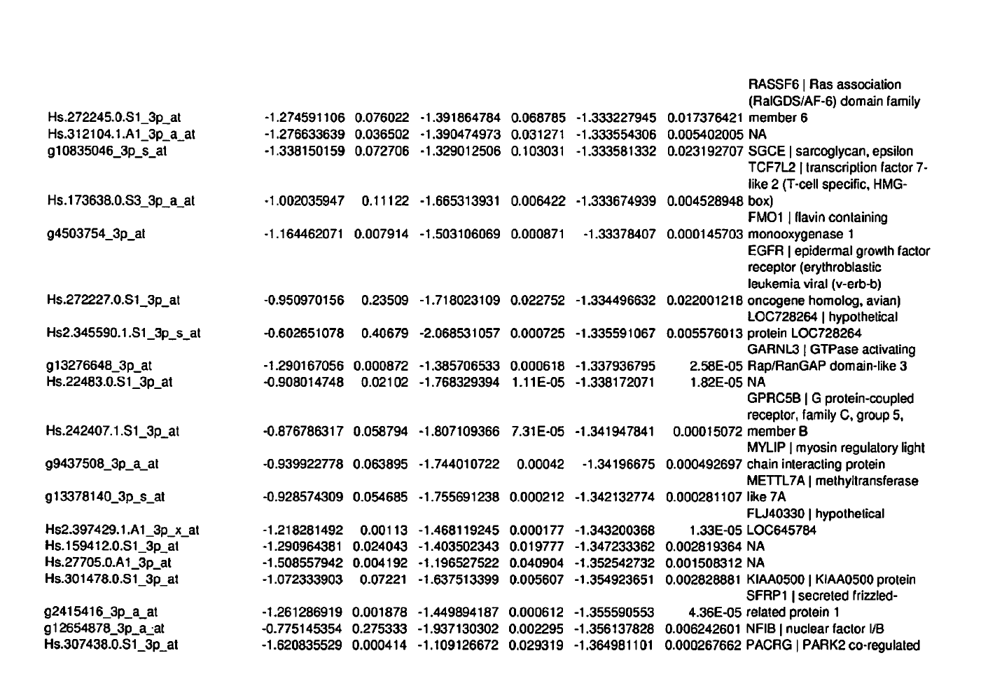

Additional genes differentially expressed in the epithelium and stroma are identified in FIG. 7 (Table 8) and FIG. 8 (Table 9), respectively. In these tables, and in addition to the dominant features of cell cycle-related genes in the epithelium and ECM genes in the stroma discussed earlier, several additional genes important in cell signaling pathways were identified. Two antagonists of WNT receptor signaling, WIF1 and SFRP1, were down-regulated in both the tumor epithelium and the stroma. In addition, two members of the TGFα superfamily, GREM1 and INHBA, showed markedly increased expression specifically in the tumor stroma (Table 5).

### Example 4: Stromal Gene Expression Signature Associated with Tumor Invasion

The gene expression patterns associated with the DCIS to IDC transition within each compartment was compared. In the tumor epithelium, there were only 3 genes (POSTN, periostin; SPARC, osteoconectin; SPARCL1, SPARC-like 1) that were significantly up-regulated in IDC relative to DCIS. All three genes were previously reported to be specifically expressed in the stroma (Kanno et al. Int. J Cancer, 122(12):2707-2718 (2008); Coutu et al., J. Biol. Chem., 283(26):17991-18001 (2008); Framson et al., J. Cell Biochem., 92(4):679-690 (2004)) and were indeed strongly expressed in the stroma samples in our dataset. Thus their apparent over-expression in IDC relative to DCIS might be due to contaminating stromal cells in the procured epithelial cell populations in the IDC samples but not in DCIS samples. The lack of significant changes in gene expression in the epithelium associated with the DCIS-IDC transition seen here was consistent with our previous study (Ma, supra.).

However, in the stroma, there were more significant changes in comparing IDC-S with DCIS-S, with 76 up-regulated and 229 down-regulated genes (FIG. 2).

Table 6 lists the top 50 differentially expressed genes between DICS-S and IDC-S (see Supplemental Table 1 for full listing).

TABLE 6
Top 50 genes differentially expressed in invasive stroma compared to in situ stroma

| | Log2 | | |
|---|---|---|---|
| | (Fold | Adjusted p | |
| Probeset ID | Change) | value | Gene Description |
| Hs2.434299.1.S1_3p_at | 1.61 | 8.58E−03 | — |
| g13027795_3p_s_at | 1.45 | 1.74E−02 | MMP11\|matrix metallopeptidase |
| | | | 11 (stromelysin 3) |
| Hs.50081.1.S1_3p_a_at | 1.36 | 5.47E−03 | KIAA1199\|KIAA1199 |
| g11641276_3p_s_at | 1.24 | 3.71E−02 | PDE4DIP\|phosphodiesterase 4D |
| | | | interacting protein (myomegalin) |
| Hs.169517.0.S1_3p_a_at | 1.16 | 5.72E−03 | ALDH1B1\|aldehyde |
| | | | dehydrogenase 1 family, member |
| | | | B1 |
| Hs.98523.0.A1_3p_at | 1.13 | 5.32E−03 | FAT3\|FAT tumor suppressor |
| | | | homolog 3 (Drosophila) |
| g10938018_3p_at | 1.04 | 1.74E−02 | EPYC\|epiphycan |
| Hs2.350890.1.S1_3p_s_at | 1.03 | 3.72E−02 | GABRB2\|gamma-aminobutyric |
| | | | acid (GABA) A receptor, beta 2 |
| g4507922_3p_at | 0.98 | 1.70E−02 | WISP2\|WNT1 inducible signaling |
| | | | pathway protein 2 |
| g11342665_3p_at | 0.98 | 4.53E−02 | MMP2\|matrix metallopeptidase 2 |
| | | | (gelatinase A, 72 kDa gelatinase, |
| | | | 72 kDa type IV collagenase) |
| g13124890_3p_a_at | 0.93 | 3.28E−02 | GALNT1\|UDP-N-acetyl-alpha-D- |
| | | | galactosamine:polypeptide N- |
| | | | acetylgalactosaminyltransferase 1 |
| | | | (GalNAc-T1) |
| Hs.98523.0.A1_3p_x_at | 0.87 | 3.15E−02 | FAT3\|FAT tumor suppressor |
| | | | homolog 3 (Drosophila) |
| Hs.238532.0.A1_3p_at | 0.76 | 3.71E−02 | GALNTL2\|UDP-N-acetyl-alpha- |
| | | | D-galactosamine:polypeptide N- |
| | | | acetylgalactosaminyltransferase- |
| | | | like 2 |
| Hs.42927.0.S1_3p_at | 0.75 | 3.39E−02 | ANTXR1\|anthrax toxin receptor 1 |
| Hs2.359399.1.S1_3p_at | 0.74 | 3.71E−02 | LOC285758\|hypothetical protein |
| | | | LOC285758 |
| Hs.235795.0.A1_3p_at | 0.74 | 1.38E−02 | — |
| Hs2.46679.2.S1_3p_s_at | 0.66 | 2.11E−03 | — |
| g469044_3p_a_at | 0.60 | 3.22E−02 | CNTN1\|contactin 1 |
| g4758607_3p_at | 0.53 | 1.20E−02 | — |
| Hs.288553.0.S1_3p_s_at | 0.52 | 2.82E−02 | — |
| 200661_3p_at | 0.51 | 4.16E−02 | CTSA\|cathepsin A |
| Hs.2399.1.S1_3p_s_at | 0.51 | 4.48E−02 | MMP14\|matrix metallopeptidase |
| | | | 14 (membrane-inserted) |
| Hs.98183.0.A1_3p_at | 0.48 | 8.58E−03 | RSPO4\|R-spondin family, member 4 |
| 208756_3p_at | 0.48 | 2.28E−02 | EIF3I\|eukaryotic translation |
| | | | initiation factor 3, subunit I |
| Hs.162647.0.S1_3p_at | 0.48 | 2.69E−02 | DKFZP547L112\|hypothetical |
| | | | protein DKFZp547L112 |
| Hs.22968.0.S1_3p_a_at | −2.00 | 8.37E−06 | FLT1\|fms-related tyrosine kinase |
| | | | 1 (vascular endothelial growth |
| | | | factor/vascular permeability factor |
| | | | receptor) |
| g11545907_3p_at | −2.03 | 2.68E−05 | ELTD1\|EGF, latrophilin and seven |
| | | | transmembrane domain containing 1 |
| g5032094_3p_at | −2.03 | 3.64E−07 | SLCO2A1\|solute carrier organic |
| | | | anion transporter family, member |
| | | | 2A1 |
| Hs.8707.0.S1_3p_at | −2.07 | 1.29E−05 | HECW2\|HECT, C2 and WW |
| | | | domain containing E3 ubiquitin |
| | | | protein ligase 2 |
| Hs.134970.0.S1_3p_a_at | −2.07 | 6.66E−03 | KIF26A\|kinesin family member |
| | | | 26A |
| Hs2.420404.1.S1_3p_at | −2.08 | 7.70E−06 | PELO\|pelota homolog |
| | | | (Drosophila) |
| g4504850_3p_a_at | −2.11 | 3.62E−02 | KCNK5\|potassium channel, |
| | | | subfamily K, member 5 |
| Hs.288681.0.S1_3p_at | −2.13 | 4.98E−04 | THSD7A\|thrombospondin, type I, |
| | | | domain containing 7A |
| g4557546_3p_at | −2.15 | 1.58E−03 | EDNRB\|endothelin receptor type B |
| g11321596_3p_at | −2.19 | 1.64E−03 | KDR\|kinase insert domain |
| | | | receptor (a type III receptor tyrosine |
| | | | kinase) |
| Hs.124675.0.A1_3p_at | −2.21 | 2.21E−04 | GIMAP7\|GTPase, IMAP family |
| | | | member 7 |
| Hs.211388.0.S1_3p_at | −2.24 | 5.72E−03 | RUNDC3B\|RUN domain |
| | | | containing 3B |
| g4885556_3p_at | −2.25 | 2.13E−03 | PODXL\|podocalyxin-like |
| Hs.26530.0.S2_3p_at | −2.30 | 1.60E−03 | SDPR\|serum deprivation response |
| | | | (phosphatidylserine binding |
| | | | protein) |
| g13518036_3p_a_at | −2.41 | 7.19E−03 | MATN2\|matrilin 2 |
| Hs.102415.0.S1_3p_at | −2.43 | 2.67E−08 | EMCN\|endomucin |
| Hs.61935.0.S1_3p_at | −2.45 | 2.68E−05 | PCDH17\|protocadherin 17 |
| g8547214_3p_at | −2.46 | 3.74E−05 | EMCN\|endomucin |
| g4520327_3p_at | −2.48 | 2.67E−03 | IL33\|interleukin 33 |
| Hs.10587.0.S1_3p_at | −2.66 | 2.29E−03 | DMN\|desmuslin |
| g3644039_3p_a_at | −2.67 | 1.43E−02 | TP63\|tumor protein p63 |
| Hs.78344.1.S2_3p_a_at | −2.87 | 2.13E−03 | MYH11\|myosin, heavy chain 11, |
| | | | smooth muscle |
| g6580814_3p_s_at | −2.93 | 8.90E−05 | INMT\|indolethylamine N- |
| | | | methyltransferase |
| Hs.173560.0.S1_3p_at | −2.94 | 3.58E−02 | ODZ2\|odz, odd Oz/ten-m homolog |
| | | | 2 (Drosophila) |
| g4506870_3p_at | −3.23 | 1.80E−03 | SELE\|selectin E (endothelial |
| | | | adhesion molecule 1) |

Among genes with increased expression in IDC-S, three matrix metalloproteases (MMP11, MMP2 and MMP14) were notable. Indeed, one additional MMP, MMP13, had higher expression in IDC-S than in DCIS-S with an adjusted p value 0.06. These genes have been reported to be involved in tumor invasion (Liotta, supra.). In comparison, genes with decreased expression in IDC-S included many genes involved in vasculature development (e.g., EMCN, FLT1, KDR, SELE, MYH11, EDNRB and PODXL), a process expected to increase in invasive cancer. Without being bound by theory, this unexpected finding may be due to the decreased vascular density in the leading invasive front from which microdissection of the stroma relative the stroma surrounding DCIS occurred.

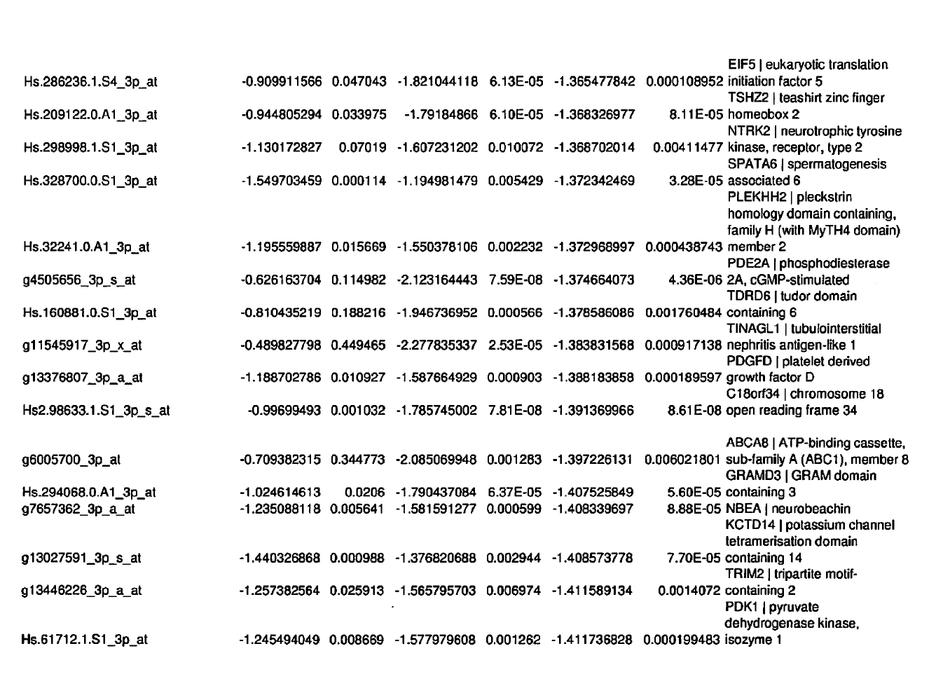

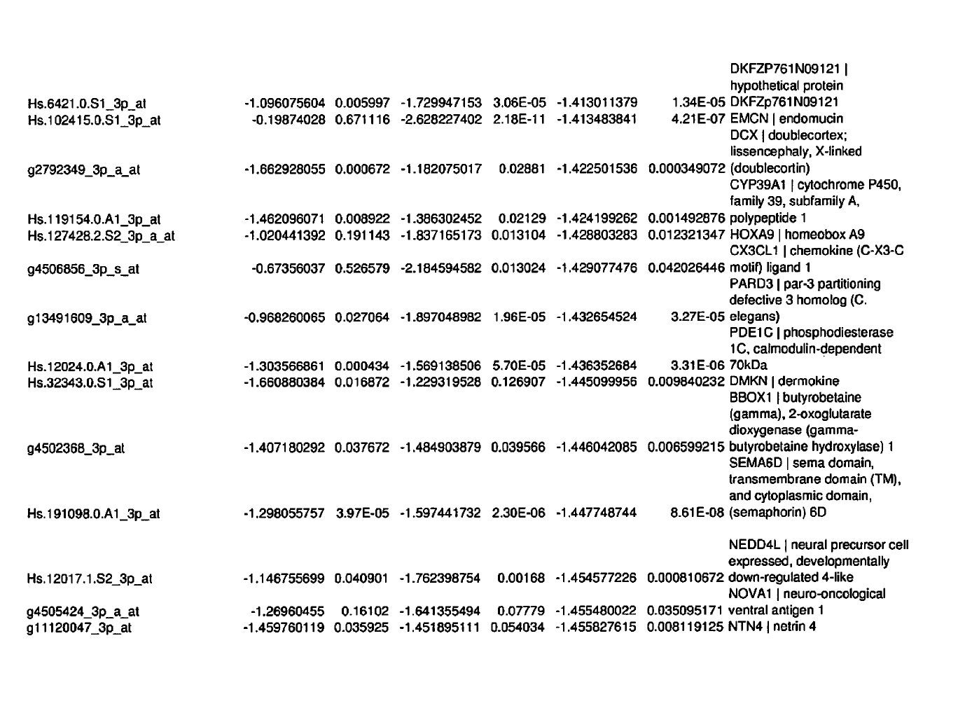

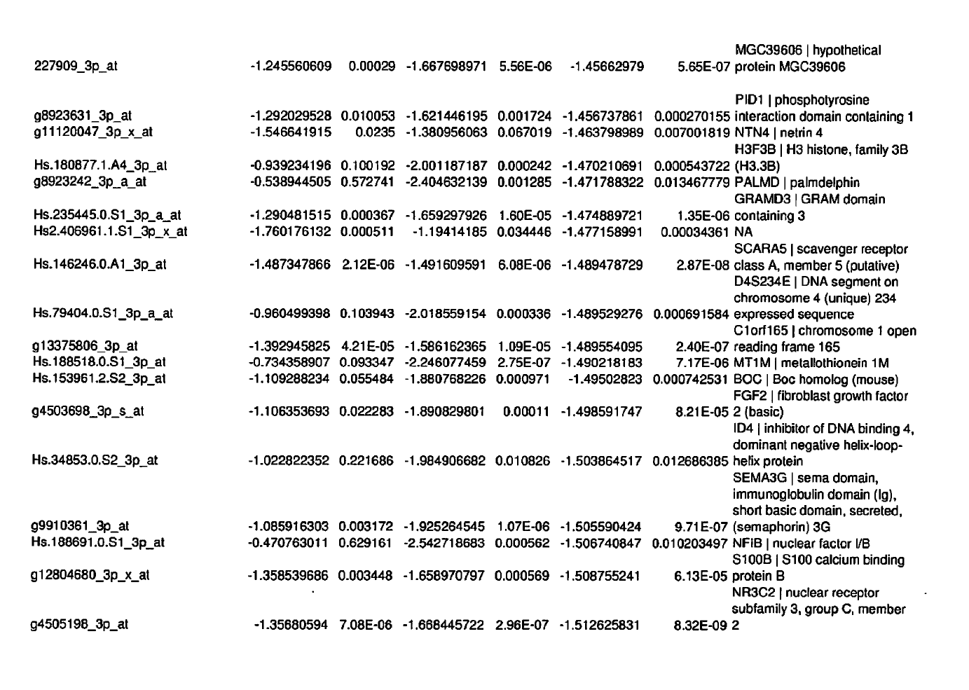

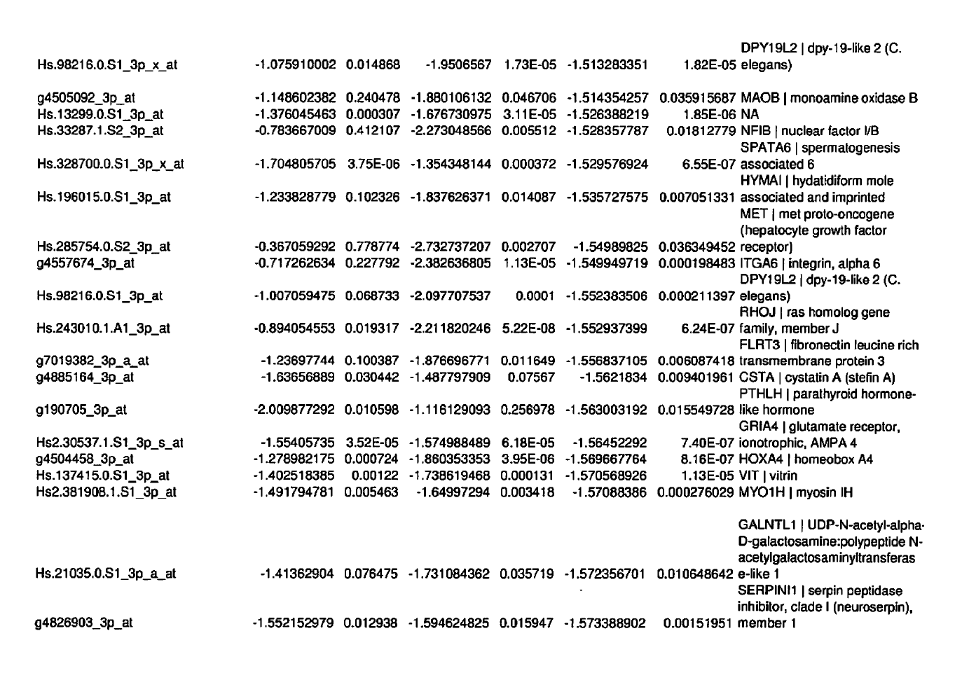

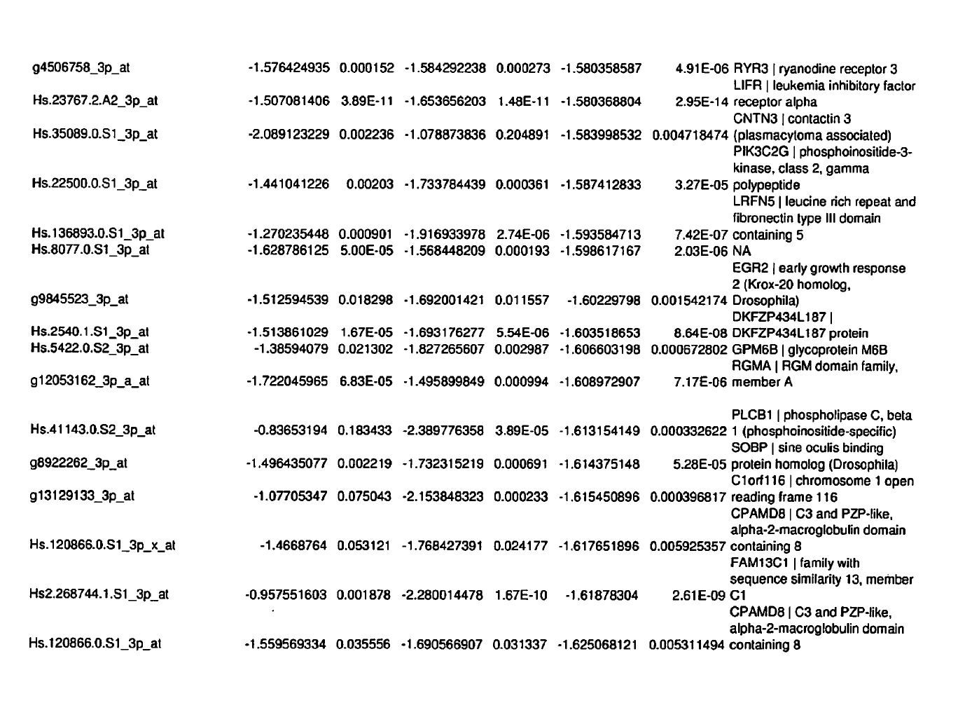

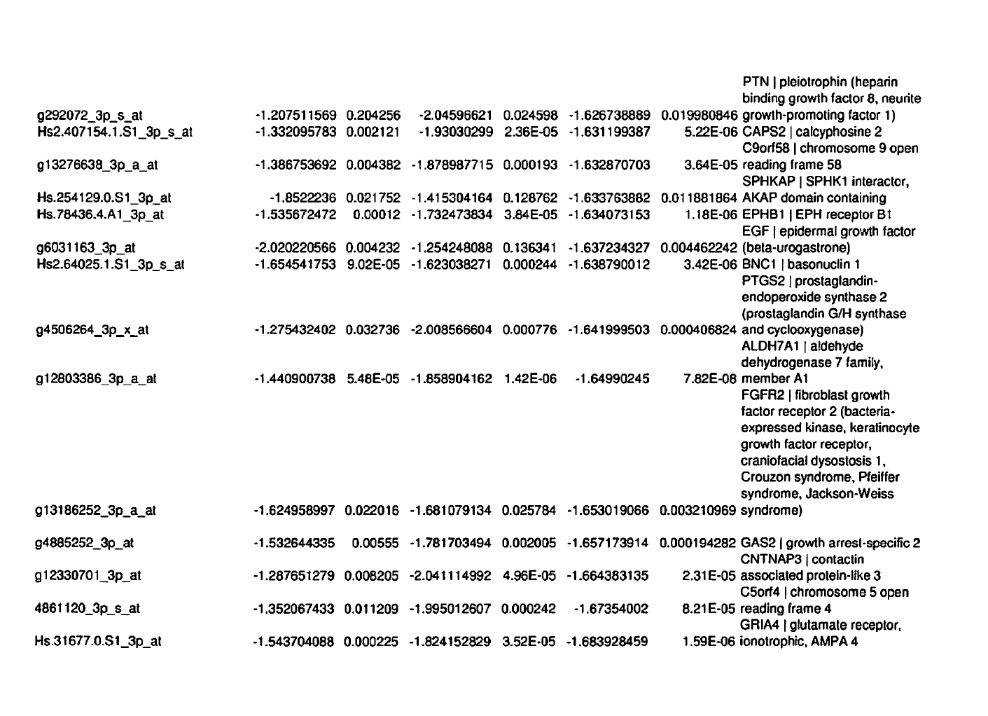

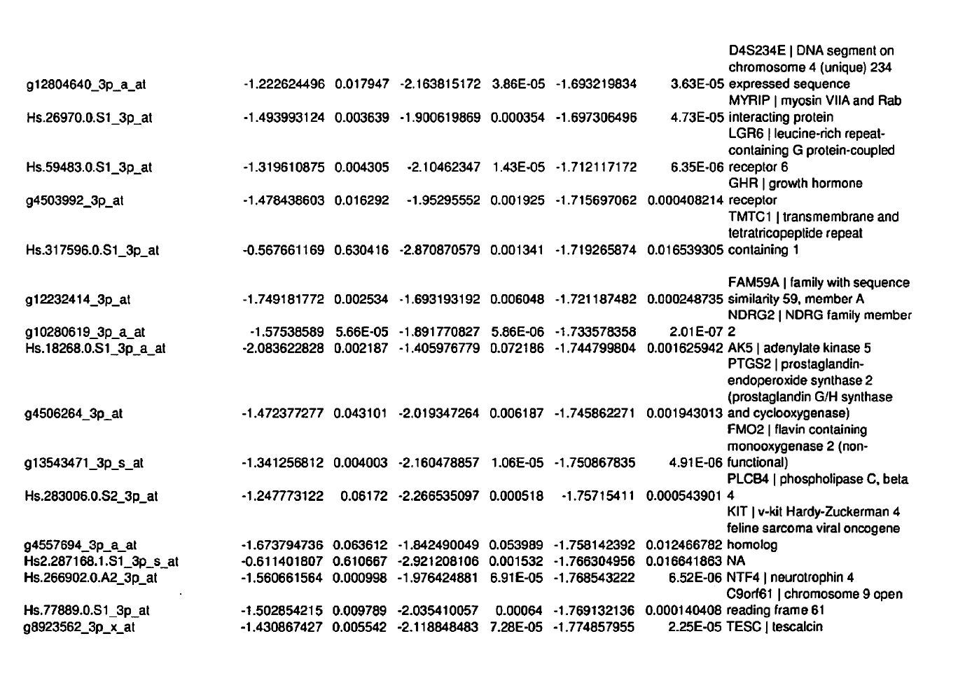

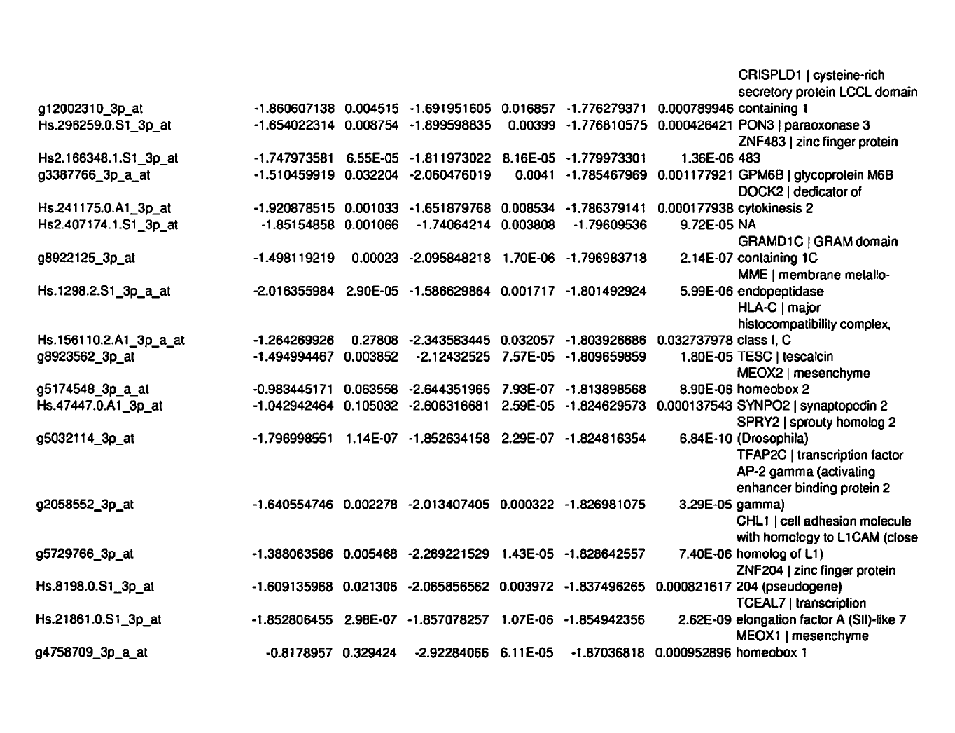

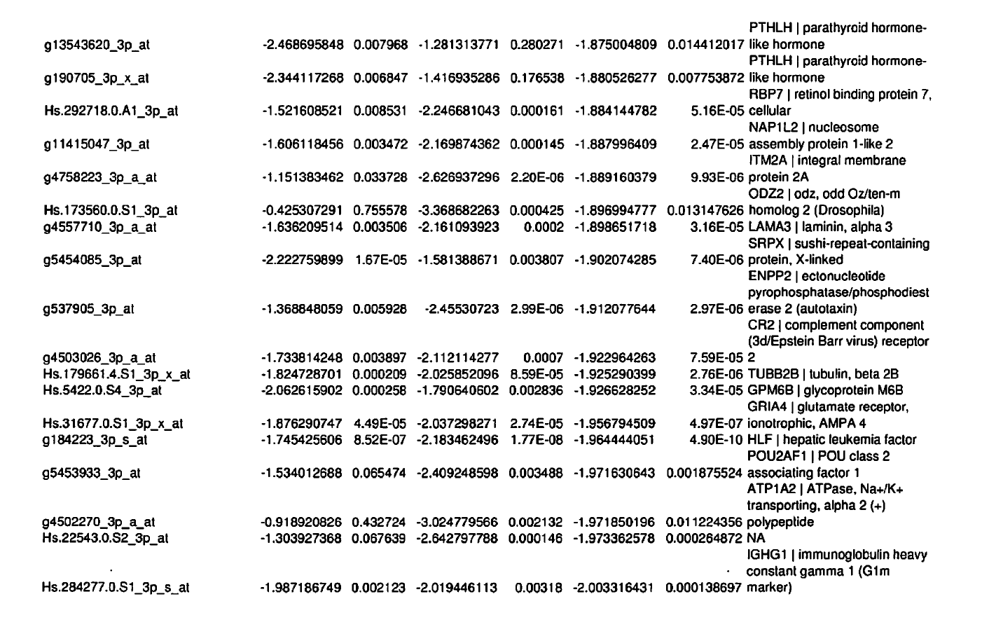

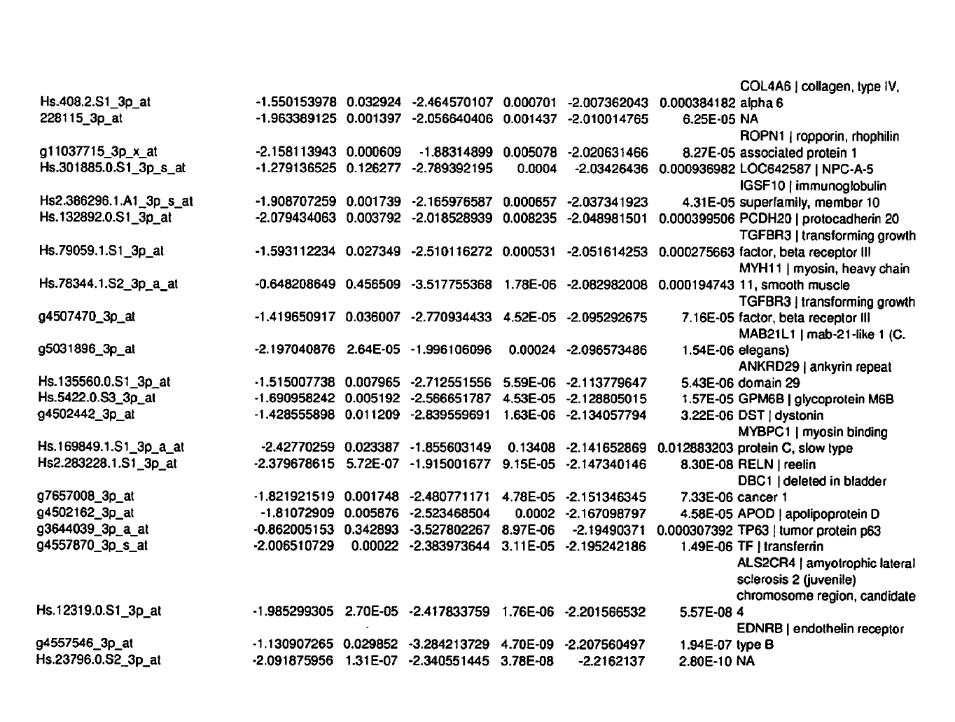

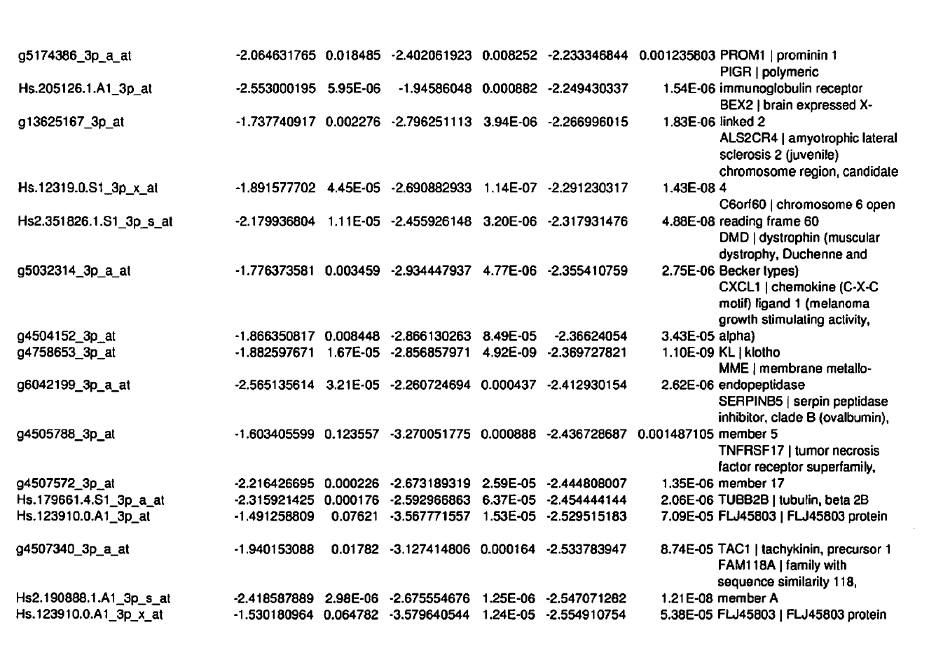

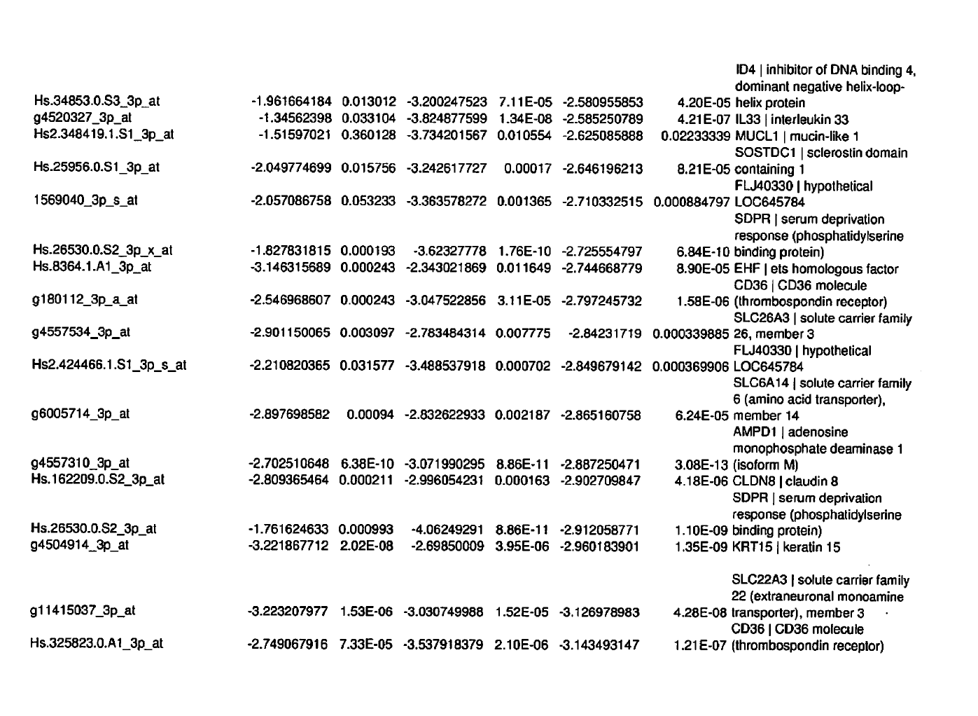

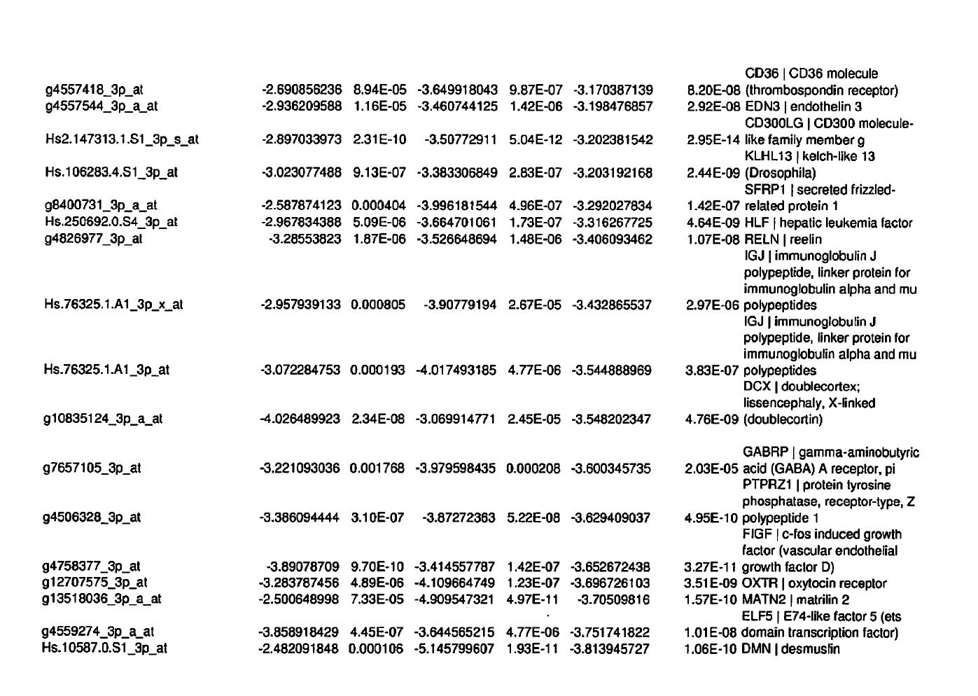

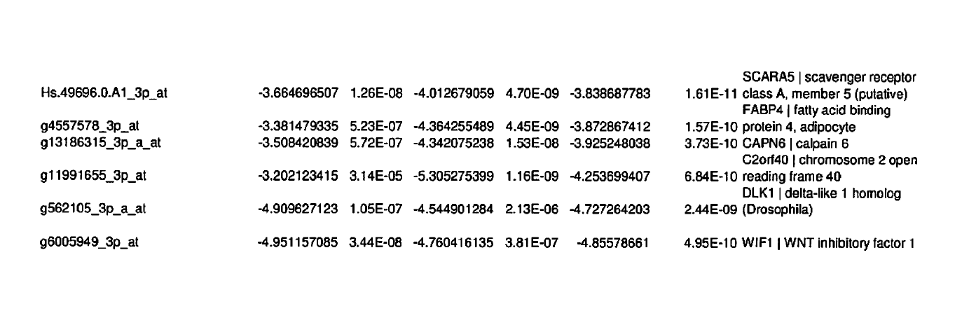

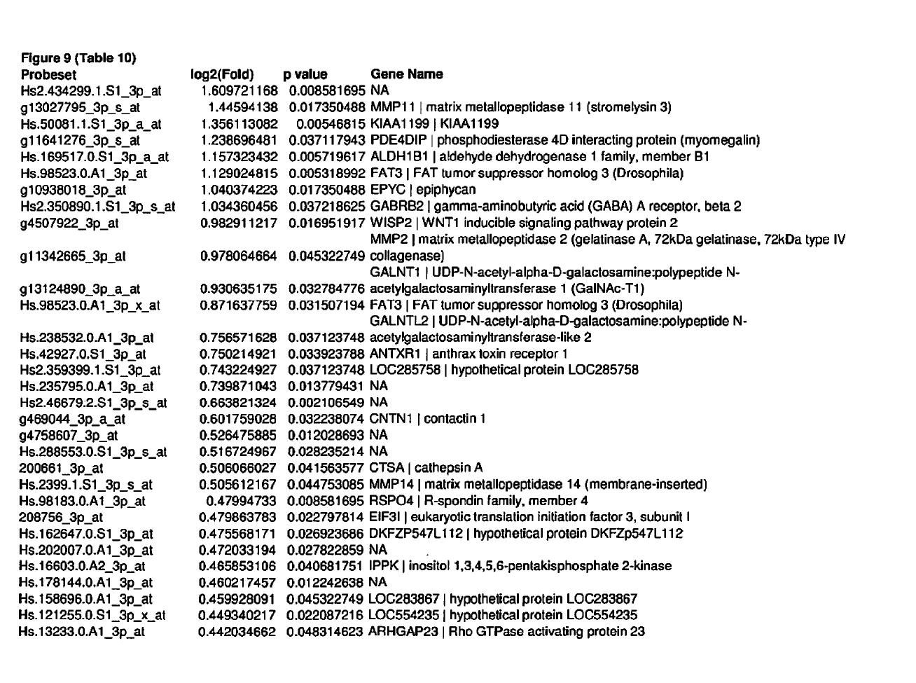

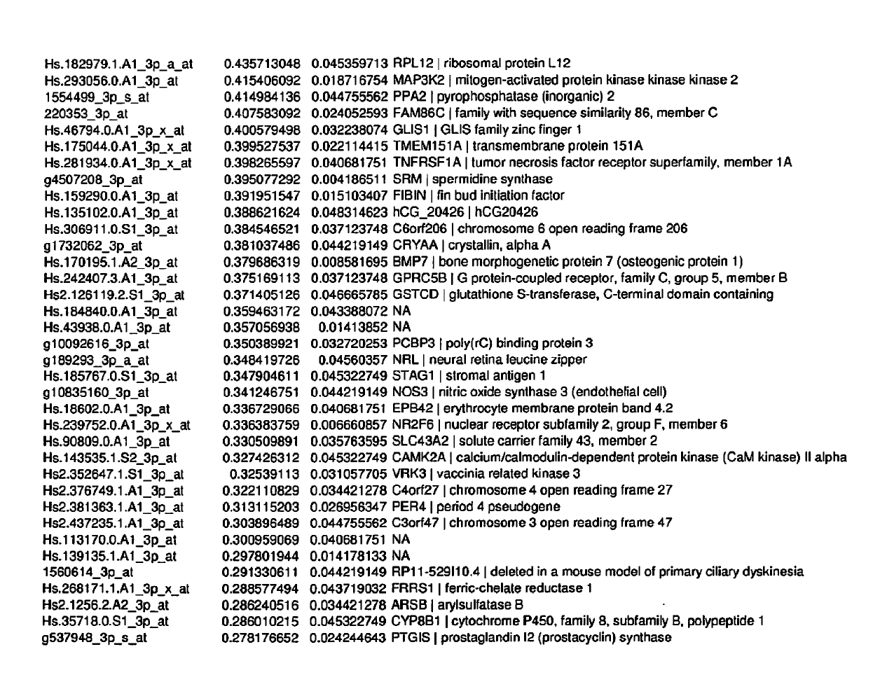

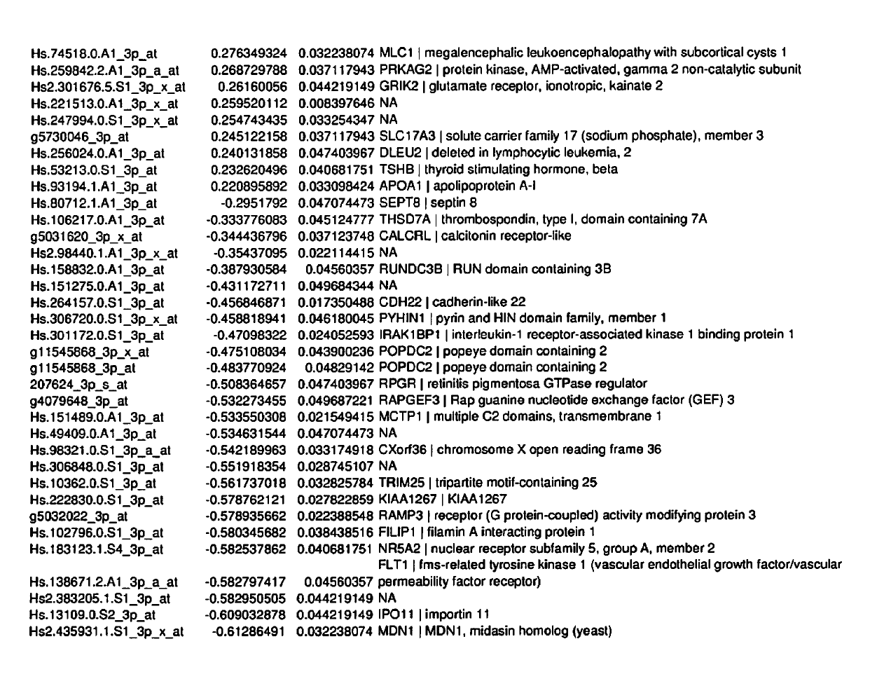

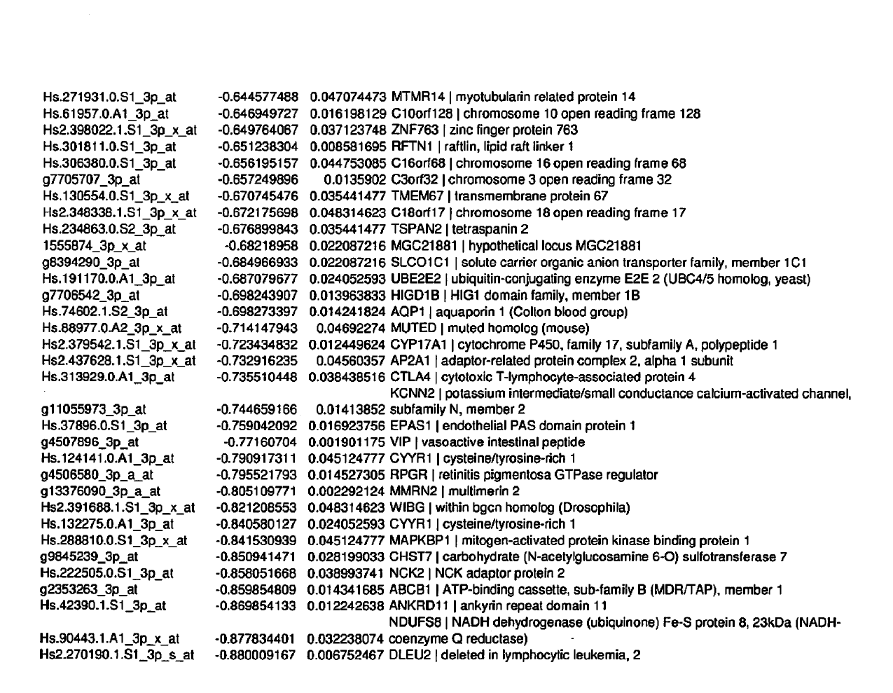

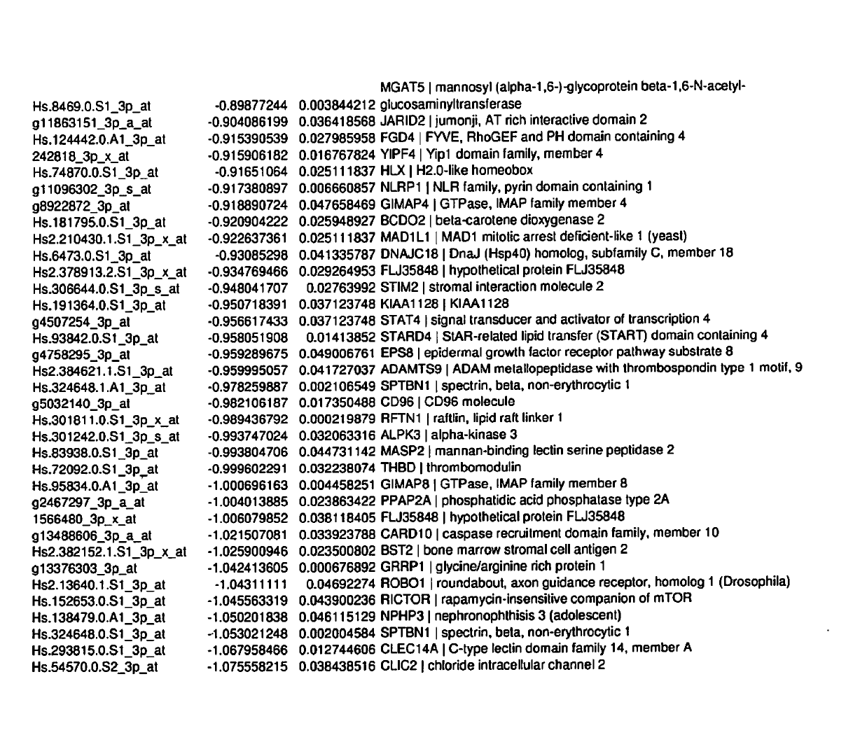

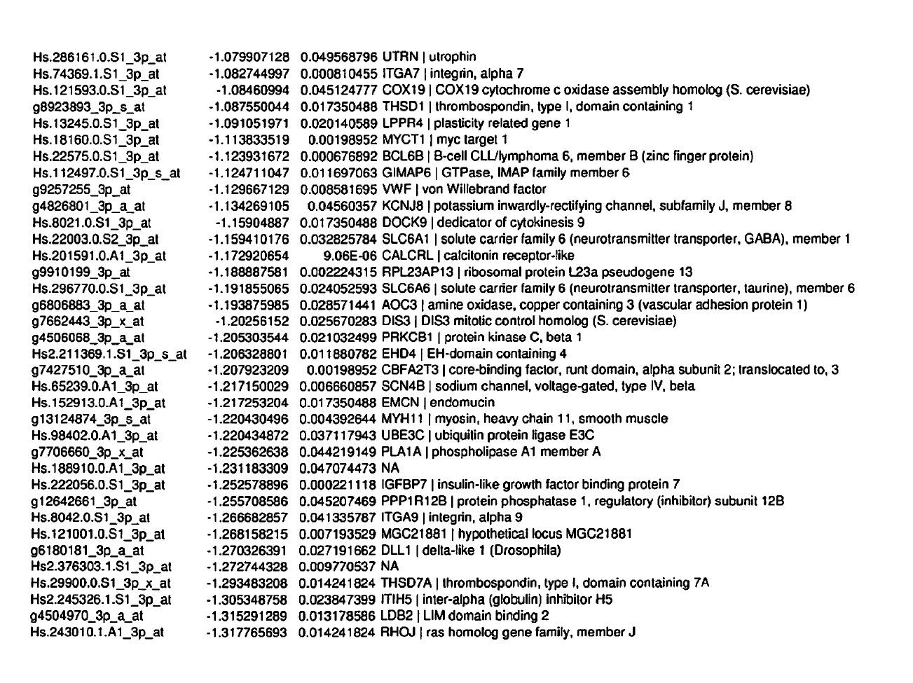

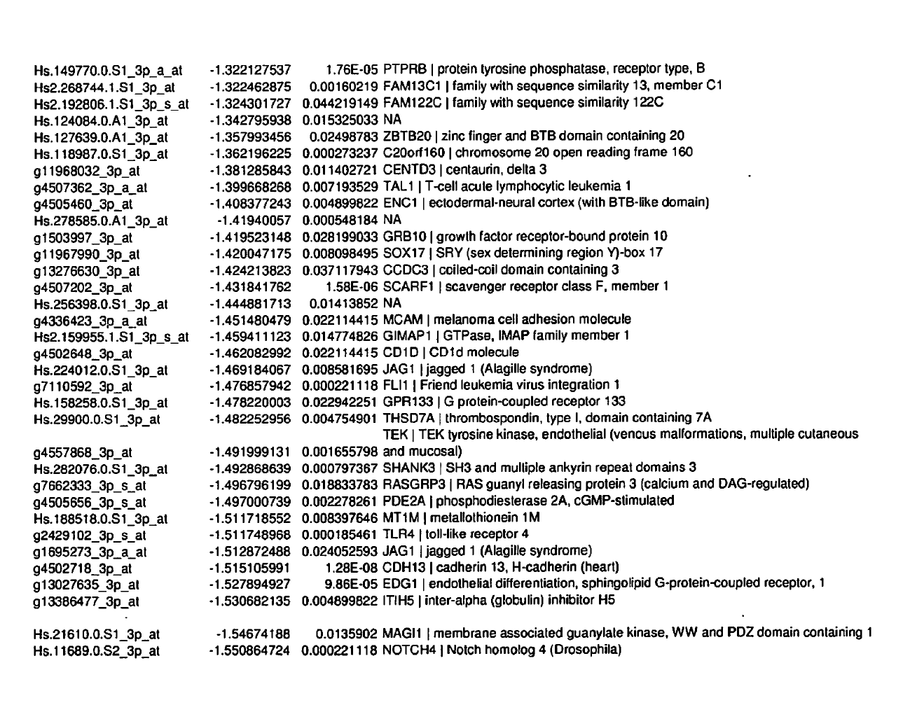

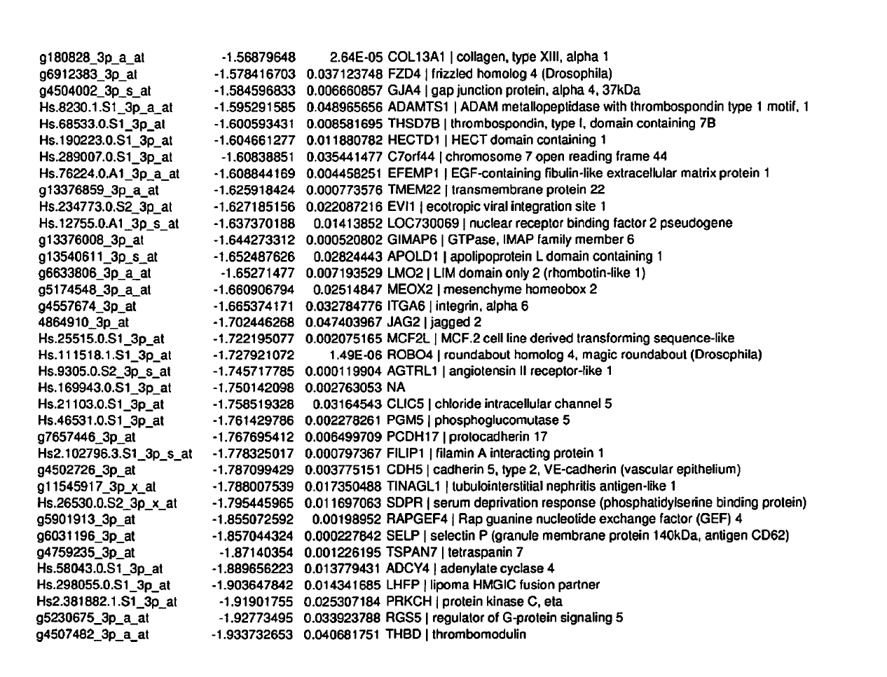

A more extensive list of the genes identified is presented in FIG. 9 (Table 10).

### Example 5: Stromal Gene Expression Signature Associated with Tumor Grade

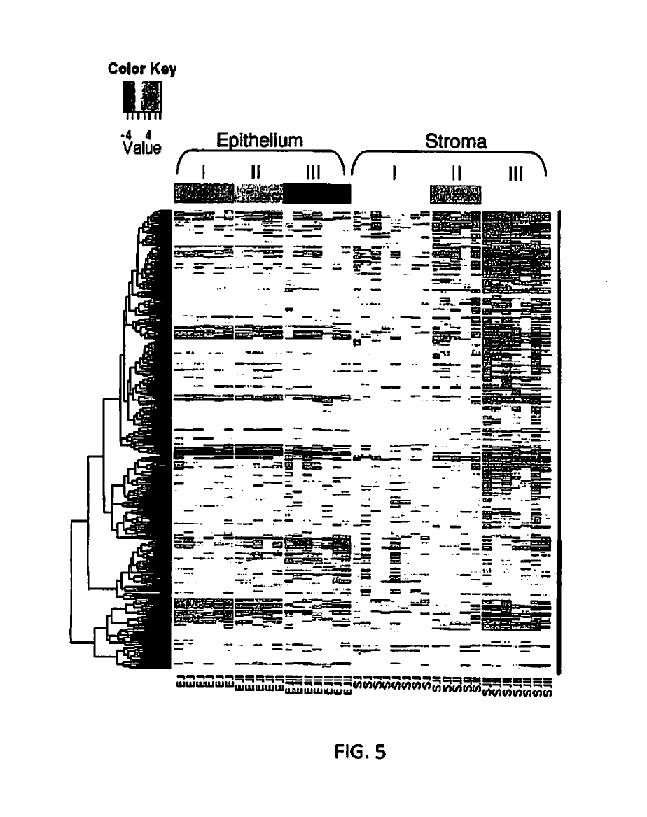

Previously, the present inventors have shown that tumor grade is associated with a strong gene expression signature in malignant breast epithelial cells (Ma, supra.). Therefore, the question of whether a similar signature also exists in the tumor stroma was examined. Comparing grade I (n=8) and grade III (n=7) tumor-associated stroma samples (DCIS-S and IDC-S), the identification of 526 up-regulated and 94 down-regulated genes in grade III samples (FIG. 5) was made. GSEA analysis indicated that the tumor stroma in Grade III tumors were associated a strong immune response signature (interferon signaling, activation of leukocytes and T cells) and increased mitotic activity (Table 7).

TABLE 7
Top 20 gene sets enriched in grade III-associated stroma.

| | | | FDR |
|---|---|---|---|
| NAME | SIZE | NES | q-val |
| CELLULAR_DEFENSE_RESPONSE | 52 | 2.31 | 0 |
| IMMUNE_RESPONSE | 220 | 2.17 | 0 |
| IMMUNE_SYSTEM_PROCESS | 312 | 2.16 | 0 |
| T_CELL_ACTIVATION | 42 | 2.14 | 0 |
| LEUKOCYTE_ACTIVATION | 67 | 2.09 | 0 |
| JAK_STAT_CASCADE | 28 | 2.05 | 6.82E−04 |
| LYMPHOCYTE_ACTIVATION | 59 | 2.05 | 5.85E−04 |
| CELL_ACTIVATION | 73 | 2.04 | 5.12E−04 |
| M_PHASE_OF_MITOTIC_CELL_CYCLE | 78 | 2.04 | 4.55E−04 |
| RESPONSE_TO_VIRUS | 48 | 2.04 | 5.12E−04 |
| SPINDLE | 39 | 2.03 | 5.60E−04 |
| MITOSIS | 75 | 2.02 | 5.99E−04 |
| INTERLEUKIN_RECEPTOR_ACTIVITY | 20 | 2.01 | 6.33E−04 |
| POSITIVE_REGULATION_OF_IMMUNE_RESPONSE | 28 | 2.00 | 7.35E−04 |
| REGULATION_OF_IMMUNE_SYSTEM_PROCESS | 66 | 1.99 | 7.54E−04 |
| POSITIVE_REGULATION_OF_IMMUNE_SYSTEM_PROCESS | 50 | 1.99 | 7.07E−04 |
| RESPONSE_TO_BIOTIC_STIMULUS | 112 | 1.99 | 6.65E−04 |
| REGULATION_OF_I_KAPPAB_KINASE_NF_KAPPAB_CASCADE | 89 | 1.99 | 6.85E−04 |
| MRNA_PROCESSING_GO_0006397 | 67 | 1.97 | 0.001135 |
| RESPONSE_TO_OTHER_ORGANISM | 76 | 1.96 | 0.001282 |

Abbreviations:
NES, normalized enrichment score;
FDR, false discovery rate.

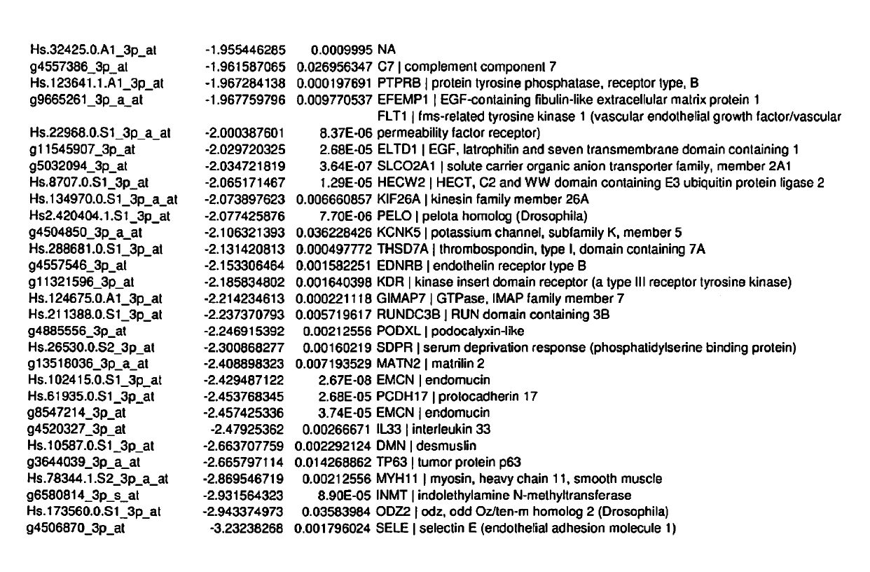

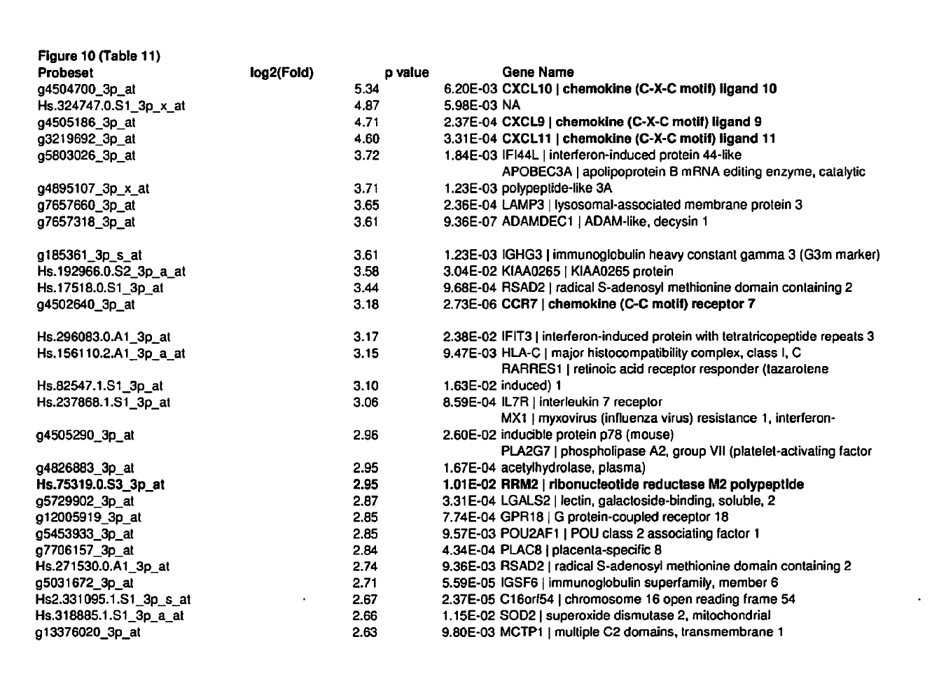

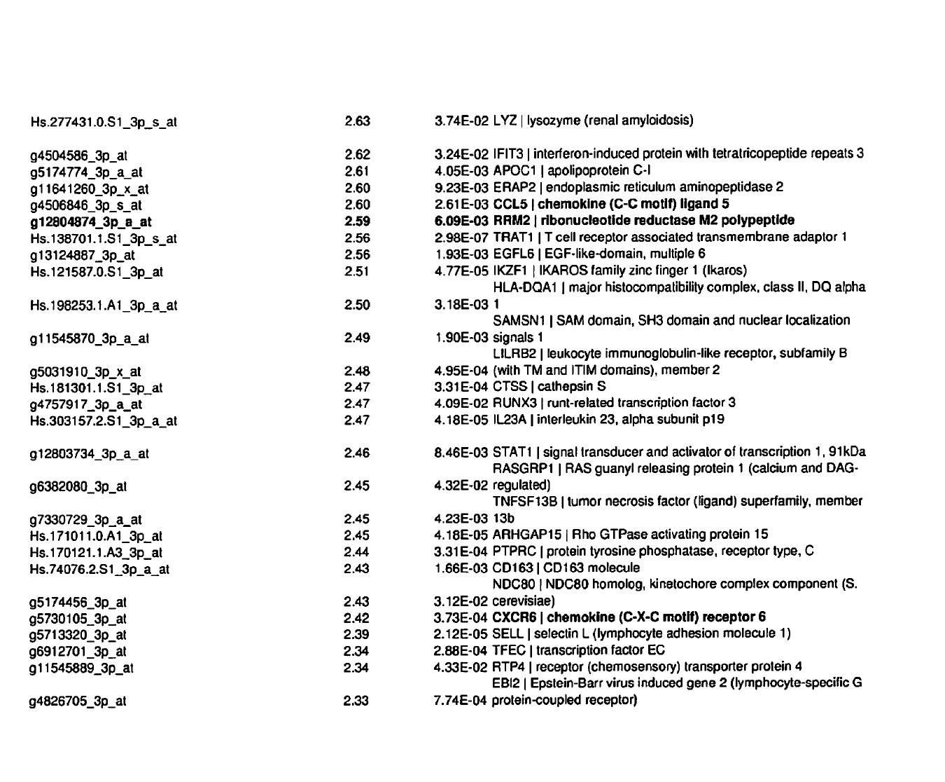

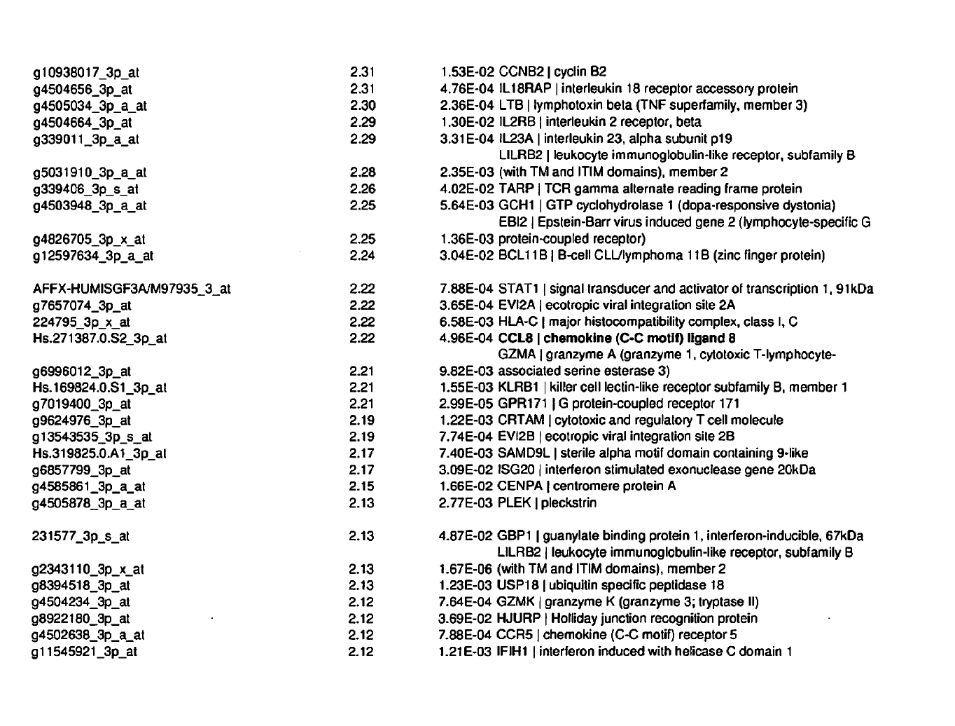

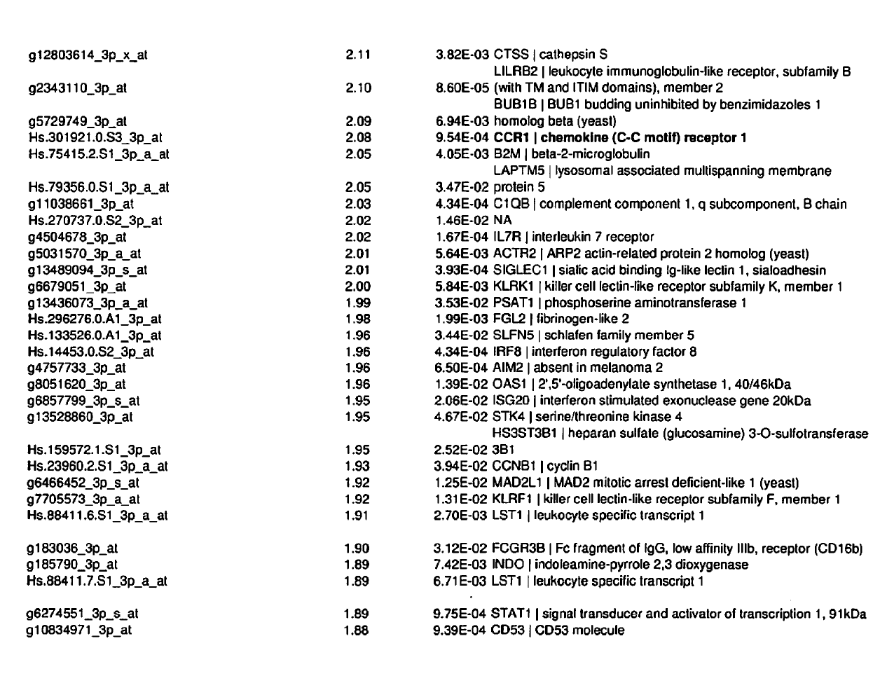

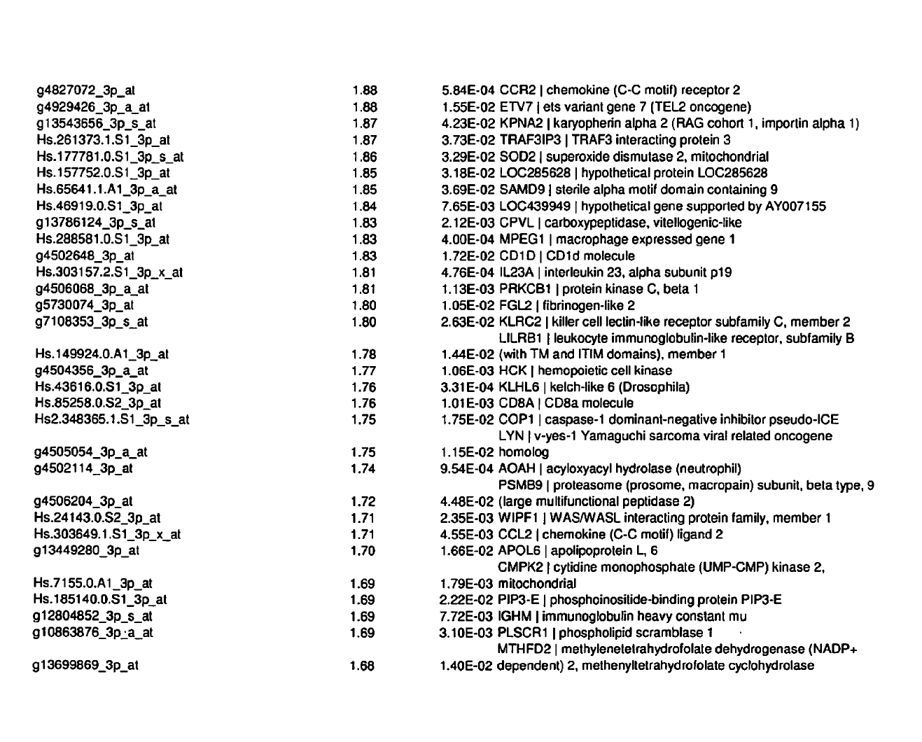

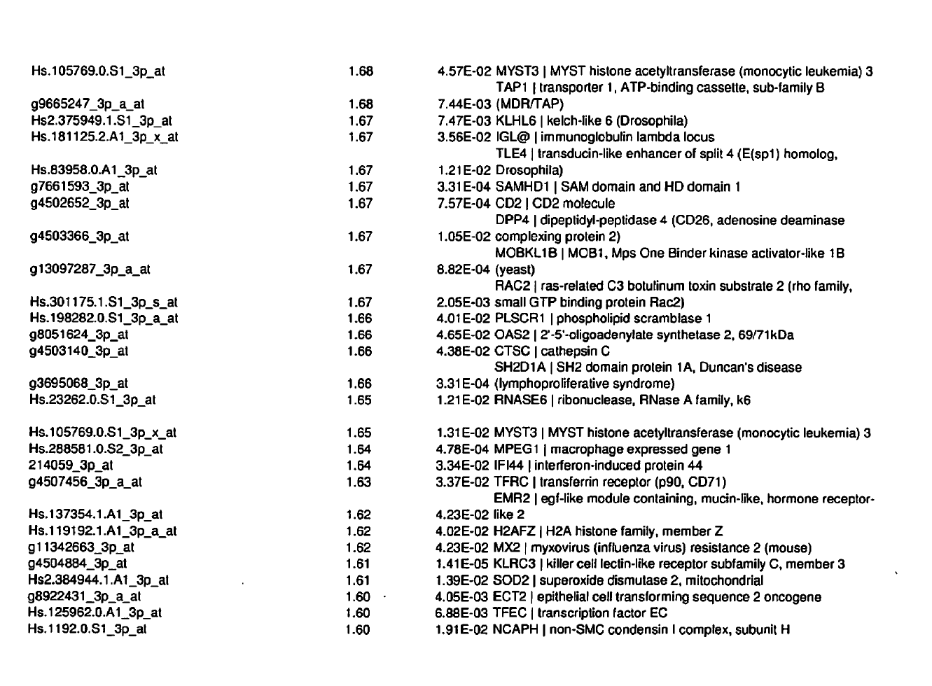

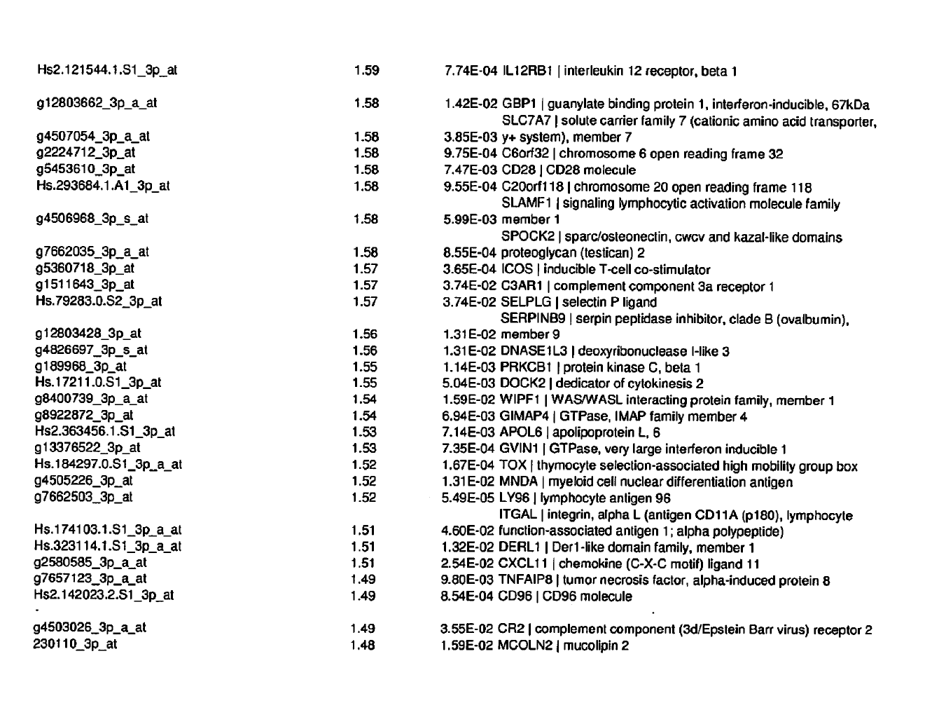

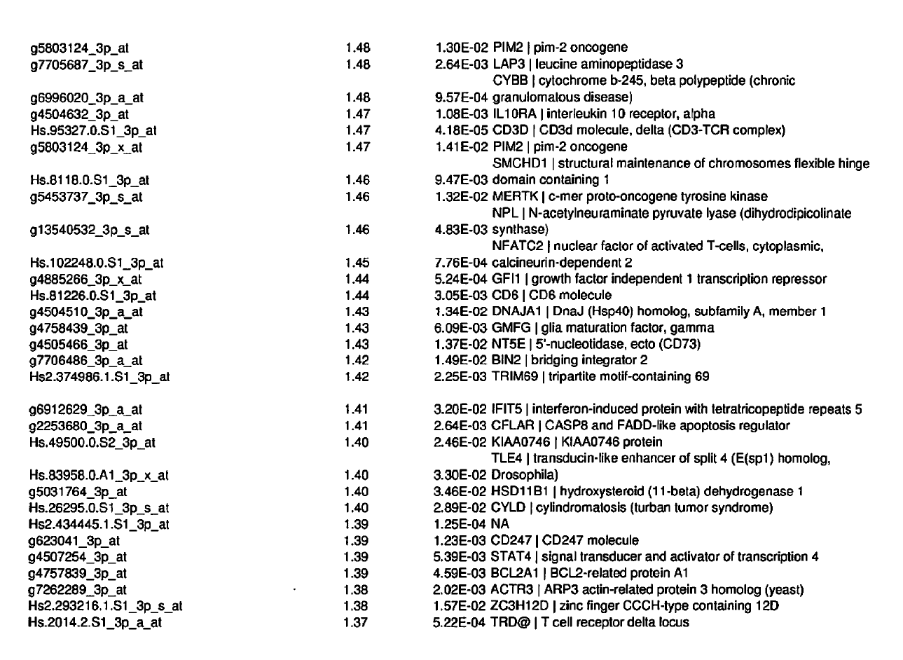

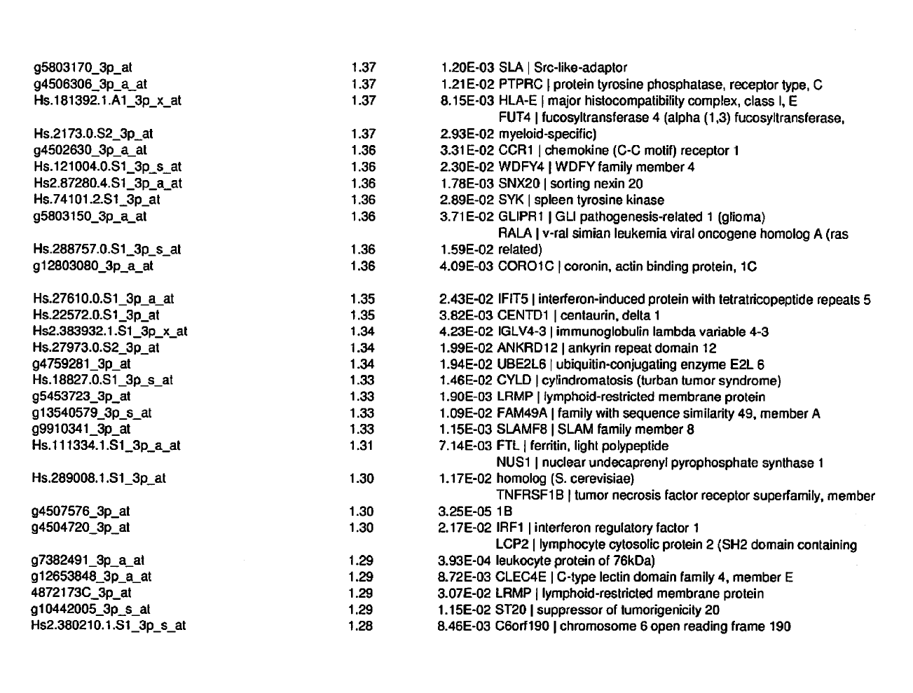

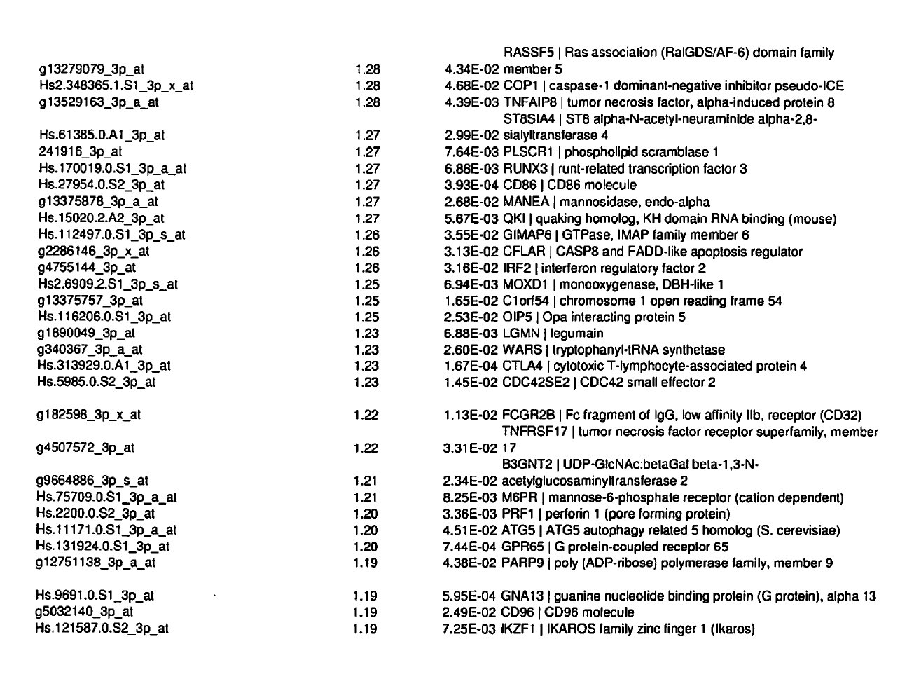

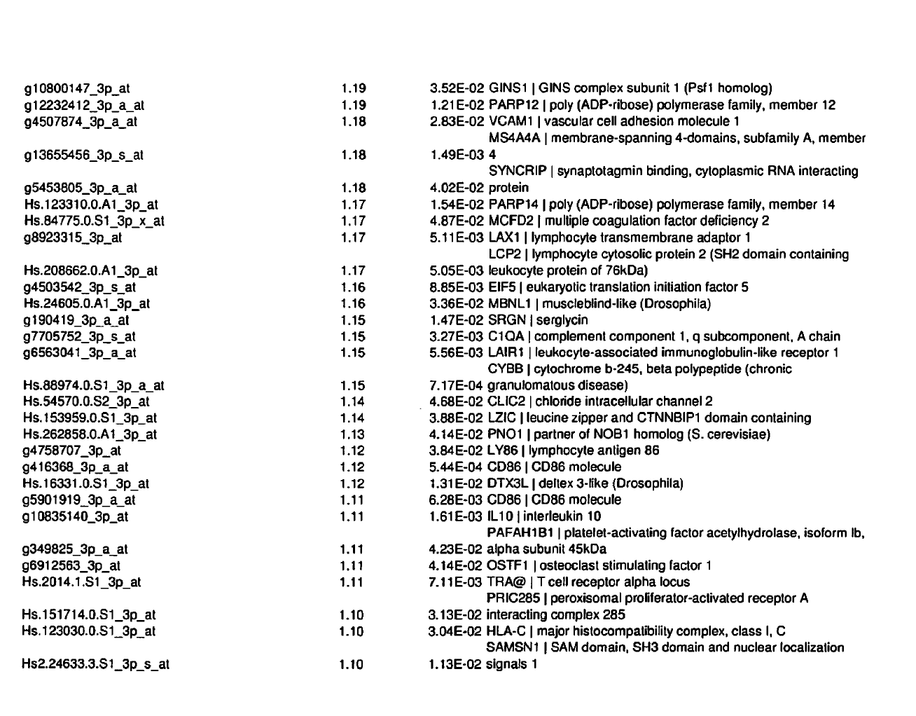

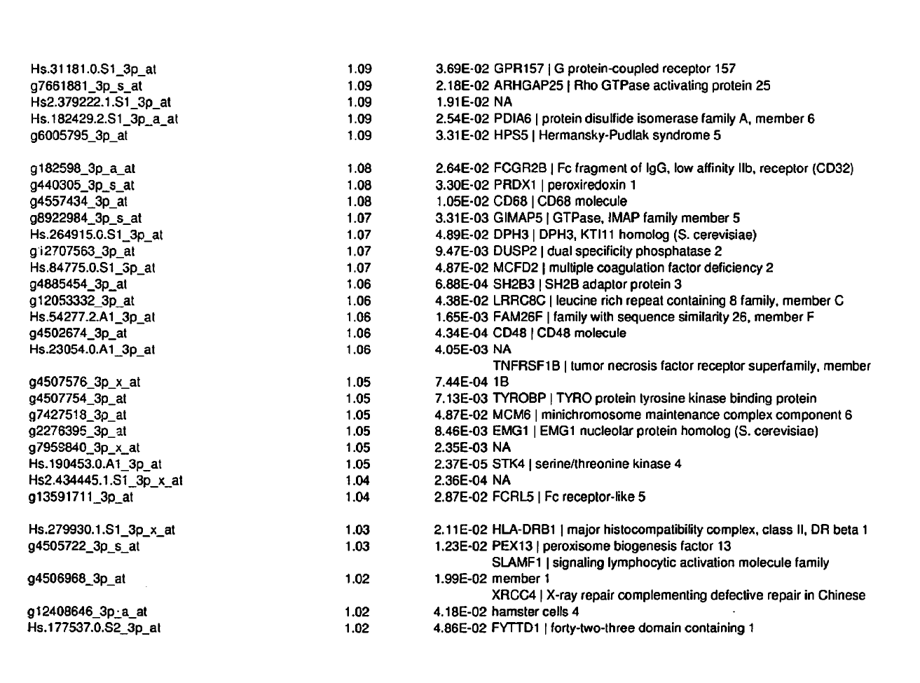

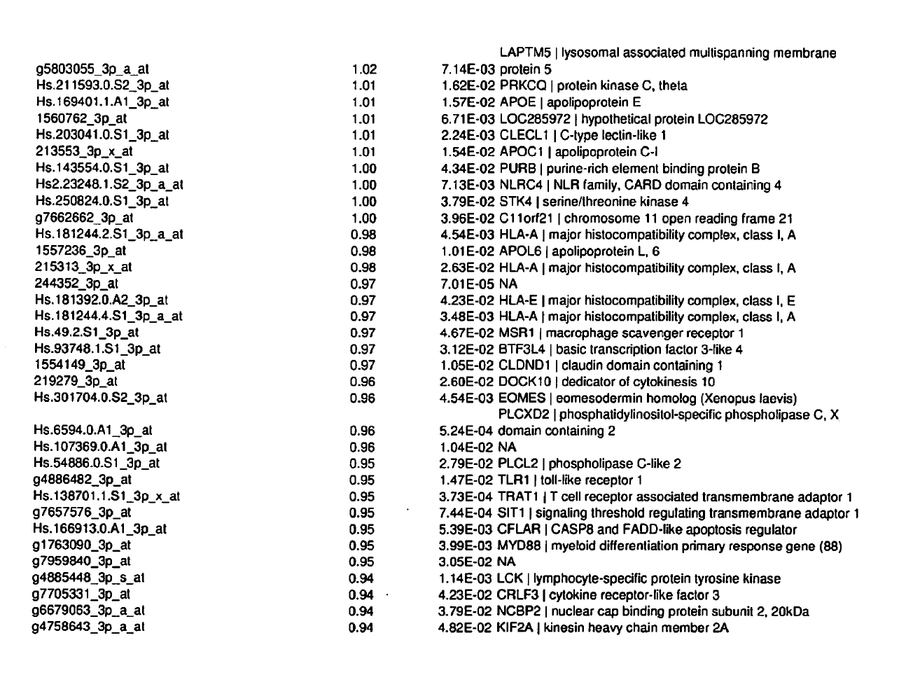

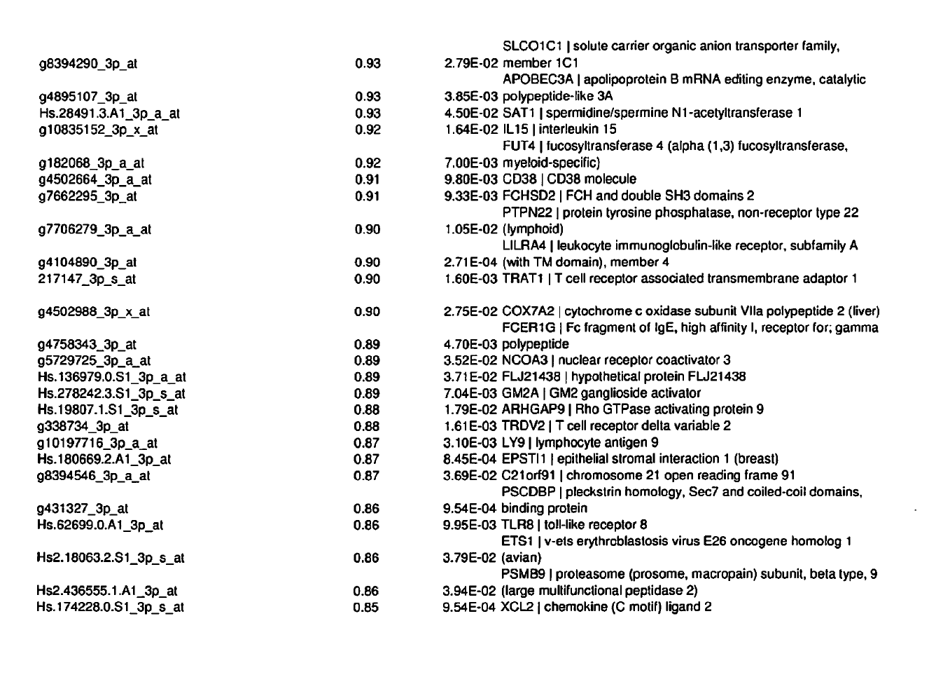

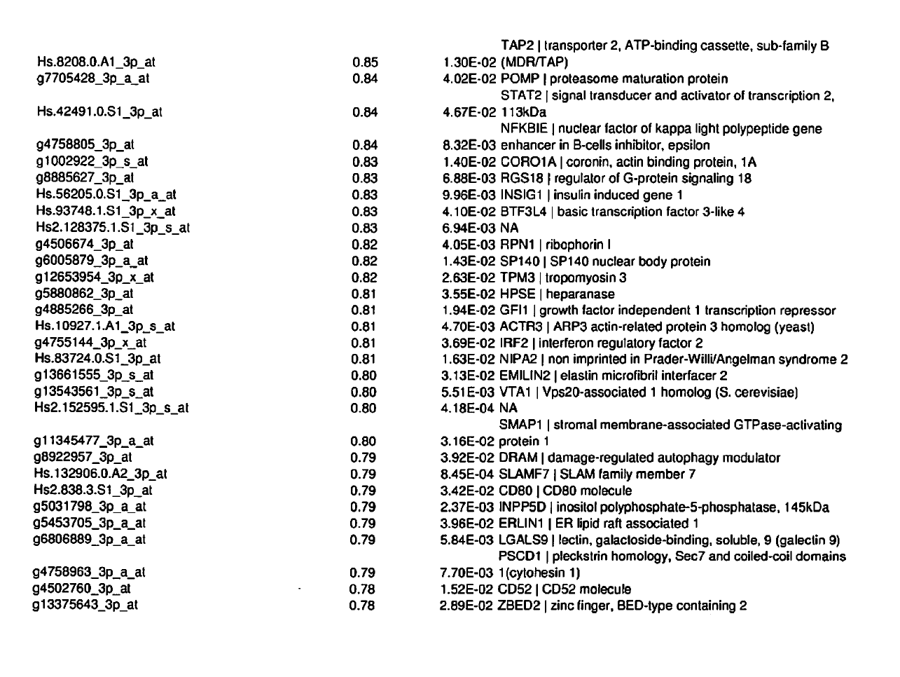

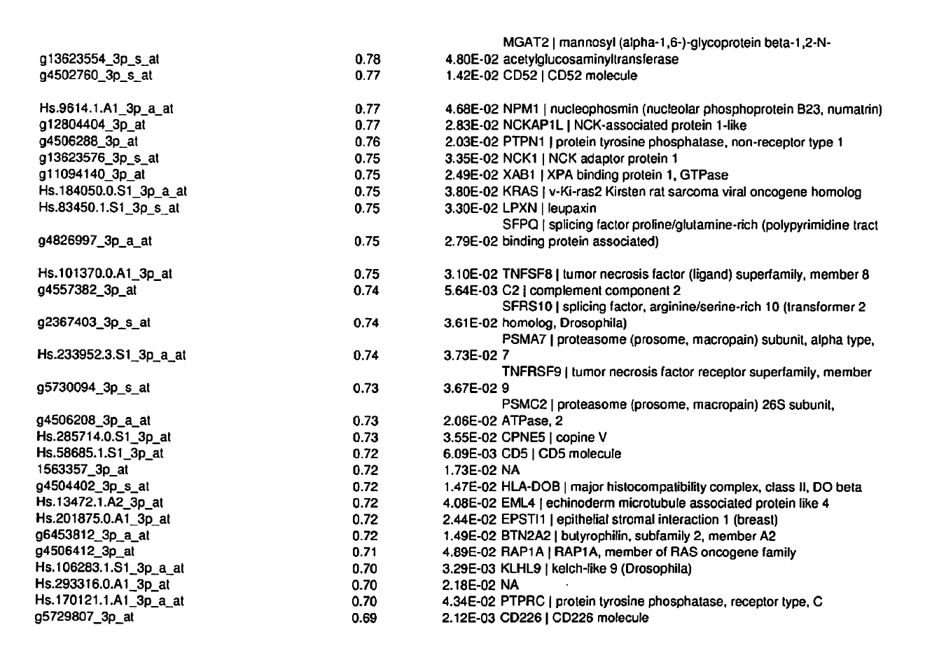

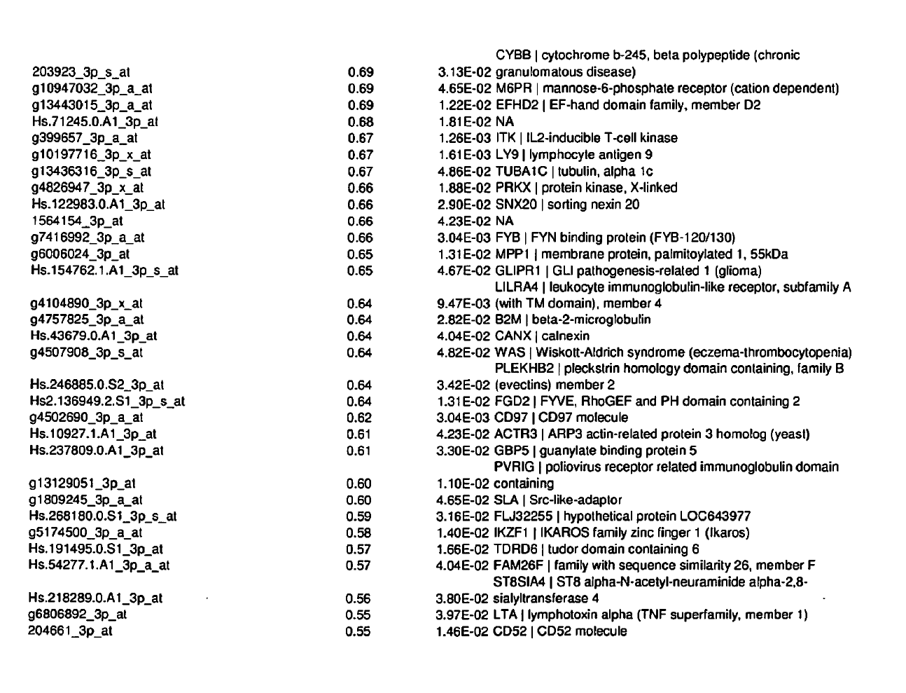

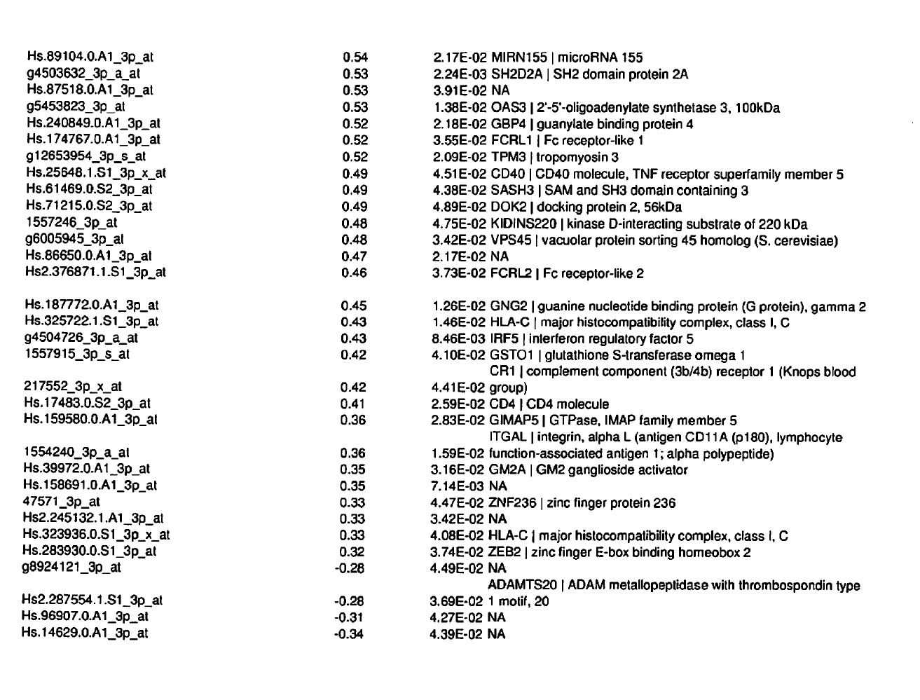

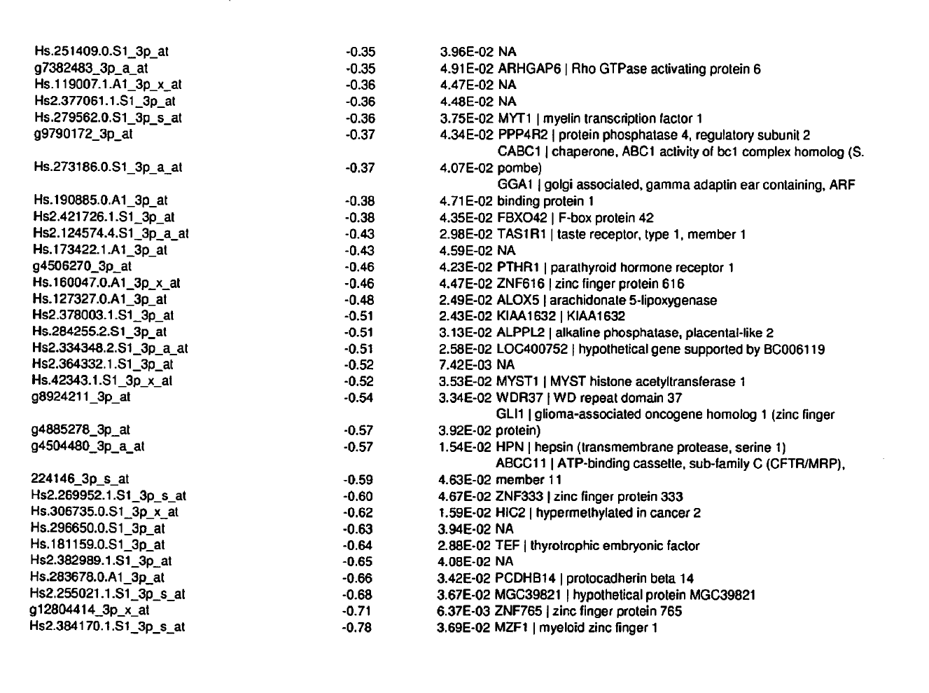

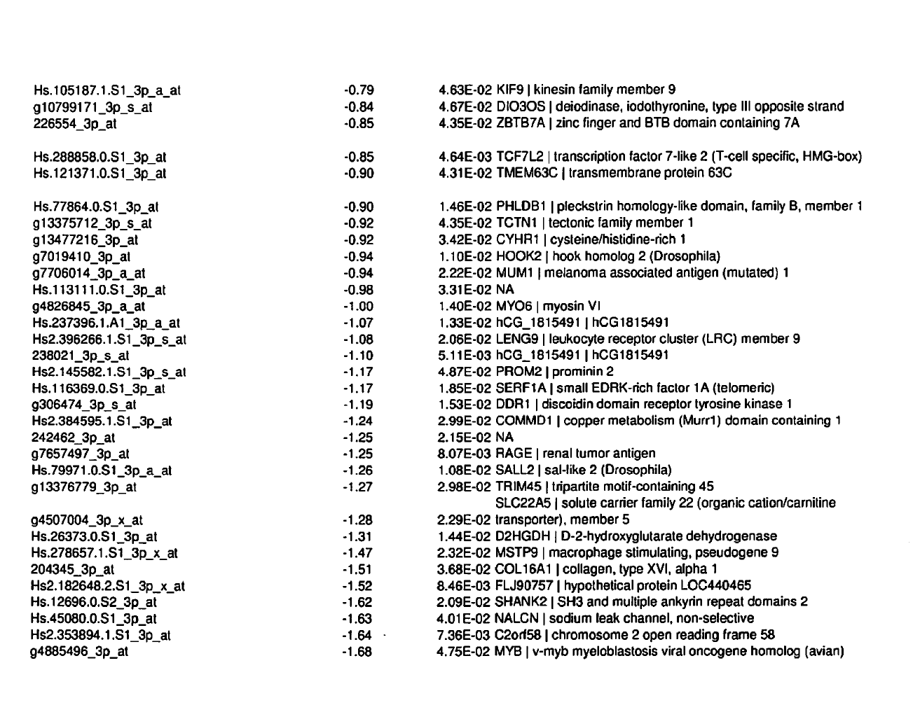

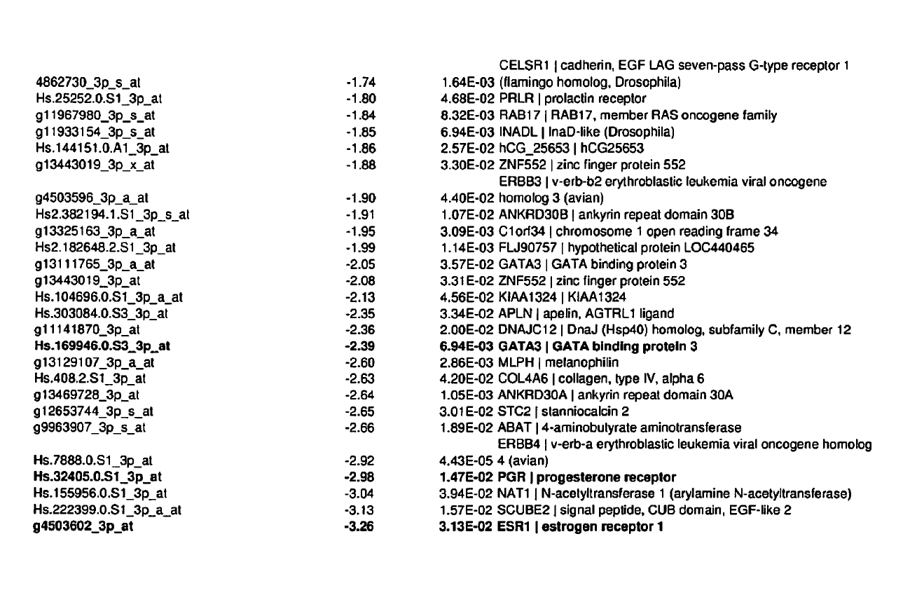

A more extensive list of the genes identified is presented in FIG. 10 (Table 11).

### Example 6: Validation of Selected Differentially Expressed Genes

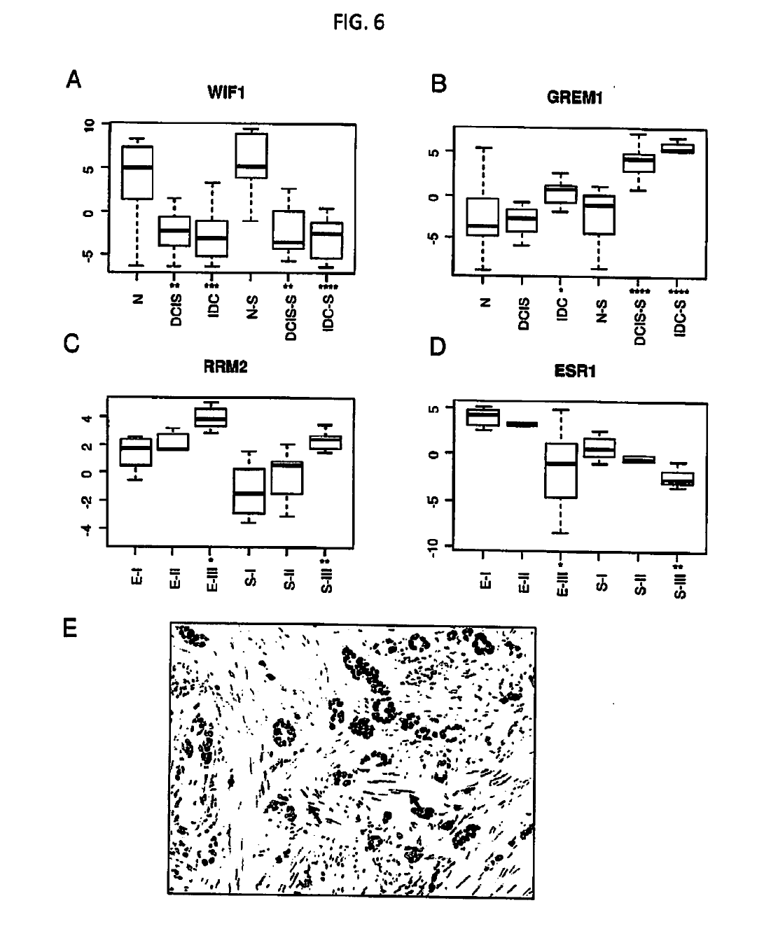

Quantitative real time PCR (QRT-PCR) was used to validate selected genes differentially expressed in the various comparisons presented above. QRT-PCR analysis of the same samples used in microarray analysis confirmed the marked down-regulation of WIF1 in both neoplastic epithelium and tumor stroma (FIG. 6A) and the marked up-regulation of GREM1 in both DCIS- and IDC-associated stroma (FIG. 6B). In addition, two representative genes (ESR1, estrogen receptor α or ERα, and RRM2, ribonucleotide reductase M2 subunit) differentially expressed in the stroma between grade III and grade I tumors (see Supplemental Table 2) were also confirmed by QRT-PCR. In both epithelium and stroma, RRM2, a cell proliferation marker, was more highly expressed in grade III tumors (FIG. 6C), whereas ESR1 was more highly expressed in grade I tumors (FIG. 6D). Although expression of ERα is thought to be restricted to the tumor epithelial cells in human breast cancer (Jensen et al., Proc. Natl. Acad. Sci. USA, 98(26):15197-15202 (2001)), we confirmed the low but detectable levels of ERα expression in stromal fibroblasts by immunohistochemical staining (FIG. 6E).

### ADDITIONAL REFERENCES

1. Kasai et al., J. Histochem. Cytochem., 51(5):567-574 (2003)

2. Amsterdam et al., PLoS Biol, 2(5):E139 (2004)

3. Dai et al. Embo. J., 26(14):3332-3345 (2007)

4. Carew and Huang, Mol. Cancer, 1:9 (2002)

5. Hagland et al., Expert Opin Ther Targets, 11(8):1055-1069 (2007)

6. Ugolini et al., Oncogene, 20(41):5810-5817 (2001)

7. Wissmann et al., J. Pathol. 201(2):204-212 (2003)

8. Klopocki et al., Int. J. Oncol. 25(3):641-649 (2004)

9. Sneddon et al., Proc. Natl. Acad. Sci. USA, 103(40):14842-14847 (2006)

10. Mylonas et al., Oncol. Rep., 13(1):81-88 (2005)

11. Hu et al, Cancer Cell, 13(5):394-406 (2008)

12. Basset et al., Nature, 348(6303):699-704 (1990)

13. de Visser et al., Nat. Rev. Cancer, 6(1):24-37 (2006)

14. Clement et al., Cell Res., 18:889-899 (2008)

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

1. A method of detecting the presence or occurrence of breast cancer in a subject, said method comprising
detecting, in an epithelial or stromal cell from said subject, an increased or decreased expression of a gene in Table 4 or 8 for an epithelial cell, or Table 5 or 9 for a stromal cell, respectively, relative to a normal epithelial or stromal cell.

2. The method of claim 1 wherein the cell is an epithelial cell in a cell containing sample from said subject, said gene is in Table 4, and said expression is relative to a normal epithelial cell from said subject.

3. The method of claim 1 wherein the cell is a stromal cell in a cell containing sample from said subject, said gene is in Table 5, and said expression is relative to a normal epithelial cell from said subject.

4. The method of claim 1 wherein said detecting comprises preparing RNA from said cell.

5. The method of claim 4 wherein said RNA is used for PCR (polymerase chain reaction).

6. The method of claim 1 wherein said detecting comprises using an array.

7. The method of claim 1 wherein said cell is dissected from tissue removed from said subject.

8. The method of claim 5 wherein said PCR is RT-PCR (reverse transcription-PCR), optionally real time RT-PCR.

9. The method of claim 1 wherein said cell is from a formalin fixed paraffin embedded (FFPE) sample.

10. The method of claim 1 wherein said cancer is identified as ductal Amended in situ (DCIS) or invasive ductal carcinoma (IDC) but not both.

11. The method of claim 1 wherein said normal cell is from said subject.

12. A method of detecting the presence or occurrence of breast cancer in a subject, said method comprising
detecting, in a biological fluid from said subject, an increase or decreased expression of a polypeptide encoded by a gene in Table 4, 5, 8, or 9 relative to a normal subject.

13. The method of claim 12 wherein said polypeptide is an extracellular matrix constituent or a matrix metalloprotease, such as MMP2, MMP11, or MMP14.

14. A method to determine therapeutic treatment for a cancer patient, said method comprising
detecting the presence or occurrence of breast cancer in a patient according to claim 1; and
selecting treatment for a patient with said breast cancer.

15. The method of claim 14 wherein said detecting is for ductal carcinoma in situ (DCIS).

16. The method of claim 14 wherein said detecting is for invasive ductal carcinoma (IDC).

17. A method of determining breast cancer and breast cancer grade in a subject, said method comprising
detecting, in a stromal cell from said subject, an increase or decreased expression of a gene in Table 11 relative to a normal stromal cell.

18. The method of claim 15 wherein said breast cancer is DCIS and said grade is grade III.

19. The method of claim 15 wherein said breast cancer is IDC and said grade is grade III.

20-22. (canceled)


SEQUENCE LISTING (12 sequences)

SEQ ID NO: 1 - DNA, 19 bp, Artificial Sequence
  PCR Primer
atgatcaactgggcgaaga

SEQ ID NO: 2 - DNA, 18 bp, Artificial Sequence
  PCR Primer
ggtggacctgatcatgga

SEQ ID NO: 3 - DNA, 16 bp, Artificial Sequence
  PCR Primer
tgccaggctttgtgga

SEQ ID NO: 4 - DNA, 22 bp, Artificial Sequence
  PCR Primer
cctttaaccagcacagccagtt

SEQ ID NO: 5 - DNA, 26 bp, Artificial Sequence
  PCR Primer
ttatttgtttgtaaagtgccaggttt

SEQ ID NO: 6 - DNA, 23 bp, Artificial Sequence
  PCR Primer
tgcagcctcactgcttcaacgca

SEQ ID NO: 7 - DNA, 30 bp, Artificial Sequence
  PCR Primer
acggcaaagaattatatagactatgaggta

SEQ ID NO: 8 - DNA, 27 bp, Artificial Sequence
  PCR Primer
ttttatgagactatcaactcccctttc

SEQ ID NO: 9 - DNA, 17 bp, Artificial Sequence
  PCR Primer
cttgctgtgtaggagga

SEQ ID NO: 10 - DNA, 27 bp, Artificial Sequence
  PCR Primer
cactgtggtagtggcatttaaacaata

SEQ ID NO: 11 - DNA, 24 bp, Artificial Sequence
  PCR Primer
gccaatgcaaaaagttcatacatt

SEQ ID NO: 12 - DNA, 23 bp, Artificial Sequence
  PCR Primer
ttctaaacacaatgaaataggga